(19) United States
(12) Patent Application Publication
(10) Pub. No.: US 2011/0171749 A1
(43) Pub. Date: Jul. 14, 2011

(54) NANOPARTICLE TRACER-BASED ELECTROCHEMICAL DNA SENSOR FOR DETECTION OF PATHOGENS-AMPLIFICATION BY A UNIVERSAL NANO-TRACER (AUNT)

(75) Inventors: Evangelyn Alocilja, East Lansing, MI (US); Deng Zhang, East Lansing, MI (US)

(73) Assignee: Board of Trustees of Michigan State University

(21) Appl. No.: 12/715,929

(22) Filed: Mar. 2, 2010

Related U.S. Application Data
(60) Provisional application No. 61/156,722, filed on Mar. 2, 2009.
(60) Provisional application No. 61/178,397, filed on May 14, 2009.

(51) Int. Cl. G01N 33/53; C07H 21/04; B32B 5/16

(52) U.S. Cl. 436/501; 536/23.1; 536/24.3; 428/403

(57) ABSTRACT

The present invention relates to methods and compositions for identifying a pathogen. The inventions provide an antibody-based biosensor probe comprising (AUNT) in combination with a polymer-coated magnetic nanoparticle. In particular, a nanoparticle-based biosensor was developed for detection of Escherichia coli O157:H7 bacterium in food products. Further described are biosensors for detecting pathogens at low concentrations in samples. Even further, a gold nanoparticle-based electrochemical biosensor detection and amplification method for identifying the insertion element gene of Salmonella enterica Serovar Enteritidis is described. The present invention provides compositions and methods for providing a handheld potentiostat system for detecting pathogens outside of the laboratory. The AUNT biosensor system has applications detecting pathogens in food, water, beverages, clinical samples, and environmental samples.

### GOVERMENT INTERESTS

The present invention was made with government support from the Department of Homeland Security: DHS Science and Technology Assistance Agreement No. 2007-ST-061-0000 03 and the Department of Homeland Security through the Department of National Center for Food Protection and Defense under contract number R9106007101 in addition to the United States Environmental Protection Agency through award number RD83300501 and the National Science Foundation: NSF ECCS-0622056. The United States Government has certain rights in the invention.

### FIELD OF THE INVENTION

The present invention relates to methods and compositions for identifying a pathogen. The inventions provide an antibody-based biosensor probe comprising (AUNT) in combination with a polymer-coated magnetic nanoparticle. In particular, a nanoparticle-based biosensor was developed for detection of Escherichia coli O157:H7 bacterium in food products. Further described are biosensors for detecting pathogens at low concentrations in samples. Even further, a gold nanoparticle-based electrochemical biosensor detection and amplification method for identifying the insertion element gene of Salmonella enterica Serovar Enteritidis is described. The present invention provides compositions and methods for providing a handheld potentiostat system for detecting pathogens outside of the laboratory. The AUNT biosensor system has applications detecting pathogens in food, water, beverages, clinical samples, and environmental samples.

### BACKGROUND

A surveillance report from the 2004 Food Diseases Active Surveillance identified Salmonella as the most common bacterial infection reported, accounting for 42% of the foodborne illness in the United State [CDC, Salmonella surveillance summary, 2004. 2005, United States Department of Health and Human Services]. The Centers for Disease Control and Prevention (CDC) estimates that 1.4 million people in the United States are infected, and that 1,000 people die each year with salmonellosis [CDC, Salmonella surveillance summary, 2004. 2005, United States Department of Health and Human Services]. A total of 36,184 Salmonella isolates were reported from participating public health laboratories in 2005 [CDC, Salmonella surveillance summary, 2004. 2005, United States Department of Health and Human Services]. Further, there is a widespread occurrence in animals, especially in poultry and swine.

Environmental sources of the organism include water, soil, insects, factory surfaces, kitchen surfaces, animal feces, raw meats, raw poultry, and raw seafoods. One of the most common serotypes is Salmonella enterica serovar Enteritidis (Salmonella Enteritidis). Persons infected with Salmonella Enteritidis usually develop diarrhea, fever, and abdominal cramps 12 to 72 hours after infection. In some patients, especially for infants and young children, pregnant women and their unborn babies, and older adults, Salmonella infection may spread from the intestines to the blood stream, and then to other body sites and can be life-threatening unless the person is treated promptly with antibiotics. Therefore Salmonella is a major threat to food safety and public health.

Common detection methods for Salmonella Enteritidis include microbiological, immunological and molecular biological techniques. Although microbiological detection is accurate, it often relies on time-consuming growth in culture media, followed by isolation, biochemical identification, and sometimes serology, and need special reagents and facilities. Immunological detection systems are specific but their sensitivity is low. Molecular PCR-based detection method is sensitive however PCR is often criticized for its complex, expensive, time-consuming, and labor-intensive procedure and narrow target DNA (tDNA) quantification range after PCR amplification.

Thus a rapid, sensitive detection and valid identification of Salmonella Enteritidis is vital within the overall context of food safety and public health.

### SUMMARY OF THE INVENTION

The present invention relates to methods and compositions for identifying a pathogen. The inventions provide an antibody-based biosensor probe comprising (AUNT) in combination with a polymer-coated magnetic nanoparticle. In particular, a nanoparticle-based biosensor was developed for detection of Escherichia coli O157:H7 bacterium in food products. Further described are biosensors for detecting pathogens at low concentrations in samples. Even further, a gold nanoparticle-based electrochemical biosensor detection and amplification method for identifying the insertion element gene of Salmonella enterica Serovar Enteritidis is described. The present invention provides compositions and methods for providing a handheld potentiostat system for detecting pathogens outside of the laboratory. The AUNT biosensor system has applications detecting pathogens in food, water, beverages, clinical samples, and environmental samples.

The present invention provides biosensor compositions and methods of use for identifying a pathogen. Specifically described is an antibody-based biosensor probe comprising Amplification by Universal Nano-Tracer (AUNT) in combination with a polyaniline coated magnetic nanoparticle. In particular, a nanoparticle-based biosensor was developed for detection of Escherichia coli O157:H7 bacterium in food products. Additionally, an AUNT biosensor system of the present inventions has applications for detecting pathogens in food, water, beverages, clinical samples, and environmental samples. Further described are biosensors for detecting pathogens at low concentrations in samples.

The present invention provides methods and compositions for identifying a pathogen, specifically described is a DNA biosensor for Amplification by Universal Nano-Tracer (AUNT). In particular, a gold nanoparticle-based electrochemical biosensor detection and amplification method for identifying the insertion element gene of Salmonella enterica Serovar Enteritidis is described. The present invention further provides compositions and methods for providing a handheld potentiostat system for detecting pathogens outside of the laboratory. The AUNT biosensor system has applications for detecting pathogens in food, beverages, clinical samples, and the environment.

The invention provides, an isolated nucleic acid sequence selected from the group consisting of SEQ ID NOs:1-2 and 12-18.

The invention provides a composition, comprising a DNA sequence selected from the group consisting of SEQ ID NOs: 1-2 and 14-18. In one embodiment, said composition further comprising a nanoparticle. In one embodiment, said nanoparticle is selected from the group consisting of a precious metal, base metal, metal ion, metal salt, polystyrene, and silicon. In one embodiment, said composition further comprising a silent DNA molecule, wherein said DNA molecule is attached to a nanoparticle tracer particle. In one embodiment, said nanoparticle tracer particle is selected from the group consisting of a fluorescence molecule and a metal particle. In one embodiment said silent DNA molecule is selected from the group consisting of SEQ ID NOs:01-2, 6, 8, and 9.

The invention provides a method, comprising, a) providing, i) a first probe DNA sequence selected from the group consisting of SEQ ID NOs: 14 and 17, ii) a silent (universal) DNA sequence attached to biotin, wherein said silent DNA molecule is selected from the group consisting of SEQ ID NOs:01-2, 6, 8 and 9, iii) a detection nanoparticle, and iv) a nanoparticle tracer particle conjugated to streptavidin I, and b) attaching said first DNA sequence and said silent DNA sequence to a nanoparticle, and c) binding said streptavidin I conjugated nanoparticle tracer molecule to biotin of the silent DNA attached to the nanoparticle tracer particle. In one embodiment, the method further comprises provides a metal nanoparticle and a second probe DNA, and attaching said metal nanoparticle to said second probe DNA sequence. In one embodiment, said second probe DNA is selected from the group consisting of SEQ ID NOs: 15, 16 and 18. In one embodiment, the method further comprises provides a sample containing a target DNA sequence such that said first probe is capable of hybridizing to an area of said target DNA said second probe is capable of hybridizing to an area of said target DNA and step d) adding said detection nanoparticle and said metal nanoparticle to said sample in solution under conditions allowing hybridization of said first probe DNA sequence and said second probe DNA sequence to said target sequence. In one embodiment, the method, further comprises, provides a magnet and step e) using said magnet for removing magnetic nanoparticles from solution. In one embodiment, the method further provides a wash buffer and step f) releasing said nanoparticle tracer particle into said wash buffer and step g) measuring the concentration of isolated nanoparticle tracer particle in solution. In one embodiment, said nanoparticle tracer particle is selected from the group consisting of a fluorescence molecule and a metal particle. In one embodiment, said fluorescence molecule is optically measured for extrapolating into a target DNA concentration. In one embodiment, said metal particle is electrochemically measured for extrapolating into a target DNA concentration. In one embodiment, said metal particle is measured using cyclic voltammetry. In one embodiment, said measuring comprises identification of the target DNA. In one embodiment, said measuring is measuring using a hand-held device. In one embodiment, said measuring metal particle concentration is measured using a hand-held potentiostat device.

The invention provides a composition, comprising a silent DNA sequence attached to a nanoparticle tracer. In one embodiment, said silent DNA sequence is selected from the group consisting of SEQ ID NOs:01-2, 6, 8 and 9. In one embodiment, said composition further comprises a detection nanoparticle, wherein said detection nanoparticle is selected from the group consisting of gold, polystyrene, and silicon. In one embodiment, said detection nanoparticle further comprises a first probe DNA molecule capable of hybridizing to a 5′ area of a single strand of a target DNA. In one embodiment, said first probe DNA molecule is selected from the groups consisting of SEQ ID NO:14 and SEQ ID NO:17. In one embodiment, said first probe DNA molecule is attached to said detection nanoparticle. In one embodiment, said detection nanoparticle ranges from 5 nm to 25 nm in diameter. In one embodiment, said composition is soluble. In one embodiment, said composition is in solution. In one embodiment, said nanoparticle tracer is selected from the group consisting of a fluorescence molecule and a metal particle. In one embodiment, said metal particle is capable of conducting electricity. In one embodiment, said metal particle is capable of releasing metal cations. In one embodiment, said metal is selected from the group consisting of a base metal, a precious metal, a metal composite, a metal ion, a metal salt, and an alloy. In one embodiment, said base metal is selected from the group consisting of Lead, Cadmium, Zinc, Copper, sulfates therof, chlorides therof, salts therof, ions thereof, and iostopes therof. In one embodiment, said precious metal is selected from the group consisting of gold, silver, sulfates therof, chlorides therof, salts therof, ions thereof, and iostopes therof. In one embodiment, said fluorescence molecule is selected from the group consisting of carboxyfluoresceins, for example, 6-FAM™. In one embodiment, said nanoparticle tracer is a quantum dot.

The invention provides a composition, comprising a magnetic nanoparticle attached to a probe DNA, wherein said probe DNA is named a second probe DNA. In one embodiment, said second probe DNA is selected from the group consisting of SEQ ID NO:15, 16 and SEQ ID NO:18. In one embodiment, said magnetic nanoparticle comprises iron. In one embodiment, said magnetic nanoparticle ranges from 75 nm to 125 nm in diameter. In one embodiment, said composition is soluble. In one embodiment, said composition is in solution.

The invention provides a silent DNA sequence selected from the group consisting of SEQ ID NO:1-11 and 21.

The invention provides a probe DNA sequence selected from the group consisting of SEQ ID NOs:14-18.

The invention provides a complex comprising a magnetic nanoparticle, a target DNA molecule, and a detection nanoparticle, wherein said detection nanoparticle further comprises a silent DNA sequence attached to nanoparticle tracer, wherein said complex is soluble. In one embodiment, said complex is in solution. In one embodiment, said nanoparticle tracer is selected from the group consisting of a tracer fluorescence molecule and a tracer metal particle. In one embodiment, said tracer metal particle is selected from the group consisting of Lead, Cadmium, Zinc, Copper, isotopes, salts, and derivatives thereof. In one embodiment, said fluorescence molecule is selected from the group consisting of carboxyfluoresceins. In one embodiment, said nanoparticle tracer is a quantum dot. In one embodiment, said silent DNA sequence is selected from the group consisting of SEQ ID NO:1-11 and 21. In one embodiment, said detection nanoparticle is selected from the group consisting of gold, silicon and polystyrene. In one embodiment, said detection nanoparticle comprises a first probe DNA sequence, wherein said first sequence is capable of hybridizing to a target DNA sequence. In one embodiment, said first probe DNA sequence is selected from the group consisting of SEQ ID NO:14 and SEQ ID NO:17. In one embodiment, said magnetic nanoparticle comprises a second probe DNA sequence, wherein said second probe DNA sequence is capable of hybridizing to a target DNA sequence simultaneously with said first probe DNA sequence. In one embodiment, said second probe DNA sequence is selected from the group consisting of SEQ ID NO:15, 16 and SEQ ID NO:18. In one embodiment, said magnetic nanoparticle is attached to the detection nanoparticle by the hybridization of the target DNA to the first DNA sequence of the detection nanoparticle and the hybridization of the target DNA to the second DNA sequence of the magnetic nanoparticle. In one embodiment, said target DNA molecule is derived from a pathogen. In one embodiment, said derived from is selected from the group consisting of DNA isolated from a pathogen, DNA synthetically duplicated from pathogen DNA, and DNA representative of DNA from a pathogen. In one embodiment, said pathogen is selected from the group consisting of a bacterium, a virus, and a fungus.

The invention provides a method for detecting a target DNA, comprising: a) providing: i) a sample comprising a target DNA, wherein said sample is treated under conditions for providing single stranded target DNA, ii) a detection nanoparticle, wherein said nanoparticle comprises a silent DNA sequence attached to a nanoparticle tracer and a first probe DNA sequence complimentary to a portion of the target DNA, and iii) a magnetic nanoparticle, wherein said magnetic nanoparticle comprises a second probe DNA sequence which is different from the first probe DNA sequence, complimentary to a portion of the target DNA, iv) a solution, b) mixing at least a portion of the treated sample, the detection nanoparticle, and the magnetic nanoparticle under DNA-DNA hybridization conditions such that said single stranded molecule of said target DNA hybridizes to both the first probe DNA sequence of said detection nanoparticle and the second probe DNA sequence of said magnetic nanoparticle forming a complex in said solution. In one embodiment, said method further comprises a step c) isolating said complex from said solution using a magnetic field. In one embodiment, said method further comprises, provides, a wash solution, and step d) releasing the nanoparticle tracer into solution from isolated complexes. In one embodiment, said method further comprises, provides, a potentiostat and step e) measuring the concentration of nanoparticle tracer in said nanoparticle solution. In one embodiment, said silent DNA sequence is selected from the group consisting of SEQ ID NOs:01-2, 6, 8 and 9. In one embodiment, said first probe DNA sequence is selected from the group consisting of SEQ ID NO:14 and SEQ ID NO:17. In one embodiment, said second probe DNA is selected from the group consisting of SEQ ID NO:15, 16 and SEQ ID NO:18. In one embodiment, said nanoparticle tracer is selected from the group consisting of a tracer fluorescence molecule and a tracer metal particle. In one embodiment, said tracer fluorescence molecule is selected from the group consisting of carboxyfluorescein (6-FAM™). In one embodiment, said tracer metal particle is selected from the group consisting of Lead, Cadmium, Zinc, Copper, ions therof, salts thereof, and iostopes thereof. In one embodiment, said nanoparticle tracer is a quantum dot. In one embodiment, said method further comprises, provides, a handheld potentiostat comprising a disposable screen-printed carbon electrode (SPCE) for electrochemically measuring the concentration of tracer metal particles in said wash solution. In one embodiment, said handheld potentiostat comprises software capable of converting the measured concentration of metal particles into a target DNA concentration. In one embodiment, said handheld potentiostat comprises software capable of identifying a pathogen. In one embodiment, said handheld potentiostat is a device attached to computer selected from the group consisting of a pocket, laptop, netbook, and desktop computer. In one embodiment, said sample is turbid.

The present invention provides biosensor compositions and methods of use for identifying a pathogen. Specifically described is an antibody-based biosensor probe comprising Amplification by Universal Nano-Tracer (AUNT) in combination with a polymer-coated magnetic nanoparticle. In particular, a nanoparticle-based biosensor was developed for detection of Escherichia coli O157:H7 bacterium in food products. Additionally, an AUNT biosensor system of the present inventions has applications for detecting pathogens in food, water, beverages, clinical samples, and environmental samples. Further described are biosensors for detecting pathogens at low concentrations in samples.

In one embodiment, the inventions provide a composition, comprising, a probe molecule and a magnetic nanoparticle having a core and a shell, wherein said core comprises a magnetic material, and wherein said shell comprises a conducting polymer. In one embodiment, said probe molecule is an antibody. It is not meant to limit the type of antibody. Indeed, an antibody includes but is not limited to a whole antibody and a fragment of antibody, such as an antigen-binding fragments thereof, including, for example, Fab, F(ab′)2, Fab fragments, Fd fragments, and Ev fragments of an antibody. In one embodiment, said antibody is a polyclonal antibody. In a preferred embodiment, said antibody is a monoclonal antibody. Regardless of the type of antibody, said antibody should be specific for binding to a target pathogen. Thus in a preferred embodiment, said antibody specifically binds to a target pathogen particle, wherein said target pathogen includes but is not limited to a bacterium, such as an E. coli 157:H7, Bacillus cereus, Bacillus antracis, a Bacillus antracis spore, a Salmonella sp., a virus, such as bovine viral diarrhea virus, etc., fragments thereof, proteins thereof, DNA sequences thereof, and the like. In one embodiment, said magnetic material comprises a ferromagnetic material including but not limited to maghemite, and mixtures such as silica and maghemite, gold and maghemite, and the like.

In one embodiment, the inventions provide a particulate composition, comprising, a) a conductive polymer bound to a magnetic nanoparticle and b) a binding pair member bound to the conductive polymer of the particulate composition. In one embodiment, said binding pair member is an antibody. In another embodiment, said binding pair member is a probe DNA sequence. In another embodiment, said binding pair member is an aptamer.

In one embodiment, the inventions provide a composition, comprising, a conducting polymer-coated magnetic nanoparticle conjugated with a probe molecule. It is not meant to limit the type of conducting polymer. Indeed, a conducting polymer includes but is not limited to a polyaniline, polypyrrole, Poly(3,4-ethylenedioxy-thiophene)- Polystyrene Sulfonate (PEDOT-PSS), and the like, mixtures thereof, derivatives thereof, for example, derivatives of polyaniline such as sulfonated polyaniline (SPAN), doped polyanilinepolythiophene, etc. In one embodiment, said conducting polymer is polyaniline. In one embodiment, said probe molecule is an antibody.

In one embodiment, the inventions provide a probe composition, comprising, a conducting polymer-coated magnetic nanoparticle conjugated with a probe molecule.

In one embodiment, the inventions provide a composition, comprising, a probe polyaniline-coated magnetic nanoparticle conjugated with an antibody.

In one embodiment, the present invention provides a complex, comprising a conducting polymer-coated magnetic nanoparticle noncovalently bound to a pathogen. in one embodiment, the inventions provide a target complex, comprising, a mixture of a conducting polymer-coated magnetic nanoparticle conjugated with a probe molecule and a target, wherein said probe molecule is noncovalently bound to a target pathogen. In one embodiment, said complex is soluble. In one embodiment, said complex is isolated. In one embodiment, said isolated is by magnetic isolation.

In one embodiment, the inventions provide a target complex, comprising, a probe polyaniline-coated magnetic nanoparticle noncovalently bound to a target pathogen. In one embodiment, said target pathogen is E. coli O157:H7.

In one embodiment, the inventions provide a composition, comprising a silent DNA sequence attached to a nanoparticle tracer, wherein said silent DNA sequence is selected from the group consisting of SEQ ID NOs:01 and 02. In one embodiment, said composition further comprising a nanoparticle. In one embodiment, said nanoparticle is selected from the group consisting of gold, polystyrene, and silicon.

In one embodiment, the inventions provide a detection nanoparticle, comprising a probe antibody, and a silent DNA sequence attached to a nanoparticle tracer. In one embodiment, said nanoparticle tracer is selected from the group consisting of a fluorphore (i.e. a fluorescent molecule) and a metal particle. In one embodiment, said fluorophore is selected from the group consisting of fluorescein, fluorescein derivatives 5 (6)-carboxyfluorescein, cyanine, Alexa Fluors and DyLight Fluors.

In one embodiment, the present invention also provides a complex, comprising a conducting polymer-coated magnetic nanoparticle (first nanoparticle) noncovalently bound to a pathogen, said pathogen further bound to a detection nanoparticle (second nanoparticle). In one embodiment, the pathogen is sandwiched between said first and second nanoparticles). In one embodiment, the inventions provide a biosensor complex, comprising, a mixture of a conducting polymer-coated magnetic nanoparticle conjugated with a first probe molecule is noncovalently bound to a target pathogen, and a detection nanoparticle, comprising a second probe antibody, and a silent DNA sequence attached to a nanoparticle tracer, wherein said second probe antibody is capable of binding to the target pathogen. In one embodiment, said target pathogen is bound to said first probe molecule and said second probe molecule. In one embodiment, said first probe molecule and said second probe molecule are antibodies capable of binding to said target pathogen. In one embodiment, said first antibody is a monoclonal antibody. In one embodiment, said second antibody is a polyclonal antibody. In one embodiment, said first antibody and said second antibody recognize an E. coli O157:H7 bacterium. In one embodiment, said first antibody and said second antibody recognize a Bacillus anthracis bacterium. In one embodiment, said first antibody and said second antibody recognize a spore of Bacillus anthracis. In one embodiment, said complex is soluble. In one embodiment, said complex is isolated. In one embodiment, said isolated is by magnetic isolation.

In one embodiment, the inventions provide a method, comprising, a) providing, i) a composition comprising, a mixture of a conducting polymer-coated magnetic nanoparticle conjugated with a first probe molecule and a target pathogen, and ii) a sample, wherein said sample comprises a target pathogen, and b) forming a complex of said conducting polymer-coated magnetic nanoparticle with the target pathogen, c) magnetically isolating said conducting polymer-coated magnetic nanoparticles from the mixture, d) adding said detection nanoparticle to said conducting polymer-coated magnetic nanoparticle under conditions allowing said second probe molecule to bind to the target pathogen, wherein said detection nanoparticle comprises a nanoparticle tracer molecule, and e) detecting said nanoparticle tracer molecule for determining the concentration of said target pathogen in said sample. In one embodiment, said first probe molecule is selected from the group consisting of antibodies, aptamers, and DNA. In one embodiment, said second probe molecule is selected from the group consisting of antibodies, aptamers, and DNA. In one embodiment, said nanoparticle tracer molecule is selected from the group consisting of fluorescent molecules and metal particles. In one embodiment, said target pathogen is selected from the group consisting of a bacterium, fungi, and virus. In one embodiment, said target pathogen is selected from the group consisting of a pathogen spore, pathogen tissue, pathogen whole cell, pathogen protein, and pathogen DNA. In one embodiment, said pathogen is a bacterium. In one embodiment, said pathogen is a viral antigen, wherein said viral antigen is a bovine viral diarrhea virus (BVDV). In one embodiment, said concentration of viral antigens in a sample ranges from 101-less than 10³ cell culture infective dose per milliliter (CCID/ml) of BVDV antigens. In one embodiment, said concentration of o viral antigens in a sample ranges from 10¹-10² cell culture infective dose per milliliter (CCID/ml) of BVDV antigens.

In one embodiment, the present invention contemplates a method for creating a complex, comprising, a) providing a pathogen and two types of nanoparticles, wherein a first nanoparticle is a conducting polymer-coated magnetic nanoparticle comprising a first probe molecule capable of binding said pathogen, and wherein a second nanoparticle is a detection nanoparticle comprising a second probe molecule and a nanoparticle tracer molecule, wherein said probe molecule is capable of binding the pathogen; and b) mixing said pathogen and said two types of nanoparticles in order to create a complex. In one embodiment, the inventions provide a method for creating a complex, comprising, a) providing two nanoparticles, wherein a first nanoparticle is a conducting polymer-coated magnetic nanoparticle comprising a first probe molecule attached to a target pathogen, and b) adding a second detection nanoparticle, comprising a second probe molecule and a nanoparticle tracer molecule, wherein said probe molecule is capable of binding the pathogen.

In one embodiment, the inventions provide a method, comprising, a) providing, i) a food sample, wherein said sample comprises a foodborne pathogen, ii) a biosensor, wherein said biosensor comprises a conducting polymer-coated magnetic nanoparticle, wherein said magnetic nanoparticle further comprises a first probe molecule capable of binding to a foodborne pathogen, and b) adding said food sample to said biosensor under conditions for binding said first probe molecule to said pathogen, c) determining the presence or absence of a foodborne pathogen in said food sample. In one embodiment, said food sample is collected by a food inspector. In one embodiment, said sample is selected from the group consisting of vegetable products, plant products, egg products, poultry products, fish products, crustacean products, shell food products, sea food, meat products, beverages, food ingredients, food additives, vitamin products, and any product meant for contact with animals. In one embodiment, said method further comprises a step between b and c wherein said magnetic nanoparticle is isolated from the food sample. In one embodiment, said method further comprises a following step of adding a detection nanoparticle comprising a second detection molecule capable of binding to a pathogen particle attached to the first detection molecule. In one embodiment, said method further comprises step d) when a foodborne pathogen is present, determining the concentration of pathogen particle per milliliter of liquid. In one embodiment, said concentration of pathogen bacterium ranges from 1 colony forming unit (CFU) per 2 milliliters to 1000 CFU per milliliter of sample. In one embodiment, said concentration of pathogen bacterium ranges from 1 colony forming unit (CFU) per 2 milliliters to 100 CFU per milliliter of sample. In one embodiment, said concentration of pathogen bacterium ranges from 1 colony forming unit (CFU) per 2 milliliters to 10 CFU per milliliter of sample.

In one embodiment, the inventions provide a composition, comprising, a polyaniline coated magnetic (EAPM) nanoparticle and a detection molecule that binds to a pathogen particle, wherein said detection molecule is selected from the group consisting of a probe DNA molecule and an aptamer.

In one embodiment, the inventions provide a method, comprising, a) providing, i) a composition, comprising, a polyaniline coated magnetic nanoparticle and a detection molecule that binds to a pathogen particle, wherein said detection molecule is selected from the group consisting of a probe DNA molecule and an aptamer, and ii) a sample, wherein said sample comprises a pathogen particle, and b) incubating said polyaniline coated magnetic nanoparticle composition with said sample for detecting a pathogen particle. In one embodiment, said aptamer comprises SEQ ID NO: 12 (5′-AGA GGA ATG TAT AAG GAT GTT CCG GGC GTG TGG GTA AGT C-3′). In one embodiment, said probe DNA comprises SEQ ID NO: 13 (5′-GG AAGAGTGAGG GTGGATACAG GCTCGAACTG GAGTGAAGTG TTACCGCA-3′).

In one embodiment, the inventions provide a composition, comprising, a bacterium capable of green production of a nanoparticle of the present inventions. The composition, wherein said green is biological synthesis of a nanoparticle. The composition, wherein said bacterium is capable of providing a nanoparticle of the present inventions. The composition, wherein said bacterium is capable of bioreduction of AuCl₄ ions into gold nanoparticles. Wherein said nanoparticles range in diameter from 2 nanometers to 50 nanometers. Wherein said nanoparticles range in diameter from 8 nanometers to 20 nanometers. The composition, wherein said bacterium is a Thermomospora curvata.

### Definitions

To facilitate an understanding of the present invention, a number of terms and phrases are defined below:

The use of the article “a” or “an” is intended to include one or more. As used in this application, the singular form “a,” “an,” and “the” include plural references unless the context clearly dictates otherwise. For example, the term “a nanoparticle” includes a plurality of nanoparticles, including mixtures thereof.

As used herein, the term “Universal Nano-Tracer Amplification” or “UNTA” or “Amplification by Universal Nano-Tracer (AUNT)” refers collectively to compositions, methods, assays, biosensors and systems of the present inventions, i.e. for quantifying and identifying pathogens in food, beverages, environment, clinical fluids, etc., in other words, quantifying and identifying pathogens in a sample. “AUNT” in reference to a system refers to a device comprising a biosensor, wherein said biosensor further comprises an AUNT nanoparticle, and associated electronics or signal processors that is primarily responsible for the display of the results, in a preferred embodiment; the associated electronics and signal processors are handheld.

As used herein, “AUNT” in reference to a nanoparticle refers to a nanoparticle comprising a silent DNA sequence bound to a nanoparticle tracer while “AUNT” in gerneral refers to a composition comprising a nanoparticle, wherein said nanoparticle comprises a silent DNA sequence attached to a nanoparticle tracer, i.e. a label, such as a fluorphore, a metal particle, and the like, and further comprises a probe, for example, a DNA sequence, antibody, aptamer, etc., for specifically recognizing a pathogen. In one embodiment, the AUNT nanoparticle further comprises a probe antibody. In another embodiment, the AUNT nanoparticle further comprises a probe DNA sequence.

As used herein, “detection nanoparticle” refers to a nanoparticle used for providing an AUNT nanoparticle of the present inventions. A detection nanoparticle consists of a material selected from the group consisting of a precious metal, a base metal, metal ion, metal salt, polystyrene, and silicon, and combinations thereof. In a preferred embodiment, the precious metal is gold (for example, a AuNP, etc.), however any other material or substance may be used to form a detection nanoparticle. In a preferred embodiment, a detection nanoparticle ranges in diameter from 10-50 nm, and more preferably 10-25 nm.

As used herein, the term “biosensor” reference to a device of the present inventions refers collectively to the detection of an analyte or target DNA molecule by combining a biological component (biological material (eg. tissue, microorganisms, organelles, cell receptors, enzymes, antibodies, nucleic acids, as probe DNA, or biomimic etc), with a physicochemical transducer element (i.e. an element that works in a physicochemical way; optical, electrical, piezoelectric, electrochemical, etc.) that transforms the signal resulting from the interaction of the analyte with the biological element into another signal (i.e., transducers) that can be measured and quantified. In some embodiments, the transducers act as a means for amplifying a low number or low concentraion of analytes in a sample into a detectable and repeatable (meaningful) signal.

As used herein, “silent” or “universal” or “bio-barcode” in reference to a DNA sequence refers to a DNA sequence of the present inventions that does not hybridize to a target DNA sequence in such a way to interfere with the function of a biosensor of the present inventions. Further, a silent DNA sequence may not bind to or hybridize with any component of a sample or reagent used in an assay comprising a silent DNA sequence in a manner that would result in a false positive test result or identify an organism not present in the sample or falsely cause a significant increase in the amount of measured target DNA. In other words, a silent or universal DNA sequence refers to a nucleic acid sequence used for attaching a label onto a gold particle, such as a fluorescent label, nanoparticle tracer label, et cetera. Examples of such silent DNA sequences include but are not limited to SEQ ID NO:01 of the present inventions. Examples of such silent DNA sequences include but are not limited to Universal DNA,such as 5′-TTA TTC GTA GCT AAA AA AAA-3′ A, SEQ ID NO:01, labeled with 6-FAM™; 5′-/56-carboxyfluorescein (FAM)/TTA TTC GTA GCT AAA AA AAA A/3ThioMC3-D/-3′(SEQ ID NO:21), and SEQ ID NO:10 (5′-TTATTCGTAGCTAAAAAAAAAA -SH-3′) and SEQ ID NO:5 (5′-TTATTCGTAGCT AAAAAAAAAA-6-FAM-3′) of the present inventions. Examples of such universal DNA sequences of the present inventions include but are not limited to a “Universal” barcode sequence or b5′-AGC TAC GAA TAA-3′ (SEQ ID NO:06) and 5′-thiol modifier-AAA AAA AAA ATT ATT CGT AGC T-3′, Hill and Mirkin, et al., 2006 (SEQ ID NO:07), 1 CGTCGCATTC AGGATTCTCA ACTCGTAGCT AAAAAAAAAA; Accession DL005587 (SEQ ID NO:08); 1 TCTCAACTCG TAGCTAAAAA AAAAA, Accession DL005584 (SEQ ID NO:09); Bio-Barcode Based Detection Of Target Analytes at GenBank, Au nanoparticle sequence, Sequence 32603, in U.S. Pat. No. 7,214,786, herein incorporated by reference, and the like.

As used herein, “bDNA” or “barcode DNA” as shown in figures and described in the present inventions refers to a silent DNA for attaching a nanoparticle tracer to a detection nanoparticle. As used herein, “bDNA” or “barcode DNA” or “barcode” in reference to an AUNT nanoparticle, such as a nanoparticle as shown schematically and by TEM in figures and described in the present inventions refers to a silent DNA for attaching a nanoparticle tracer to a detection nanoparticle.

As used herein, “nanoparticle” in general refers to any particle with a diameter ranging between 1 and 1000 nanometers (nm). In a preferred embodiment, a nanoparticle ranges between 1 and 100 nanometers (nm).

As used herein, the term “nanoparticle tracer” or “nanoparticle tracer particle” refers to a measurable label or tag for representing and amplifying the presence of target DNA in a sample, for example, a tracer fluorescence molecule, a tracer metal particle, a quantum dot, and the like.

As used herein, “functionalization” or functionalized” in reference to a nanoparticle refers to adding DNA sequences to nanoparticles or nanoparticles comprising probe DNA sequences of the present inventions, respectively.

As used herein, “detection nanoparticle” refers to a nanoparticle used for attaching a silent DNA sequence bound to a nanoparticle tracer and a probe DNA sequence of the present inventions. A detection nanoparticle consists of a material selected from the group consisting of a precious metal, a base metal, metal ion, metal salt, polystyrene, and silicon, and combinations thereof. In a preferred embodiment, the precious metal is gold (AuNP), however any other material or substance may be used to form a detection nanoparticle. In a preferred embodiment, a detection nanoparticle ranges in diameter from 10-50 nm, and more preferably 10-25 nm.

As used herein, “magnetic nanoparticle” or “MNP” or “MMP” refers to a nanoparticle ranging in diameter from 50-150 nm, more preferably 75-125 nm, comprising magnetic material (e.g., ferromagnetite) for example, magnetic nanoparticles of the present inventions with an average diameter of 100 nm.

As used herein, the term “magnetic nanoparticle” or “MMP” or “MNP” in reference to a magnetic nanoparticle comprising a probe DNA sequence refers to a composition of the present inventions, in other words a functionalized MNP.

As used herein, the term “magnetic nanoparticle” in reference to a conducting polymer coated magnetic particle, in one embodiment, refers to a magnetic nanoparticle coated with polyaniline.

As used herein, “polyaniline” or “PANI” refers to a conducting polymer of the semi-flexible rod polymer family, see, structure in FIG. 2A.

As used herein, the term “magnetic” refers to any compound that is capable of being altered in a magnetic field. In some embodiments, the magnetic compound is permanently magnetized. In other embodiments, the magnetic compound is magnetized in the presence of an external magnetic field, for example, Iron or Fe.

As used herein, the term “nanoparticle tracer” or “nanoparticle tracer particle” refers to a measurable label or tag for representing and amplifying the presence of target DNA in a sample, for example, a tracer fluorescence molecule, a tracer metal particle, a quantum dot, and the like.

As used herein, the term “releasing the nanoparticle tracer” refers to releasing charged ions from metal nanoparticles into a solution, in particular releasing ions from isolated complexes.

As used herein, the term “measuring the concentration” in reference to a nanoparticle tracer in solution refers to a fluorescent or electrochemical measurement, see detecting.

As used herein, the term “fluorescence” or fluorescent” or “fluorophore” in reference to a molecule or substance or compound refers to the capability of that molecule or substance to absorb a photon of a particular wavelenght that in turn triggers the emission of a photon with a longer (less energetic) wavelength, for example, carboxyfluorescein (6-FAM).

As used herein, “nanowire” or “quantum wire” refers to an electrically conducting nanostructure, with a “thickness” or “diameter” in the range of nanometers (10⁻⁹ meters), in other words, a thickness or diameter constrained to tens of nanometers or less and an unconstrained length.

As used herein, “nanowire” also refers to a wire comprising conducting polymer coated magnetic nanoparticles, in other words aggregated magnetic nanoparticles or nanobeams.

As used herein, “nanobeam” refers to biologically enhanced electrically active magnetic nanoparticles, in other words, a conducting polymer coated magnetic nanoparticle.

As used herein, “wire” refers to an elongated string of metal, in particular a string of metal capable of conducting an electric current, for example, a string of magnetic nanoparticles attached to each other by a conducting polymer.

As used herein, “polymer” refers to a structure made of up a string of attached repeating monomers, wherein the attached monomer is a natural or synthetic material.

As used herein, “conducting polymer” refers to a polymer that is capable of conducting electricity or that acts as electrical semiconductor, for example, polyaniline, polythiophene, polypyrrole, Poly(3,4-ethylenedioxythiophene)-Polystyrene Sulfonate (PEDOT-PSS), and the like.

As used herein, “polymer wire” refers to a wire coated with a conducting polymer. A “polymer wire” in reference to a biosensor, refers to a biosensor comprising a polymer wire, in other words a “polymeric wire biosensor,” for example, a “polyaniline-based polymeric wire biosensor.”

As used herein, “performance enhancement” in reference to an electrode refers to increasing reproducibility and reliability over an electrode used for comparison, for example, by using screen-printed silver electrodes, using pulse mode measurements for electrode signals, and the like.

As used herein, “detecting” in reference to light emitted a fluorescent compound refers to the capability of sensing an optical signal emitted from the fluorescent compound. As used herein, “detecting” also refers to the capability of a device for sensing an optical signal emitted from the fluorescent compound, such detecting includes devices such as fluorescence spectroscopy, such as fluorometry, including fluorometers or fluorimeters, or spectrofluorometry which analyzes or measures fluorescence from or in a sample.

As used herein, “quantum dot” refers to a semiconductor whose excitations are confined in three spatial dimensions. As a result, they have properties that are between those of bulk semiconductors and those of discrete molecules. Examples of quantum dots include small regions of one material buried in another with a larger band gap, such as core-shell structures, e.g., with CdSe in the core and ZnS in the shell or from special forms of silica called ormosil, further examples include cadmium sulphide quantum dots, (CdTe quantum dots), Li et al., Journal of Electroanalytical Chemistry 625(1) 1 Jan. 2009, Pages 88-91, quantum dot (QD) nanocrystals (ZnS, CdS, and PbS) (Kin et al., Engineering in Medicine and Biology Society, 2004. IEMBS apos; 04. 26th Annual International Conference of the IEEE Volume 1, Issue, 1-5 Sep. 2004 Page(s):137-140), cadmium sulfide quantum dots (CdS QDs) Marin, et al., Nanotechnology 20 (2009) 055101 (6 pp), and the like. A quantum dot may emit fluorescent light. A quantum dot may be capable of providing an electrochemical signal.

As used herein, the term “metal” is a chemical element whose atoms readily lose electrons including semi-metals, for examples of metals, a substance that conducts electricity, a substance that is magnetic, a substance selected from metals as shown on a Periodic Table.

As used herein, the term “base metal” refers to a substance which oxidizes when heated in air, e.g. lead, copper, tin, zinc, as opposed to noble metals such as gold and platinum.

As used herein, the term “precious metal” refers to a substance such as gold, silver, platinum and palladium, including platinum group metals: ruthenium, rhodium, palladium, osmium, iridium, etc.

As used herein, the term “noble metal” refers to a substance such as tantalum, gold, platinum, and rhodium.

As used herein, the term “metal nanoparticle” in reference to a tracer nanoparticle refers to a metal that forms negative ions, including base metals such as Lead, Cadmium, Zinc, Copper, a precious metal, a metal composite, an alloy, sulfates thereof, chlorides thereof, salts thereof, ions thereof, and iostopes thereof, etc.

As used herein, the term “ion” refers to an atom or a group of atoms or a molecule with a net electric charge, such as a compound which has lost or gained one or more electrons, giving it a positive or negative electrical charge. As used herein, the term “anion” refers to a negatively charged ion, formed when an atom gains electrons in a reaction. Anions are negatively charged because there are more electrons associated with them than there are protons in their nuclei. As used herein, the term “cation” refers to a positively charged ion, formed when an atom loses electrons in a reaction, forming an ‘electron hole’. Cations are the opposite of anions, since cations have fewer electrons than protons.

As used herein, the term “(III)” or “3” or “³” or “+++” in reference to a metal, such as Pb, refers to a charge on that metal, for example, Pb(III) or ³Pb has a positive charge of 3.

As used herein, the term “sub-nanometer particle” refers to a particle that is smaller than a nanometer in diameter, i.e. ion, and is capable of being measured (e.g., by cyclic voltammetry as disclosed herein).

As used herein, “soluble” refers to solubility of a substance which is a physical property describing the ability of a given substance, the solute, to dissolve in a solvent.

As used herein, “solvent” refers to a liquid substance capable of dissolving or dispersing one or more substances. It is not intended that the present invention be limited by the nature of the solvent used, for example, water, a buffer solution, a wash solution, etc.

As used herein, “solution” refers to a homogeneous mixture composed of two or more substances. In such a mixture, a solute is dissolved in another substance, known as a solvent. A common example is a solid, such as salt or sugar, dissolved in water, a liquid, as used in the present inventions, a solid, such as a nanoparticle or complex or nanoparticle tracer of the present inventions suspended or dissoved in a solution, as opposed to being attached to an electrode or microarray.

As used herein, “liquid” refers to a state of matter characterized by fluidity such that a liquid takes on the shape of its container, unlike a solid which retains its shape despite the shape of its containiner, in other words, a “solid” refers to a state of matter characterized by resistance to deformation and changes of volume.

As used herein, “solid-based” in reference to an assay or biosensor refers to an element, such as a metal nanoparicle, attached to a solid surface, for example, an electrode, a plastic plate, and the like, as opposed to liquid-base assays of the present inventions, wherein attachment to a solid surface, such as a magnet, is reversable or temporary.

As used herein, “concentration” or “level” of a substance refers to a measure of an amount of a given substance that is mixed with another substance or how much of a given substance was present in a sample, such as an amount of target DNA in a sample as measured by a biosensor of the present inventions. Typically, the amount is measured with reference to a volume.

As used herein, the term “binding” or “attaching” refers to any means of attaching one compound or substance or molecule to another compound or substance or molecule. Binding may be a chemical binding, such as a thiol linkage, for example attaching a thiol linked probe DNA to a nanoparticle of the present inventions, attaching a silent DNA molecule to a metal tracer molecule using biotin-strepavidin binding, and the like.

As used herein, the term “hybridization” refers to an attachment of one DNA molecule to another through specific base pairing.

As used herein, the term “streptavidin” refers to various forms of a compound that is or derives from a 53000 dalton tetrameric protein originally purified from the bacterium Streptomyces avidinii which binds tightly to a small molecule, biotin, examples include, recombinant streptavidin and derivatives of strepavidin retaining biotin binding regions, i.e. streptavidin I.

As used herein, the term “biotin” also known as “Coenzyme R” or “vitamin H” or “B7” refers to a small molecule with a chemical formula C₁₀H₁₆N₂O₃S which is also a water-soluble B vitamin-complex, biotin binds strongly to strepavidin.

As used herein, the terms “purified” and “to purify” and “wash” refer to the removal of contaminants from a sample, such as removing unhybridized detection nanoparticles from sample.

As used herein, “cyclic voltammetry” or “CV” refers to a type of potentiodynamic electrochemical measurement. Cyclic voltammetry is generally used to study the electrochemical properties of an analyte, such as a charged metal ion, in solution. The method uses a reference electrode, working electrode, and counter electrode which in combination are sometimes referred to as a three-electrode setup. Common materials for working electrodes include glassy carbon, screen printed carbon, platinum, gold, and the like.

The term “gene” refers to a nucleic acid (e.g., DNA) sequence that comprises coding sequences necessary for the production of a polypeptide or precursor. It is intended that the term encompass polypeptides encoded by a full length coding sequence, as well as any portion of the coding sequence, so long as the desired activity and/or functional properties (e.g., enzymatic activity, ligand binding, etc.) of the full-length or fragmented polypeptide are retained. The term also encompasses the coding region of a structural gene and the sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as “5′ untranslated sequences.” The sequences that are located 3′ (i.e., “downstream”) of the coding region and that are present on the mRNA are referred to as “3′ untranslated sequences.” The term “gene” encompasses both cDNA and genomic forms of a gene. A genomic form of a genetic clone contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where “amino acid sequence” is recited herein to refer to an amino acid sequence of a naturally occurring protein molecule, “amino acid sequence” and like terms, such as “polypeptide” and “protein” is not meant to limit the amino acid sequence to the complete, native amino acid sequence associated with the recited protein molecule. In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence. DNA molecules are said to have “5′ ends” and “3′″ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” and “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification and hybridization reactions, as well as detection methods that depend upon binding between nucleic acids. Equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

As used herein, the term “hybridization” is used in reference to the pairing of complementary nucleic acids, A to T, and G to C. Hybridization and the strength of hybridization (i.e., the strength of the association between the nucleic acids) is impacted by such factors as the degree of complementary between the nucleic acids, stringency of the conditions involved, the Tₙ, of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“Amplification” in reference to PCR refers to a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques were designed primarily for this sorting out. Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q-replicase, MDV-I RNA is the specific template for the replicase (Kacian et al, Proc. Natl. Acad. Sci. USA, 69:3038 [1972]; herein incorporated by reference). Similarly, in the case of T7 RNA polymerase, this amplification enzyme has a stringent specificity for its own promoters (Chamberlin et al, Nature, 228:227 [1970]; herein incorporated by reference). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics, 4:560 [1989]; herein incorporated by reference). Finally, Tag and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (Erlich (ed.), PCR Technology, Stockton Press [1989]; herein incorporated by reference).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “polymerase chain reaction” or “PCR” refers to the methods described in U.S. Pat. Nos. 4,683,195, 4,683,202, and 4,965,188, all of which are hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications. With PCR, it is possible to amplify a single copy of a specific target sequence in genomic DNA to a level detectable by the device and systems of the present invention.

As used herein, the terms “PCR product,” “PCR fragment,” and “amplification product” refer to the resultant mixture of compounds from at least two or more cycles of the PCR steps of denaturation, annealing and extension are complete. These terms encompass the case where there has been amplification of one or more segments of one or more target sequences.

As used herein, the terms “thermal cycler” or “thermalcycler” refer to a programmable thermal cycling machine, such as a device for performing PCR.

As used herein, the term “amplification reagents” in reference to PCR-based DNA amplification refers to those reagents (such as, DNA polymerase, deoxyribonucleotide triphosphates, buffer, etc.), necessary for PCR-based DNA amplification.

As used herein, the terms “reverse-transcriptase” and “RT-PCR” refer to a type of PCR where the starting material is mRNA. The starting mRNA is enzymatically converted to complementary DNA or “cDNA” using a reverse transcriptase enzyme. The cDNA is then used as a “template” for a “PCR” reaction.

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule that is comprised of segments of DNA joined together by means of molecular biological techniques. The terms “recombinant protein” and “recombinant polypeptide” as used herein refer to a protein molecule that are expressed from a recombinant DNA molecule.

As used herein the term “coding region” when used in reference to a structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets that specify stop codons (i.e., TA, TAG, TGA).

The term “recombinant DNA molecule” as used herein refers to a DNA molecule that is comprised of segments of DNA joined together by means of molecular biological techniques.

As used herein the term “portion” or “fragment” when in reference to a nucleotide sequence (as in “a portion of a given nucleotide sequence”) refers to fragments of that sequence. The fragments may range in size from four nucleotides to the entire nucleotide sequence minus one nucleotide.

As used herein, the term “test DNA” or “sample DNA” or “target DNA” or tDNA” refer to the DNA to be analyzed using the biosensor methods of the present inventions. In preferred embodiments, this target DNA is tested for hybridization to probe DNA for forming a complex of the present invention, for example, an insertion element (Iel) gene of Salmonella Enteritidis, B. anthracis fragment of pagA gene (Gene Bank Accession no. M22589), etc. In some embodiments, more than one target DNA is present in a sample.

As used herein, the term “isolated nucleic acid sequence” The term “isolated” when used in relation to a nucleic acid, as in “an isolated DNA sequence” or “isolated oligonucleotide” or “isolated polynucleotide” refers to a nucleic acid sequence that is identified and separated from at least one contaminant nucleic acid with which it is ordinarily associated in its natural source. Thus in one form, an isolated nucleic acid is present in a form or setting that is different from that in which it is found in nature. An isolated DNA sequnece may also refer to a synthetic DNA sequence separated from shorter contaminant sequences. In contrast, non-isolated nucleic acids are nucleic acids such as DNA and RNA found in the state they exist in nature. For example, a given DNA sequence (e.g., a gene) is found on the host cell genome in proximity to neighboring genes; RNA sequences, such as a specific mRNA sequence encoding a specific protein, are found in the cell as a mixture with numerous other mRNAs that encode a multitude of proteins. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein, the term “probe” refers to a molecule (e.g., an oligonucleotide, a DNA sequence, whether occurring as a wild-type or natural sequence as sequenced from a purified restriction digest or produced synthetically based upon a natural sequence, recombinantly or by PCR amplification), that is capable of hybridizing to another molecule of interest (e.g., another oligonucleotide or a target DNA molecule). When probes are oligonucleotides they may be single-stranded or double-stranded. Probes are useful in the detection, identification and isolation of particular target (e.g., gene sequences). In some embodiments, it is contemplated that probes used in the present invention are labeled with any “reporter molecule,” that is detectable in any detection system, including, but not limited to enzyme (e.g., ELISA, as well as enzyme-based histochemical assays), fluorescent, radioactive, luminescent systems, electrochemical systems, etc. It is not intended that the present invention be limited to any particular label. With respect to microarray biosensor, the term probe is used to refer to any hybridizable material that is affixed to the detection nanoparticle or magnetic particle for the purpose of detecting a “target” sequence in a sample.

As used herein “probe element” and “probe site” refer to a plurality of probe molecules (e.g., identical probe molecules) affixed to a substrate, such as a nanoparticle. Probe elements containing different characteristic molecules are contemplated for use in biosensors of the present inventions, such that a specific tracer nanoparticle identifies each different probe.

As used herein, the term “target” when used in reference to hybridization assays, such as target DNA of the present inventions, refers to the molecules (e.g., nucleic acid) to be detected. Thus, the “target” is sought to be sorted out from other molecules (e.g., isolated nucleic acid sequences) or is to be identified as being present in a sample through its specific interaction (e.g., hybridization) with another agent (e.g., a probe DNA” or “probe oligonucleotide”).

A “segment” or “area” refers to a region of nucleic acid within the target sequence.

As used herein, the term “oligonucleotides” or “oligos” refers to short sequences of nucleotides, such as probe DNA sequences of the present inventions or fragments of target DNA.

As used herein “probe molecule” or detection molecule” refer to a molecule for identifying a target, such as a pathogen target. Speicfically a probe molecule, such as a DNA sequence, an anibody, an aptamer, and the like, is capable of selectively binding to a target pathogen, such that a target pathogen is identified.

As used herein, the term “first probe DNA sequence” refers to a DNA sequence capable of hybridizing to a 5′ portion or area of said single stranded target DNA, for example, SEQ ID NOs: 2 and 4 for hybridizing to a lei target gene of Salmonella and pagA gene of Bacillius, respectively. In a preferred embodiment, the first probe DNA is attached to a detection nanoparticle of the present inventions.

As used herein, the term “second probe DNA sequence” refers to a DNA sequence capable of hybridizing to a 3′ portion or area of said single stranded target DNA, for example, SEQ ID NOs: 3 and 5 for hybridizing to a target gene of Salmonella and pagA gene of Bacillius, respectively.

As used herein, the term “derived” in reference to a DNA molecule refers to a DNA sequence with high identity or capable of hybridization at high stringency to the wild-type form of a DNA sequence or isolated target DNA sequence or synthetic target DNA sequence, including, for example, DNA isolated from a pathogen, DNA synthetically duplicated from a pathogen's DNA (i.e. PCR amplified), DNA representative of DNA from a pathogen (cDNA, synthetic fragment), and the like.

As used herein, the terms “microbe” and “microbial” refer to microorganisms. In particularly preferred embodiments, the microbes identified using the present invention are bacteria (i.e., eubacteria and archaea). However, it is not intended that the present invention be limited to bacteria, as other microorganisms are also encompassed within this definition, including fungi, viruses, and parasites (e.g., protozoans and helminths).

As used herein, the term “pathogen” refers to any organism or microbe that may cause a disease, death, adverse health effects to an animal, including humans, fish, birds, and reptiles, such as a bacterium, a fungi, a virus and the like.

As used herein, the term “pathogens of concern” refer to bacterium including, Bacillus anthracis; E. coli O157:H7; Salmonella Typhimurium; S. Enteritidis; Helicobacter pylori; Mycobacterium tuberculosis, Mycobacterium bovis, Mycobacterium avium; paratubeculosis (MAP), etc. and Viruses, such as Bovine viral diarrhea virus; Adenoviruses (HAdV 40/41); Avian influenza virus, HIV, etc.

As used herein, the term “foodborne pathogen” refers to a microorganism found in food that causes illness or death in animals or humans.

The term “microorganism” as used herein means an organism too small to be observed with the unaided eye and includes, but is not limited to bacteria, viruses, protozoans, fungi, and ciliates.

The term “bacterium” refers to an organism of any bacterial species including eubacterial and archaebacterial species.

The term “virus” refers to obligate, ultramicroscopic, intracellular parasites incapable of autonomous replication (i.e., replication requires the use of the host cell's machinery).

As used herein, the term “reference DNA” refers to DNA that is obtained from a known organism (i.e., a reference strain). In some embodiments of the invention, the reference DNA comprises random genome fragments. In particularly preferred embodiments, the genome fragments are of approximately 1 to 2 kb in size. Thus, in preferred embodiments, the reference DNA of the present invention comprises mixtures of genomes from multiple reference strains.

As used herein, the term “multiple reference strains” refers to the use of more than one reference strains in an analysis. In some embodiments, multiple reference strains within the same species are used, while in other embodiments, “multiple reference strains” refers to the use of multiple species within the same genus, and in still further embodiments, the term refers to the use of multiple species within different genera.

As used herein, the term “treated under conditions for providing single stranded DNA” refers to conditions for separating double strands of DNA, such as high salt concentrations, heating above 95° C., and the like.

As used herein, the term “detection nanoparticle” wherein said nanoparticle comprises a SEQ ID NO:05 attached to a nanoparticle tracer and a first probe DNA sequence complimentary to a portion of the target DNA.

As used herein, the term “solution” refers to any liquid used in the present inventions. Solution in reference to collecting tracer metal particles refers to a acid wash or wash solution or any solution for releasing tracer metal particles into solution.

As used herein, the term “DNA-DNA hybridization conditions” refers to conditions allowing a single stranded molecule of the target DNA to hybridize to both the first probe DNA sequence of said detection nanoparticle and the second probe DNA sequence of said magnetic nanoparticle forming a complex in solution.

As used herein, the term “isolating said complex from said solution” and similar terms refer to using a magnetic field for capturing metal nanoparticles, including any bound material onto a magnet or coated surface of a magnet, or magnetized surface.

As used herein, the term “magnet” refers to a material or object that produces a magnetic field. A magnetic field is invisible but is responsible for the most notable property of a magnet: a force that pulls on other ferromagnetic materials, i.e materials strongly attracted to a magnet, including but not limted to compositions comprising iron,nickel, cobalt, some rare earth metals, alloys, and some naturally occurring minerals such as lodestone.

As used herein, the term “alloy” refers to a partial or complete solid solution of one or more elements in a metallic matrix.

As used herein, the term “label” or “label molecule” refers to any particle or molecule or agent or inherent property of a composition that provides a detectable signal, such that it can be used to provide a detectable (preferably quantifiable) effect. In some embodiments, labels, such as nanoparticle tracers, utilized in the present invention provide an amplification effect representing the presence and amount of target DNA of the present inventions. In some embodiments, the label comprises indicator dyes, enzymes, molecular recognition elements capable of providing an electrochemical signal, releasing electron, synergistic sensing mechanisms and non-perturbative measurements, as well as fluorescent molecules, fluorescent quantum dots, electrical quantum dots, reflective gold and silver nanoparticles. In some embodiments, the label is integral to a probe. In other embodiments, it is attached to the surface of a probe (e.g., a “labeling particle”). In some embodiments, the label is an “indicator dye.” In some embodiments, the label is a “metal particle.” In some embodiments, labels attached to a nanoparticle in the presence of a probe (e.g., fluorescent labeled DNA attached to a nanoparticle also attached to a probe DNA of the present inventions). In some embodiments, the label is a Raman active molecule.

As used herein, “electrode” refers to an electrical conductor used to make contact with a nonmetallic part of a circuit (e.g. a semiconductor, an electrolyte, etc.).

As used herein, “screen-printing” in reference to an electrode, refers to a process of placing a conductive material onto a surface for use as an electrode i.e. a “screen printed electrode” or “SPE,” for examples of screen printed electrodes used in biosensor production, see, Grennan, et al., Electroanalysis, 13(8-9):745-750, herein incorporated by reference in its entirety).

As used herein, the term “screen-printed carbon electrode” or “SPCE” refers to an electrode capable of electrochemically measuring the concentration of tracer metal particles used in the present inventions. For example, see, SPCEs described in WO 1997032203, DropSens SPCE (Palm), a SPCE obtained from GWENT GROUP, Product: BE2030408D7, comprising Working Electrode material: product C2030519P4-Carbon/graphite ink, Common Reference and Counter Electrode material: product C61003P7-Ag/AgCl:60/40 ink, Dielectric material: product D2040917D2-grey insulator, Substrate: Polyester (Valox FR1) for use with gold plated connectors connecting the screen printed connector pads to a potentiostat.

As used herein, the term “disposable” in reference to a SPCE refers to a single use electrode.

As used herein, the term “software” refers to a general term used to describe a collection of computer programs, procedures and documentation that perform a task on a computer system.

As used herein, the term “software capable of converting the measured concentration of metal particles into a target DNA concentration” refers to capability to provide for examples, charts, graphs, tables and the like as readouts of the present inventions, i.e. a task.

As used herein, the term “software capable of identifying a pathogen” refers to software capable of converting nanoparticle tracer molecules or signals into an output showing the presence or absence of a pathogen in a sample, and further, the name of a pathogen whose DNA was detected in a sample, i.e. a task.

As used herein, the term “attached” in reference to hardware, such a potentiostat attached to a computer, a disposable screen-printed carbon electrode (SPCE) attached to a potentiostat, and the like, refers to capability to communicate through a cable or by wireless.

As used herein, the term “computer” refers to a machine that manipulates data according to a list of instructions selected from the group consisting of a pocket, laptop, netbook, and desktop computer.

As used herein, “peripheral” refers to a device, such as a computer device, for example, a CD-ROM drive or wireless communication chip, that is not part of the essential computer, i.e., the memory and microprocessor. Peripheral devices can be external, such as a mouse, keyboard, printer, monitor, external Zip drive or scanner or internal, such as a CD-ROM drive, CD-R drive or internal modem. Internal peripheral devices may be referred to as “integrated peripherals.”

As used herein, the term “processor” refers to a device that performs a set of steps according to a program (e.g., a digital computer). Processors, for example, include Central Processing Units (“CPUs”), electronic devices, and systems for receiving, transmitting, storing and/or manipulating digital data under programmed control.

As used herein, the terms “memory device,” and “computer memory” refer to any data storage device that is readable by a computer, including, but not limited to, random access memory, hard disks, magnetic (e.g., floppy) disks, zip disks, compact discs, DVDs, magnetic tape, and the like.

As used herein, “chamber” or “holder” in reference to a sample, such as a biological sample chamber, refers to an area capable of comprising a biological sample, such as a special area, actual holder, and the like.

As used herein, the term “handheld” in reference to a detection device for tracer metal particles refers to a portable potentiostat, such as a PalmSens device. A handheld device of the present inventions is capable of providing a readout of a tracer metal particle of the present inventions.

As used herein, the term “portable” refers to a device with an independent power supply. A handheld device of the present inventions is capable of providing a readout of a tracer metal particle of the present inventions.

As used herein, the term “turbid” in reference to a liquid sample refers to the cloudiness or haziness of a fluid caused by individual particles (suspended solids such as particles of dirt, particles of pollution, contaminating metal particles, and the like) that are generally invisible to the naked eye, similar to smoke in air. In reference to the present inventions, pathogens, such as viruses or bacteria may be attached to a suspended solid.

As used herein, the term “dirty” in reference to a liquid or solid or air sample refers to samples containing contaminating particles such as proteins, lipids, dust, soil, metal particles, and the like.

As used herein, the term “background” or “background artifacts” include signal components caused by undesired optical emissions from the microarray. These artifacts arise from a number of sources, including: non-specific hybridization, intrinsic fluorescence of the substrate and/or reagents, incompletely attenuated fluorescent excitation light, and stray ambient light. In some embodiments, the noise of an optical detector system is determined by measuring the noise of the background region and noise of the signal from the microarray feature.

As used herein, the term “noise” in its broadest sense refers to any undesired disturbances (i.e., signal not directly resulting from the intended detected event) within the frequency band of interest. One example of noise is the summation of unwanted or disturbing energy introduced into a system from man-made and natural sources. In another example, noise may distort a signal such that the information carried by the signal becomes degraded or less reliable.

As used herein, the term “signal-to-noise ratio” refers to the ability to resolve true signal from the noise of a system. One example of computing a signal-to-noise ratio is by taking the ratio of levels of the desired signal to the level of noise present with the signal. In preferred embodiments of the present invention, phenomena affecting signal-to-noise ratio include, but are not limited to, detector noise, system noise, and background artifacts.

As used herein, the term “detector noise” refers to undesired disturbances (i.e., signal not directly resulting from the intended detected energy) that originate within the detector. Detector noise includes dark current noise and shot noise. Dark current noise in an optical detector system results from the various thermal emissions from the photodetector. Shot noise in an optical system is the product of the fundamental particle nature (i.e., Poisson-distributed energy fluctuations) of incident photons as they pass through the photodetector.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like, in addition to food, (including vegetables, fruit, meat, and processed products, at any stage of production, sale, and consumption, such as at processing facilities, restaurants, grocery stores and the like), milk, beverages, Environmental samples include environmental material such as surface matter, (i.e. paper, swabs of paper, swabs of a surface, such as a surface of a desk, a surface of a piece of equipment, a surface of a table, and the like), water, soil, air, and industrial samples. Such examples are not however to be construed as limiting the sample types applicable to the present invention.

As used herein, the term “localized surface plasmons” refers to a charge density oscillation confined to coinage metal nanoparticles and nanoislands [Endo, et al., 2005, Sci. Technol. Adv. Mater. 6 491-500, herein incorporated by reference].

As used herein, the term “antibody” includes polyclonal antibodies, monoclonal antibodies, naturally occurring antibodies as well as non-naturally occurring antibodies, including, for example, single chain antibodies, chimeric, bifunctional and humanized antibodies, as well as antigen-binding fragments thereof, including, for example, Fab, F(ab′)2, Fab fragments, Fd fragments, and Ev fragments of an antibody, as well as a Fab expression library. It is intended that the term “antibody” encompass any immunoglobulin (e.g., IgG, IgM, IgA, IgE, IgD, etc.) obtained from any source (e.g., humans, rodents, non-human primates, caprines, bovines, equines, ovines, etc.). The term “polyclonal antibody” refers to an immunoglobulin produced from more than a single clone of plasma cells; in contrast “monoclonal antibody” refers to an immunoglobulin produced from a single clone of plasma cells. Monoclonal and polyclonal antibodies may or may not be purified. For example, polyclonal antibodies contained in crude antiserum may be used in this unpurified state. Naturally occurring antibodies may be generated in any species including murine, rat, rabbit, hamster, human, and simian species using methods known in the art. Non-naturally occurring antibodies can be constructed using solid phase peptide synthesis, can be produced recombinantly or can be obtained, for example, by screening combinatorial libraries consisting of variable heavy chains and variable light chains as previously described (Huse et al. Science 246:1275-1281, 1989). These and other methods of making, for example, chimeric, humanized, CDR-grafted, single chain, and bifunctional antibodies are well known to those skilled in the art (Winter and Harris (1993) Immunol Today 14:243-246; Ward et al. (1989) Nature 341:544-546; Hilyard et al. Protein Engineering: A practical approach (IRL Press 1992); and Borrabeck, Antibody Engineering, 2d ed. (Oxford University Press 1995)). Those skilled in the art know how to make polyclonal and monoclonal antibodies, which are specific to a desirable cell or polypeptide. For the production of monoclonal and polyclonal antibodies, various host animals can be immunized by injection with the peptide corresponding to any molecule of interest in the present invention, including but not limited to rabbits, mice, rats, sheep, goats, chickens, etc. In one preferred embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum. For preparation of monoclonal antibodies directed toward molecules of interest in the present invention, any technique that provides for the production of antibody molecules by continuous cell lines in culture may be used (See, e.g., Harlow and Lane, Antibodies: A Laboratory Manual, Cold Spring Harbor Laboratory Press, Cold Spring Harbor, N.Y.). These include but are not limited to the hybridoma technique originally developed by Kohler and Milstein (Kohler and Milstein, Nature 256:495-497, 1975), as well as the trioma technique, the human B-cell hybridoma technique (See e.g., Kozbor et al. Immunol. Today 4:72, 1983), and the EBV-hybridoma technique to produce human monoclonal antibodies (Cole et al., in Monoclonal Antibodies and Cancer Therapy, Alan R. Liss, Inc., pp. 77-96, 1985). In additional embodiments of the invention, monoclonal antibodies can be produced in germ-free animals utilizing technology such as that described in PCT/US90/02545. Furthermore, techniques described for the production of single chain antibodies (See, e.g., U.S. Pat. No. 4,946,778; herein incorporated by reference) can be adapted to produce single chain antibodies that specifically recognize a molecule of interest, i.e. a pathogen or component or product thereof. An additional embodiment of the invention utilizes the techniques described for the construction of Fab expression libraries (Huse et al. Science 246:1275-1281, 1989) to allow rapid and easy identification of monoclonal Fab fragments with the desired specificity for a particular protein or epitope of interest (e.g., a pathogen or component or product thereof).

As used herein, “green” in reference to production refers to biologically synthesized nanoparticles of the present inventions, for example, nanoparticle compositions, such as gold nanoparticles of the present inventions, synthesized by a bacterium. See, FIG. 1.

### BRIEF DESCRIPTIONS OF THE DRAWINGS

FIG. 1 shows several exemplary schematics of functionalization of Au nanoparticles: (A) AuNPs and AuNP-DNA probe conjugation (B and C) Characterization of AuNPs: I) TEM image of AuNPs showing approximately 15 nm in diameter; II) Absorbance spectrum of AuNPs showing a peak at 520 nm; (D) Fluorescence signal of supernatant solution for evaluation of conjugation efficiency between Au-NPs and barcode DNA; (E) fluorescence signal of released barcode DNA with different concentrations of target DNA; and (E) shows exemplary “green' production of gold nanoparticles for use in the present inventions.

FIG. 2 shows several exemplary schematics of functionalization of MNP nanoparticles: (A) MNPs and MNP-DNA probe conjugation (B and C) Characterization of MNPs: I) TEM image of MNPs showing approximately 100 nm in diameter; II) magnetic profile of MNPs Magnetic hysteresis loop of MNPs; magnetic saturation at approximately 80 emu/g; (C and D) probe conjugation efficiency of MNP and DNA probes.

FIG. 3 shows an exemplary schematic of the biosensor detection: (A) formation of AU NPs with probe and silent DNA with an exemplary fluorescein label (B) MNP-2nd pDNA-tDNA-1st pDNA-AuNPs sandwich complex with tDNA separation C and D showing exemplary results.

FIG. 4 shows an exemplary embodiment of the present inventions (AUNT) using fluorescent labeling of silent DNA: (A) determination of amount of polyclonal antibody for conjugation and UV-vis absorption spectra of AuNPs. One of following amounts of pAb was added into the microcentrifuge tubes of mL AuNPs: 0 μg; 1 μg; 2 μg; 3 μg; 4 μg; and 5 μg. Absorbance was measured with pure 30 nm AuNP (solid line) and the 30 nm AuNP conjugated with pAb (2 μg/ml; dashed line). (B) shows an exemplary UV-vis absorption spectra of barcode DNA labeled with 6-FAM™ and AuNP probe. The absorption spectrum of the fluorophore-labeled barcode DNA is displayed with solid line. The absorption spectrum of the AuNP probe which was functionalized by the barcode DNA and pAb was displayed with dashed line. (C) shows exemplary fluorescence before/after released barcode DNA from AuNP probes. Pure 30 nm AuNPs were used as a reference (con) to monitor fluorescence signal of the AuNP probe.

FIG. 5 shows an exemplary TEM image of magnetic polyaniline and UV-vis absorption spectra of MPANI probe: (A) The size of iron (III) oxide magnetic polyaniline was less than 100 nm. (B) shows an exemplary absorbance measured with pure polyaniline-coated magnetic nanoparticle (solid line) and polyaniline-coated magnetic nanoparticle conjugated with mAb (dashed line). C) shows an exemplary schematic of bio-barcode assay for E. coli O157:H7 detection. Gold nanoparticles and polyaniline-coated magnetic nanoparticles were used to detect E. coli O157:H7.

FIG. 6 shows exemplary fluorescence measurements as a function of E. coli O157:H7 concentration: (A) Target complex with various concentrations of E. coli O157:H7 bound with AuNP probe was separated by magnetic field and the fluorophore-labeled barcode DNA was released from AuNP probe by using 0.5 M DTT. Fluorescence measurements were performed by VICTOR3 multilabel plate reader as a function of E. coli O157:H7 concentration. Target complex without E. coli O157:H7 incubated with the AuNP probe was used as a reference (con) to monitor fluorescence signal changes. (B) For the quantitative analysis; averages of fluorescence intensity increment from the reference were calculated from the color spectra in (A).

FIG. 7 shows one embodiment of exemplary schematics of a universal (bio-barcode assay) (AUNT bioassay) of the present inventions. (A) exemplary schematics of functionalization of nanoparticles AuNPs (B) hybridization between probes and target DNA; (D) separation; release of nanoparticle tracers and electrochemical measurement of ions on a screen printed electrode; alternatively the Au particles may be electrochemically measured.

FIG. 8 shows exemplary schematics of and actual electrochemical measurements of AuNPs and metal ions on a screen printed electrode (A) schematic SWASV measurement of a gold (Au) readout from one target DNA; B) schematic of contemplated SWASV measurement of multiplex DNA targets with Pb as nanoparticle tracers; C) schematic of a SPCE-based biosensor; D) exemplary SWASV measurement of amount of AuNPs released from a biosensor comprising MNPS and a target DNA (PCR amplified Iel Salmenella gene) of the present inventions (AUNT) showing a strong signal with at least 0.01 ug/ml of target DNA (0.001; 0.01; 0.1 and 1 ug/ml of a Salmonella Iel gene) DPV hybridization response of different concentrations of DNA targets on SPCE; DPV scan from +1.25 V to 0 V; step potential 10 mV; modulation amplitude 50 mV; scan rate 50 mV/s; nonstirred solution; matching a-d concentration points in E) showing a calibration plot between peak current at 0.4V (vs. Ag/AgCl) and the target DNA concentration; and F) an exemplary demonstration of a detection of at least 0.02 ng/ml (or 20 pg/ml) of a pagA gene 40 uL of DNA sample; 40 uL of AuNPs/CdS; 0.8 mg of MNPs; 160 uL of assay buffer for hybridization After washing; dissolved in 20 uL of 1 M of nitric acid for 5 min; then add 80 uL of acetate buffer (pH=4.5) and apply this 100 uL of solution on SPCE for detection. The data is shown after subtraction of negative control. Time detection: hybridization time: 45 min; +10 min deposition+0.5 min measurement

FIG. 9 shows an exemplary CV response of unmodified AuNPs (1.17×10-8 M) on SPCE.: (a) AuNPs in 0.1M HCl; (b) 0.1 M HCl. CV scan from 1.4V to 0.0; scan rate 100 mV/s after 1.25 V electrooxidation for 2 min.

FIG. 10 shows an exemplary CV response of probe-modified AuNPs in 0.1 M HCl on SPCE: (a) accumulation for 20 min; (b) accumulation for 10 min; (c) no accumulation; (d) control; no AuNPs and no accumulation. Conditions: electrooxidation potential; +1.25 V; electrooxidation time; 2 min; CV scan from +1.4 V to 0 V; scan rate 100 mV/s; nonstirred solution.

FIG. 11 shows an exemplary differential pulse voltammetry (DPV) hybridization response of different concentrations of DNA targets on SPCE: (A) and the calibration plot between peak current and the tDNA concentration (B). DPV scan from +1.25 V to 0 V; step potential 10 mV; modulation amplitude 50 mV; scan rate 50 mV/s; nonstirred solution.

FIG. 12 shows another embodiment of the present inventions; an exemplary schematic of a contemplated multiple universal (barcode) assay: (A) MNP conjugation to 2^{nd} DNA probes with several types of target DNA probes for hybridizing to target DNA molecules and forming sandwiches with AuNPS; each AuNP conjugated to a unique target probe and one type of fluorescent molecule for separation and read out of each type of florescence; (B) an embodiment using nanoparticle tracer molecules and (C) Schematic of the composition of integrated miniaturized electrochemical sensor for use in the present inventions; a) top view; b) cross section view; (D) Cyclic voltammogram of bare electrochemical sensor in a 5 mM K₃Fe(CN)₆ solution with 1M KCl1; Scan rate is 50 mV/s; (E) The repeatability test of 1 mg/L of bismuth film-coated electrochemical sensor and bare electrochemical sensor; (F) Stripping voltammogram of 5 mg/L of lead(II) with various concentrations of bismuth on electrochemical sensor after 2 min deposition at −1;2 V; (G) Stripping voltammogram on bismuth film-coated electrochemical sensors with various deposition times; (H) Stripping voltammogram of a mixture of lead(II) and cadmium(II) on bare electrochemical sensors; I) Stripping voltammogram of a mixture of lead(II) and cadmium(II) on bismuth film-coated electrochemical sensors; (J) The peak currents of stripping voltammogram with different concentrations of lead(II) and Cadmium(II).

FIG. 13 shows an exemplary solid-phase assay (Detection platform: membrane-based) of the present inventions. This schematic shows a solid-phase assay (Detection platform: membrane-based) with immobilized AuNps conjugated to a DNA probe; wherein the DNA probe comprises a hybridization region for a Barcode DNA; and a method wherein the barcode DNA of the AuNP hybridizes to a region of the capture probe DNA attached to the immobilized capture pad between electrodes; After hybridization; the bound Au particle is coated with Ag (silver ions) for completing an electrical circuit triggering an electrochemical signal; Alocilja; et al.; Nano-Biosensors for Food Defense; PITTCON 2008; Mar. 2-7, 2008; New Orleans; La.; herein incorporated by reference in its entirety.

FIG. 14 shows an exemplary schematic of a handheld potentiostat (i.e. PalmsS ens by Palm) attached to a SPCE biosensor showing a cable connection to a computer (pocket PC) for contemplated on location (field) providing readouts (detection (yes or no); if yes identity of pathogen and concentration of pathogen DNA and extrapolated concentration of numbers of pathogens of the present inventions.

FIG. 15 shows an exemplary general illustration of complex-target aptamer selection strategies: (A) SELEX procedure: the oligonucleotide pool (random library) was incubated with the target. Binding sequences are partitioned from the non-binding sequences and amplified by PCR. The enriched pool was incubated again with the target. After several iterative cycles; the selected oligonucleotides are cloned and sequenced. Counter-selection step: the random library is incubated with a counter-target (usually closely related to the target) to eliminate the sequences that bind this counter-target and increase the specificity of the random library (remaining sequences) that was incubated with the target. This step was usually conducted over several cycles either before or after the target incubation. Deconvolution step: the cloned and sequenced specific oligonucleotides are analyzed to evaluate binding efficiency and/or determine specific binding sites within the cell wall (usually proteins) or the complex mixture. (B) shows exemplary schematics of aptamer identification of Bacillus anthracis. Schematic of gold nanoparticle aptasensor with colorimetric-based detection (adapted from Liu and Lu; 2006). Aptamer-functionalized gold nanoparticles were aggregated using aptamer complementary oligonucleotide sequences. In the presence of the target molecule; the aptamer changed its structure for the binding event. This resulted in disassembly of the aggregates which produced a change in color. (C) Schematic of gold nanoparticle aptasensor with bio-barcode-based detection (adapted from He et al.; 2007). First; the target (thrombin) was captured by the immobilized antibodies. This Ab-target complex was then recognized in a “sandwich” type assay by the aptamer-bio-barcode functionalized gold nanoparticles. The aptamer-bio-barcodes were released and chemically degraded. The free bio-barcode nucleobases produced a specific signal detected by differential pulse voltammetry.

FIG. 16 shows exemplary (A) Schematic of gold nanoparticle aptasensor with colorimetric-based detection (adapted from Liu and Lu; 2006). Aptamer-functionalized gold nanoparticles were aggregated using aptamer complementary oligonucleotide sequences. In the presence of the target molecule; the aptamer changed its structure for the binding event. This resulted in disassembly of the aggregates which produced a change in color. (B) Schematic of gold nanoparticle aptasensor with bio-barcode-based detection (adapted from He et al.; 2007). First; the target (thrombin) was captured by the immobilized antibodies. This Ab-target complex was then recognized in a “sandwich” type assay by the aptamer-bio-barcode functionalized gold nanoparticles. The aptamer-bio-barcodes were released and chemically degraded. The free bio-barcode nucleobases produced a specific signal detected by differential pulse voltammetry.

FIGS. 17A-D shows embodiments of exemplary Polymeric nanowires-Ab biosensor of the present inventions.

FIGS. 18A-D shows exemplary results using a Nano-BEAM-Ab biosensor of the present inventions.

FIGS. 19A-D shows exemplary characterization results using a Nano-BEAM-Ab biosensor of the present inventions.

FIGS. 20A-D shows exemplary embodiments of magnetic characterizations using a Nano-BEAM-Ab biosensor of the present inventions.

FIG. 21 shows exemplary efficiency results measuring B. anthracis spores using a Nano-BEAM-Ab biosensor of the present inventions.

FIGS. 22A-D shows exemplary results measuring B. anthracis spores using a Nano-BEAM-Ab biosensor of the present inventions.

FIGS. 23A-D shows exemplary schematics of a Nano-BEAM-DNA biosensor of the present inventions.

FIGS. 24A-D shows exemplary schematics of a Nano-BEAM-DNA biosensor of the present inventions

FIG. 25 shows exemplary: (a) Biosensor architecture and dimensions and (b) schematic representation of the biosensor detection system.

FIG. 26 shows exemplary: A) (a) Experimental M-H curves of the synthesized EAPM and unmodified Fe₂O₃ nanoparticles at 300 K and (b) electrical conductivity measurements for the EAPM and Fe₂O₃ nanoparticles at room temperature. B) TEM images of (c) unmodified Fe₂O₃ nanoparticles and (d) synthesized EAPM nanoparticles. C) The EAPM nanoparticle-based direct-charge transfer biosensor resistance responses in (e) romaine lettuce; (f) whole milk and (g) ground beef samples contaminated with Bacillus anthracis spores. Average resistances with the same letters are not significantly different (P>0.05). (h) Comparison of the EAPM nanoparticle-based direct-charge transfer biosensor resistance response between pure spore suspensions of B. anthracis and pure cultures of generic Escherichia coli and S. Enteritidis.

FIG. 27 shows exemplary: A) a schematic diagram of polyaniline-based polymeric wire biosensor. (A) Components of the biosensor: (1) sample pad of cellulose membrane; (2) nitrocellulose membrane (NC) with screen-printed silver electrodes; (3) absorbent pad of cellulose membrane. (B) Cross section of the biosensor to illustrate electric signal generation: (a) before and (b) after applying the analyte onto the sample pad. (C) An equivalent RC circuit model when the electrical response was measured using the biosensor. Characteristics of screen-printed silver electrodes on the NC membrane. (D) Photograph of the screen-printed electrode. (E) I-V characteristic of the silver electrode measured by the 4192A LF impedance analyzer. (F) Histogram of resistances for the number of 30 samples.

FIG. 28 shows exemplary: A) TEM image of polyaniline-coated magnetic nanoparticles; The size of iron(III) oxide magnetic nanoparticles was less than 100 nm; which were connected with each other by the medium of the polyaniline; B) Analysis of streptavidin-biontin interactions with pulse mode and non-pulse mode measurement; Resistances were measured every 1 s after applying the analyte with pulse current (10 μA) (solid line) and non-pulse current (dashed line) applied; electrical responses became stabilized approximately after 2 min after applying the analyte; C) Dose-dependent response according to the concentrations of the analyte; Streptavidin was immobilized on the NC membrane modified with 1% glutaraldehye and incubated with the indicated mixture concentrations of the magnetic composite Fe2O3/polyaniline particles-biotinylated IgG. Then; the polyaniline-based biosensors were analyzed by the pulse mode voltammetry. Sign of * is represented as a reference signal of the phosphate buffer to monitor resistance changes.

FIG. 29 shows exemplary: (A) architecture of a model multiplexed biosensor: agglomeration of polyaniline-antigen-antibody complex between the electrodes which was indicative of presence or absence of pathogen; (B) The principle of operation of the model biosensor; (C) Copper masks which were used for making electrodes

FIG. 30 shows exemplary: (A) Conductance measured across two antibody capture zones (marked by 1 and 2) when an analyte containing B. cereus is applied (B) Conductance measured across two antibody capture zones (marked by 1 and 2) when an analyte containing E. coli is applied (C) Conductance measured across two antibody capture zones (marked by 1 and 2) when both pathogens (B. cereus; E. coli) are present in an analyte; (D) Conductance measured across the single antibody capture zone for different concentration of pathogens (B: B. cereus; E: E. coli)

FIG. 31 shows exemplary: (A) Conductometric biosensor response on purified IgG from serum of JD-infected and JD-free cattle (eight replications per experimental set); B) Influence of biosensor reading time and ELISA OD on the biosensor resistance; ELISA OD<1=JD negative; >1=JD positive; (C) Schematic of the fabricated immunosensor strip (D) conjugate membrane containing Pani-AB/IgG (B) capture membrane with immobilized MAPPD; B) Cross-section of a capture membrane (BA) before (BB) after positive JD sample application.

FIG. 32 shows an exemplary nanoporous biosensor: (A) Electrochemical cell used for porous silicon fabrication; (B) Schematic of the porous silicon chip functionalization and testing; (C) (A) SEM image of porous silicon from P-type silicon (0.01 W cm); (B) photoluminescence of porous silicon; 1) porous silicon under UV light; 2) planar silicon under UV light; 3) porous silicon under white light; 4) planar silicon under white light; Anodizing condition of porous silicon was 14.2% ethanoic HF; 5 mA/cm for 1 h; The wavelength of UV light was 254 nm; (D) Schematic of a biotin-streptavidin system for the evaluation of DNA hybridization; E) Absorbance reading at 410 nm after HRP-ABTS reaction on porous silicon (nonbiotinylated versus biotinylated DNA sample).

FIG. 33 shows exemplary nanoporous biosensor (A) Absorbance reading at 410 nm after HRP-ABTS reaction from biotiny-lated DNA samples (porous silicon versus planar silicon). (B) Absorbance reading at 410 nm from different biotinylated DNA samples after HRP-ABTS reaction on porous silicon chips. (C) Cyclic voltammograms of nonbiotinylated DNA samples on PS biosensor (0.01 W cm) in a 5 mM K Fe(CN) solution with 1 M KCl. Scan rate was 50 mV/s.

FIG. 34 shows exemplary: (A) Schematic illustration of the fabrication process for the development of PS-based DNA chip. (a) Bare p-Si substrate 19×19×0.5 mm, (b) PS formation. (c) PPy electropolymerization. (d) pDNA immobilization. (B) (a) Photograph of bare p-Si, (b) p-Si/nPS, and (c) p-Si/nPS/Ppy and (C) Cross-sectional SEM image of a nPS/Ppy+pDNA+tDNA substrate. (a) Bulk p-Si. (b) nPS layer anodized in an ethanolic HF solution with 0 mA 1 cm of constant forcing function for 30 min. (c) Directly electropolymerized PPy layer doped with pDNA and tDNA. 10 nm of Au layer was deposited on the top surface for imaging.

FIG. 35 shows an exemplary: (A) Plot of iₚ versus tDNA concentration after 1 h of incubation time (closed circle). iₚ observed at ca. 0.2 V versus Ag/AgCl were selected for the calibration. Open circles mean background signals obtained from nPS/PPy+tDNA substrates. (B) Plot of iₚ versus incubation time, where tDNA concentration is 0.909 microM. iₚ observed at ca. 0.2 V versus Ag/AgCl were selected for the calibration.

### DETAILED DESCRIPTION OF THE INVENTION

The present invention relates to methods and compositions for identifying a pathogen. The inventions provide an antibody-based biosensor probe comprising (AUNT) in combination with a polymer-coated magnetic nanoparticle. In particular, a nanoparticle-based biosensor was developed for detection of Escherichia coli O157:H7 bacterium in food products. Further described are biosensors for detecting pathogens at low concentrations in samples. Even further, a gold nanoparticle-based electrochemical biosensor detection and amplification method for identifying the insertion element gene of Salmonella enterica Serovar Enteritidis is described. The present invention provides compositions and methods for providing a handheld potentiostat system for detecting pathogens outside of the laboratory. The AUNT biosensor system has applications detecting pathogens in food, water, beverages, clinical samples, and environmental samples.

In addition to other pathogens, Escherichia coli (E. coli) O157:H7 has emerged as a major foodborne and waterborne pathogenic bacterium isolated with high frequency in outbreaks of bloody diarrhea and in sporadic community-acquired diarrhea in Western Europe in addition to North and South America [Neill, et al., 1994, Foodborne Disease Handbook vol 1. ed Hui, et al., (New York: Marcel Dekker) p. 184, herein incorporated by reference]. While virulent E. coli strains typically cause no more than a bout of diarrhea in healthy adult humans, specific variants, such as E. coli O157:H7 which often encode a toxin similar in its amino acid sequence to Shigella toxin, causes serious illness and sometimes death in the elderly, the very young or the immunocompromised [Hudault, et al., 2001, Gut 49 47-55, herein incorporated by reference]. The Center for Disease Control and Prevention estimates that E. coli O157:H7 causes 73,000 human infections in the United States each year, of which approximately 62,000 are foodborne and 11,000 are waterborne [Mead, et al., 1999, Emerg. Infect. Dis. 5 607-625, herein incorporated by reference]. Sources of human infections with E. coli O157:H7 are beef, sprouts, lettuce, salami, unpasteurized milk, and juice contaminated with pathogens [Lekowska-Kochaniak, et al., 2002, Acta Microbiol. Pol. 51 327-337, herein incorporated by reference]. Pathogen detection has been a key issue for public health and safety.

Currently, detection and identification of foodborne pathogens in food rely upon assays involving conventional culturing techniques, such as using agar media in procedures that take up to several days in order to obtain results. Further, these techniques generally require intensive labor and long time-consuming work prior to obtaining results which may reveal the presence of a general type bacteria, such as E. coli but not specific pathogenic strains, such as E. coli O157:H7, which may be almost identical if not identical to nonlethal E. coli strains with the exception of an additional toxin gene. During this identification time period significant numbers of additional people may be exposed to a lethal pathogen from a source whose sample, along with many others, is undergoing testing.

Therefore a need remains for new techniques for detecting foodborne pathogens, in particular for specifically identifying lethal strains such as E. coli O157:H7 and in a timely manner for preventing further exposure to humans.

The common detection methods of pathogens causing bacterial diarrhea, such as Salmonella Enteritidis and Bacillus anthracis, include microbiological, immunological and molecular biological techniques. Although microbiological detection accurate, it often relies on time-consuming growth in culture media, followed by isolation, biochemical identification, and sometimes serology, and need special reagents and facilities. Therefore these assays are not able to quantify the amount or number of actual microorganisms or pathogens in the sample. Further, immunological detection systems are specific but their sensitivity is low. Molecular polymerase chain reaction (PCR)-based detection method is sensitive, however, PCR is complex, expensive, time-consuming, and labor-intensive procedure.

Further, the majority of assays used to detect specific pathogens require some growth in an enrichment medium before analysis. Therefore a direct rapid, sensitive, multiplex identification of Salmonella Enteritidis and Bacillus anthracis, and the like, without the enrichment step is vital within the overall context of point-of-care (POC) diagnosis of foodborne illness and bioterrorism event. A biosensor is an analytical device that integrates a biological sensing element with a transducer to quantify a biological event into an electrical output. The nanoparticle tracer-based electrochemical universal (bio-barcode) DNA sensor described herein, makes full use of the high amplification capability of a universal (bio-barcode) assay combined with an attractive non-overlapping stripping behavior of nanoparticle tracers in square wave anodic stripping voltammetry (SWASV). The biosensor of the present inventions is much cheaper, more rapid, more sensitive, easy-to-miniaturize, and adaptable for integration into micro-systems and field-portable devices.

Salmonella enterica serovar Enteritidis is one of the most frequently reported causes of foodborne illness. It is a major threat to the food safety chain and public health. A rapid and sensitive detection of the insertion element (Iel) gene of Salmonella Enteritidis on screen-printed carbon electrodes (SPCE) is reported herein. The biosensor system includes two nanoparticles: gold nanoparticles (AuNPs) and magnetic nanoparticles (MNPs). The AuNPs are coated with the target-specific DNA probe (pDNA) which can recognize the target DNA (tDNA) and the MNPs are coated with the 2nd target-specific pDNA. After mixing the nanoparticles with the tDNA, the sandwich complex (MNPs-2nd pDNA/tDNA/1st pDNA-AuNPs) is formed. The whole complex is applied on a SPCE after washing away the unreacted AuNPs. Following oxidative gold metal dissolution in an acidic solution, the released Au3+ ions are directly quantified by differential pulse voltammetry (DPV) at a potential of +0.4 V (Vs. Ag/AgCl).

Using AUNT of the present inventions with metal nanoparticles, the detection limit using an SPCE biosensor for detecting the insertion element (Iel) gene of Salmonella Enteritidis is as low as 0.7 ng/mL.

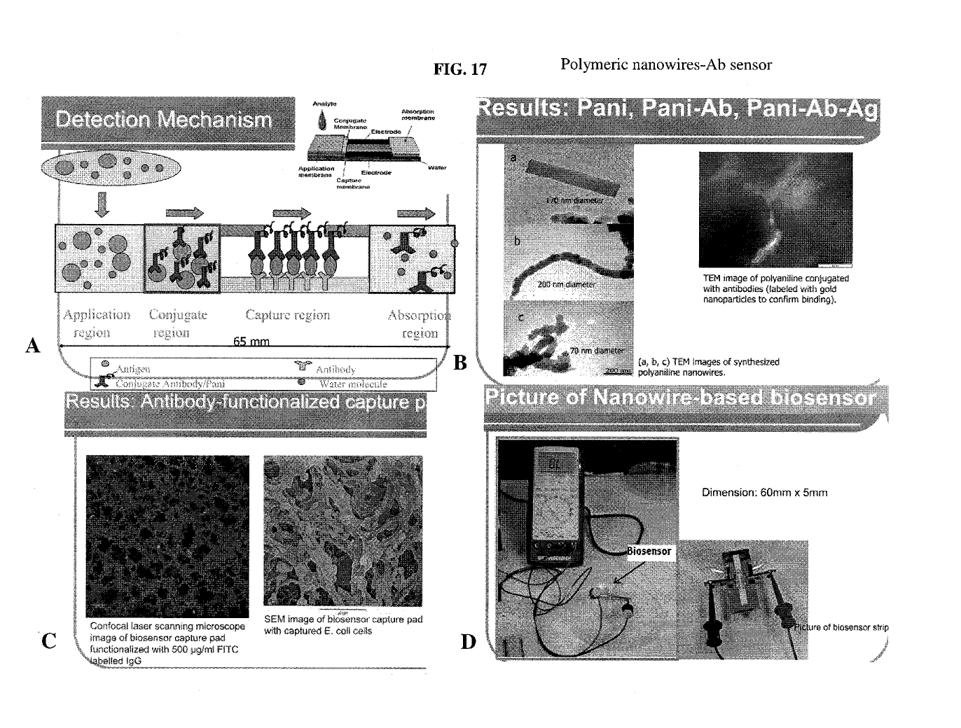

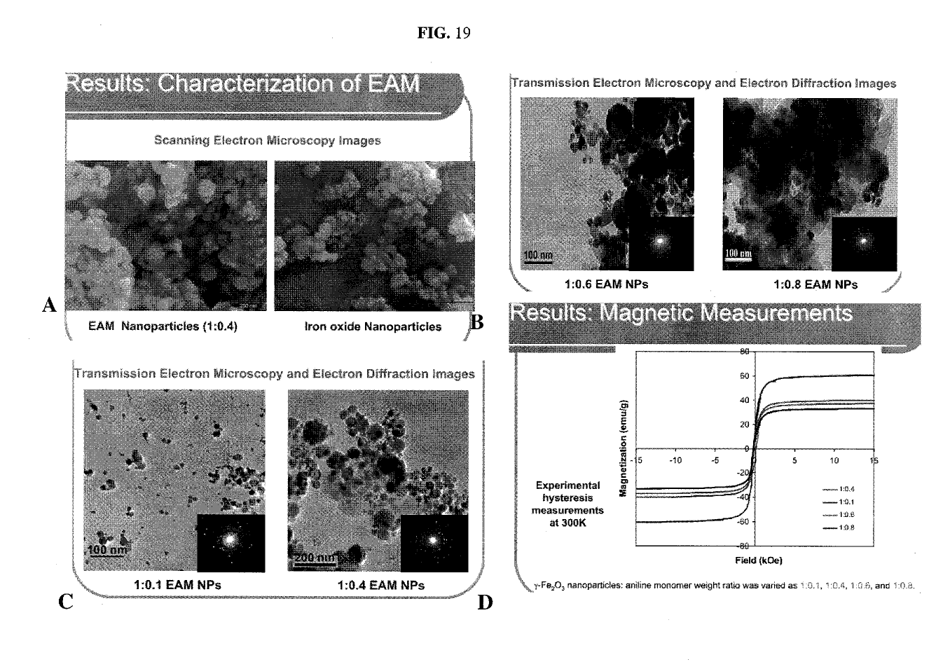

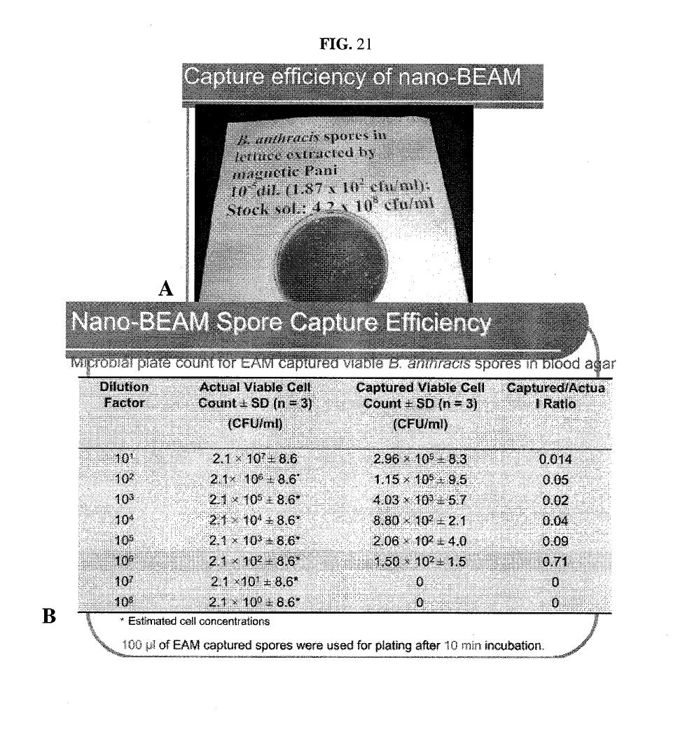

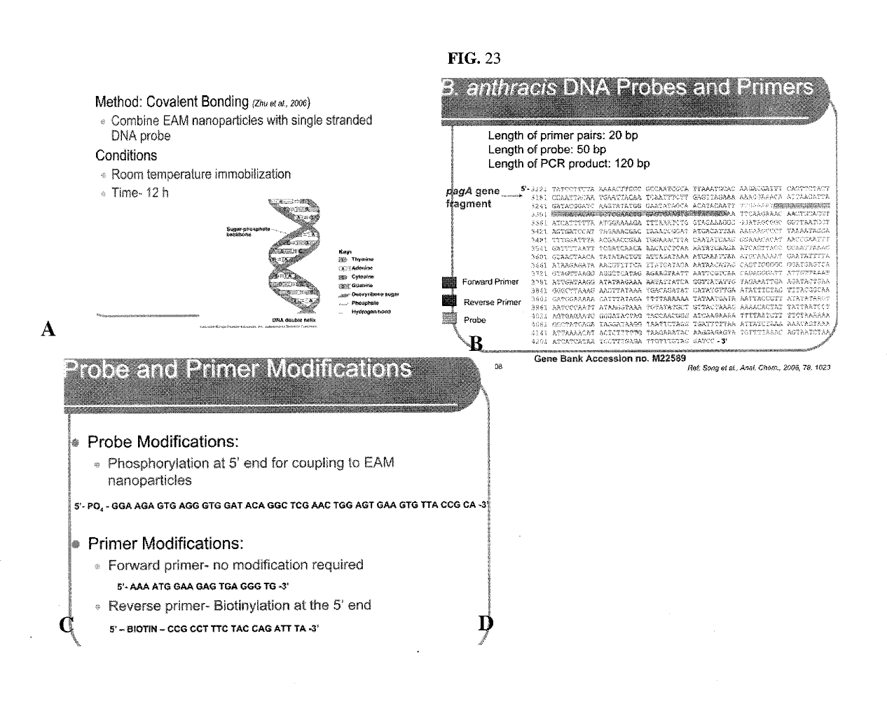

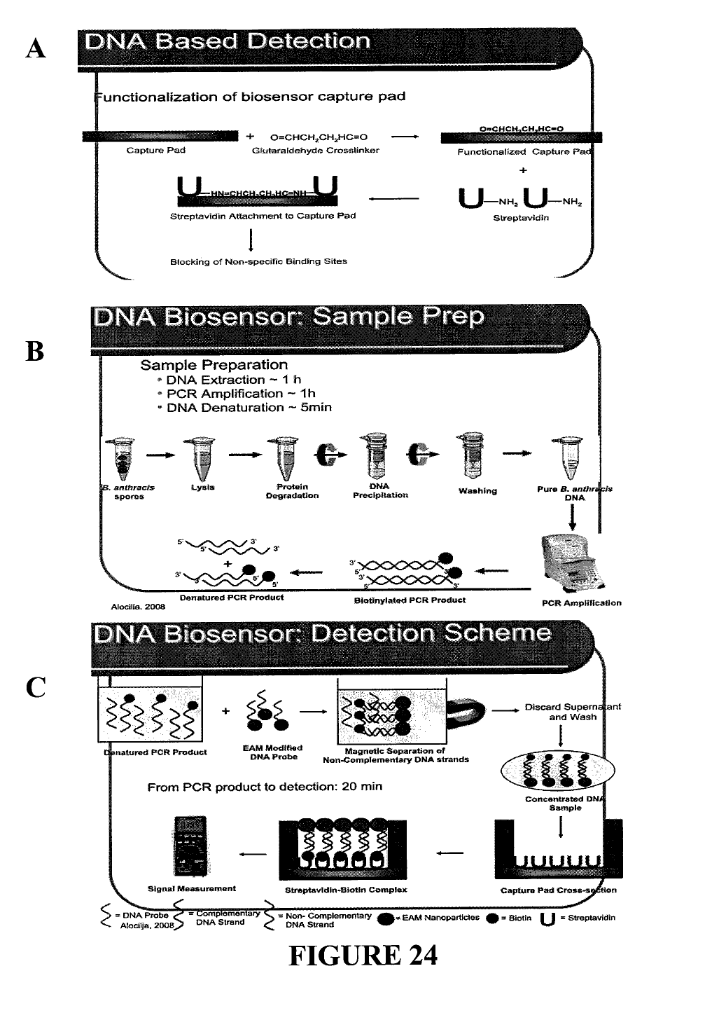

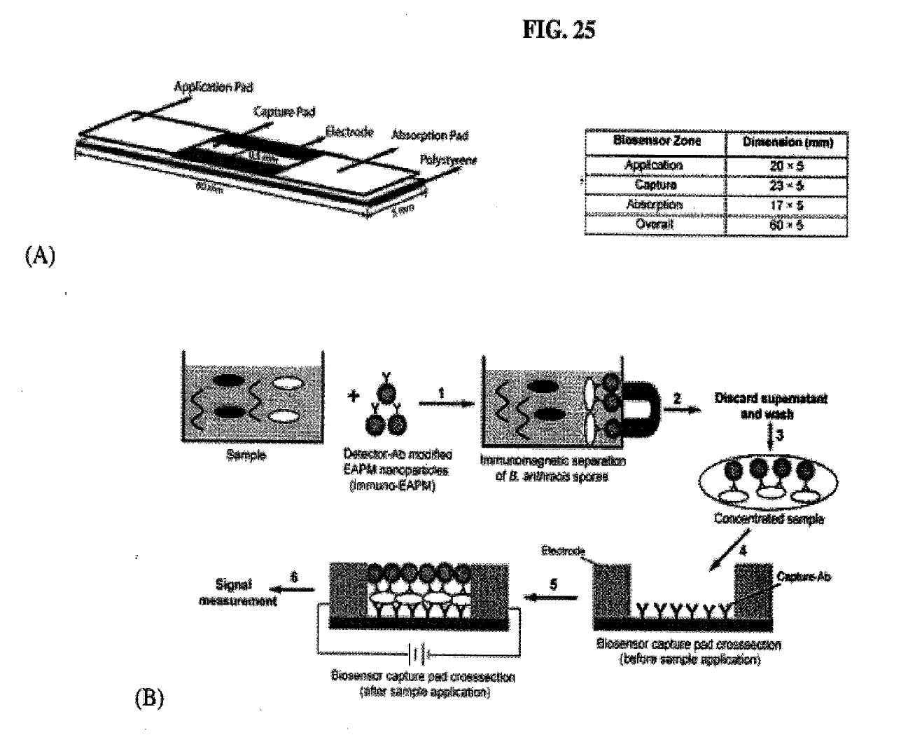

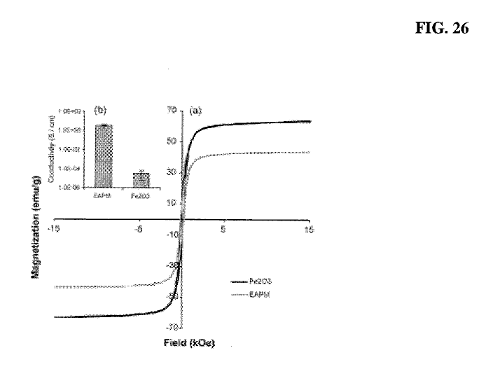

Inventions provided herein, further describe several types of DNA biosensor and DNA assay methods for sensing the presence of a pathogen, see for example, Figures herein. These include a solid-phase assay (Detection platform: membrane-based) with immobilized AuNps conjugated to a DNA probe, wherein the DNA probe comprises a hybridization region for a Barcode DNA, and a method wherein the barcode DNA of the AuNP hybridizes to a region of the capture probe DNA attached to the immobilized capture pad between electrodes. After hybridization, the bound Au particle is coated with Ag (silver ions) for completing an electrical circuit triggering an electrochemical signal. A Polymeric nanowires-Ab sensor is described and shown (see for example, FIGS. 17-24), wherein tested samples included; Water, Milk, Fruits, strawberries, Vegetables, Alfalfa sprouts, Lettuce, Tomatoes, Ground meat, Cooked foods, Fried rice, Corn (also contemplated for testing by the AUNT biosensors of the present inventions). Exemplary results are shown in FIGS. 17-24. Conducting Polymers are contemplated as described in Ahuja et al., Biomaterials, 2007, Stejskal & Gilbert, Pure Appl. Chem., 2002, 74, 857, Stejskal & Gilbert, Pure Appl. Chem., 2002, 74, 857, in addition to using polyaniline as a nanowire of the present inventions. Further contemplated is a magnetic polyaniline that can be used both as cell concentrator and transducer at the same time. Also contemplated is a Nano-BEAM-Ab sensor; Advantages include Electrical and magnetic properties in biosensor transduction, Achieve faster assay kinetics due to close proximity of EAM nanoparticles to targets, Provide increased surface area for biological events to occur, Minimize matrix interference due to improved separation, Magnetically manipulate the EMP nanoparticles, Avoid pre-enrichment and pre-treatment steps, Reduce background signal in detection devices, Design cheap, sensitive and specific detection devices. A Nano-BEAM-DNA sensor is described and shown in see for example, FIGS. 17-24 for detecting a Bacillus gene.

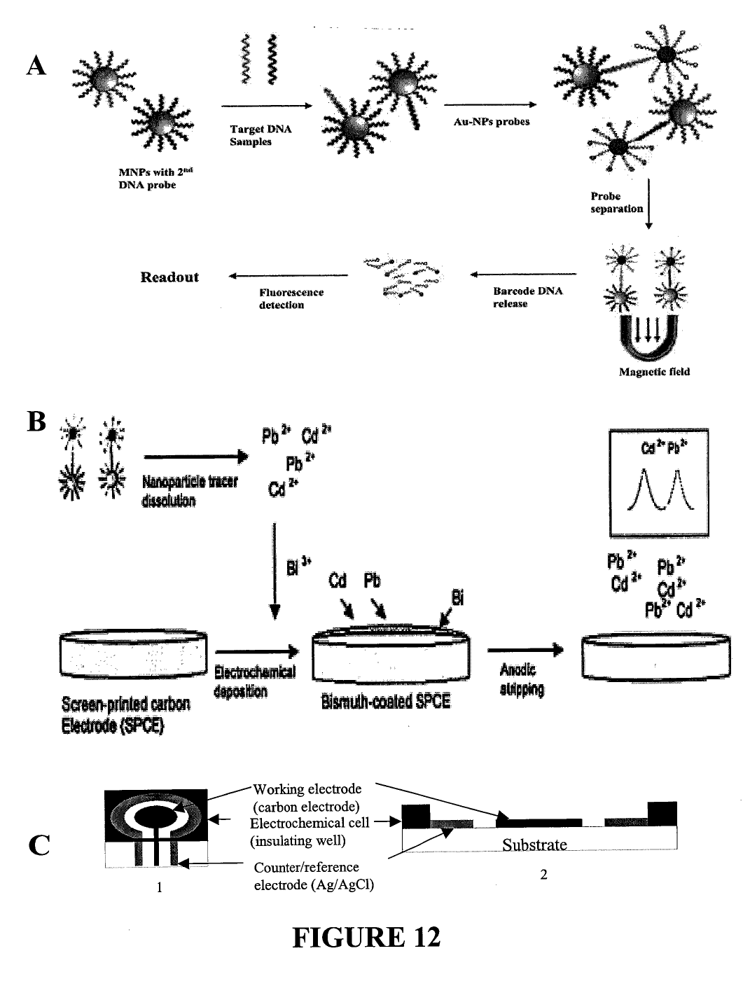

In a preferred embodiment, a handheld multiplex DNA biosensor is contemplated for rapid differential diagnosis of pathogen infections in animals or the presence of a pathogen in a sample, such a biological or environmental samples. A universal (bio-barcode) DNA assay is based herein upon electrochemical detection of specific DNA binding events, utilizing magnetic nanoparticles (MNPs) for target DNA (tDNA) separation from the sample and nanoparticle tracers (NPTs) conjugated to barcode oligonucleotides attached to gold (or a metal chosen from Zn, Cd, and Pb, See, FIG. 12 for examples) nanoparticles (i.e. AuNPs, SiNPs, polystyreneNPs, and the like) [termed detection nanoparticles] for signal amplification AUNT. A disposable screen-printed carbon electrode (SPCE) and a handheld potentiostat powered by a pocket PC are contemplated as the signal generator and detection system.

Using this technique, the detection limit of this SPCE biosensor for the insertion element (Iel) gene of Salmonella Enteritidis is as low as 0.7 ng/mL (using a bench-top potentiostat for measurements).

As described herein, a novel design for a handheld multiplex DNA-based diagnostic system was provided. A diagnostic square wave anodic stripping voltammetry (SWASV) signal generated from the NPTs on SPCE and used for the electrochemical measurement of pathogen DNA. This technology is contemplated as an inexpensive alternative to the traditional PCR technique with comparable sensitivity, especially in point-of-care scenarios (on location) and facilities with limited resources. Thus, some of the major advantages of the handheld multiplex DNA-based diagnostic system are listed below: 1. Minimized matrix interference due to improved separation and washing steps. 2. Ability to achieve higher sensitivity due to the high amplification ratio of bio-barcode DNA vs. DNA probe on AuNPs. 3. Ability to perform multiple detection in the same sample since the different NPTs show non-overlapping stripping behavior on SWASV. 4. No surface modification of electrode and resulting longer shelf-life of a disposable SPCE. The shelf-life of the disposable SPCE is about 2 years if properly stored. 5. The diagnostic system is cheap: it includes a one-time investment of a handheld potentiostat at $5,000. Each analysis thereafter will cost an estimated $1.50 per sample. 6. Ability to perform rapid detection. After the DNA sample is ready, the whole detection time is around 1 hour. 7. The SPCE is easy-to-miniaturize, and adaptable for integration into micro-systems and field-portable devices.

In a preferred embodiment, the invention provides and uses a handheld electrochemical sensor for detecting and quantitating pathogens. The response time of the device should be short enough to apply fast analytical techniques such as square wave voltammetry. Combined with a battery-powered, handheld potentiostat, the whole biosensor diagnostic system should provide rapid diagnosis for bacterial diarrheas in clinical sites and POC (point-of-care) facilities, such as clinics, hospitals and home care.

An example of a handheld electrochemical sensor that is contemplated for use in the present inventons is a PalmSens Handheld Electrochemical Sensor Interface, by Palm Instruments BV, The Netherlands (http://www.palmsens.com/index.html). Website© 2007 Palm Instruments BV. PalmSens claims it can be extended for use as a multichannel potentiostat including applicable techniques such as differential pulse and square wave (stripping) voltammetry, normal pulse and ac voltammetry, stripping chronopotentiometry or PSA, amperometric and pulsed amperometric detection,potentiometry, and linear sweep and cyclic voltammetry. PalmSens is stated to be controlled by a Pocket PC as well as a laptop or desktop PC. Additionally described by Palm is “The EmStat, as an embedded potentiostat, is the smallest, commercially available computer controlled potentiostat.” Further provided are companies providing Screen-Printed Carbon Electrodes, such as “DropSens Screen-Printed Carbon Electrodes” with a link to “world wide web.dropsens.com/en/productos.html” (© 2006 All Rights Reserved DropSens). Additionally, this reference discloses hardware and software for measuring heavy metals in wastewater, such as Pb, Cd, etc., see examples for measuring Pb and Cd in FIG. 12.

### I. Types of Biosensors.

In contrast to DNA based biosensors, there are numerous types of biosensors incorporating biological components for recognizing proteins. Theses biosensors primarily rely upon antibody recognition of proteins with a variety of transducer systems. Several types of biosensors use DNA recognition elements combined with optical transducers and a few use DNA recognition elements combined with electrochemical transducers. However, the DNA recognition elements of these biosensors do not contain the elements of the present inventions as described herein.

The following are exemplary biosensors containing various types of DNA recognition elements. In particular, publications by Mirkin et al., describe a DNA biobarcode wherein the DNA recognition elements include probe sequences containing DNA sequences for hybridizing to both a target DNA and a universal DNA sequence also termed AuNP sequences. The biobarcode DNA that hybridizes to target DNA is then used in a variety of solid-based assays with optical readouts. For example, Mirkin, et al., WO 2008157649; discloses a bio-barcode assay for detecting Bacillus subtilis DNA comprising AuNP probes and Magnetic Microparticles (MMPs) with the use of probe sequences for hybridizing to Bacillus DNA, the use of Bacillus subtilis DNA as target DNA, and unspecified DNA sequences used as a “barcode.” The MMPs with Bacillus probe were added to and hybridized to the target Bacillus DNA in solution prior to the addition of the AuNP with Bacillus probes (which are the “barcode” or recognition element) of which the Au binding end of the barcode contained a complementary region to the universal barcode), forming a complex prior to magentic isolation. The “barcode” DNA was released from the AuNP and attached to a solid surface through hybridization of the Bacillus “barcode” DNA to a complementary strand pre-attached to the solid surface. Then a “universal AuNP” comprising a Universal barcode DNA sequence comlementary to the free end of the Bacillus “barcode” sequence was hybridized to the Bacillus barcode DNA sequence preattached to a solid surface and used in combination with nonspecific silver deposition ontop of the bound AuNP prior to optical detection with a Verigene system (Nanosphere, Inc.). Though Hill and Mirkin, 2006, was one of the many references cited, the universal barcode sequence was not provided in the applciation, nor in the sequence listing nor in the two priority provisional applications. In Hill and Mirkin, 2006, Nature Protocols, 1:324-336, a “bio-barcode assay for the detection of nucleic acid and protein targets without PCR was described for both nucleic acid and protein targets. Two types of particles are used in the assay: (i) a magnetic microparticle with recognition elements for the target of interest; and (ii) a gold nanoparticle (Au-NP) with a second recognition agent (which can form a sandwich around the target in conjunction with the magnetic particle) and hundreds of thiolated single-strand oligonucleotide barcodes comprising sequences specific for the target DNA. After reaction with the analyte, a magnetic field is used to localize and collect the sandwich structures, and a DTT solution at elevated temperature was used to release the barcode strands. The barcode strands were identified on a microarray via scanometric detection or in situ if the barcodes carry with them a detectable marker. AuNPs consisting of universal barcode “Universal probe”5′-thiol modifier-AAA AAA AAA ATT ATT CGT AGC T-3′,” were used to bind to barcodes containing universal recognition sequences preattached to a solid surface for optical readouts. However, actual data for DNA recognition elements was not shown or described. An additional publication, Park, Mirkin et al., 2002, SCIENCE 295:1503-1506, describes electrochemical detection as a transducer however this assay is a solid based assay. Further, an exemplary reference by Jaffrezic-Renault, et al., Published: 30 Apr. 2007, Sensors 2007, 7:589-614 describes two separate assays 1) a genomagnetic electrochemical bioassay (GEME), based on a PNA [Peptide nucleic acid] probe and an electroactive intercalator wherein metal particles are in solution with various types of electrochemical detection methods (See, Table 3) and 2) a bio-bar code assay wherein a bio-bar code complement is attached to a AuNP, forming sandwiches of DNA recognition elements on Au nanoparticles attached to target DNA and magnetic beads for scannometric and optical detection that are different from the biosensors of the present inventions. Additional publications describing nanoparticle biosensors with electrochemical detection methods that are different from the present inveitons include Wang, et al., 2001, Anal. Chem., 73 (22):5576-5581), which also suggests the development and use of a Hand-held, battery-operated PSA instruments for on-site detection of trace metals, [Yarnitzky, et al., Talanta 2000, 51, 333, herein incorporated by reference] and suggesting the use of a second labeled probe in a sandwich assay format (i.e., a three-component assembly).

The use of screen printed electrodes and handheld devices for detecting metal nanoparticles in solution were described by Merkoçi, et al., Published 3 Jan. 2007, Nanotechnology 18: 035502 (6 pp), A simple method based on screen-printed electrodes and a handheld potentiostatic device is reported for the detection of water soluble CdS quantum dots [QD] modified with glutathione by using square wave voltammetry. AndSharma, et al., Epub 2008 Sep. 17, J Clin Microbiol. 2008 November; 46(11):3759-65 discloses a “disposable amperometric immunosensor was developed for the detection of Plasmodium falciparum histidine-rich protein 2 (PfHRP-2) in the sera of humans with P. falciparum malaria . . . disposable screen-printed electrodes (SPEs) were modified with multiwall carbon nanotubes (MWCNTs) and Au nanoparticles. While Yeung, et al., Published online 25 Sep. 2006, Nucleic Acids Research, 2006, Vol. 34, No. 18, e118 and Lee, et al., First published as an Advance Article on the web 17 Apr. 2003, Lab Chip, 2003, 3, 100-105, herein incorporated by reference. These references describe silicon-glass based microchambers for DNA-based detection of pathogens, E. coli and B. subtilis cells in Yeung, using electrochemical detection of metals in solution, these references do not describe the compositions and methods of the present inventions. A current review of Biosensors for detecting pathogens is found in Palchetti and Mascini, Chapter title: Amperometric Biosensor for Pathogenic Bacteria Detection in Principles of Bacterial Detection: Biosensors, Recognition Receptors and Microsystems; by Zourob (Editor), Elwary (Editor), Turner (Editor), Publisher: Springer; 1 edition (Sep. 30, 2008) including a statement of the “possibility” of an electrochemical biosensor to operate in turbid media in the cited chapter.

II. Detection of a Nanoparticle-Based Electrochemical DNA Sensor for the Detection of the Insertion Element (Iel) gene of Salmonella Enteritidis Using Compositions and Methods of the Present Inventions.

A biosensor is an analytical device that integrates a biological sensing element with a transducer to quantify a biological event into an electrical output. It is a promising device for rapid detection of pathogenic microorganisms. Based on different sensing elements, the biosensor can be divided into immuno-sensor (Pal, et al., Biosensors and Bioelectronics, 2007. 22(9-10):2329-2336; Pal, et al., Biosystems Engineering, 2008. 99(4): 461-468; Muhammad-Tahir, et al., IEEE Sensors Journal, 2005. 5(4):757-762), DNA sensor (Zhang, et al., IEEE Sensors Journal, 2008. 8(6):775-780), cell-based biosensor (Wanzenboeck, et al., 2004. Pennington, N.J. 08534-2896, United States: Electrochemical Society Inc., herein incorporated by reference), aptasensor (Torres-Chavolla, et al., Biosens Bioelectron. 2009 Jul. 15; 24(11):3175-82. Epub 2008 Nov. 25, herein incorporated by reference), and enzymatic sensor (Mathew, et al., Biosensors and Bioelectronics, 2005, 20(8 SPEC ISS):1656-1668, herein incorporated by reference). In recent years, the interest for DNA-based diagnostic tests has been growing. The development of systems allowing DNA detection is motivated by applications in many fields: DNA diagnostics, gene analysis, fast detection of biological warfare agents, and forensic applications (Sassolas, et al., Chem Rev, 2008, 108(1):109-39, herein incorporated by reference). DNA sensor usually relies on the immobilization of a single-stranded DNA (ssDNA) probe onto a surface to recognize its complementary DNA target sequence by hybridization. Transduction of hybridization of DNA can be measured electronically (Lee, et al., Anal Chem, 2001. 73(22):5629-32; Yuk, et al., Biosensors and Bioelectronics, 2009. 24(5):1348-1352, herein incorporated by reference), optically (Sassolas, et al., Chem Rev, 2008, 108(1):109-39, Pu, et al., Journal of Physical Chemistry B, 2008. 112(31):9295-9300, Dubertret, et al., Nature Materials, 2005. 4(11):797-798, herein incorporated by reference) or electrochemically (Zhang, et al., IEEE Sensors Journal, 2008. 8(6):775-780, Drummond, et al., Nat Biotech, 2003. 21(10): p. 1192-1199; Weng, et al., Anal Chem, 2008. 80(18):7075-83; Jin, et al., IEEE Sensors Journal, 2008. 8(6):891-895; Huang, et al., Analyst, 2006. 131(3):382-7, herein incorporated by reference), or by using mass-sensitive devices [Huang, et al., Analyst, 2006. 131(3):382-7, herein incorporated by reference). In this paper, a nanoparticle-based electrochemical DNA sensor for the detection of the insertion element (Iel) gene of Salmonella Enteritidis is described. The biosensor utilizes oligonucleotide-modified gold nanoparticles (AuNPs) for electrochemical indicator and magnetic nanoparticles (MNPs) for easy and clean separation from the sample. After hybridization with the tDNA, a magnetic field is used to separate the sandwich complex consisting of MNP-2nd pDNA/tDNA/1st pDNA-AuNP. Following oxidative gold metal dissolution in an acidic solution, the released Au3+ ions can be measured by differential pulse voltammetry (DPV) on a disposable screen-printed carbon electrode (SPCE).

Additional descriptions of materials and methods of the present inventions are provided below, thus the following papers by the inventors are herein included by reference in their entirety: Zhang, Carr, and Alocilja, Fluorescent Bio-barcode DNA Assay for the Detection of Salmonella enterica Serovar Enteritidis, poster presentation at the Tenth World Congress on Biosensors, Shangai, China, May 14-16, 2008; Alocilja, Nano-Biosensors for Food Defense, PITTCON 2008, Mar. 2-7, 2008, New Orleans, La. Published online at least in part as a: Presentation: Nano-Biosensors For Food Defense, Jul. 7, 2008; Zhang, Carr, and Alocilja, Selected Papers from the Tenth World Congress on Biosensors, available online in part, 19 Aug. 2008, Fluorescent bio-barcode DNA assay for the detection of Salmonella enterica serovar Enteritidis, Biosensors and Bioelectronics, 1 Jan. 2009, 24(5):1377-1381 and exemplary poster presentations of universal (bio-barcode, biobarcode) biosensors of the present inventions, Zhang, et al., Highly Amplified Bio-barcode DNA Biosensor for the Detection of Salmonella Enteritidis, The Tenth World Congress on Biosensors, Shanghai, China, May 2008 and 19B: Deng Zhang, Connie Shi and Evangelyn C. Alocilja, Nanoparticle-based Electrochemical Detection of the Insertion Element Gene of Salmonella enterica Serovar Enteritidis, The 2008 National Center for Food Protection and Defense (NCFPD) Annual Meeting. Chaska, Minn., September 2008.

A. Nanoparticle Bio-Barcode Dna Assay for the Detection of Pathogens.

A bio-barcode assay is provided based on polyaniline-coated magnetic nanoparticles for detection of Escherichia coli (E. coli) O157:H7. The bio-barcode assay used two types of particles. One particle was 30 nm gold nanoparticle (AuNP) conjugated with both polyclonal antibody and fluorophore-labeled barcode DNA for a signal reporter. The other particle was polyaniline-coated magnetic nanoparticles conjugated with monoclonal antibody for the same target of E. coli O157:H7. AuNP probes were conjugated to 2.5 μg/ml polyclonal antibody was used by considering space to conjugate the barcode DNA. Polyaniline-coated magnetic nanoparticles were conjugated with the monoclonal antibody by using glutaraldehyde, which was confirmed by an absorption peak at 280 nm. E. coli O157:H7 was dectected in order of 1 CFU/ml with the bio-barcode assay based on polyaniline-coated magnetic nanoparticles. The inventors contemplate additional embodiments, as described herein, for using nanoparticle tracer particles as a read out for target DNA detection.

1. Fluorescent Bio-Barcode DNA Sssay for the Detection of Salmonella enterica serovar Enteritidis.

Salmonella enterica serovar Enteritidis is one of the most frequently reported causes of foodborne illness. It is a major threat to the food safety chain and public health.

The annual cases of salmonellosis are around 40,000 in the United States from 1995 to 2005 (CDC, 2007. Salmonella Surveillance: Annual Summary, 2005. United States Department of Health and Human Services, Atlanta, Ga.). Commercially available detection methods for Salmonella enterica serovar Enteritidis include microbiological, immunological and molecular techniques. Although microbiological detection is accurate, it takes several days for incubation and needs special reagents and facilities. Commercially available immunological detection systems are specific but their sensitivity is low. Though PCR-based molecular detection method is very sensitive, PCR is often criticized for its complex, expensive, time-consuming, and labor-intensive procedure and narrow target DNA quantification range. Thus a rapid detection and valid identification of this foodborne pathogen in food and beverages is vital for food safety and public health.

A highly amplified bio-barcode DNA assay for the rapid detection of the insertion element (Iel) gene of Salmonella Enteritidis is reported herein. The biosensor transducer was composed of two nanoparticles: gold nanoparticles (Au-NPs) and magnetic nanoparticles (MNPs). The Au-NPs are coated with the target-specific DNA probe which recognized the target gene, and fluorescein-labeled barcode DNA in a 1:100 probe-to-barcode ratio. The MNPs are coated with the 2nd target-specific DNA probe. After mixing the nanoparticles with the 1st target DNA, the sandwich structure (MNPs-2nd DNA probe/Target DNA/1st DNA probe-Au-NPs-barcode DNA) is formed. A magnetic field is applied to separate the sandwich from the unreacted materials. Then the bio-barcode DNA is released from the Au-NPs. Because the Au-NPs have a large number of barcode DNA per DNA probe binding event, there is substantial amplification. The released barcode DNA is measured by fluorescence. Using this technique, the detection limit of this bio-barcode DNA assay is as low as 2.15×10-16 mol (or 1 ng/mL).

A biosensor is an analytical device that integrates a biological sensing element with a transducer to quantify a biological event into an electrical output. It is a promising device for rapid detection of pathogenic microorganisms. Use of nanomaterials in biosensors was contemplated by the inventors to allow the development of new signal transduction technologies in their manufacture. Many biosensors based on nanotechnology for the detection of foodborne pathogens and bioterrorism agents can be found in the literature. These detection techniques include conductive polymer nanowire (Pal, et al., 2007. Biosens. Bioelectron. 22(9):2329-2336), nano-porous silicon (Mathew, et al., 2005. Biosens. Bioelectron. 20(8):1656-1661), gold nanoparticles (Liu, et al., 2004. Biochem. Biophys. Res. Commun. 313(1):3-7) and carbon nanotube (Davis, et al., 2003. Chem. Eur. J. 9(16):3732-373).

In contrast, the bio-barcoded DNA assay described herein, utilizes oligonucleotide-modified gold nanoparticles (Au-NPs) for signal amplification and magnetic nanoparticles (MNPs) for easy separation from the samples with a higher level of detection, i.e. higher level of sensitivity, in addition to providing a more accurate and faster assay for pathogen detection and identification.

Oligonucleotide-functionalized gold nanoparticles were demonstrated to have advantages in terms of sensitivity and selectivity over conventional probes in a variety of biodetection schemes (Cao, et al., 2002. Science 297:1536-1540). Their outstanding performance stems from cooperative binding properties, unique optical properties, catalytic properties, and the existence of robust and versatile surface-functionalization methods (Rosi, et al., 2005. Chem. Rev. 105(4):1547-1562). The bio-barcode assay is based on Au-NPs functionalized with a large number of oligonucleotide strands (the barcodes) and a corresponding recognition agent. The large barcode DNA-to-recognition agent ratio provides a means of signal amplification.

At present, considerable attention is being paid to solid-phase extraction as a way to isolate and pre-concentrate desired components from a sample matrix (Tartaj, et al., 2003. J. Phys. D: Appl. Phys. 36(13):R182-R197). MNPs were widely used as a universal separation tool to purify nucleic acids (Obata, et al., 2002. Pharmacogenomics 3(5),697-708), proteins and peptides (Safarik, et al., 2004. BioMag. Res. Technol. 2:7), cells (Pamme, et al., 2005. Lab Chip-Miniaturisation Chem. Biol. 6:24) and other biological agents from crude samples. An attractive advantage of these nanoparticles is that it can eliminate nonspecific adsorption of unwanted biological agents by controlling the surface chemistry of the particles (Hsing, et al., 2007. Electroanalysis 19(7-8):755-768). The MNPs here provide a fast, convenient and efficient method for the separation and preconcentration of the substance from large volumes of solution.

This bio-barcoded assay is particularly promising as it allows the rapid detection of protein targets at low-attomolar concentrations (Chang et al., 2007, Biosens. Bioelectron. 22(12):3139-3145) and nucleic acids at high-zeptomolar levels under optimized conditions (Nam et al., 2004). In addition, enzymatic amplification of the target sequence prior to detection is not necessary. Demonstrated herein for the first time is the feasibility of the bio-barcode assay to detect oligonucleotide targets from one of the most important foodborne pathogens—Salmonella Enteritidis. In this embodiment, two DNA probes associated with the insertion element (Iel) gene of the organism were used.

Several embodiments of a bio-barcoded DNA assay are described herein for the detection of the insertion element (Iel) gene of S. Enteritidis. The system is based on two nanoparticles: gold nanoparticles and magnetic nanoparticles. The Au-NPs work as transducer and signal amplifier and the MNPs act as separator and pre-concentrator. Gold nanoparticles were synthesized by a chemical reduction method. The conjugation reaction between the two nanoparticles and thiolated oligonucleotides was efficient. After mixing the nanoparticles with the target DNA, the sandwich structure (MNPs-2nd DNA probe/Target DNA/1st DNA probe-Au-NPs-barcode DNA) was formed. The fluorescence signal of released barcode DNA had an exponential relationship with target DNA concentration. The results show that the detection limit of this bio-barcoded DNA assay is 1 ng/mL (or 2.15×10-16 mol).

2. Nanoparticle Bio-Barcode DNA Assay for the Detection of Pathogen.

The inventors further contemplates various types of conjugation of metal tracer particles to gold particles (in some cases polystyrene particles). In one embodiment, the inventors contemplate using different silent DNA sequences for attaching read out molecules, such as nanoparticle tracers and/or fluorescent molecules. In one embodiment, the inventors contemplate testing AUNT in PCR samples of amplified tDNA, using washed and unwashed PCR amplified DNA. In another embodiment, the inventors further contemplate a multiple pathogen detection system, such as detecting Salmonella Enteritidis and Bacillus anthracis in the same sample (see, FIG. 11).

B. Miniaturized Electrochemical Detection for Use with Bioassyas of the Present Inventions.

A disposable integrated miniaturized electrochemical sensor was developed and tested using low-cost screen-printing (thick-film) technology for the determination of trace amounts of exemplary metals, such as lead and cadmium. The screen-printed carbon electrode (SPCE) was used as a substrate for in situ deposition of a metallic film of bismuth, which allowed the electrochemical preconcentration of metal ions. Lead(II) and cadmium(II) were simultaneously detected using square wave anodic stripping voltammetry on bismuth film-coated electrochemical sensors and bare electrochemical sensors. Operational parameters, such as bismuth concentration and deposition time, were optimized for the purpose of determining trace amount of metals in acetate buffer solution (0.1M, pH 4.5). In 100 μL of sample solution, the detection limits of cadmium(II) were 0.5 mg/L on bismuth film-coated electrochemical sensors and 2 mg/L on bare electrochemical sensors. The detection limits of lead(II) were 0.5 mg/L on bismuth film-coated electrochemical sensors and 0.5 mg/L on bare electrochemical sensors. Cadmium(II) detection benefited from the addition of bismuth. This integrated miniaturized electrochemical sensor has provided a relatively inexpensive on-site detection for trace levels of heavy metals, coupled with a portable electrochemical potentiostat. It can be potentially applicable to the medical diagnosis in a very small volume of samples, such as body fluids.

Heavy metals, such as lead and cadmium, are one class of contaminants that can produce undesirable effects even if they are present in trace quantities. They are the most insidious pollutants because they are resistant to biodegradation and can persist in the environment for a long period of time. Square wave anodic stripping voltammetry (SWASV) is a powerful tool for measuring trace metals. It can be viewed as combining the best aspects of square wave voltammetry and anodic stripping voltammetry. The SWASV technique includes the background suppression and sensitivity of differential pulse voltammetry and the diagnostic value of normal pulse voltammetry (Bard, and Faulkner, Electrochemical Methods: Fundamentals and Applications, Wiley, New york, 2000). The remarkable sensitivity of SWASV technique is attributed to the combination of an effective pre-concentration step with advanced measurement procedure that generates an extremely favorable signal-to-background ratio. Four to six metals can be measured simultaneously in various matrices at concentration levels as low as 10⁻¹⁰ M (Wang, Analytica Chimica Acta 500(1-2) (2003) 247-257). In addition, the possibility of portable and compact instruments for stripping analysis coupled with their low power requirement makes SWASV attractive for on-site monitoring of trace metals.

Two commonly used electrodes in the development of stripping voltammetry are the mercury-film electrode (MFE) and hanging mercury drop electrode (HMDE). However, their use as an electrode material has been severely restricted because of the toxicity of mercury and occupational health considerations. Bismuth is an environmentally friendly element with low toxicity and a widespread pharmaceutical use (Rodilla, et al., Chemico-Biological Interactions 115(1) (1998) 71-83). In addition, bismuth electrodes can be prepared by simultaneous deposition with the target heavy metals, in a manner analogous to in situ plated mercury-film electrodes. Such electrodes display well-defined, sharp and highly reproducible stripping peaks for low (ppb) concentrations of lead, cadmium, or zinc over a low background current (Wang, et al., Anal Chem 72(14) (2000) 3218-3222).

Screen-printing technology represents a promising route for the mass production of inexpensive, reproducible and sensitive disposable electrochemical sensors for the determination of trace levels of important compounds. Compared with conventional electrodes, screen-printed carbon electrodes have several advantages, such as simplicity, convenience, low cost, and the avoidance of contamination between samples (Miscoria, et al., Analytica chimica acta 578(2) (2006) 137-144; Susmel, et al., Biosensors & bioelectronics 18(7) (2003) 881-889). Solid carbon electrodes are commonly used in electrochemical analysis due to their broad potential window, low background current, rich surface chemistry, low cost, chemical inertness, and suitability for various sensing and detection applications Kissinger and Heineman, Laboratory Techniques in Electroanalytical Chemistry, Marcel Dekker, New york, 1996). Sensors based on screen-printed carbon electrode (SPCE) were extensively used for detection of biological agents, such as glucose (Xu, et al., Conf Proc IEEE Eng Med Biol Soc 2 (2005) 1917-1920; Guan, et al., Biosensors & bioelectronics 21(3) (2005) 508-512), cholesterol (Carrara, et al., Biosensors & bioelectronics 24(1) (2008) 148-150), antigens (Kim, et al., IEEE Sensors Journal 6(2) (2006) 248-252) and DNA (Hernandez-Santos, et al., Analytical chemistry 76(23) (2004) 6887-6893; Ruffien, et al., Chemical communications (Cambridge, England) (7) (2003) 912-913). There has been a lot of published work on the detection of metals with screen-printed carbon electrodes using stripping analysis techniques (Desmond, et al., Sensors and Actuators, B: Chemical B48(1-3 pt 4) (1998) 409-414.; Kadara, et al., Analytical and Bioanalytical Chemistry 378(3) (2004) 770-775; Hwang, et al., Talanta 76(2) (2008) 301-308. However, these electrochemical detection systems with electrochemical sensors need additional counter and reference electrodes and a relatively large volume of electrolyte solution. This limits potential applications of electrochemical sensors on the portable detection, especially for samples with a very small volume requirement such as body fluids.

Described here a disposable integrated miniaturized electrochemical sensor for the determination of trace amount of lead and cadmium. The cyclic voltammogram of ferricyanide shows that the voltammetric response of electrochemical sensor is irreversible. The reusability test shows the electrochemical sensors are only good for one-time use. Due to the limitation of stirring, the experimental condition was optimized. Deposition potential of −1.2 V, 1 mg/L of bismuth and 10 min deposition time were applied, to the SWASV measurement. The detection limits of cadmium(II) were 0.5 mg/L on bismuth film-coated electrochemical sensors and 2 mg/L on bare electrochemical sensors in 100 μL of sample solution. The detection limits of lead(II) were 0.5 mg/L on bismuth film-coated electrochemical sensors and 0.5 mg/L on bare electrochemical sensors in 100 μL of sample solution. Cadmium(II) detection benefited from the addition of bismuth. The integrated miniaturized electrochemical sensor described here offers a convenient and inexpensive means of miniaturization and mass production, and can be used for on-site measurements of trace metals. It is potentially applicable to the detection of a very small volume of body fluids, when combined with the portable potentiostat.

Thus a disposable integrated miniaturized electrochemical sensor was developed using low-cost screen-printing (thick-film) technology for the determination of trace lead and cadmium in 100 μL of sample solution. Due to the limitation of stirring, operational parameters, such as bismuth concentration and deposition time, were optimized. Both the electrochemical sensor modified with in situ plated bismuth film and the bare electrochemical sensor were evaluated in 100 μL of sample solution for the determination of lead and cadmium. A disposable integrated miniaturized electrochemical sensor using low-cost screen-printing (thick-film) technology was employed for the determination of trace amount of lead and cadmium. The screen-printed carbon electrode (SPCE) was used as a substrate for in situ deposition of a metallic film of bismuth, which allowed the electrochemical preconcentration of metal ions. Lead(II) and cadmium(II) were simultaneously detected using square wave anodic stripping voltammetry on bismuth film-coated electrochemical sensors and bare electrochemical sensors. Operational parameters, such as bismuth concentration and deposition time, were optimized for the purpose of determining trace amount of metals in acetate buffer solution (0.1M, pH 4.5). In 100 μL of sample solution, the detection limits of cadmium(II) were 0.5 mg/L on bismuth film-coated electrochemical sensors and 2 mg/L on bare electrochemical sensors. The detection limits of lead(II) were 0.5 mg/L on bismuth film-coated electrochemical sensors and 0.5 mg/L on bare electrochemical sensors. Cadmium(II) detection benefited from the addition of bismuth. This integrated miniaturized electrochemical sensor has provided a relatively inexpensive on-site detection for trace levels of heavy metals, coupled with a portable electrochemical potentiostat. It can be potentially applicable to the medical diagnosis in a very small volume of samples, such as body fluids.

C. Aptamers.

Additional contemplated embodiments for use in the present inventions include a biosensor comprising an aptamer, which are specific nucleic acid sequences that can bind to a wide range of non-nucleic acid targets with high affinity and specificity (Torres-Chavolla, Aptasensors for detection of microbial and viral pathogens. Biosens Bioelectron, 2008 Nov. 25, herein incorporated by reference in its entirety.

### Aptasensor Platforms.

Aptamers represent an alternative to antibodies as recognition elements in biosensors. The aptamer selection can be performed under real matrix conditions, which is particularly useful for environmental and food samples. Aptamers can be modified for immobilization purposes and labeled with reporter molecules, without affecting their affinity. As nucleic acid sequences, aptamers can be subjected to repeated cycles of denaturation and renaturation; this makes it possible to regenerate the immobilized biocomponent function for reuse (Jayasena, 1999. Clin. Chem. 45 (9), 1628-1650; O'Sullivan, 2002. Anal. Bioanal. Chem. 372 (1), 44-48). Besides the advantages of specificity discussed above, aptamer immobilization characteristics are crucial for aptasensor applications. Aptamers can be chemically modified and labeled more easily than antibodies. These modifications facilitate the functionalization of nanoparticles and surfaces. Also, aptamers can undergo conformational changes and become reusable, allowing some of the aptasensor platforms to be recyclable. A major disadvantage is that most of the available aptamers are RNA structures, which are highly sensitive to nuclease degradation. Alternatives to overcome this problem include chemical modifications of the ribose at the 2′ position (Pagratis, et al., 1997. Nat. Biotechnol. 15 (1), 68-73; Kusser, 2000. Rev. Mol. Biotechnol. 74 (1), 27-38) and the use of mirror-image analogs that are nuclease resistant (spiegelmers) (Eulberg and Klussmann, 2003).

The introduction of aptamers as potential biorecognition molecules in biosensors was first reviewed by O'Sullivan (2002). Since then, electrical-based (Lee, et al., 2008. Anal. Bioanal. Chem. 390 (4), 1023-1032; Willner, et al., 2007. Angew. Chem. Int. Ed. 46 (34), 6408-6418) and optical-based platforms (Deisingh, 2006. Handb. Exp. Pharmacol. 173, 341-357; Fischer, et al., 2007. Curr. Opin. Chem. Biol. 11 (3), 316-328; Li, et al., 2008. Sci. Chin. Ser. B-Chem. 51 (3), 193-204) were reviewed, including aptasensors for biosecurity (Fischer, et al., 2007. Curr. Opin. Chem. Biol. 11 (3), 316-328) and clinical (Deisingh, 2006) applications. For clinical diagnostics, several aptamer-based biosensors (aptasensors) were developed. Examples include quartz crystal aptasensors to detect IgE (Liss, et al., 2002. Anal. Chem. 74 (17), 4488-4495) and the protein trans-activator of transcription (Tat protein) of human immunodeficiency virus type 1 (HIV-1) (Minunni, et al., 2004. Biosens. Bioelectron. 20 (6), 1149-1156); a fiber-optic system to detect thrombin (Lee and Walt, 2000); and a multiplex cancer marker detection system (McCauley et al., 2003). In the electrochemical-based detection systems, few aptasensors were reported (Willner and Zayats, 2007; Lee et al., 2008). Most of these aptasensors use thrombin as a target model for detection. Electrical aptasensors include: (a) electrochemical platforms, using enzyme labeling detection systems (Ikebukuro et al., 2005; Mir et al., 2006), aptamers functionalized with redox reporters (Bang et al., 2005; Baker et al., 2006), and label-free impedance spectroscopy transduction (Rodriguez et al., 2005; Cai et al., 2006); (b) field-effect transistors (Zayats et al., 2006), using single-walled carbon nanotubes (So et al., 2005); and (c) piezoelectric quartz crystals, using microgravimetric analysis (Liss et al., 2002; Hianik et al., 2005). One of the main advantages of electrical aptasensors is that sensitivity can be enhanced by attaching biocatalytic labels to the aptamer-target complexes, to amplify the detection signal. Furthermore, electrical aptasensors are more convenient for on-field detection applications, since they do not require expensive optical instruments (Willner and Zayats, 2007; Lee et al., 2008). Additionally, it is possible to use label-free and reusable detection systems. In most cases, protein electrical aptasensors can be easily reused after washing off the target protein. This cannot be done with immunological biosensors because both target and sensing element (antibodies) are proteins.

Optical-based aptasensors include aptamers labeled with fluorophores (signaling aptamers) (Jhaveri et al., 2000; Merino and Weeks, 2005), fluorescence resonance energy transfer (FRET) platforms using aptabeacons (Nutiu and Li, 2003; Heyduk and Heyduk, 2005), and optical fibers (Spiridonova and Kopylov, 2002). One of the main disadvantages of using fluorescent labels in optical aptasensors is that their application in complex matrices is limited, due to the interference and quenching of fluorophores by biological components present in the matrix (Li et al., 2008). 4.1. Aptasensors for detection of microorganisms and viruses Several aptasensors were developed to detect viral proteins. Minunni et al. (2004) developed an aptasensor platform to detect HIV-1 Tat protein by immobilizing an RNA aptamer on a piezoelectric quartz crystal. Sensitivity, specificity, and reproducibility parameters were quantified. The aptasensor was also compared with the corresponding immunosensor with immobilized anti-Tat antibodies. Both the optimized aptasensor and the immunosensor showed a detection limit of 0.25 ppm (Minunni et al., 2004; Tombelli et al., 2005a). The quartz crystal microbalance (QCM)-based aptasensor has also been compared with the corresponding surface plasmon resonance (SPR)-based aptasensor. The two aptasensors were constructed using biotin-avidin linking onto the gold surface of the transducers (quartz crystals or chips) for the immobilization chemistry. Both platforms showed similar reproducibility, sensitivity and specificity. The linear range of SPR (1-2.5 ppm) was higher that of QCM (0-1.25 ppm) (Tombelli et al., 2005a).

Another viral aptasensor example is the heptatitis C virus (HCV) core antigen detector. Lee et al. (2007) selected and tested the binding affinity of several aptamer sequences. After selection, the core specific aptamer was immobilized in a 96-well plate, using the sol-gel-based immobilization method. Then, the immobilized aptamers on the chip were incubated with recombinant core antigens. After antigen binding, the aptamer-core complexes were incubated with Cy3-labeled secondary antibodies. The platform was able to detect core-specific interaction with the aptamers, using pure recombinant protein as well as human sera matrixes. The results showed that this platform can specifically detect the core antigen from HCV-infected patients' sera (Lee et al., 2007). Detection of bacteria using aptasensors is a relatively new area. Recently, two different strategies for whole-cell detection, using quantum dots (QDs) and carbon nanotubes (CNTs), were reported. Aptamer-functionalized QDs were used to detect Bacillus thuringiensis spores (Ikanovic et al., 2007). In the study, zinc sulfide-capped cadmium selenide QDs were functionalized with a specific aptamer selected to detect B. thuringiensis. After QD-aptamer incubation with the target, the spores were washed and collected for fluorescence measurement. Several controls with non-functionalized QDs and without spores were tested to measure the non-specific attachment of the QDs to the spores and the fluorescence background noise. The reported sensitivity was 103 CFU/ml. For specificity purposes, spores from B. globigii (B. subtilis var. niger) were also tested. The system could differentiate B. thuringiensis from B. globigii at concentrations above 105 CFU/ml. The second strategy was developed to detect E. coli DH5, using aptamer-functionalized single-walled carbon nanotube field-effect transistor (SWNT-FET) arrays (So et al., 2008). The binding event between E. coli cells and the aptamer-functionalized FET produced a drop in conductance (>50%) in culture samples with concentrations between 10⁵ and 10⁷ CFU/ml. Specificity assays were conducted with S. Typhimurium.

Aptamer-conjugated nanoparticles were developed to collect and detect cancer cells from complex matrices (Herr et al., 2006; Smith et al., 2007). Aptamer-magnetic nanoparticles were used for selective cell isolation, and aptamer-fluorescence nanoparticles were used to amplify the detection signal. Fluorescence was detected by confocal microscopy. The system was developed to detect leukemia and lymphoma cells with a detection limit of approximately 250 cells (Smith et al., 2007). These strategies can be potentially applied to the collection, concentration, and detection of microorganisms from complex matrices. Whole-cell aptamer detection platforms represent a promising alternative not only for clinical diagnostics, but also for foodborne and environmental pathogen detection. The principal challenge is the presence of multiple target proteins in the cell wall, which produces difficulty in the post-SELEX evaluation and binding standardization. Future successful application of aptamers in biosensors for whole-cell detection will be dependent upon the SELEX standardization for complex targets and the post-SELEX characterization of the obtained aptamers and their binding reaction with the cells.

### Nanoparticle-Based Aptasensors.

Recently, several aptasensor platforms were developed that include the use of nanoparticles, particularly gold nanoparticles (AuNPs) and QDs (Fischer et al., 2007; Li et al., 2008). The optical properties of AuNPs are commonly used in colorimetric detection. The surface resonance frequency of AuNPs can be modified, resulting in different detectable colors (Li et al., 2008). The chromatic changes result from the aptamer-functionalized AuNP aggregation after target recognition. Colorimetric assays do not require sophisticated detection apparatus (Balamurugan et al., 2008), and the detection can be performed in solution, avoiding the disadvantages of platform immobilization.

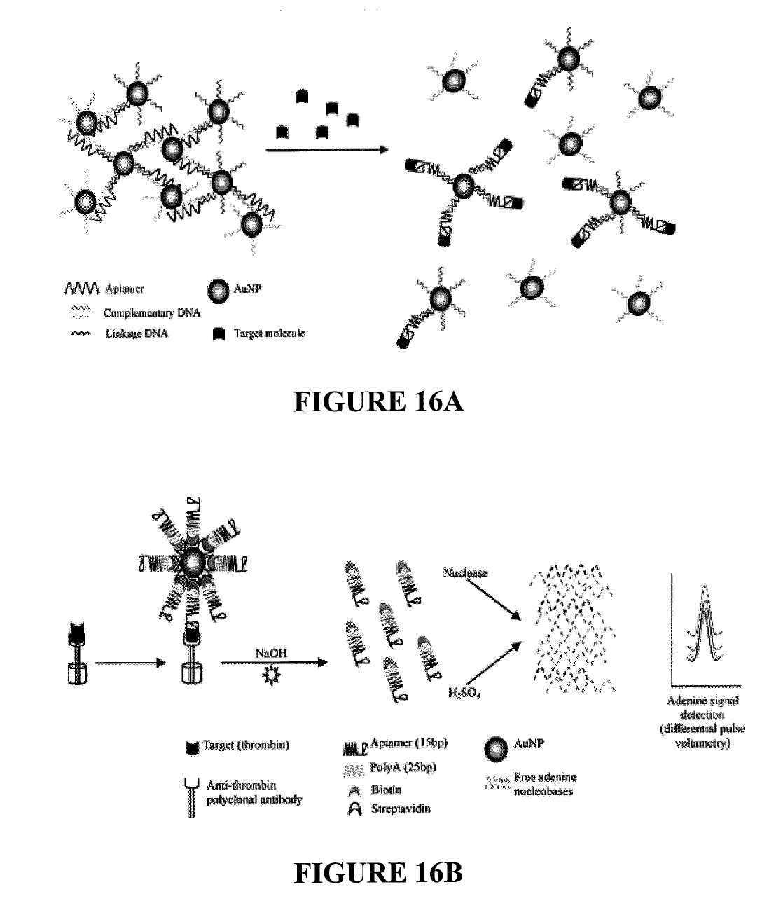

Liu and Lu (2006) developed two solution-based colorimetric detection systems, using AuNPs functionalized with aptamers to detect adenosine and cocaine. AuNP aggregates linked by oligonucleotide sequences containing the specific aptamer were constructed. In the presence of adenosine or cocaine, the aptamer changed its structure to bind to the target molecule. This resulted in disassembly of the aggregates, which produced a change in the color system from purple to red (FIG. 16A). The color change was instantaneous in the presence of 2 mM adenosine and 1 mM cocaine (Liu and Lu, 2006). In a similar system, successful colorimetric detection was described for platelet-derived growth factor (Huang et al., 2005).

AuNPs can also be used as fluorescence quenchers for optical detection. Using thrombin as a detection model, Wang et al. (2008) investigated three different strategies (adsorption, covalent immobilization, and hybridization) for the AuNP surface modification with aptamers. The thiolated aptamer immobilization provided the best results with the highest constant affinity and the most sensitive detection limit (0.14 nM). For detection, after the thiolated aptamer was immobilized onto gold nanoparticles, dye-labeled complementary DNA was hybridized with the aptamer, which resulted in fluorescence quenching. With the addition of thrombin, the aptamer adopted a different conformation in order to bind with the target. Hence, the dye-labeled DNA was released from the AuNP surfaces, producing a detectable fluorescence signal (Wang et al., 2008). Nanoparticles functionalized with bio-barcodes (small oligonucleotide sequences) were used as antibody labels in sandwich detection systems to amplify detection and enhance sensitivity (Nam et al., 2003). The use of AuNP in the amplification of aptamer bio-barcodes has been investigated, using thrombin as target model (He et al., 2007). The proposed platform was based on a sandwich, label-free electrochemical detection.

First, thrombin was captured by polyclonal antibodies immobilized in microtiter plates. The Ab-target complex was then recognized by biotin-polyA-aptamer-AuNP bio-barcodes. The aptamer was then released and collected from the AuNPs. Finally, the modified aptamers with the polyadenine oligonucleotides (bio-barcodes) were degraded by nuclease or acid, and differential pulse voltammetry was used to detect the well-defined adenine signal (FIG. 16B). The thrombin detection limit was 0.1 ng/ml (He et al., 2007). Another sandwich-type assay to detect thrombin was used to develop a microgravimetric quartz crystal microbalance (QCM) platform, using AuNP for the detection. The thiolated capture aptamer was immobilized on the QCM electrode. After incubation with the target (thrombin), the aptamer-thrombin complex was recognized by the AuNPs functionalized with the detection aptamer. The AuNP attachment to the QCM surface provided initial thrombin analysis amplification, resulting in a frequency change. Secondary analysis amplification was obtained with the catalytic enlargement of AuNP in the presence of HAuCl4 and NADH, with a sensitivity of 20 nM. The use of aptamer-functionalized AuNPs as catalytic labels for thrombin analysis amplification in solution was also established (Pavlov et al., 2004).

A sandwich-type assay was developed using platinum nanoparticles (PtNP) functionalized with a detection thrombin aptamer. The capture aptamer was immobilized on a gold electrode. After the aptamer-thrombin complex formation on the gold electrode surface, the secondary aptamer attached to the PtNP recognized the complex, and the PtNP catalyzed the H2O2 reduction process used for amperometric detection. This electrocatalytic nanoparticle system showed an improved sensitivity (1 nM) compared with the previous electrochemical aptasensors used for detecting thrombin (Polsky et al., 2006). Semiconductive nanocrystals (QDs) were widely used as fluorescent labels in several aptasensor applications. Liu et al. (2007) used two aptamer-functionalized QDs, with different fluorescence emission wavelength for simultaneous detection of adenosine and cocaine, containing AuNPs as quenchers. QDs were assembled with AuNPs by the specific aptamer, quenching the QD fluorescence emission. Addition of the targets (adenosine and cocaine) disassembled the aggregates, resulting in increased fluorescence. The targets were simultaneously detected by fluorescence and colorimetric measurements in solution (Liu et al., 2007). QD-aptamer systems have also been used to detect thrombin (Levy et al., 2005; Choi et al., 2006). Hansen et al. (2006) developed an electrochemical aptasensor using QDs to detect thrombin and lysozyme simultaneously. A gold electrode was functionalized with thiolated aptamers for each protein. QD-tagged proteins (CdS for thrombin and PbS for lysozyme) were bound to the corresponding aptamer into the gold substrate. In the presence of the target proteins, the QD-protein complexes were displaced and the remaining nanocrystals were electrochemically detected. The position and size of the corresponding metal peaks (Cd and Pb) in the voltammograms corresponded to the type and amount of the respective protein target (thrombin and lysozyme) (Hansen et al., 2006).

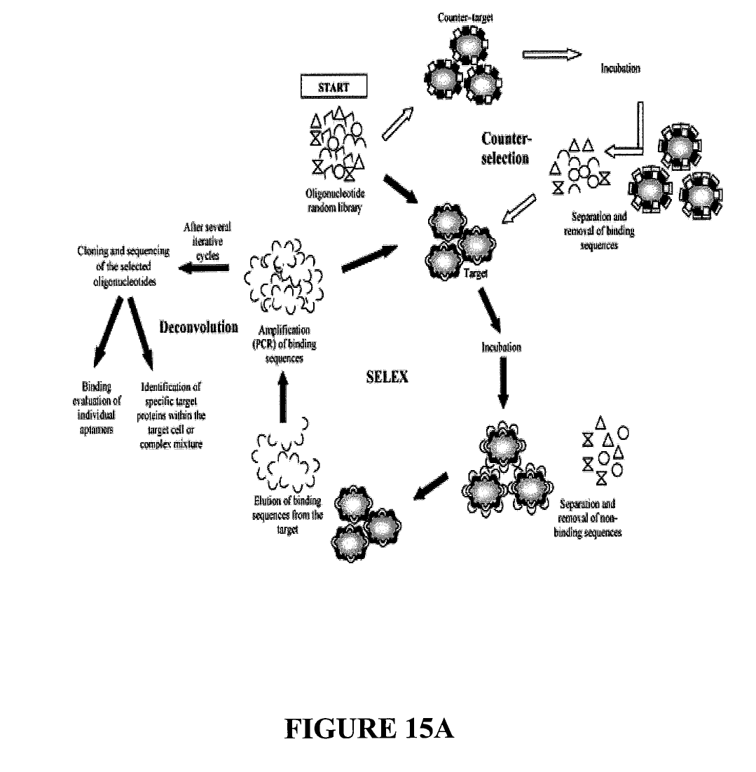

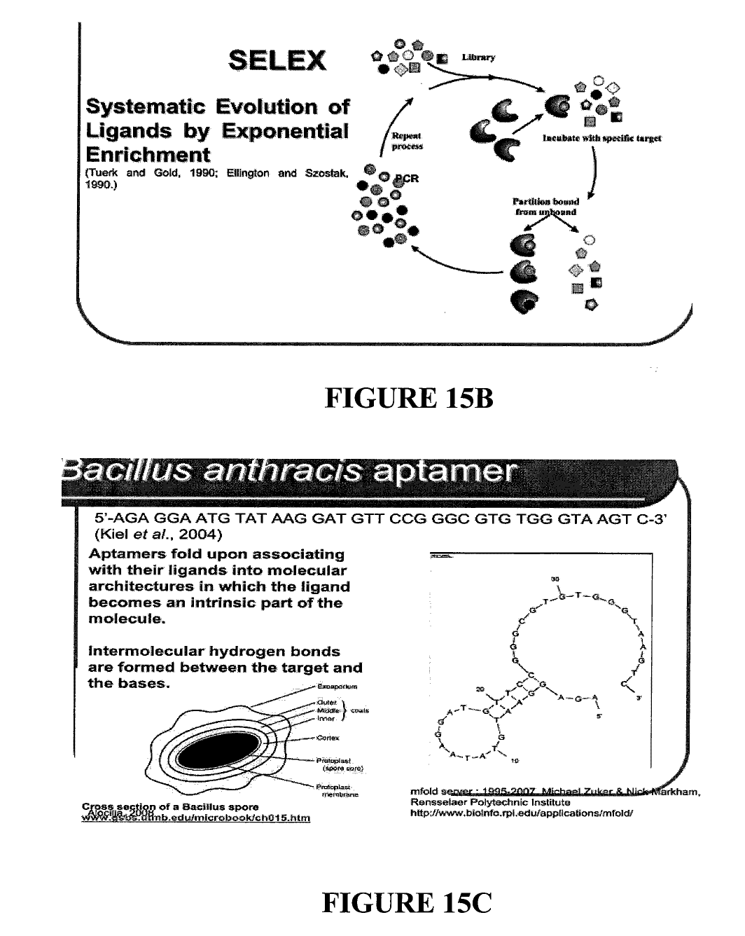

Exemplary schematics and results of aptamer identification of Bacillus anthracis exemplary is shown in FIGS. 15 and 16, Alocilja, et al., Nano-Biosensors for Food Defense, PITTCON 2008, Mar. 2-7, 2008, New Orleans, La., herein incorporated by reference in its entirety.

The inventors contemplated the use of complex-target SELEX (systematic evolution of ligands by exponential enrichment) for isolating specific aptamers against microbial pathogens, for a review see, Edith Torres-Chavolla et al., Biosensors and Bioelectronics, 2008 Nov. 25, herein incorporated by reference in its entirety. These isolated aptamers would be used as probes in embodiments of the present inventions in addition to use in molecular diagnostic platforms as well as infection inhibitor agents. One of the main disadvantages of using a whole-cell target approach in SELEX is that the specific target molecule is initially unknown, which would potentially cause specificity problems and produce difficulty in the post-SELEX optimization of the binding assay between the obtained aptamer sequence and the target. Some alternatives to overcome these issues are contemplated including the use of counter-selection steps with closely related species to the target organism, and the use of deconvolution-SELEX steps to partition the initial aptamer pools against the multiple targets present in cell surfaces.

To date, most of the isolated aptamers against microorganisms were selected for clinical applications. Aptamer application to detect environmental and foodborne pathogens is a promising area of research. Matrix complexity is one of the greatest challenges in molecular diagnostic application to foodborne pathogen detection. Several components regularly present in food samples can produce interference or cross-reaction in both immunology-based and nucleic acid-based detection systems. Therefore, additional isolation, concentration and/or purification of the microbial target is required before molecular detection, which increases the assay time and cost. However, the ability to select aptamers in real complex matrices represents a way to overcome these issues. Several aptasensor platforms for detecting pathogenic proteins were developed, especially for viral proteins detection. Aptasensors for microbial whole-cell identification are a promising and challenging application, with potential advantages over existing immunological whole-cell biosensors.

### Aptamer Development for Pathogenic Microorganisms and Viruses.

Aptamers targeting microbial and viral pathogens were developed for two main purposes: as therapeutics and for pathogen detection. Whole-cell and non-whole-cell targeting approaches were applied in microbial SELEX. Examples of non-whole-cell approaches include viral and bacterial lysates, and cell membrane preparations. Viral protein aptamers were used in the molecular analysis of virus replication and in the development of antiviral agents (Zhang et al., 2004; James, 2007; Jang et al., 2008). A summary of the available reports on microbial targets (including whole-cell, non-whole-cell, and toxin targets) is provided in Table 1. Representative examples of viral aptamers developed for detection or infection inhibition are also included. Whole-cell strategies and targets are discussed in the following section.

Whole-cell targets, Protozoa: African trypanosomes aptamer selection is one of the first reports for microbial whole-cell targets that included the obtained aptamer sequences, the identification of the particular target molecule location, and the aptamer's secondary structure prediction (Homann and Goringer, 1999). Trypanosoma brucei was used as the model organism for African trypanosomes and extracellular parasites. Three different classes of RNA aptamers were selected.

The specific binding site was identified as a protein molecule located in the parasite flagellar pocket. The selected aptamers were not able to specifically identify T. brucei among other trypanosome strains tested (Homann and Goringer, 1999). Further binding analysis and internalization capabilities of the African trypanosomes aptamers were published (Homann and Ulrich, 2001).

Ulrich and collaborators (2002) reported the use of live Trypanosoma cruzi parasites as SELEX targets for the selection of RNA aptamers with inhibitory activity on T. cruzi cell invasion. T. cruzi is an intracellular parasite for which mediating parasite-host cell molecules that play an important role in the cell adhesion and invasion process were identified (laminin, thrombospondin, heparin sulfate, and fibronectin) (Ulrich et al., 2002). During the selection cycles, a selective displacement step was applied to the RNA molecules that bound to the T. cruzi surfaces. The four host cell molecules were incubated with the parasite-aptamer complexes and the displaced RNA molecules were used as starting library for the next selection cycle. The obtained RNA ligands specifically bound to the parasite receptors of the host cell matrix molecules. Also, aptamer classes obtained were able to inhibit T. cruzi invasion in vitro (Ulrich et al., 2002). The development of potential anti-parasitic drugs has been the main application of the trypanosome aptamer research. Post-SELEX optimization has been reported to improve in vivo functionality of the RNA aptamer against African trypanosomes (Adler et al., 2008). 3.1.2. Bacteria Bacillus anthracis Sterne strain spores have also been used as whole-cell targets in SELEX (Bruno and Kiel, 1999; Zhen et al., 2002; Kiel et al., 2004). Further post-SELEX optimization (i.e., characterization of aptamer-spore binding reaction, identification of specific spore target molecules, specificity and sensitivity assays) is necessary in order to use the isolated DNA aptamers in the development of detection platforms.

Stratis-Cullum et al. (2005) reported the use of aptamers for the detection of Campylobacter jejuni whole-cells. In the report, aptamer specificity assays showed no cross-reactivity with Salmonella Typhimurium, but limited cross-reactivity with Escherichia coli O157:H7. However, some cross-reactivity was shown with Helicobacter pylori and Listeria sp. at high concentrations (McMasters and Stratis-Cullum, 2006; Stratis-Cullum et al., 2007) In these studies, neither the SELEX process nor the C. jejuni aptamer sequences were detailed in the report.

Aptamers with potential therapeutic application were identified for Mycobacterium tuberculosis, using a whole-bacterium SELEX strategy (Chen et al., 2007). The selected aptamer sequence, which constituted 30% of the final pool, specifically distinguishes M. tuberculosis cells (H37Rv strain) from M. bovis (the counter-selection cells). The specific target molecules were partially identified as membrane proteins, using a proteinase analysis. M. tuberculosis cells were treated with trypsin and proteinase K before the incubation step with the aptamer sequence. Negative results were obtained for the binding reaction between the treated cells and the aptamer.

This suggests that the binding sites were removed by the proteinase treatment and that they were most likely membrane proteins. This proteinase assay can be used for the initial identification of target molecules in whole-cell SELEX approaches.

Viruses. Whole-cell strategies have also been used to obtain aptamers against viruses. The human influenza A virus RNA aptamers obtained by Gopinath et al. (2006) specifically bound and differentiated strains within subtype N3N2. The binding analysis suggested that the aptamers were specific for the haemagglutinin (HA) membrane glycoprotein of the A/Panama virus strain and were able to distinguish this HA from those of other influenza viruses, including strains of the same subtype N3N2 (A/Aichi strain). Post-SELEX optimization assays included determination of binding kinetics, identification of aptamer minimal RNA motif, and determination of inhibitory effect. The obtained aptamers have potential application in influenza A virus genotyping, and inhibition of HA-mediated membrane fusion (Gopinath, et al., 2006. J. Gen. Virol. 87 (3), 479-487).

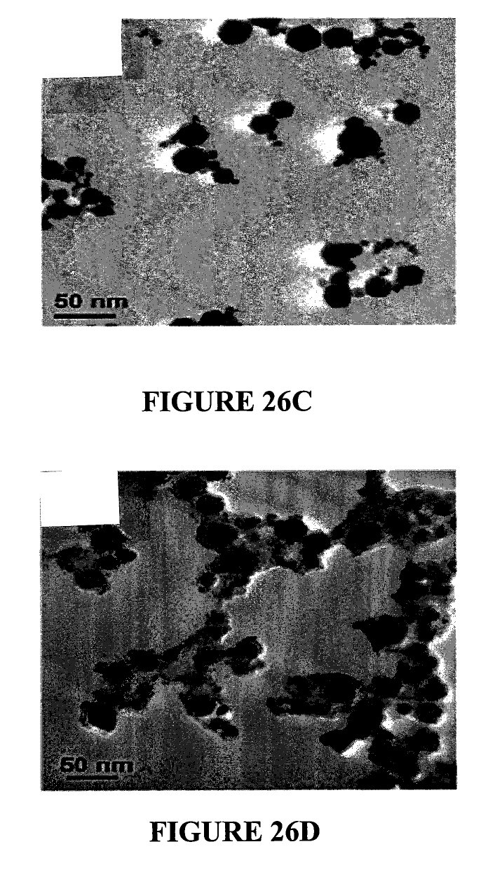

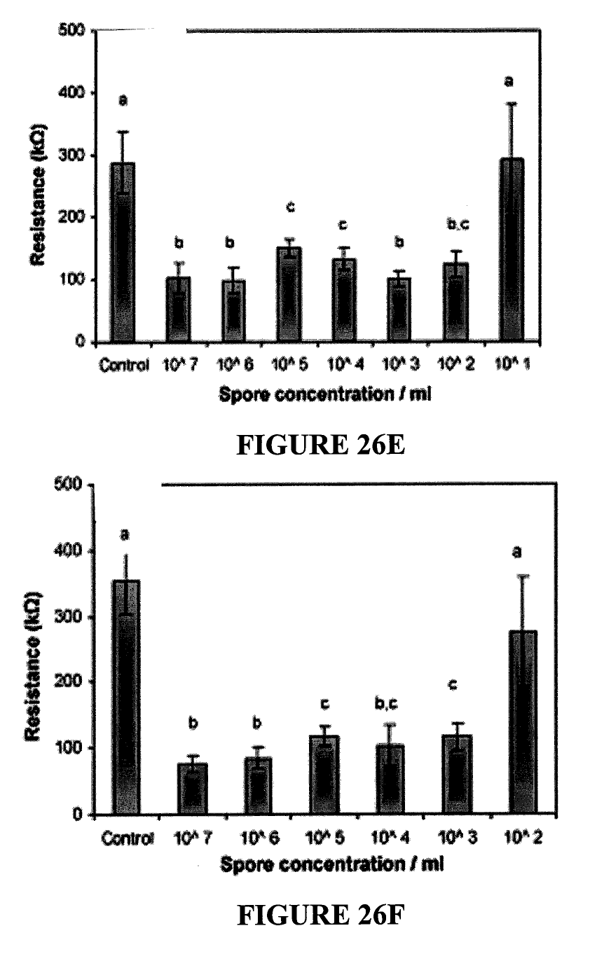

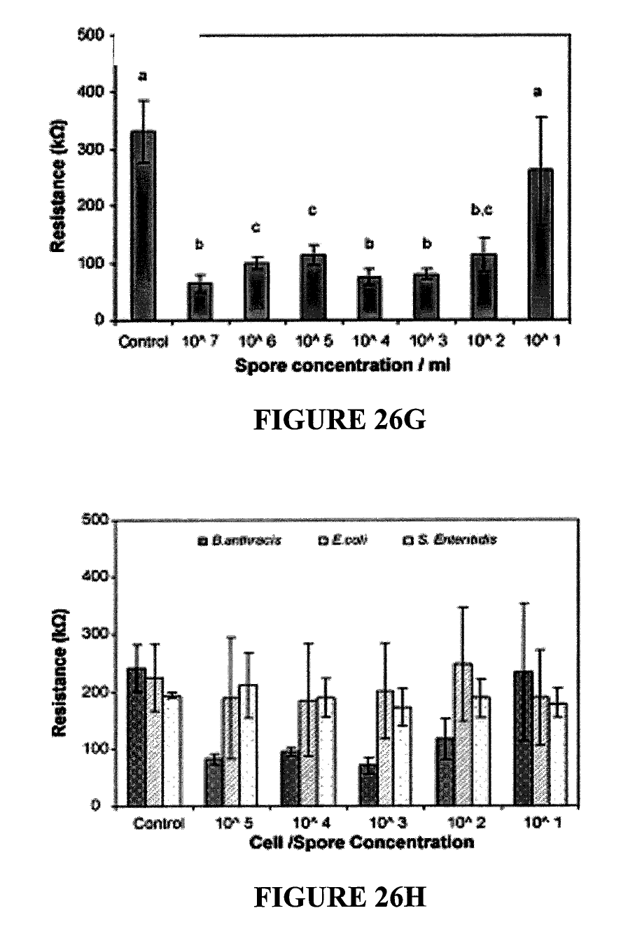

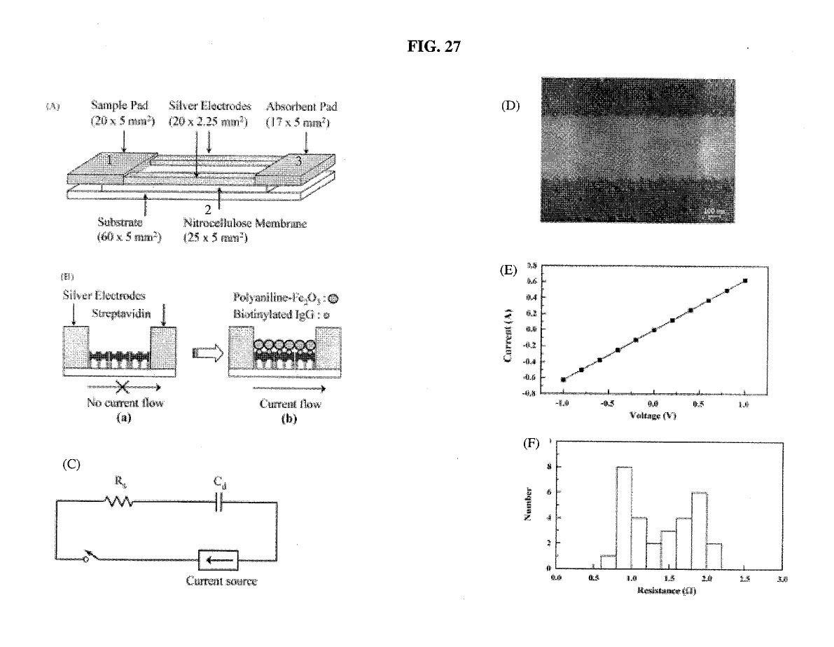

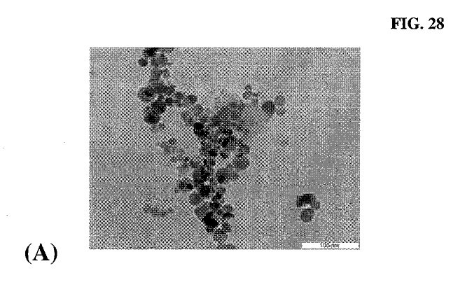

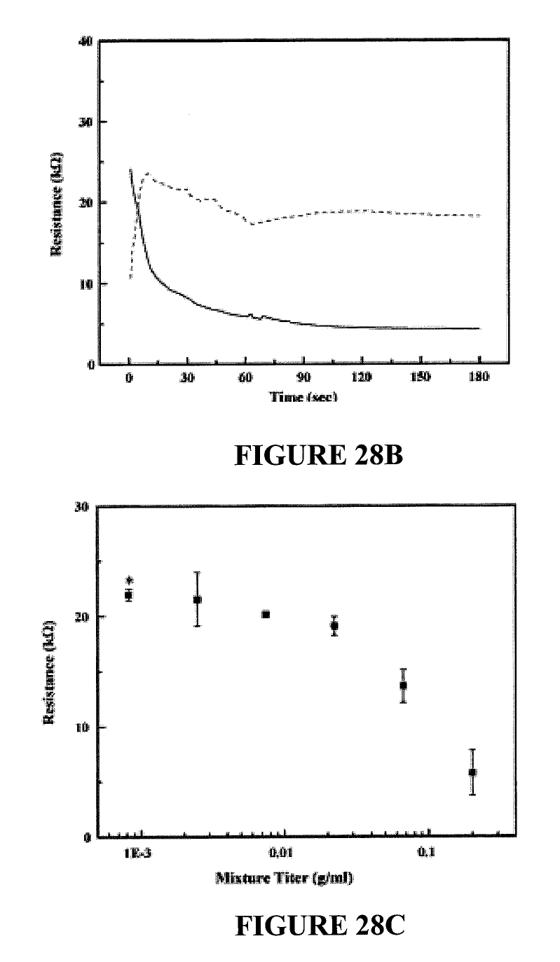

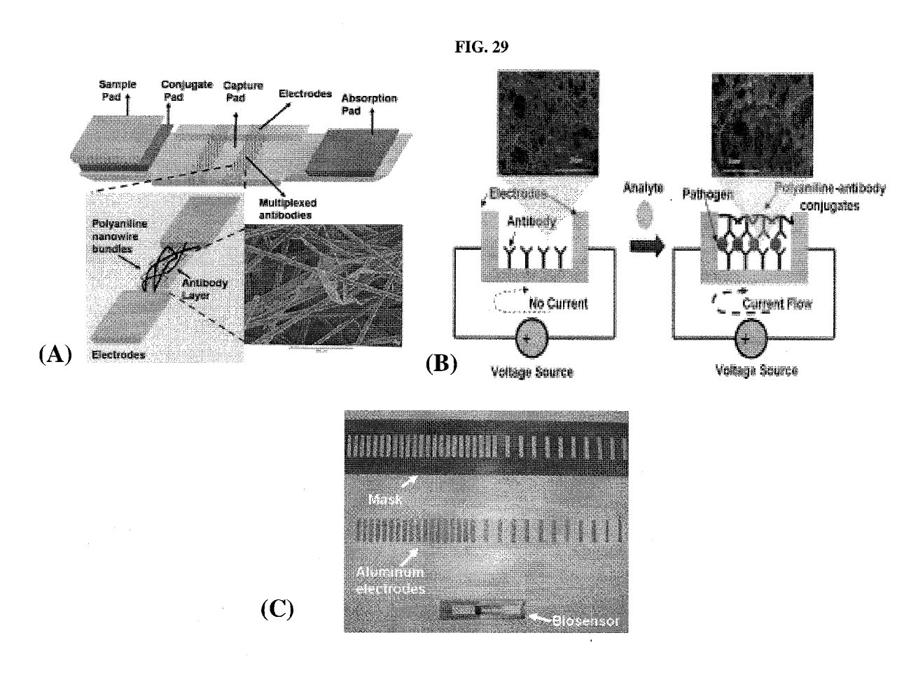

The following presentations and publications by the inventors describing and demonstrating biosensors of the present inventions, (as shown in FIGS. 26-33) are herein incorporated by reference in their entirety:

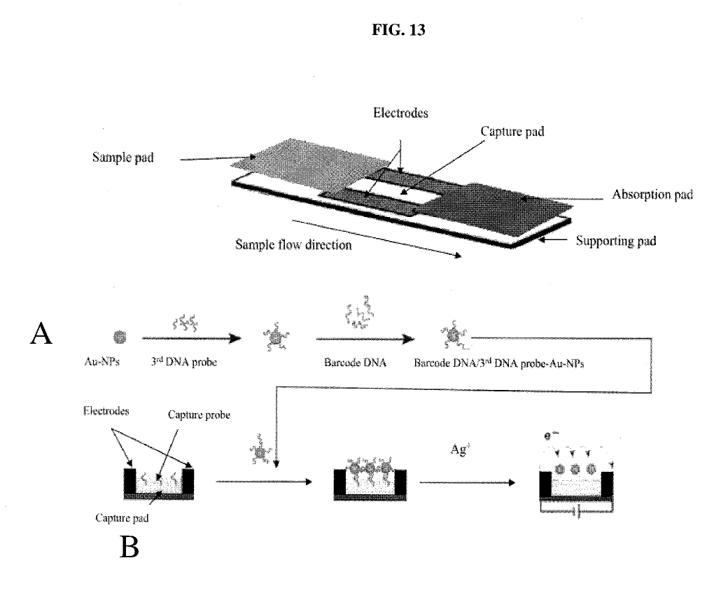

FIG. 13 shows an exemplary solid-phase assay (Detection platform: membrane-based) of the present inventions. This schematic shows a solid-phase assay (Detection platform: membrane-based) with immobilized AuNps conjugated to a DNA probe, wherein the DNA probe comprises a hybridization region for a Barcode DNA, and a method wherein the barcode DNA of the AuNP hybridizes to a region of the capture probe DNA attached to the immobilized capture pad between electrodes. After hybridization, the bound Au particle is coated with Ag (silver ions) for completing an electrical circuit triggering an electrochemical signal, Alocilja, et al., Nano-Biosensors for Food Defense, PITTCON 2008, Mar. 2-7, 2008, New Orleans, La., herein incorporated by reference in its entirety.

A direct-charge transfer (DCT) biosensor was developed for the detection of the foodborne pathogen, Bacillus cereus. The biosensor was fabricated using antibodies as the sensing element and polyaniline nanowire as the molecular electrical transducer. The sensor design consisted of four membrane pads, namely, sample application, conjugate, capture and absorption pads. Two sets of polyclonal antibodies, secondary antibodies conjugated with polyaniline nanowires and capture antibodies were applied to the conjugate and the capture pads of the biosensor, respectively. The detection technique was based on capillary flow action which allowed the liquid sample to move from one membrane to another. The working principle involved antigen-antibody interaction and direct electron charge flow to generate a resistance signal that was being recorded. Detection from sample application to final results was completed in 6 min in a reagentless process. Experiments were conducted to find the best performance of the biosensors by varying polyaniline types and concentrations. Polyaniline protonated with hydrochloric acid, emeraldine salt and polyaniline protonated with perchloric acid were the three kinds of polyaniline used in this study. The biosensor sensitivity in pure cultures of B. cereus was found to be 10(1) to 10(2) CFU/ml. Results indicated that using emeraldine salt at a concentration of 0.25 g/ml gave the best biosensor performance in terms of sensitivity. The biosensor was also found to be specific in detecting the presence of B. cereus in a mixed culture of different Bacillus species and other foodborne pathogens. The speed, sensitivity and ease-of-use of this biosensor make it a promising device for rapid field-based diagnosis towards the protection of our food supply chain. The phenotypic and genotypic similarities between B. cereus and Bacillus anthracis will also allow this biosensor to serve as an excellent model for the detection of B. anthracis. Biosens Bioelectron, 2007, Apr. 15; 22(9-10):2329-36. Epub 2007 Jan. 25.

In addition, the following publications are herein incorporated by reference in their entirety for contemplated use in the compositins and methods of the present inventions: Muhammad-Tahir, et al., Biosens Bioelectron. 2003 May; 18(5-6):813-9; Alocilja, et al., Biosens Bioelectron. 2003 May; 18(5-6):841-6. Review, Younts, et al., J Food Prot. 2003 August; 66(8):1455-8, Mathew, et al., Luminescence. 2004 July-August; 19(4):193-8, Mathew, et al., Biosens Bioelectron. 2005 Feb. 15; 20(8):1656-61, Radke, et al., Biosens Bioelectron. 2005 Feb. 15; 20(8):1662-7, Tahir, et al., Biosens Bioelectron. 2005 Feb. 15; 20(8):1690-5, Kindschy, et al., Biosens Bioelectron. 2005 Apr. 15; 20(10):2163-7, and Pal, et al., Biosens Bioelectron. 2007 Apr. 15; 22(9-10):2329-36. Epub 2007 Jan. 25, Gore, et al., Conf Proc IEEE Eng Med Biol Soc. 2006; Suppl:6489-92.

Additional contemplated embodiments include a biosensor comprising a polyaniline coated magnetic (EAPM) nanoparticle. Specifically, Electrically active polyaniline coated magnetic (EAPM) nanoparticle-based biosensor has been developed for the detection of Bacillus anthracis endospores in contaminated food samples. The 100 nm-diameter EAPM nanoparticles are synthesized from aniline monomer (made electrically active by acid doping) coating the surface of gamma iron oxide cores. The magnetic, electrical, and structural characteristics of the synthesized EAPM nanoparticles were studied using superconducting quantum interference device (SQUID), four-point probe, and transmission electron microscopy (TEM). Room temperature hysteresis of the synthesized nanoparticles shows a saturation magnetization value of 44.1 emu/g. The EAPM nanoparticles are biologically modified to act as an immunomagnetic concentrator of B. anthracis spores from lettuce, ground beef and whole milk samples and are directly applied to a direct-charge transfer biosensor. The detection mechanism of the biosensor depends on the capillary flow of the captured spores on the biosensor surface along with direct-charge transfer across the EAPM nanoparticles. Experimental results indicate that the biosensor is able to detect B. anthracis spores at concentrations as low as 4.2×10(2) spores/ml from the samples. The EAPM-based biosensor detection system is fast and reliable with a total detection time of 16 min. (Pal, et al., Electrically active polyaniline coated magnetic (EAPM) nanoparticle as novel transducer in biosensor for detection of Bacillus anthracis spores in food samples, Biosens Bioelectron. 2009 Jan. 1; 24(5):1437-44. Epub 2008 Aug. 22, herein incorporated by reference in its entirety). Which was enhanced using screen-printing technology and pulse mode measurement technique. Screen-printed silver electrodes were made on a nitrocellulose membrane and the distance between the two electrodes was approximately 550 microm. Resistance of the electrodes had an average of 1.4 Omega with a standard deviation of +/−0.4 Omega. The surface of nitrocellulose membrane was modified by glutaraldehyde to immobilize streptavidin. Biotinylated anti-mouse IgG was conjugated with polyaniline-coated magnetic nanoparticles. Formation of polyaniline-coated magnetic nanoparticles was confirmed by a transmission electron microscope image. The polyaniline was used as an electric signal transducer for the monitoring of the biospecific binding event. An electrical response induced by the streptavidin-biotin interaction was measured by pulse mode measurement. This measurement method reduced the resistance caused by interfacial capacitance. Dose-dependent resistance changes were also successfully analyzed by the pulse mode polymeric wire biosensor. Results showed that the pulse mode measurement technique enhanced the performance of the polyaniline-based polymeric wire biosensor by reducing the interfacial effects. This approach could be helpful in samples with high interfering background materials, such as food and clinical specimens (Yuk J S, Jin J H, Alocilja E C, Rose J B, Performance enhancement of polyaniline-based polymeric wire biosensor, Biosens Bioelectron. 2009 Jan. 1; 24(5):1348-52. Epub 2008 Aug. 19, herein incorporated by reference in its entirety) Descriptions of a polyaniline-based biosensor are also provided in FIGS. 15A-E, Alocilja, et al., Nano-Biosensors for Food Defense, PITTCON 2008, Mar. 2-7, 2008, New Orleans, La., herein incorporated by reference in its entirety).

Additional contemplated embodiments for use in the present inventions include a biosensor comprising a Nano-BEAM-Ab biosensor of the present inventions. An exemplary Nano-BEAM-Ab biosensor of the present inventions are shown in FIG. 16 A-L, Alocilja, et al., Nano-Biosensors for Food Defense, PITTCON 2008, Mar. 2-7, 2008, New Orleans, La., herein incorporated by reference in its entirety.

Additional contemplated embodiments for use in the present inventions include a biosensor comprising a Nano-BEAM-DNA biosensor. An exemplary Nano-BEAM-DNA biosensor of the present inventions, are shown in FIGS. 17A-G, Alocilja, et al., Nano-Biosensors for Food Defense, PITTCON 2008, Mar. 2-7, 2008, New Orleans, La., herein incorporated by reference in its entirety.

The inventors provided compositions and methods of using several types of biosensors for detecting bacterial pathogens, including whole cells, fragments of cells, proteins (antigens) and spores in food samples and animal serum. Examples of these biosensors are called AUNT biosensors and further embodiments of polymeric nanowire biosensors, such as a Polymeric Nanowire-Ab sensor (comprising an electrically active polyaniline coated magnetic (EAPM) nanoparticle); a Nano-BEAM-Ab biosensor; a Nano-BEAM-aptamer biosensor. E. coli O157:H7 were detected by a biosensor of the present inventions on the order of 1 CFU/ml with the bio-barcode assay based on polyaniline-coated magnetic nanoparticles, as described herein, in addition to the ranges shown below.

TABLE 1
Sensitivity and specificity of biosensors of the present
inventions for particular pathogens.

| Target pathogen | Sensitivity Range | | Specificity |
|---|---|---|---|
| E. coli O157:H7 | 10¹ | CFU/ml | specific |
| Generic E. coli | | | |
| Salmonella spp | 10¹ | CFU/ml | specific |
| BVDV | 10² | CCID/ml | specific |
| Bacillus cereus | 10¹-10² | CFU/ml | specific |

Inventions provided herein, describe several types of biosensors and assay methods for detecting the presence of a pathogen and measuring the amount of pathogen from a sample. These include biosensors comprising particulate nanoparticles (i.e. a mixture of nanoparticles, nanoparticles in a solution, and the like) and a solid-phase assay (Detection platform: membrane-based) with immobilized nanoparticles. Specifically, embodiments of AUNT, aptamer, nanowire and nanobeam biosensors, in particular nanoparticles, are described herein. Further, embodiments for enhancing the performance of conducting polymer, (for example, a polyaniline) coated nanowire biosensor, and multiple sensing devices are provided. As opposed to other types of biosensors and test devices such as those shown in Kim et al., Biosensors and Bioelectronics, Volume 14, Issue 12, February 2000, Pages 907-915, and U.S. Pat. No. 5,958,791, Roberts, et al., each of which are herein incorporated by reference. Additionally, a label-free DNA Sensor is provided herein.

The use of microorganisms as biological weapons has long been reported in history. One of the first major attacks occurred in the 14th century with Yersenia pestis during the siege of Kaffa (Inglesby, et al., 2000, JAMA-Journal of the American Medical Association 283 (15), 1963; JAMA-Journal of the American Medical Association 283 (17), 2281-2290). The most recent was the deliberate release of B. anthracis spores through the postal system in the United States in October 2001, resulting in 22 cases of anthrax and 5 deaths (Jernigan et al., 2001, Emerging Infectious Diseases 7(6):933-944). An optimal biological weapon is characterized by its high lethality, easy production in large quantities, environmental stability, and its capability to be readily dispersed into aerosols (i.e. 1-10 μm particle size) (Peruski and Peruski, 2003, Clinical and Diagnostic Laboratory Immunology 10(4):506-513). When reviewed for these characteristics, B. anthracis is the most likely to be used as a biological weapon since the microorganism can form spores that can be easily aerosolized, the spore forms can survive harsh environmental conditions, and inhalational anthrax caused by B. anthracis has high mortality rates nearing 100% (Edwards et al., 2006, Analytical and Bioanalytical Chemistry 384 (1):73-84). Specific identification of the microorganism involves complex and laborious microbiological methods requiring anywhere between 2 and 4 days (Inglesby, 2000, JAMA-Journal of the American Medical Association 283(17):2281-2290). A rapid, onsite, and sensitive, detection system for B. anthracis has become imperative.

Biosensors are contemplated as alternatives to slower and less accurate methods of pathogen detection by providing early identification of such harmful pathogens. Several biosensor technologies using fluorescence resonance energy transfer (FRET), field effect transistors (FET), microcantilevers, porous silicon, quantum dots, and microimpedance were developed for the detection of pathogens (Goldman et al., 2004, Analytical Chemistry 76 (3), 684-688; Gupta et al., 2004, Applied Physics Letters 84 (11), 1976-1978; Mathew and Alocilja, 2005, Biosensors & Bioelectronics 20 (8), 1656-1661; Patolsky et al., 2004, Proceedings of the National Academy of Sciences of the United States of America 101 (39), 14017-14022; Radke and Alocilja, 2004, IEEE Sensors Journal 4 (4), 434-440). Recent trends indicate that the biosensor technology is the fastest growing technology in the field of rapid microbial pathogen detection (Lazcka et al., 2007, Biosensors & Bioelectronics 22(7):1205-1217).

Conducting polymers or polymers with a π-electron backbone have attracted considerable interest over the past two decades due to their unusual electronic properties such as, high electrical conductivity, low ionization potential, high electron affinity, and non-linear optical properties. Such polymers have found a number of practical applications in molecular electronics, electronic displays, telecommunication, electrochemical storing systems and most recently in biosensors (Hohnholz et al., 2005; Kaneto et al., 2007; Ramanavicius et al., 2006; Toyoda et al., 2005). The popularity of conducting polymers in biosensor applications can be attributed to a number of factors, such as their compatibility with biomolecules, efficient electrical charge transfer from biochemical reactions to electronic circuits, their ability to be deposited on electrode surfaces, and the ability to have control over polymer layer thickness, electrical properties, and bio-reagent loading (Ahuja et al., 2007 Biomaterials 28 (5), 791-805; Ryder et al., 1997). Current research interests are driven toward magnetic polymer nanostructures, a new class of materials, where magnetic nanoparticles are embedded in conducting polymer matrices.

These magnetic nanostructures have immense potential for applications in electromagnetic devices, drug targeting, and electromagnetic interface shielding (Asmatulu et al., 2005, Journal of Magnetism and Magnetic Materials 292, 108-119; Boissiere et al., 2006, Journal of Materials Chemistry 16 (12), 1178-1182; El-Tantawy et al., 2004, European Polymer Journal 40 (2), 415-430). They have the advantages of non-corrosiveness, light-weight, mechanical strength, and dielectric tunability along with novel magnetic and optical properties (Poddar et al., 2004, Nanotechnology 15 (10):S570-S574). Polyaniline is the most studied conducting polymer in these multi-component systems due to its unique and controllable electrical and chemical properties, excellent environmental stability, and simple and inexpensive synthetic procedures (chemical and electrochemical) (Stejskal and Gilbert, 2002, Pure and Applied Chemistry 74 (5):857-867). Recent literature shows that different types of magnetic cores and doping agents were used for the synthesis of magnetic polyaniline nanoparticles (Dallas et al., 2006, Nanotechnology 17(19):5019-5026; Jiang et al., 2006, Chinese Journal of Chemistry 24 (12):1804-1809; Li et al., 2006, Colloids and Surfaces A: Physicochemical and Engineering Aspects 276 (1-3):40-44, 2007, Synthetic Metals 157(13-15):575-579; Xue et al., 2006, Synthetic Metals 156 (11-13):833-837; Zhang et al., 2005, Nanotechnology 16 (12):2827-2832). Typical examples of the magnetic cores include iron(II, III) oxide, hydroxyl iron, Ni Zn Ferrite and Li Ni Ferrite. Whereas, hydrochloric acid, β-napthalene sulfonic acid, dodecylbenzoylsulfonic acid, toluene, and phosphoric acid are commonly used doping agents in these synthetic procedures. As reported by previous authors, these magnetic polyaniline nanoparticles have sizes in the range of 30 nm and 5 μm and their magnetization values vary between 0.76 and 181 emu/g. Literature suggests that magnetic micro- and nanoparticles have widespread application as separation tools in the purification of nucleic acids, proteins and peptides, bacteria, and metals, such as copper, because of their ability to quickly agglomerate and resuspend in response to changes in magnetic forces (Liang et al., 2007, Colloid and Polymer Science 285 (11):1193-1199; Park and Chang, 2007, Materials Science & Engineering C: Biomimetic and Supramolecular Systems 27 (5-8):1232-1235). The inventors used electrical and magnetic properties of electrically active polyaniline coated magnetic (EAPM) nanoparticles as a novel tool for magnetic concentration of targets (in one embodiment, nucleic acid targets, in other embodiments protein targets) and signal transduction in biosensors. These EAPM nanoparticles in biosensors can provide several advantages, such as increased surface to volume ratio for biological events owing to their nanoscale dimensions, increased assay kinetics due to close proximity to targets, magnetic manipulation of the nanoparticles, and minimized matrix interference from complex samples (such as food and clinical specimens). EAPM nanoparticle-based direct-charge transfer biosensors for concentration and detection of B. anthracis spores from complex food matrices are described herein. This description includes characterization studies for the synthesized EAPM nanoparticles, the biosensor performance of the nanoparticles in lettuce, whole milk, and ground beef samples, and the specificity of the biosensor in non-target pathogens. The efficiency of the EAPM nanoparticles used in magnetic concentration of B. anthracis spores from the food samples is provided herein.

Polymeric nanowire-Ab sensors are described and shown herein, where tested samples included; Water, Milk, Fruits, strawberries, Vegetables, Alfalfa sprouts, Lettuce, Tomatoes, Ground meat, Cooked foods, Fried rice, Corn.

Nano-BEAM-Ab sensors; advantages include electrical and magnetic properties in biosensor transduction, for achieving faster assay kinetics due to close proximity of conducting polymer coated magnetic nanoparticles to target pathogens, providing increased surface area for biological events to occur for faster kinetics, minimizing matrix interference due to improved separation, capability for magnetically manipulating the conducting polymer coated magnetic nanoparticles, avoiding pre-enrichment and pre-treatment steps, reducing background signals in detection devices etc.

Conducting Polymers for use in the present inventions, such as those described in Ahuja et al., Biomaterials, 2007, Stejskal & Gilbert, Pure Appl. Chem., 2002, 74, 857, Stejskal & Gilbert, Pure Appl. Chem., 2002, 74, 857, each of which are herein incorporated by reference in their entirety, are contemplated for use in addition to using polyaniline as a conducing polymer for nanobeams and nanowires of the present inventions. Contemplated embodiments including doping of the conducting polymer, for example, a polyaniline was doped with protonic acids, such as aqueous hydrochloric acid, to give its conductivity (Muhammad-Tahir and Alocilja, 2003, IEEE Sens. J. 3:345-351, herein incorporated by reference). Further contemplated are magnetic polyanilines for use as both a cell concentrator and a transducer at the same time.

Embodiments of polyaniline-based polymeric wire biosensors are provided. Previous polyaniline-based biosensors have suffered from irreproducibility and unreliability due to manually made electrodes and signal processing (Muhammad-Tahir and Alocilja, 2003, Biosens. Bioelectron. 18:813-819, herein incorporated by reference). Screen-printing technology can be an alternative proposal to fabricate homogeneous and reproducible electrodes. Screen-printed electrodes are produced by printing inks of various types, such as gold, platinum, silver, palladium and graphite. Polyester screens are generally used for printing with patterns designed by the analyst in accordance with the analytical purpose. Thus, the screen-printing technology can provide high-volume production of inexpensive and highly reproducible and disposable low-cost electrodes (Renedo et al., 2007, Talanta 73:202-219; Tudorache and Bala, 2007, Anal. Bioanal. Chem. 388:565-578, all of which are herein incorporated by reference). Further described is performance enhancement of the polyaniline-based polymeric wire biosensor based on screen-printing technology and pulse mode measurement technique by analyzing streptavidin-biotin interactions as a model system. Streptavidin has extraordinarily high affinity toward biotin (Kd≈10-14 to 10-16 M). In addition, they are also unusually stable against heat, denaturants, extreme pH, and to the activity of proteolytic enzymes (Laitinen et al., 2007, Trends Biotechnol. 25:269-277, herein incorporated by reference). Screen-printed silver electrodes were fabricated on nitrocellulose (NC) membranes, which had a uniform space between electrodes. The NC membranes were modified with glutaraldehyde for use as a crosslinker in the chemical immobilization. Biotinylated anti-mouse IgG was conjugated with polyaniline-coated magnetic nanoparticles. Resistance changes induced by the streptavidin-biotin interaction were investigated by pulse and non-pulse mode measurements. Dose-dependent responses according to various concentrations of biotinylated IgG conjugated with polyaniline coated magnetic nanoparticles were analyzed by the pulse mode measurement technique.

In particular, demonstrating performance enhancement of a polyaniline-based biosensor is provided herein, using screen-printing technology and pulse mode measurement technique. Screen-printed silver electrodes were made on a nitrocellulose membrane and the distance between the two electrodes was approximately 550 μm. Resistance of the electrodes had an average of 1.4Ω with a standard deviation of ±0.4Ω. The surface of nitrocellulose membrane was modified by glutaraldehyde to immobilize streptavidin. Biotinylated anti-mouse IgG was conjugated with polyaniline-coated magnetic nanoparticles. Formation of polyaniline-coated magnetic nanoparticles was confirmed by a transmission electron microscope image. The polyaniline was used as an electric signal transducer for the monitoring of the biospecific binding event. An electrical response induced by the streptavidin-biotin interaction was measured by pulse mode measurement. This measurement method reduced the resistance caused by interfacial capacitance. Dose-dependent resistance changes were also successfully analyzed by the pulse mode polymeric wire biosensor.

Results showed that the pulse mode measurement technique enhanced the performance of the polyaniline-based polymeric wire biosensor by reducing the interfacial effects. This approach could be helpful in samples with high interfering background materials, such as food and clinical specimens. See, Example IV.

Embodiments of electrically active polyaniline coated magnetic (EAPM) nanoparticle-based biosensor was developed for the detection of Bacillus anthracis endospores in contaminated food samples. The 100 nm-diameter EAPM nanoparticles are synthesized from aniline monomer (made electrically active by acid doping) coating the surface of gamma iron oxide cores. The magnetic, electrical, and structural characteristics of the synthesized EAPM nanoparticles were studied using superconducting quantum interference device (SQUID), four-point probe, and transmission electron microscopy (TEM). Room temperature hysteresis of the synthesized nanoparticles shows a saturation magnetization value of 44.1 emu/g. The EAPM nanoparticles are biologically modified to act as an immunomagnetic concentrator of B. anthracis spores from lettuce, ground beef and whole milk samples and are directly applied to a direct-charge transfer biosensor. The detection mechanism of the biosensor depends on the capillary flow of the captured spores on the biosensor surface along with direct-charge transfer across the EAPM nanoparticles. Experimental results indicate that the biosensor is able to detect B. anthracis spores at concentrations as low as 4.2×10² spores/ml from the samples. The EAPM-based biosensor detection system is fast and reliable with a total detection time of 16 min.

Physical adsorption has been used for bio-modification of the EAPM nanoparticles with IgG molecules because the conjugation procedure is simple and allows IgG molecules to retain their activity and conformation (Zhou et al., 2004,). The UV spectrum of pure anti-B. anthracis IgG (150 μg/ml) molecules and the unreacted IgG molecules in the supernatant after magnetic separation of immuno-EAPMs was measured by a UV-vis-NIR scanning spectrophotometer (Supplemental Information, FIG. S1). Literature suggests that IgG adsorption occurs preferentially through Fc fragment of the molecule (Buijs et al., 1996).

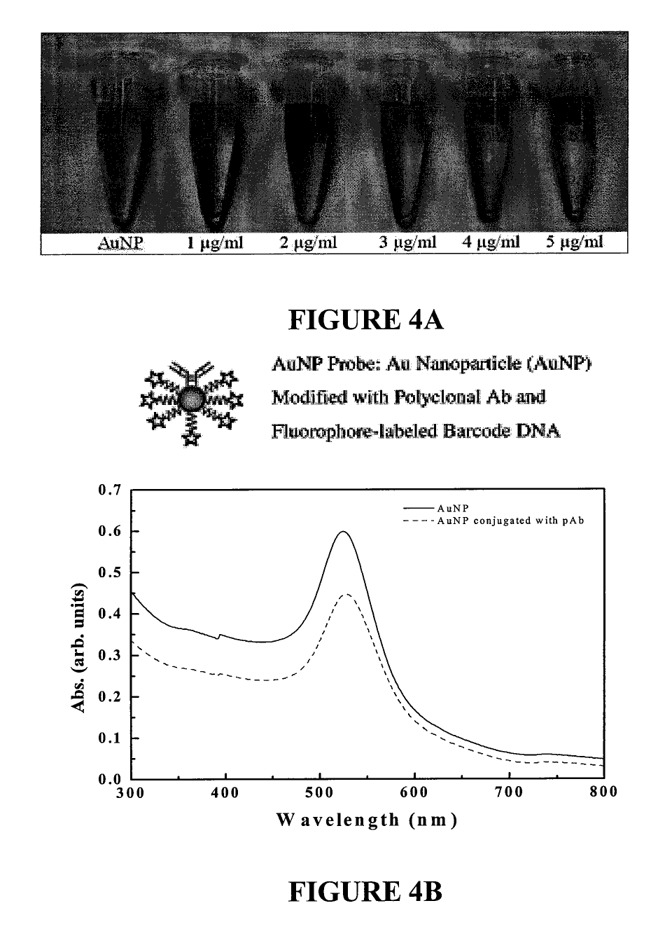

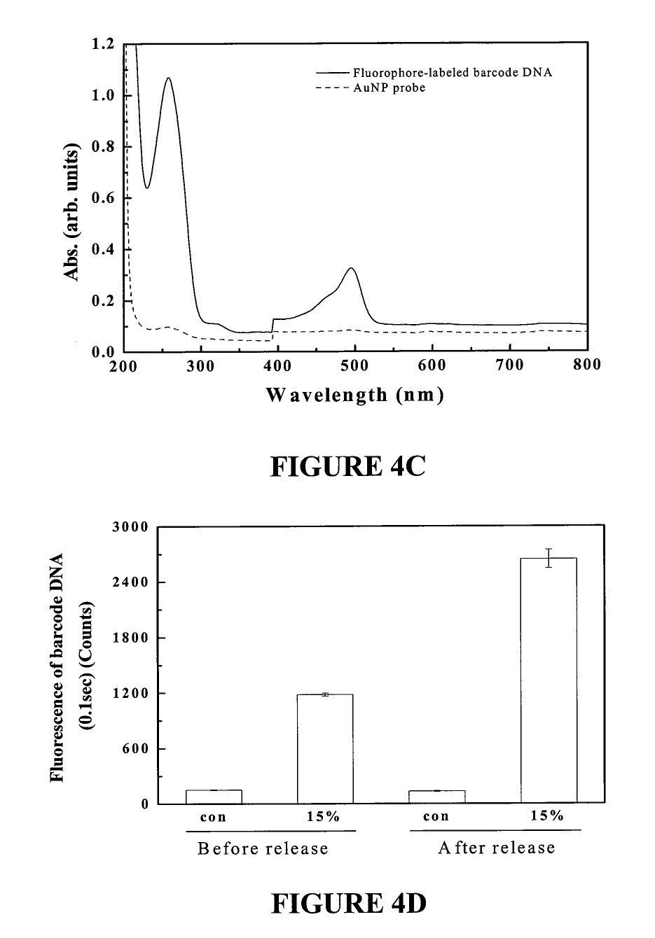

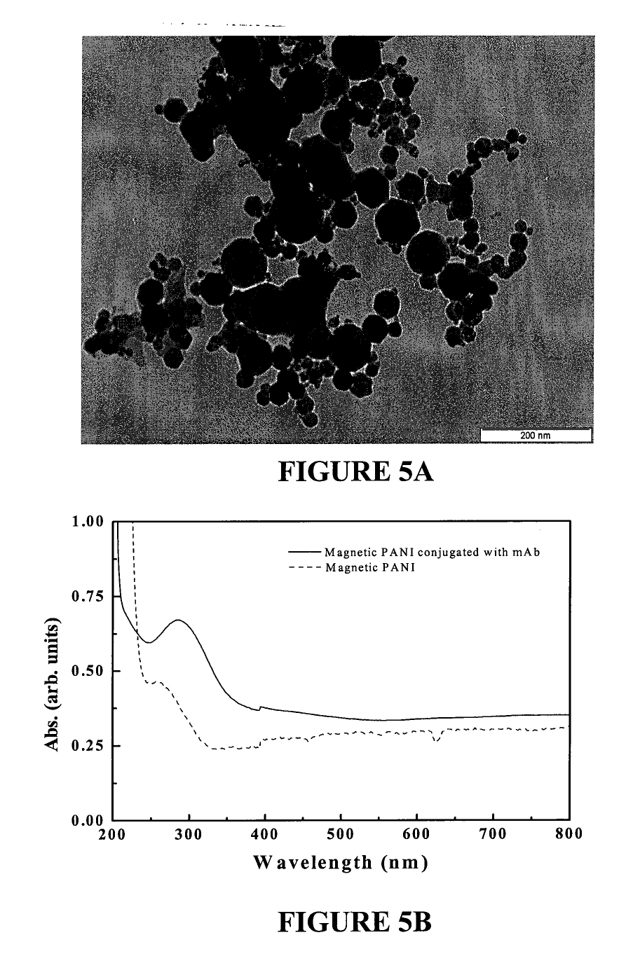

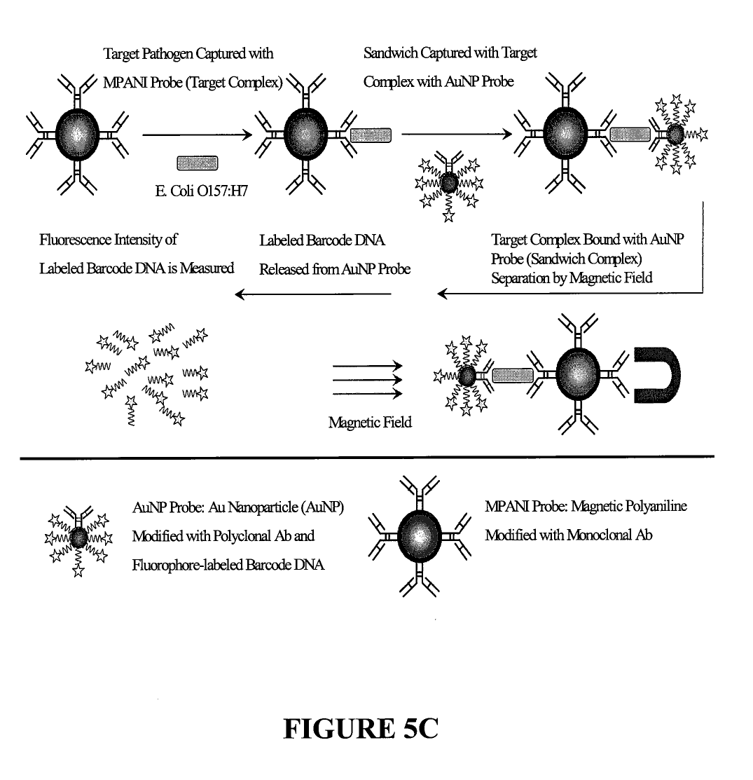

Hence, electrostatic interactions between negatively charged Fc fragment of IgG molecules and positively charged polymer surfaces in the EAPM nanoparticles might play a significant role in the adsorption process. The immobilization of polyclonal goat anti-B. anthracis IgG on the biosensor capture pad was detected by secondary FITC anti-goat IgG and confirmed by fluorescent images obtained from a laser scanning confocal microscope (Supplemental Information, FIG. S2). FIG. 4a-c show the average resistance readings measured with the EAPM nanoparticle-based direct-charge transfer biosensor in lettuce, whole milk, and ground beef samples inoculated with B. anthracis spores. The average resistance signals obtained from three replicates were plotted for the control and the food samples, contaminated with spore concentrations ranging from 4.2×10¹ to 4.2×10⁷ spores/ml.

Embodiments for providing a Multiplexed Biosensor Based on Biomolecular Nanowires is provided, Liu, et al., IEEE International Symposium on Circuits and Systems 18-21 May 2008, IEEE International Symposium on Circuits and Systems, pp. 2006-2009, Seattle, USA, 2008, herein incorporated by reference. Described herein is the fabrication and characterization of a multiplexed biosensor based on molecular bio-wires that can be used for detecting multiple pathogens in a biological sample. At the core of the proposed device is a biosensor that operates by converting binding events between antigen and antibody into a measurable electrical signal using polyaniline nanowires as transducers. By mixing and patterning antibodies at different spatial locations of the biosensor, the response of the biosensor can be configured to detect the presence of either one of several pathogens present in the analyte, thus making it ideal for rapid environment screening applications. Experiments using the biosensor array specific to B. cereus and E. coli bacteria validate the functionality of the proposed multiplexed architecture.

Due to its excellent stability in different solutions, good electronic properties, and strong biomolecular interactions [Imisides, et al., Chemtech, vol. 26, no. 1, pp. 9C25, 1996], polyaniline (PANI) has been also extensively studied in the application of electrochemical biosensors. PANI has been reported to be used in potentiometric glucose biosensor Pan, et al., Sensors and Actuators B: Chem. 2, pp. 325C330, 2004] and amino acid biosensor Singh, et al., Sensor and Actuators B: Chem, vol. 115, Iss. 1, pp. 534-541, 2006]. Several studies were reported using conducting PANI as a label in the immnuosensors. One such immunosensor which has been used in this paper was introduced in [Muhammad-Tahir, et al., IEEE Sensors Journal, vol. 5, pp. 757-762, 2005], and has been shown to achieve a detection limit of 10³ cell culture infective dose per milliliter (CCID/ml) of Bovine Viral Diarrhea Virus (BVDV) antigens in approximately 6 min. In literature, different architectures of multi-array biosensors were proposed. One of the popular detection mechanisms relies on the microarray technology and uses optical readout for detecting hybridization events [Wiese, et al., Clinical Chemistry, vol. 47:1451-1457, 2001], [Taitt, et al., Microbial Ecology, vol. 47, No. 2, pp. 175-185(11), February 2004]. Another multi-array device based on a lateral flow immunosensor has been reported [Malamud, et al., “Point Detection of Pathogens in Oral Samples,” Proceeding of a Symposium on Saliva/Oral-fluid-based Diagnostic Markers of Disease, Mar. 12, 2004] where multiple target-capture molecules were immobilized along the flow of the analyte. The technique, however, suffers from the interference of preceding target capture zones and differences in signal intensity that decays along direction of the flow. In [Gao, et al., Biomedical Microdevices, vol. 7, No. 4, pp. 301-312(12), December 2005] an electrokinetically-controlled heterogeneous immunoassay was developed for detecting multiple pathogens. Specificity experiments were reported for three different pathogens where the detection method used an optical device. However, if the objective of the biosensor is to detect the presence of any of several possible pathogens in a sample, a simplified architecture consisting of a mixture of antibodies could be used as the recognition layer. The mixture in conjunction with an electrical transducer can form an OR logical operation such that a signal is produced only in response to the presence of one or more target pathogens. The mixture of antibodies could be then patterned spatially across the biosensor to form a multiplexed architecture.

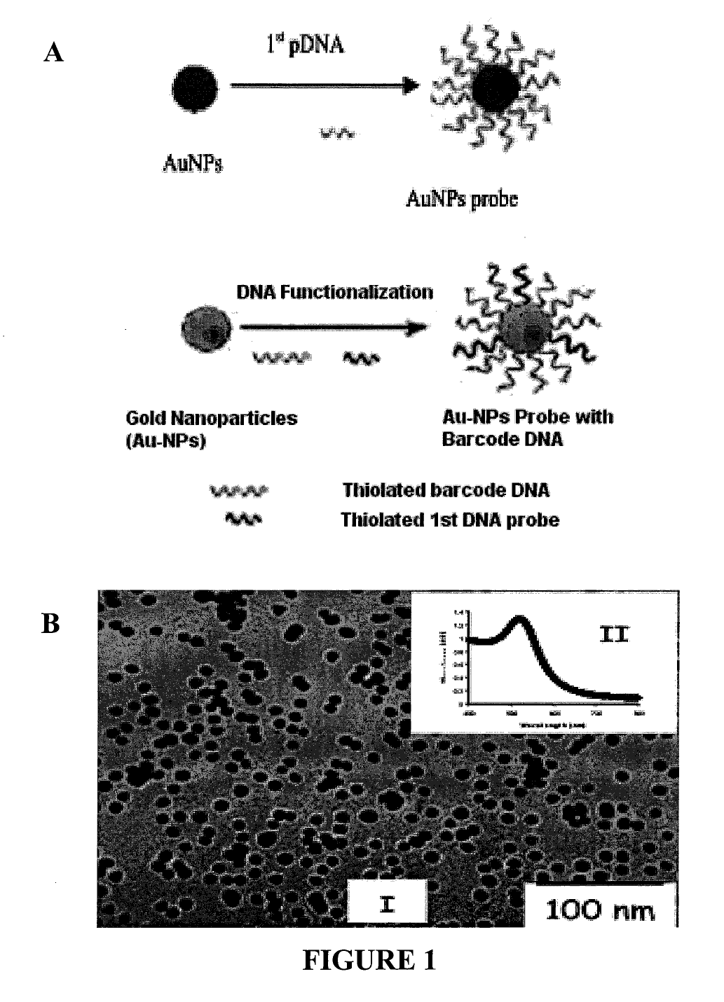

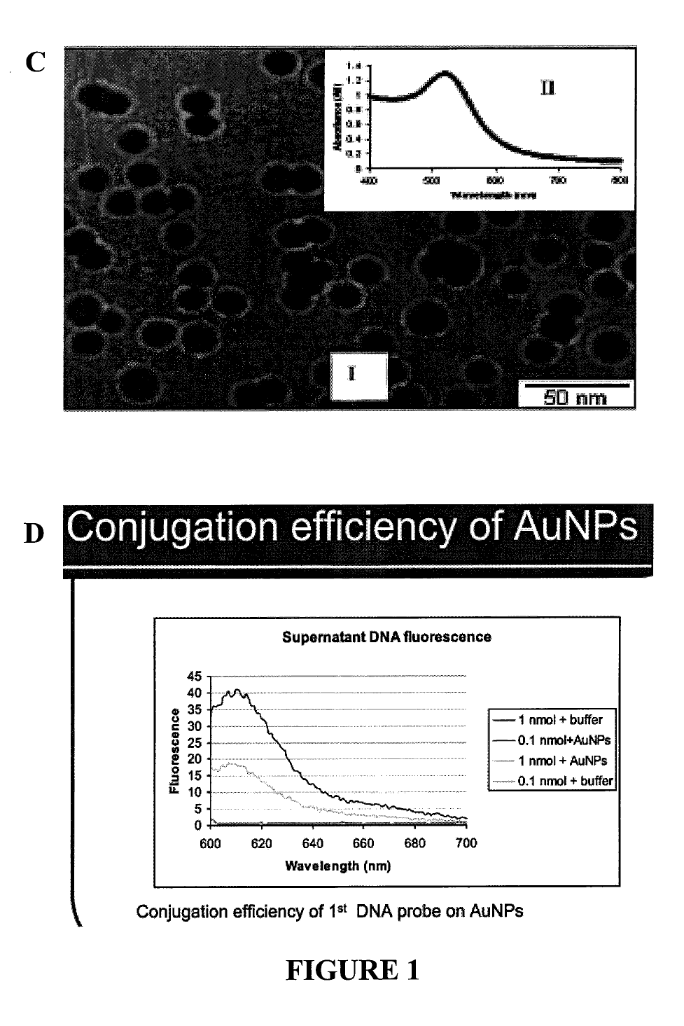

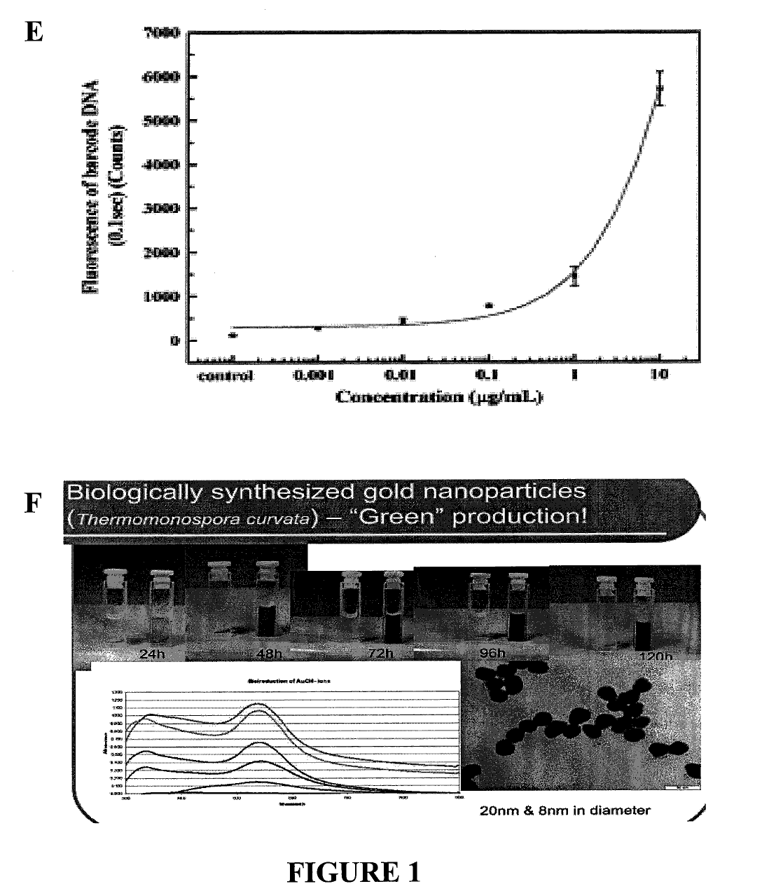

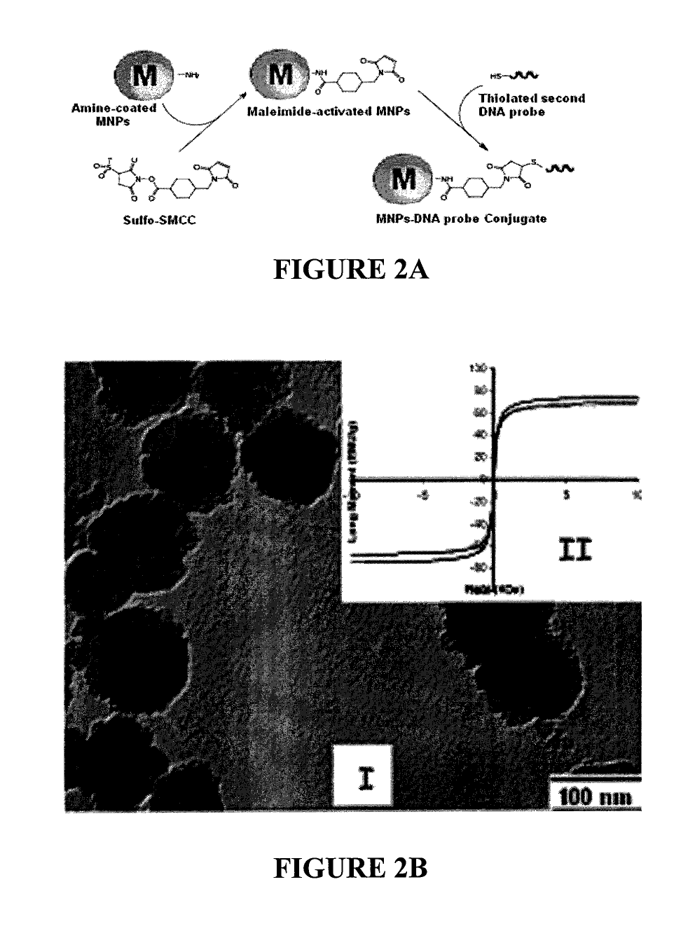

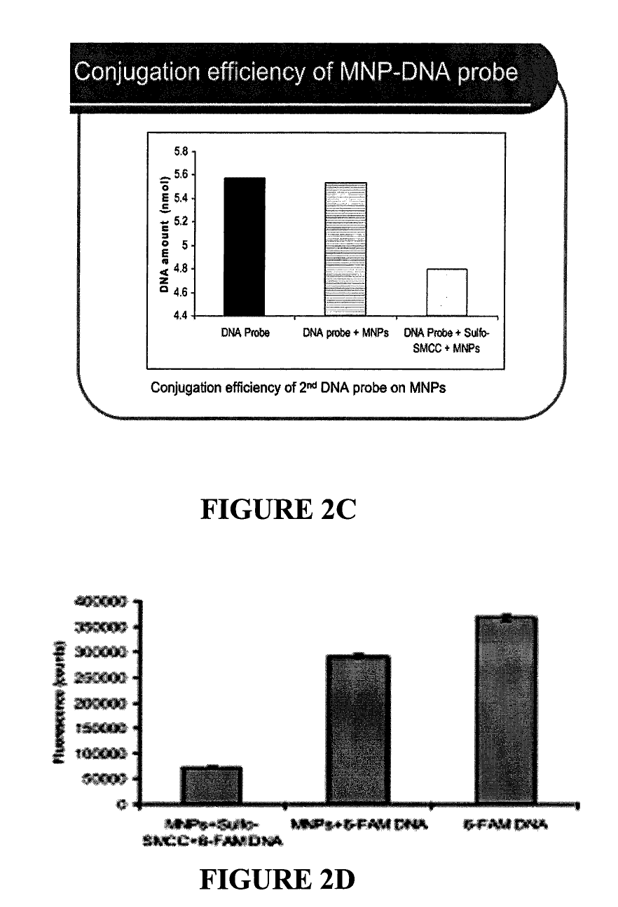

Biosensor Principle The architecture of a multiplexed biosensor is shown in FIG. 1. It consists of four pads: sample pad, conjugate pad, capture pad, and absorption pad. The capture pad, or the area that immobilizes the recognition elements consists of two strips of antibody mixture layers. The SEM image of the polyaniline antibody conjugation is also shown in the sub-image of FIG. 1, where polyaniline nanowires appear to be rod-shape. The principle of operation of a single strip (single antibody) biosensor is illustrated in FIG. 10, which shows a cross-sectional view of the antibody strip. There is an open channel between two electrodes across capture pad before any analyte is applied on the sample pad. Immediately after the analyte is applied to the sample pad, the solution containing the antigen flows to the conjugate pad, dissolves with the polyaniline-labeled antibody (Ab-P) and forms an antigen-antibody-polyaniline complex. The complex is then transported by capillary force into the capture pad containing the immobilized antibodies. If the antigens bind with their specific antibodies on the capture pad, the second antigen-antibody reaction forms a sandwich (FIG. 2).

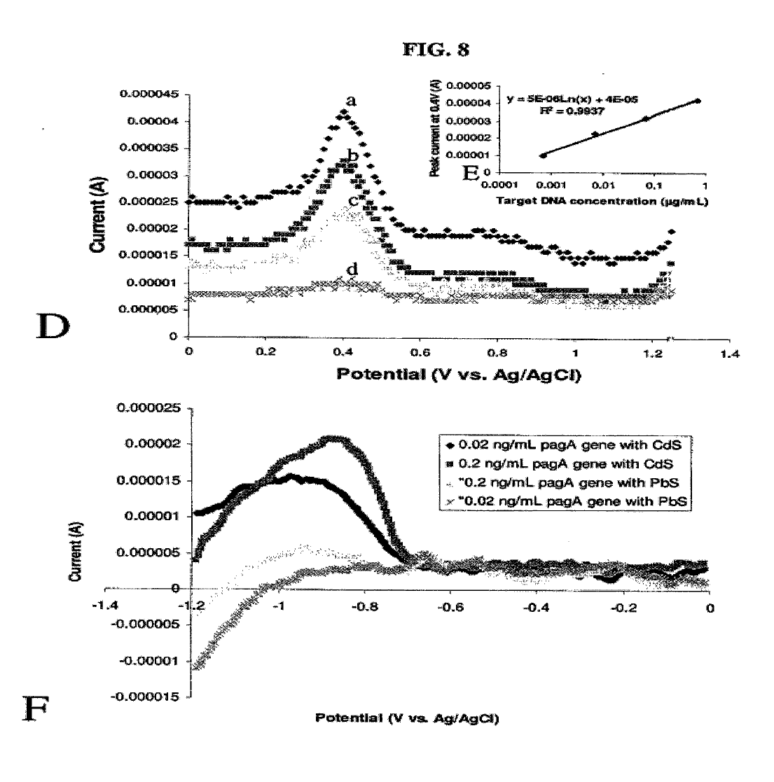

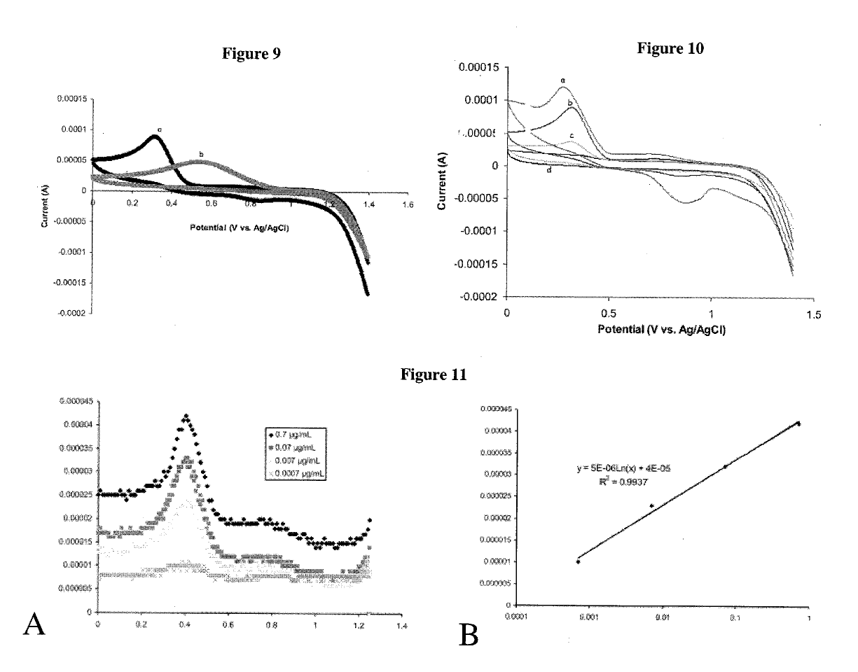

The polyaniline nanowires extend out to bridge adjacent cells and lead to the conductance change between two electrodes (FIG. 9 and FIG. 10). The conductance change is determined by the number of antigen-antibody bindings, which is related to the antigen concentration in the sample. The unbound non-target organisms are subsequently separated by capillary flow to the absorption membrane. The conductance change is sensed as an electrical signal (current) across the electrodes. FIG. 2 also show the SEM image of the capture pad before and after the analyte with pathogen has been applied. The change in material texture can be observed, which is attributed to the formation of the antibody-antigen-antibody-polyaniline complex connecting the electrodes.

Further embodiments show use of the biosensors of the present inventions for detection of virus in serum samples Okafor, Fabrication of a Novel Conductometric Biosensor for Detecting Mycobacterium avium subsp. paratuberculosis Antibodies Sensors 2008, 8, 6015-6025. In particular, Johne's disease (JD) is one of the most costly bacterial diseases in cattle. In the U.S., economic losses from the disease were estimated to exceed $1,500,000,000 per year, mainly from the effects of reduced milk production. Current diagnostic tests for JD are laboratory based and many of those tests require specialized equipment and training. Development of rapid and inexpensive diagnostic assays, which are adapted for point-of-care applications, would aid in the control of JD. In this study, a polyaniline (Pani)-based conductometric biosensor, in an immunomigration format, was fabricated for the detection of serum antibody (IgG) against the causal organism of JD, Mycobacterium avium subsp. paratuberculosis (MAP). Immobilized Mycobacterium avium purified proteins in the capture membrane were used to detect MAP IgG, previously bound with Pani/anti-bovine IgG* conjugate in the conjugate membrane. After detection, the Pani in the sandwiched captured complex bridges an electrical circuit between the silver electrodes, flanking the capture membrane. The electrical conductance, caused by Pani, was measured as drop in electrical resistance. Testing of the biosensor with known JD positive and negative serum samples demonstrated a significant difference in the mean resistance observed between the groups. This proof-of-concept study demonstrated that a conductometric biosensor could detect MAP IgG in 2 minutes. The biosensor's speed of detection and the equipment involved would, among other things, support its application towards the various point-of-care opportunities aimed at JD management and control.

Johne's disease (JD) is a chronic gastrointestinal disease of ruminants caused by Mycobacterium avium subsp. paratuberculosis (MAP). JD causes significant economic losses in the cattle industry. In the U.S., economic losses from the disease were estimated to exceed $1,500,000,000 per year [Stabel, Symposium: Biosecurity and disease—Johne's disease: a hidden threat. J. Dairy Sci.1998, 81:283-288.], mainly from the effects of reduced milk production [Losinger, J. Dairy Res. 2005, 72:425-432.]. Additional sources of losses are unrealized income related to premature culling of cattle, reduced meat quantity at slaughter and animal death. Although there is evidence that MAP may be associated with Crohn's disease in humans, MAP is not currently recognized as a zoonotic pathogen [Grant, J. Appl. Microbiol. 2005, 98:1282-1293]. Economic losses from JD and the concern that MAP may be a zoonotic pathogen have increased the urgency to control the spread of MAP in domestic animals.

Effective control of JD has been challenging. Limitations in currently available diagnostic tests contribute to this challenge. Diagnosis of JD is aimed at detecting MAP or its DNA in feces, tissues, and occasionally milk; or by detecting an immune response against MAP. Currently, bacterial culture is most commonly used in MAP detection [Whitlock, et al., Vet. Microbiol. 2000, 77:387-398]. Other commonly used methods to detect MAP or to detect infection with MAP include polymerase chain reaction (PCR) for detection of MAP DNA [Sevilla, et al., Rev. Sci. Tech. 2005, 24:1061-1066] and enzyme-linked immunosorbent assay (ELISA) for detection of antibody against MAP (IgG) [Kalis, et al., J. Vet. Diagn. Invest. 2002, 14:219-224]. However, bacterial culture is expensive and requires 7-12 weeks for completion [Kalis, et al., J. Vet. Diagn. Invest. 1999, 11:345-351, Whitlock, et al., Vet. Microbiol. 2000, 77:387-398]; PCR and ELISA require specialized equipment and training. These currently used diagnostic tests may not be easily adapted for on-site diagnosis and are not readily accessible to some developing countries. The development of new JD diagnostic assays, which are adaptable to the field and are potentially useful in point-of-care applications, would be beneficial in furthering JD control efforts.

Biosensors are among the new growing pathogen detection or disease diagnostic assays.

A biosensor is an analytical device that contains a transducer, integrated with or placed close to a biological sensing element (BSE) (i.e. antibody) such that a specific biological recognition (i.e. antigen-antibody binding) reaction produces a measurable signal change in a physicochemical detector component [Lazcka, et al., Biosens. Bioelectron. 2007, 22:1205-1217]. A biosensor can be classified based on either the BSE or the transducer components and sometimes a combination of both. Examples of classification based on BSE include antibody-based, DNA-based, enzyme-based, and antigen-based biosensors. Examples of classification based on transducer include resonant, optical, thermal, ion-sensitive field effect transistors (ISFETs), and electrochemical biosensors. Electrochemical biosensors are further classified as amperometric, potentiometric, and conductometric biosensors.

A conductometric biosensor measures electrical conductance/resistance as its signal change. There has been a considerable interest in using conductive polymers (polyaniline, polypyrrole, polyacetylene, and polythiophene) in the development of conductometric biosensors [Hoa, et al., Anal. Chem. 1992, 64:2645-2646, Sergeyeva, Sensor Actuat. B-Chem. 1996, 34:283-288]. Conductive polymers are transducers in conductometric biosensors. Polyaniline (Pani) has been among the most extensively used conductive polymers, due to its strong bio-molecular interactions [Imisides, et al., Chemtech. 1996, 26:19-25], excellent environmental stability, and good conductivity [Syed, et al., Talanta. 1991, 38:815-837]. In a conductometric biosensor, Pani is placed close to or integrated with the biological element (i.e. antibody) such that Pani relays any antigen-antibody binding as a measured electrical quantity. With increased necessity for rapid detection assays in recent times, conductometric biosensors were applied in various biological and biomedical sciences. The applications include determination of glucose and urea in blood [Shulga, et al., Biosens. Bioelectron.1994, 9:217-223.], heavy metal ions and pesticides in water [Chouteau, et al., Biosens. Bioelectron. 2005, 21:273-281], and detection of E. coli O157:H7 [Muhammad-Tahir, et al., IEEE Sens. J. 2003, 3:345-351], Bacillus cereus [Pal, et al., Biosens. Bioelectron. 2007, 22:2329-2336], and Bovine viral diarrhea virus [Muhammad-Tahir, et al., IEEE Sens. J. 2005, 5:757-762]. However, this relatively new assay has not been applied towards JD diagnosis. The development of a biosensor as a rapid, inexpensive, miniaturized, and field-based JD diagnostic assay would support more frequent and widespread testing of animals.

The objective of showing this example was to fabricate and test a conductometric biosensor for detecting IgG in sera from cattle that reacts with MAP. The basic architecture of the biosensor is based on previous publications [Muhammad-Tahir, et al., IEEE Sens. J. 2003, 3:345-351, Pal, et al., Biosens. Bioelectron. 2007, 22:2329-2336, Muhammad-Tahir, et al., IEEE Sens. J. 2005, 5:757-762], however the detection principle is a unique variation. Unlike previous biosensors, which were fabricated to detect bacterial or viral organisms, the biosensor in this study was designed to detect antibodies to a bacterium. Optimization of the fabricated biosensor for JD diagnosis would support various point-of-care applications and frequent testing of animals especially at the point-of-sale, thus guiding the making of management decisions that would improve JD control.

D. Nanoporous Silicon-Based DNA Biosensor for the Detection of Salmonella Enteritidis.

Embodiments for use in biosensors of the present inventions include a Label-Free DNA Sensor on Nanoporous Silicon-Polypyrrole Chip for Monitoring Salmonella Species are also provided herein (Zhang and Alocilja, Characterization of Nanoporous Silicon-Based DNA Biosensor for the Detection of Salmonella Enteritidis, IEEE SENSORS JOURNAL, VOL. 8, NO. 6, JUNE 2008 and Jin, et al., IEEE SENSORS JOURNAL, VOL. 8, NO. 6, JUNE 2008, herein incorporated in its entirety). Label-free detection or fabrication of PPy-based biosensor was used as a PPy-based label-free biosensor for monitoring Salmonella spp, see Example IX.

POROUS SILICON (PS) structure is contemplated to provide at least three major advantages for biosensor development. Large surface-to-volume ratio and rough surface for biological immobilization can improve the sensitivity when the PS surface is used as a sensing electrode of the sensor module [Hanein, et al., Sens. Actuators B: Chem., vol. 81, pp. 49-54, 2001; Mathew, et al., Biosens. Bioelectron., vol. 20, pp. 1656-1661, 2005; Jin, et al., Appl. Surf. Sci., vol. 252, pp. 7397-7406, 2006.]. Additionally, optical properties of PS substrate can be explored for the fabrication of optoelectronic devices based on, for example, photoluminescence of PS skeleton and on low-pass IR filtration [Min, et al., J. Korean Phys. Soc., vol. 39, pp. S63-S66, 2001; Lehmann, et al., Appl. Phys. Lett., vol. 78, pp. 589-591, 2001.]. To make a sensing electrode active, the bio-receptor molecule complementary to the target molecule should be immobilized on the sensing electrode.

Rough surface of PS layer was contemplated to give extra bonding strength between the receptor molecules and the electrode substrate. Conductive polymer (CP) can play a role as an immobilization matrix for such receptor molecules as enzyme, antibody, or DNA because positively charged CP matrix can make electrostatic binding to negatively charged biomaterials [Llaudet, et al., Biosens. Bioelectron., vol. 18, pp. 43-52, 2003; Tlili, et al., Talanta, vol. 68, pp. 131-137, 2005; Jiang, et al., Anal. Chim. Acta, vol. 537, pp. 145-151, 2005]. CP films can be synthesized by electropolymerization. Furthermore, the shape, thickness, and conductivity of electropolymerized CP can be easily modified by adjusting the electrolyte and applied potential. Bonding strength between the CP and the biomaterial is strong enough and comparable to that of covalent bonding in silanization method. Since electropolymerization is simple and reliable, the application of electropolymerized CP film can be exploited.

The inventors showed that nanoporous silicon (nPS)-based label-free DNA chips specifically fabricated and functionalized for monitoring Salmonella enterica serovar Enteritidis were successful in detecting these pathogen bacterium. To accomplish this goal, low-resistivity p-Si was electrochemically anodized to obtain the nPS layer. Conductive polypyrrole (PPy) film was directly electropolymerized on top of the nPS as a matrix for the probe DNA (pDNA) immobilization and the target (tDNA) hybridization steps. PPy has excellent electrical and physicochemical properties in addition to its outstanding compatibility with biomaterials [Ramanavicius, et al., Electrochim. Acta, vol. 51, pp. 6025-6037, 2006]. Peak current output from the label-free DNA chip was used for calibration over hybridization time and tDNA concentration.

The nanoporous silicon (nPS) layer was electrochemically formed on a p-Si wafer for the direct electropolymerization of pyrrole on the p-Si substrate without predeposition of a metallic thin film. For better electrical conductivity, low-resistivity p-Si wafer was used as starting material. Basic function of the nPS layer as described herein, was to enhance the surface roughness of the bare p-Si substrate and to modify the physicochemical property of the p-Si surface. The average size of the nPS layer grown on the p-Si substrate by applying −5 mA×cm⁻² of J in an etchant composed of HF:CH3 CH2 OH=3:7 was 12 μm in depth and 10 nm in width. Formation of the natural oxide layer is easier on nPS layer than the macro PS. Thus, the conductive PPy film could be directly deposited on the nPS layer due to higher SiO formation than bare silicon. The rough surface of PS layer enhanced the surface energy of the PS layer to form a stronger adsorption bond between nPS and PPy. Electrostatic adsorption of pDNA to the conductive PPy matrix did not require any complicated fabrication procedure unlike the silanization process [Bessueille, et al., Biosens. Bioelectron., vol. 21, pp. 908-916, 2005; Bisi, et al., Surf. Sci. Rep., vol. 38, pp. 1-126, 2000] but with comparable bonding strength. The sensitivity obtained from the plot of iₚ versus tDNA concentration was −166.6 μA×cm⁻²×μM⁻¹. The nPS-based DNA sensor shown herein was used to provide an output to determine the original amount of tDNA. Optical property of the PS layer depends on the size of the PS layer [Bisi, et al., Surf. Sci. Rep., vol. 38, pp. 1-126, 2000].

This means that the PS structure strongly affected optoelectronic signal output but has relatively less effect on the electrochemical signal. Therefore, better reproducibility in the nPS-based electrochemical sensor could be expected. The whole procedure of the nPS-based electrochemical sensor for monitoring pathogens is simple and that rapid detection is feasible compared with the other methods (impedimetric or spectroscopic methods) [Bessueille, et al., Biosens. Bioelectron., vol. 21, pp. 908-916, 2005].

Further, a biosensor for the detection of food-borne pathogens (Salmonella Enteritidis) was fabricated based on nanoporous silicon (NPS). P-type silicon wafers (100, 0.01 Ωcm) were anodized electrochemically in an electrochemical Teflon cell, containing ethanoic hydrofluoric acid solution to produce the porous layer on the silicon surface. The porous silicon surface was functionalized with DNA probes specific to the insertion element (Iel) gene of Salmonella Enteritidis. A biotin-streptavidin system was utilized to characterize the availability of the nanopores and the specificity of the DNA probe. Based on the electrical property of DNA, redox indicators and cyclic voltammetry were used for the characterization of the biosensor. Results showed that the DNA probe was specific to the target DNA, and the porous silicon-based biosensor had more active surface area and higher sensitivity (1 ng/mL) than the planar silicon-based biosensor. This simple, label-free porous silicon-based biosensor has potential applications in high-throughput detection of pathogens.

TABLE 2
DNA Sample Concentration Versus Peak Current (At 1.5 V)
and Charge Density On Nanoporous Silicon-Based Biosensor.

| | DNA Concentration (ul/ml) | | | | |
|---|---|---|---|---|---|
| | 0.1 | 0.01 | 0.001 | 0.0001 | 0 |
| Peak current | −1.59 | −0.89 | −0.37 | −0.03 | −0.086 |
| of PS (mA) | | | | | |
| Charge | 0.05353 | 0.05267 | 0.01461 | 0.00132 | 0.001378 |
| density | | | | | |
| of PS | | | | | |
| (mC cm-3) | | | | | |

Nanoporous silicon (NPS) was investigated as a potential platform for biosensing applications because of its high surface-to-volume ratio and specific photoluminescence property [Canham, Properties of Porous Silicon. London, U.K.: INSPEC Publisher, 1997, pp. 364-370]. Porous silicon-based biosensors were developed for the rapid detection of pathogens with a sensitivity of colony forming units (CFU)/mL of Escherichia coli [Mathew and Alocilja, Biosens. Bioelectr., 20:1656-1661:2005]. Jin et al. reported a poly(3-Methyl thiophene)-based enzyme biosensor on porous silicon substrate for the detection of urea. The main focus of their study was the amplified surface area, which allowed higher sensitivity, while using a smaller device [Jin, et al., Appl. Surf. Sci., vol. 252, pp. 7397-7406, August 2006]. Francia et al. utilized porous silicon (PS) as a transducer based on its photoluminescence property. Without using labels, specific hybridization was directly detected as a variation in the PS photoluminescence [Francia, et al., Biosens. Bioelectr., 21:661-665, 2005]. A multilayer porous silicon was tested utilizing microcavities for biosensing and the refractive index changed after biological molecules became attached to the surface [DeLouise, et al., Anal. Chem., 77:3222-3230, 2005]. However, understanding how to control PS morphology (porosity, thickness, and reproducibility) is difficult owing to the large number of factors. Furthermore, the availability or functionality of the nanopore surface is a major issue in nanoporous silicon chips.

In nanoporous silicon-based DNA biosensor, the main issue is the transduction of DNA hybridization. Transduction of DNA hybridization at a DNA-modified recognition interface has been achieved electrochemically [Komarova, et al., Biosens. Bioelectr., 21: pp. 182-189, 2005], optically [Major, et al., Optics Expr., 14:5285-5294, 2006], and by using mass sensitive devices [8]. However, one of the key drawbacks of the DNA biosensor concept discussed above is the requirement for an indicator to transduce the hybridization event. Mathew and Alocilja reported on porous silicon chips that were functionalized with a dioxetane-polymyxin B (cell wall permeabilizer) mixture by diffusion and adsorption onto the porous surface [Mathew and Alocilja, Biosens. Bioelectr., 20:1656-1661:200]. The reaction of beta-galactosidase enzymes from E. coli with the dioxetane substrate generated light at 530 nm. Light emission for the porous silicon biosensor chip with E. coli was significantly greater than that of the control and planar silicon chip with E. coli.

A label-free DNA electrochemical biosensor was fabricated as described herein based on nanoscale porous silicon chips using electrochemical anodization. By immobilizing the specific DNA probe onto the PS layer, the biosensor had the ability to capture complementary DNA (cDNA). Based on the formation of DNA double strand molecular wire, the affinity between redox indicators and DNA duplex changed. Different concentrations of DNA samples resulted in different peak currents using cyclic voltammetry. Biotin-streptavidin system was used to assess successful DNA hybridization in order to verify the availability of nanopore surface and specificity of the DNA probe. This paper will focus on the characterization of nanoscale porous silicon for biosensor application and the design of a label-free DNA biosensor for S. Enteritidis detection.

Nanoscale porous silicon for biosensor application and the design of a label-free DNA biosensor for S. Enteritidis detection showed the capability for use in embodiments of the present inventions. A PS layer was formed by electrochemical anodization and used as a substrate for the biosensor. The depth and diameter of the pores on the porous layer are important factors because they strongly affect DNA probe immobilization and the sensitivity of the biosensor. The anodization conditions (HF concentration in=14.2% in ethanol, anodization duration=1 h, and current density=5 mA/cm² without additional illumination) provided the desired porous silicon chip characteristics with uniform, repeatable nanoscale pore structure. SEM was utilized to evaluate the porous layer and the image showed that it was formed homogenously.

After silanization, the specific DNA probe was immobilized onto the chip surface. Hybridization quality control testing showed that the porous silicon had higher surface area compared with planar silicon by a factor of 4 and the DNA probe had high selectivity and affinity to the target DNA. The redox marker had a greater affinity for dsDNA and, therefore, a greater electrochemical response was observed when hybridization occurred. When the concentration of target DNA increased, the charge transfer between the redox marker and the PS electrode was enforced so that the peak current increased with DNA concentration. The experimental result showed that the detection limit of the PS-based label-free DNA biosensor was 1 ng/mL. More work is required for a full understanding of the working mechanism of the surface modified semiconductor electrode, especially modified with DNA sequences. At the same time, decreasing the random error during sensor fabrication to confirm the stability of the signal is a key issue. Further research will be taken to control these variables and create a more robust biosensor.

### EXPERIMENTAL

The following Examples are provided in order to demonstrate and further illustrate certain preferred embodiments and aspects of the present invention and are not to be construed as limiting the scope thereof.

Examples I-III describe the process for providing a novel biosensor comprising an antibody-based AUNT in combination with polyaniline-coated magnetic nanoparticles. The remaining examples demonstrate additional embodiments of biosensors of the present inventions.

In the experimental disclosure which follows, the following abbreviations apply: ° C. (degrees Centigrade); H₂O (water); HCl (hydrochloric acid); wt (wild-type); bp (base pair); kb (kilobase pair); kD (kilodaltons); gm (grams); μg (micrograms); mg (milligrams); ng (nanograms); μl (microliters); ml (milliliters); mm (millimeters); nm (nanometers); μm (micrometer); M (molar); mM (millimolar); μM (micromolar); U (units); V (volts); MW (molecular weight); sec (seconds); min(s) (minute/minutes); hr(s) (hour/hours); MgCl₂ (magnesium chloride); NaCl (sodium chloride); OD₂₈₀ (optical density at 280 nm); OD₆₀₀ (optical density at 600 nm); PAGE (polyacrylamide gel electrophoresis); PBS (phosphate buffered saline [150 mM NaCl, 10 mM sodium phosphate buffer, pH 7.2]); PCR (polymerase chain reaction); PEG (polyethylene glycol); RT-PCR (reverse transcription PCR); SDS (sodium dodecyl sulfate); Tris (tris(hydroxymethyl)aminomethane); w/v (weight to volume); v/v (volume to volume); reagents for nanparticle synthesis were purchased from Sigma (St. Louis, Mo.); a Nap-5 column purchased from GE Healthcare (Piscataway, N.J.) was used to purify the DNA product from DTT solution. Sulfosuccinimidyl 4-N-maleimidomethyl cyclohexane-1-carboxylate (sulfo-SMCC; Pierce, Wis.) was used as a cross-linker between thiolated pDNA and amine-coated MNPs. Sulfo-NHS acetate (Pierce, Wis.) was used to block unreacted sulfo-SMCC. Solutions used herein were prepared in double distilled water.

Apparatus. Target DNA (tDNA) was amplified by a thermocycler (Mastercycler Personal, Eppendorf). Gel electrophoresis (Runone System) was used for confirmation of PCR product. After purification of the PCR product, a spectrophotometer (SmartSpec 3000, Bio-Rad Laboratories) was used to measure the concentration of tDNA samples, as well as pDNA and barcode DNA. In some embodiments, oligonucleotides were purchased from IDTDNA Inc. (Coralville, Iowa).

In the characterization experiments of Au-NPs, a UV-vis-NIR scanning spectrophotometer (UV-3101PC, Shimadzu) was used to determine the absorbance of Au-NPs, and TEM (JEOL100 CXII) was used for characterization of the nanoparticle dimension. Magnetic separation was done using a magnetic separator (FlexiMag, SpheroTech). A centrifuge (Micro12, Fisher Scientific) was used for separation and purification of Au-NPs. An incubator (HS-101, Amerex Instrument Inc.) was used to enrich the bacteria and hybridization reaction. A fluorescent multi-label counter (Victor 3, PerkinElmer) was used for measuring fluorescence intensity.

Synthesized nanoparticles were characterized by TEM and UV-vis-NIR scanning spectrophotometer. In the characterization experiments of AuNPs, a UV-Vis-NIR Scanning Spectrophotometer (UV-3101PC, Shimadzu) was used to determine the absorbance of AuNPs, and transmission electron microscopy (TEM, JEOL100 CXII) was used to characterize the nanoparticles. A centrifuge (Micro12, Fisher Scientific) was used for separation and purification of AuNPs. MNPs were characterized by TEM and the Quantum Design MPMS SQUID magnetometer. The dimension of MNPs was characterized by TEM and their magnetic profile was characterized by Quantum Design MPMS SQUID (Superconducting quantum information device) magnetometer. Magnetic separation was done using a magnetic separator (FlexiMag, SpheroTech). A VICTOR3 multilabel plate reader from PerkinElmer (Waltham, Mass.), was used for assays. An incubator (HS-101, Amerex Instrument Inc.) was used to enrich the bacteria and hybridization reaction. Screen-printed carbon electrode (Gwent, England) and a potentiostat/galvanostat (263A, Princeton Applied Research, TN) were used for electrochemical measurement.

The following references describe materials and methods used herein and are herein incorporated by reference in their entirety: Hill and Mirkin (2006) Nat Protoc 1(1):324-36; Parak, et al. (2002) Chern. Mater. 14(5):2113-2119; Pradhan, et al. (2003), J Am Chern Soc 125(8): 2050-1; Wang, et al. (2000) Anal Chern 72(14): 3218-22; and Wang, et al. (2006) Chemistry 12(24): 6341-7.

### EXAMPLES

These Examples describes the synthesis and characterization of nanoparticles and DNA used in the present inventions. The nanoparticles were synthesized and characterized based on their magnetic properties (for MNPs), nanoscale dimensions, and optical properties (for AuNPs and NPTs) (See, FIGS. 1 and 2). Functionalization of AuNPs and MNPs was successfully carried out and analyzed (See, FIGS. 1 and 2). This assay was initially developed using fluorescent detection of released silent (bDNA) (Zhang, et al., Biosens Bioelectron. 2009 Jan. 1; 24(5):1377-81. Epub 2008 Aug. 19, herein incorporated by reference) followed by the use of metal tracer particles as described below and in the following Examples.

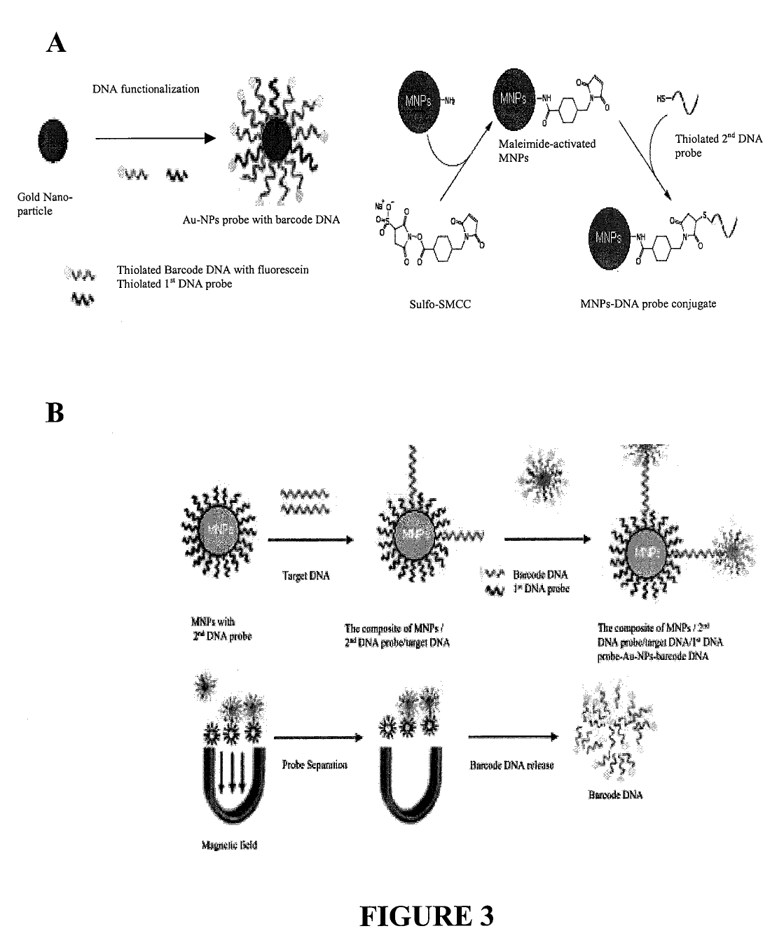

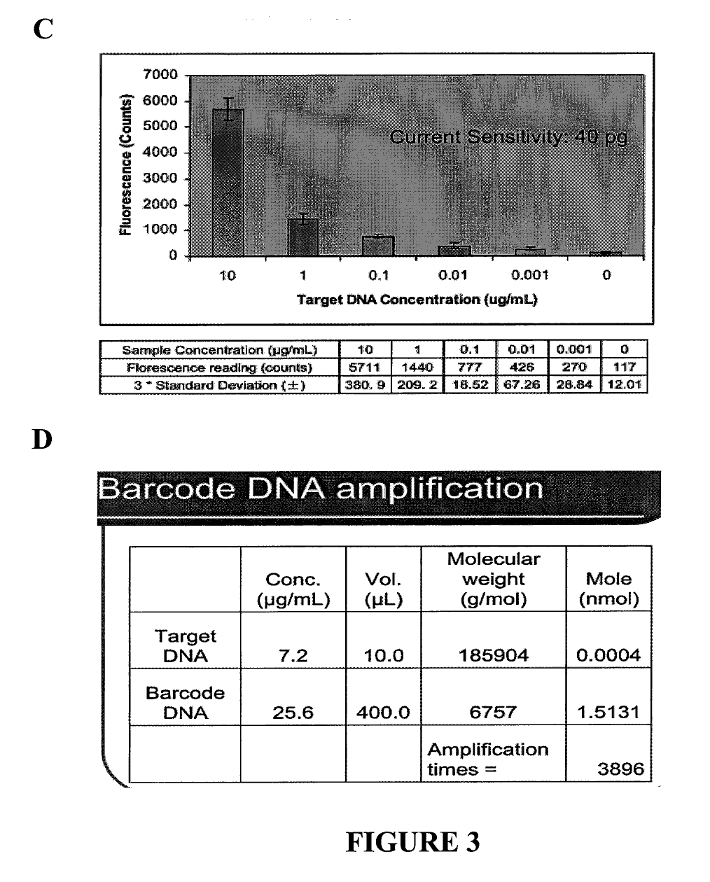

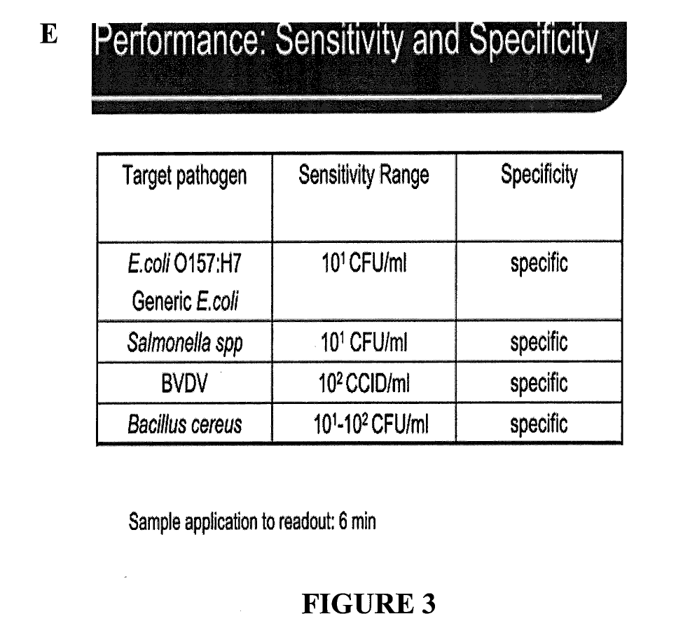

The following examples describe biosensor applications of this diagnostic system tested by evaluating the detection of the lei gene from Salmonella Enteritidis in homogenous DNA samples consisting of target lei gene. The inventors initially tested AUNT using fluorescent molecules. The results showed a molar amplification ratio (bDNA/tDNA) approximately 4000 times using fluorescent molecules with AUNT, see, FIG. 3C. Subsequent experiments used AUNT with metal tracer molecules, as described below. Initial results showed that reliability and repeatability of the SPCE measurements was established. Several metal ions from NPTs, including Cd2+ and Pb 2+, were tested on the SPCEs. They demonstrated peak current at their characteristic potentials and the concentration of ions shows good linear relationship with the peak current (FIG. 12).

### Example I

This Example describes the synthesis of AuNPs and MNPs, amine-functionalized AuNPs, amine-functionalized MNPs, metal tracer molecules and DNA targets and probes of the present inventions. In particular, descriptions include the synthesis of elements for use as AUNT detection using fluorescent molecules and nanoparticle tracers of the present inventions. Specifically, Example describes the synthesis AuNPs and MNPs, amine-functionalized AuNPs, amine-functionalized MNPs, and DNA targets and probes of the present inventions for use with embodiments of biosensors of the present inventions.

Synthesis (fabrication) and characterization of gold nanoparticles (AuNPs): Gold nanoparticles were synthesized by a chemical reduction method (Hill, et al., 2006. Nat. Protoc. 1:324-336, herein incorporated by reference in entirety). Hydrogen tetrochloroaurate (III) trihydrate and sodium citrate dehydrate were used for the synthesis of gold nanoparticles. 1,4-Dithio-DL-threitol (DTT) was used for the cleavage of oxidized thiolated oligonucleotides before conjugation.

In brief, hydrogen tetrochloroaurate (III) trihydrate aqueous solution (1 mM, 50 mL) was heated with stirring on a hotplate. Once it refluxed vigorously, the solution was slowly titrated with 5 mL of 38.8 mM sodium citrate. The solution turned from yellow to clear, to black, to purple and finally deep red. Their dimension and spectroscopic properties were characterized by a transmission electron microscopy (TEM) and UV-vis-NIR scanning spectrophotometer.

Characterization of nanoparticles: The AuNP dimension and spectroscopic properties were characterized by using a transmission electron microscope (TEM) and a UV-Vis-NIR scanning spectrophotometer, respectively. FIG. 1 shows a transmission electron microscopy (TEM) image of synthesized AuNPs with an average diameter of 15 nm and an absorption peak at 519 nm wavelength. The dimension of AuNPs is homogenous. After one month storage in room temperature, the AuNPs did not aggregate and their spectroscopic absorbance property was stable.

Synthesis (fabrication) and characterization of magnetic nanoparticles (MMPs):

Anhydrous sodium acetate, 1,6-hexanediamine, FeCl3.6H2O, and ethylene glycol were used for synthesis of amine-coated magnetic nanoparticles (MNPs).

MNPs were synthesized by following a facile one-pot template-free method [Wang, et al., Chemistry—A European Journal, 2006. 12(24):6341-6347, Wang, et al. 2006, herein incorporated by reference in entirety]. A solution of 1,6-hexanediamine (6.5 g), anhydrous sodium acetate (2.0 g) and FeCl3.6H2O (1.0 g) in ethylene glycol (30 mL) was stirred vigorously at 50° C. to give a transparent solution. This solution was then transferred into an oven and reacted at 198° C. for 6 h.

The MNPs were characterized by TEM and Quantum Design MPMS SQUID (Superconducting quantum information device) magnetometer. FIG. 2 shows an exemplary TEM image of synthesized MNPs, and their magnetic properties (inset). The diameter of MNPs is around 100 nm and the magnetic hysteresis has a maximum of 74.6 EMU/g at room temperature. Previous research by the inventors showed that the conjugation between nanoparticles and thiolated oligonucleotides was efficient [Zhang, et al., Biosensors and Bioelectronics, 2009. 24(5):1377-1381].

Functionalization of NPs: To ensure full reactivity, thiol-modified oligonucleotides were reduced by DTT immediately before use. Otherwise, the thiol group on the oligonucleotides would not form a self-assembled monolayer on the surface of AuNPs due to loss of active thiol group. The AuNPs synthesized previously (1 mL), and the purified thiolated 1st pDNA (0.05 nmol) were then mixed together. A self-assembly monolayer of thiolated DNA formed on the surface of AuNPs. FIG. 1A shows schematics of the AuNPs functionalization. After a serial salt addition [Hill, et al., Nat Protoc, 2006. 1(1):324-3], the particles were stabilized for long-time storage at room temperature. For the MNPs, the polyamine-functionalized iron oxide particles (1 mg) were reacted with 300 μg of sulfo-SMCC bifunctional linker for 2 h in 1 mL coupling buffer (0.1M PBS buffer, 0.2M NaCl, pH=7.2). The supernatant was removed after magnetic separation and the MNP-cross-linker conjugate was rinsed with the coupling buffer 3 times. The reduced thiolated 2nd pDNA (1 nmol) was added into 1 mL coupling buffer containing sulfo-SMCC-modified MNPs and reacted for 8 h. The functionalized MNPs were then suspended in 35 mL of 10 mM sulfo-NHS acetate. FIG. 1 shows schematics of the functionalization of MNPs. The solution was incubated and shaken at room temperature to block the unreacted sulfo-SMCC on the surface of MNPs. After passivation, the particles were centrifuged at 4000 rpm for 1 min and washed with passivation buffer (0.2M Tris, pH=8.5) and then with a storage buffer (10 mM PBS buffer, 0.2M NaCl, pH=7.4).

Metal sulfide nanoparticles: were synthesized using metal salts of hexadecyl xanthate (HDX) as precursors (Pradhan and Efrima (2003). J Am Chern Soc 125(8): 2050-1, herein incorporated by reference in its entirety). Scheme I shows that the preparation of NPT-streptavidin conjugate (Parak, et al,. 2002, Chern. Mater. 14(5): 2113-2119, herein incorporated by reference in its entirety).

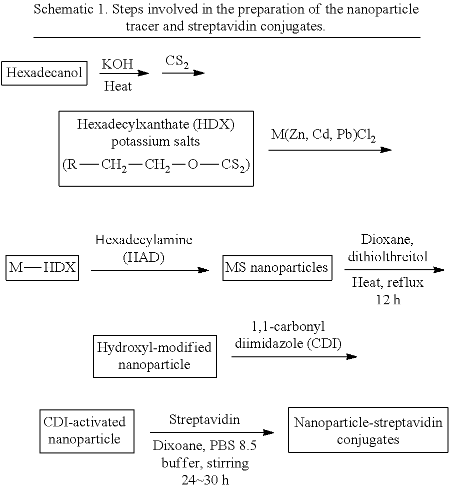

DNA probes (pDNAs) of Iel gene: were as follows: 1st pDNA on AuNPs: 5′-5′-AATATGCTGCCTACTGCCCTACGCTT-SH-3′ (SEQ ID NO:02); 2nd pDNA on MNPs: 5′-SH-[T]TTATGTAGTCCTGTATCTTCGCCGT-3′ (SEQ ID NO:03) (bold T optional).

DNA probes (pDNAs) of pagA gene are: 1^{st} DNA probe on AuNPs: 5′-SH-GGAAGAGTGAGGGTGGATACAGGCT-3′ (SEQ ID NO:04), 2^{nd} DNA probe on MNPs: 5′-AGATTTAAATCTGGTAGAAAGGCGG-SH-3′ (SEQ ID NO:05).

Functionalization of AuNPs and MNPs: A functionalixed composition for use in AUNT was a AuNP (−15 nm in diameter) were modified with a thiolated 151 DNA probe (1st pDNA). Barcode DNA (bDNA) were also labeled with a thiol group on the 3′ end and biotin on the 5′ end. The universal (bDNA; barcode) DNA on AuNP was 5′-biotin-TTATTCGTAGCTAAAAAAAAAA-SH-3′, SEQ ID NO:1 or 5′-biotin-TTATTCGTAGCT AAAAAAAAAA-6-FAM-3′ SEQ ID NO:10.

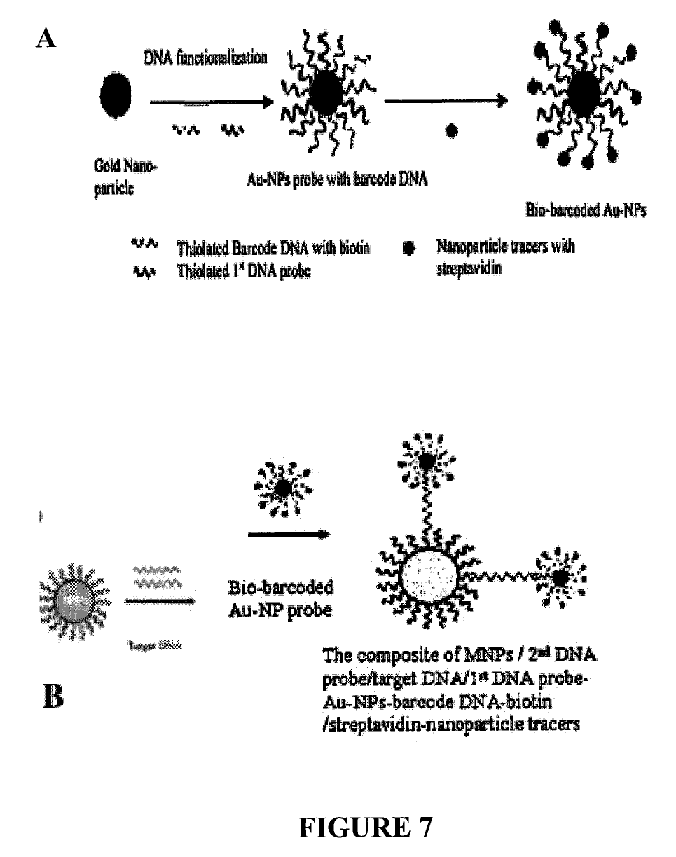

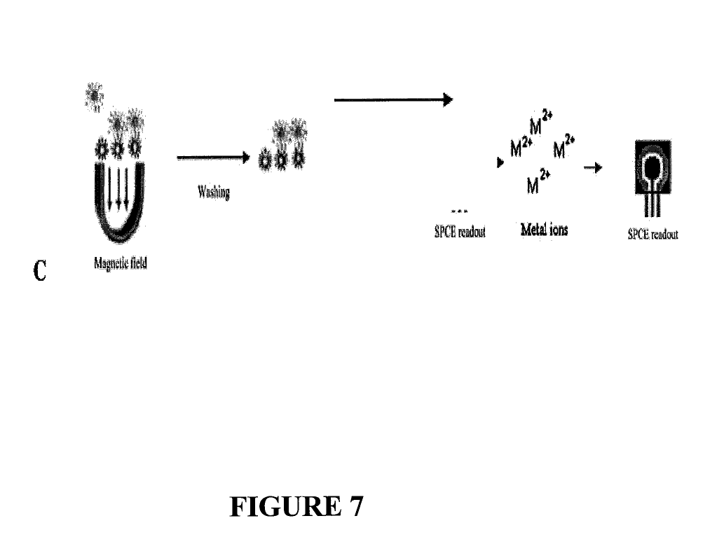

The AuNPs were loaded with 1st pDNA and bDNA by treating citrate-stabilized particles with a solution containing the two alkanethiol-capped oligonucleotides at a ratio of I pDNA to 100 bDNA (1:100). The thiol on both oligonucleotides formed a self-assembled monolayer on the AuNP surface. This ratio allowed target amplification while maintaining enough binding opportunity for pDNA. Then the NPT-streptavidin I conjugates were added into the solution. The specific binding between biotin and streptavidin win allow the composite (1st pDNA-AuNP-bDNA-NPTs) to form a complex of the present inventions, showing several schematics in FIG. 7.

A functionalixed composition for use in AUNT was a magnetic nanoparticle (MNPs, −100 nm diameter), which had a magnetic iron-oxide core and amine coating on the surface. MNPs were functionalized with alkanethiol-capped 2nd DNA probe (2pDNA) that is complementary to the other end of the tDNA (from the 1^{st} DNA probe) using a sulfosuccinimidyl 4-N-maleimidomethyl cyclohexane-1-carboxylate (sulfo-SMCC) linker (FIGS. 1B and 7).

Genomic DNA (target DNA) preparation: The pathogens were grown by the standard microbiological culture following FDA protocol [FDA. Bacteriological analytical manual online. 2001: Available from: world wide web.cfsan.fda.gov/approximatelyebam/bam-toc.html]. DNA isolation was performed from 1 mL Salmonella Enteritidis culture using the QiaAmp DNA Mini Kit (Qiagen Inc., Valencia). The forward primer used for amplifying a tDNA sequence was 5′-CTAACAGGCGCATACGATCTGACA-3′ (SEQ ID NO:11); the reverse primer was 5′-TACGCATAGC GATCTCCTTCGTTG-3′3′ (SEQ ID NO:12) [Wang, et al., Lett Appl Microbiol, 2002. 34(6): 422-7].

CV of unmodified AuNPs on SPCE: Compared with conventional electrodes, screen-printed carbon electrodes have several advantages, such as simplicity, convenience, low cost and the avoidance of contamination between samples [Miscoria, S. A, et al., Anal Chim Acta, 2006. 578(2):137-44; Susmel, et al., Biosens Bioelectron, 2003. 18(7):881-9]. Biosensors based on screen-printed carbon electrode (SPCE) have been extensively used for the detection of glucose [Xu, et al., Conf Proc IEEE Eng Med Biol Soc, 2005. 2:1917-20; Guan, et al., Biosens Bioelectron, 2005. 21(3):508-12], cholesterol [Carrara, et al., Biosens Bioelectron, 2008. 24(1):148-50], antigens [Kim, et al., IEEE Sensors Journal, 2006. 6(2):248-252] and DNA [Hernandez-Santos, et al., Anal Chem, 2004. 76(23):6887-93; Ruffien, et al., Chem Commun (Camb), 2003(7):912-3].

Oligonucleotide-functionalized AuNPs were used as signal indicator in this study because of their ease of fabrication, greater oligonucleotide binding capabilities, stability under a variety of conditions, and good electrochemical properties [Lytton-Jean, et al., J. Am. Chem. Soc., 2005. 127(37):12754-12755; Demers, et al., Am Chem Soc, 2002. 124(38):11248-9]. FIG. 9 shows that when there is no AuNPs, the carbon electrode is oxidized when performing oxidative procedure at 1.25V for 2 min. The carbon electrode can produce a reduction peak at 0.58 V. When the AuNPs exist in the solution, the AuNPs are oxidized instead of the carbon electrode under 1.25V for 2 min. The curve (FIG. 9) shows that there is a reduction peak of Au3+ at 0.35 V. Meanwhile, the background signal at 0.58 V from the carbon electrode is suppressed greatly by AuNPs. The possible reason is that the surface of SPCE is covered by a self-assembled layer of AuNPs due to physical adsorption. The SPCE is protected by AuNPs and this prevents the electrooxidation process to proceed on the carbon electrode surface.

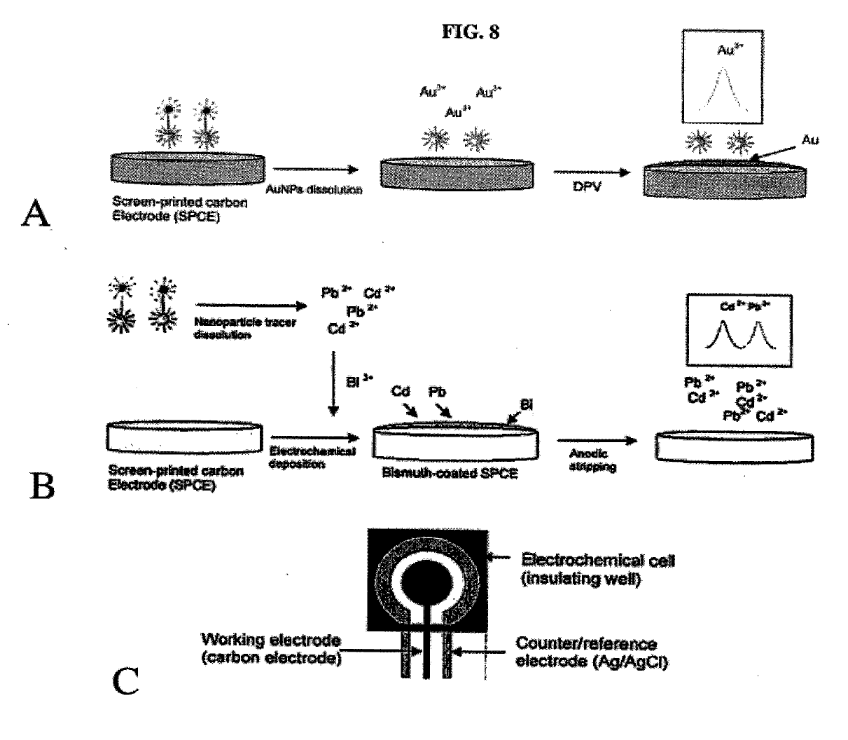

Optimization of detection of probe modified-AuNPs: The accumulation time is the time from dropping the sample on the SPCE to the beginning of electrochemical detection. It is an important factor for the electrochemical measurement of AuNPs because more AuNPs would be adsorbed on the SPCE surface when the accumulation time is longer. The electrooxidation process would generate more Au3+ ions in the solution, which could be translated to a higher DPV signal and thus improve the biosensor sensitivity. The sample solution was dried after 20 min at room temperature. The drying allowed most of the AuNPs to adsorb physically on the SPCE surface. Then 100 μL of 0.1M HCl was added again for DPV measurement. FIG. 8 shows the DPV current for 0, 10, 20 min of accumulation times. FIG. 8 has 20 min of accumulation time which allows for more AuNPs to be reduced or oxidized, leading to a comparatively high signal. With 10 min and 0 min accumulation times, peak current are at 0.86 mA and 0.36 mA, respectively. They are only 72% and 30% of the reduction peak current at 1.21 mA for the 20 min accumulation time. Drying led to more AuNPs available on the surface of SPCEs, resulting in the higher sensitivity of SPCE biosensors. Accumulation time of 20 min was used for the succeeding DPV detection of the DNA sandwich complex. FIG. 8

### Example II

This example demonstrates fluorescent labeling of silent biobarcode DNA for determining efficency of labeling and for detection of pathogens.

Hydrogen tetrochloroaurate (III) trihydrate and sodium citrate dehydrate were used for the synthesis of gold nanoparticles. 1,4Dithio-DL-threitol (DTT) was used for the cleavage of oxidized thiolated oligonucleotides and release of thiolated barcode DNA from Au-NPs surface. Amine-coated MNPs were used for separation and preconcentration. Reagents were purchased from Sigma (St. Louis, Mo., USA). Nap-5 column was purchased from GE Healthcare (Piscataway, N.J., USA), which was used to purify the DNA product from DTT solution. Sulfosuccinimidyl 4-Nmaleimidomethyl cyclohexane-1-carboxylate (sulfo-SMCC; Pierce, Milwaukee, Wis., USA) was used as a cross-linker between thiolated DNA probe and amine-coated MNPs. Sulfo-NHS acetate (Pierce, Milwaukee, Wis., USA) was used to block unreacted sulfo-SMCC. Solutions were prepared in distilled water.

Gold nanoparticles were synthesized by a chemical reduction method (Hill and Mirkin, 2006). Hydrogen tetrochloroaurate (III) trihydrate aqueous solution (1 mM, 50 mL) was prepared in a flask. The gold solution was heated while stirring on a hotplate. Once it refluxed vigorously, the solution was slowly titrated with 5 mL of 38.8 mM sodium citrate. The solution turned from yellow to clear, to black, to purple and finally deep red. The UV-vis absorption spectrum of the Au-NPs solution was measured by a UV-vis-NIR scanning spectrophotometer (UV-3101PC, Shimadzu Inc.).

In one exemplary experiment, target DNA was amplified in PCR to generate DNA used for this experiment. Target DNA was amplified by a thermo cycler (Mastercycler Personal, Eppendorf) using primers described below. PCR product was confirmed by gel electrophoresis (Runone System). After purification of the PCR product, a spectrophotometer (SmartSpec 3000, Bio-Rad Laboratories) was used to measure the concentration of DNA samples, as well as DNA probe and barcode DNA. In the characterization experiments of Au-NPs, a UV-vis-NIR scanning spectrophotometer (UV-3101PC, Shimadzu) was used to determine the absorbance of Au-NPs, and TEM (JEOL100 CXII) was used for characterization of the nanoparticle dimension. All magnetic separation was done using a magnetic separator (FlexiMag, SpheroTech). A centrifuge (Micro12, Fisher Scientific) was used for separation and purification of Au-NPs. An incubator (HS-101, Amerex Instrument Inc.) was used to enrich the bacteria and hybridization reaction. A fluorescent multi-label counter (Victor 3, PerkinElmer) was used for measuring the fluorescent signal.

A clinical strain of S. Enteritidis (strain S-64) was used in this study. The pathogen was grown in trypticase soy broth (TSB) at 37° C. for 16 h. After enrichment, the cells were enumerated by spiral plating appropriately diluted cultures on brilliant green agar. DNA isolation was performed from 1 mL S. Enteritidis culture using the QiaAmp DNA Mini Kit (Qiagen, Valencia, Calif.). Primers were designed for the detection of S. Enteritidis using the insertion element (Iel) gene (Wang and Yeh, 2002). The single stranded forward and reverse primers were IelL-5′-CTAACAGGCGCATACGATCTGACA-3′ (positions 542-565, 24 bases) and IelR-5′-TACGCATAGCGATCTCCTTCGTTG-3′ (positions 1047-1024, 24 bases). After PCR amplification, the PCR product was purified by PCR purification kits (QIAquick; QIAgen, Valencia, Calif.) and diluted serially with distilled water. The following thiolated oligonucleotides were used for conjugation with nanoparticles: The first (1^{st}) DNA probe on Au-NPs: 5′-AATATGCTGCCTACTGCCCTACGCTT-thiol-3′ (position: 919-944), The second (2^{nd}) DNA probe on MNPs: 5′-thiol-TTTATGTAGTCCTGTATCTTCGCCGT-3′ (position: 661-686), Barcode DNA on Au-NPs: 5′-TTATTCGTAGCTAAAAAAAAAA-thiol-3′ (Hill and Mirkin, 2006). TEX 613 (wavelength excitation=596 nm, wavelength emission=613 nm) was used to label the barcode DNA and 6-Carboxyfluorescein (6-FAM; wavelength excitation=495 nm, wavelength emission=520 nm) was used to label the 2^{nd} DNA probe on MNPs. Both TEX 613 and 6-FAM were used to evaluate the conjugation efficiency. Ooligonucleotides were purchased from Integrated DNA Technologies (Coralville, Iowa).

To ensure full reactivity, thiol-modified oligonucleotides were reduced immediately before use. Otherwise, the thiol group on the oligonucleotides did not form a self-assembled monolayer on the surface of Au-NPs due to loss of active thiol group. DTT solution (0.1 M) was prepared in the disulfide cleavage buffer (170 mM PBS buffer, pH 8.0). The thiolated oligonucleotide was suspended in 100 uL DTT solution and the solution was allowed to stand at room temperature for 2 h. Nap-5 columns were then used for purifying the reduced thiolated oligonucleotides. The procedure was followed according to the manufacture's manual. After purification, a UV-vis spectrophotometer was used to determine the DNA concentration. The Au-NPs synthesized previously (1 mL), the purified thiolated barcode DNA (5 nmol), and the purified thiolated 1^{st} DNA probe (0.05 nmol) were then mixed together. Thiolated DNA would form a self-assembled monolayer on the surface of Au-NPs. FIG. 1A shows a schematic of the Au-NPs functionalization. After a serial salt addition (Hill and Mirkin, 2006), the particles were stabilized for long-time storage at room temperature.

For the MNPs, the polyamine-functionalized iron oxide particles (1 mg) were reacted with 300 mg of sulfo-SMCC bifunctional linker for 2 h in 1 mL coupling buffer (0.1 M PBS buffer, 0.2 M NaCl, pH 7.2). The supernatant was removed after magnetic separation and the MNP-cross-linker conjugate was rinsed with the coupling buffer three times. The reduced thiolated 2^{nd} DNA probe (1 nmol) was added into 1 mL coupling buffer containing sulfo-SMCC-modified MNPs and reacted for 8 h. FIG. 2 shows the schematic of the MNP-DNA probe conjugation. After conjugation, the DNA probeimmobilized MNPs were rinsed with the coupling buffer three times. The unreacted DNA probe in the supernatant was collected for the evaluation of conjugation efficiency. The functionalized MNPs were then suspended in 35 mL of 10 mM sulfo-NHS acetate. The solution was incubated and shaken at room temperature to block the unreacted sulfo-SMCC on the surface of MNPs. After passivation, the particles were centrifuged at 4000 rpm for 1 min and washed with passivation buffer (0.2 M Tris, pH 8.5) and then with a storage buffer (10 mM PBS buffer, 0.2 M NaCl, pH 7.4).

Fluorescein-labeled 2nd DNA probe and barcode DNA were used to evaluate the conjugation efficiency. To separate the conjugates from unreacted oligonucleotides, different separation procedures were taken. After centrifuging at 13,000 rpm for 20 min, the fluorescence signal of the supernatant solution of 1st DNA probe/Au-NPs/barcode DNA-TEX 613 was measured. The supernatant solution of MNPs/sulfo-SMCC/2nd DNA probe-6-FAM was measured after magnetic separation. The same separation procedure was applied to their respective controls.

A solution containing target DNA in PCR tube was put in the thermocycler at 95° C. for 10 min to separate the dsDNA into ssDNA. Serially diluted DNA samples (40 μL) were mixed with 0.8 mg MNPs in 200 μL assay buffer (10 mM PBS buffer, 0.15 M NaCl, 0.1% SDS, pH 7.4). The hybridization reaction was maintained at a temperature of 45° C. for 45 min in an incubator. After hybridization, the MNPs with DNA target were washed twice with the assay buffer, then were resuspended in 200 μL assay buffer. The functionalized Au-NPs (100 μL) were centrifuged at 13,000 rpm for 20 min and the unreacted thiolated oligonucleotides in the supernatant were removed. Finally the purified Au-NPs complex was resuspended in 500 μL assay buffer. The Au-NPs complex (40 μL) was then added into 200 μL solution containing MNPs with DNA target. The hybridization was incubated at 45° C. for 2 h with shaking.

After the sandwich structure (MNPs-2nd DNA probe/Target DNA/1st DNA probe-Au-NPs-barcode DNA) was formed, the assay was put on the magnetic separator for 3 min and then the supernatant was removed. Unreacted solution components (DNA and Au-NPs) were washed away five times with 500 μL assay buffer in order to effectively remove Au-NPs that were not specifically bound to the MNPs through hybridization.

The MNP-target-Au-NP complex was resuspended in 200 μL of 0.5 M DTT solution. To release the barcode DNA from the surface of Au-NPs, the complex was incubated at 50° C. for 15 min, and then 45 min at 25° C. under vortex. After magnetic separation and centrifugation, the released barcode DNA was ready for fluorescent measurement.

Characterization of gold nanoparticles: The Au-NP dimension and spectroscopic property were characterized by using a transmission electron microscope (TEM) and a UV-vis-NIR scanning spectrophotometer, respectively. FIG. 1 shows a transmission electron microscopy image of our synthesized Au-NPs with an average diameter of 15 nm. The dimension of Au-NPs is homogenous. FIG. 2B shows the absorption peak of the Au-NPs at 519 nm wavelength. After one month storage in the room temperature, the Au-NPs did not aggregate and their spectroscopic absorbance property was stable.

The 6-FAM labeled 2nd DNA probe was used for confirming the functionalization of MNPs. Two controls were taken for evaluation of the conjugation efficiency. One was only the 6-FAM DNA probe, and the other was 6-FAM DNA probe and MNPs without sulfo-SMCC cross-linker.

Table 3 shows that the 6-FAM labeled barcode DNA has the highest fluorescence signal because no MNPs are in the solution. Magnetic separation has no effect on it. Without sulfo-SMCC as a cross-linker in solution, the DNA probe is not conjugated to the MNPs, hence the magnetic separation will not remove the 6-FAM labeled DNA probe. However, some of the DNA probe in solution could be washed out during magnetic separation, hence a decrease in fluorescence signal is observed. After adding sulfo-SMCC into the conjugation reaction, the conjugation efficiency improved greatly and the fluorescence signal deceased markedly. The result shows an exemplary sulfo-SMCC worked as an efficient cross-linker to conjugate the thiolated oligonucleotides and amine-coated MNPs. Because the synthesized Au-NPs have a peak absorbance at 519 nm, in order to avoid its interference with the excitation wavelength (520 nm) of 6-FAM, another fluorescent dye (TEX 613) was used to confirm the functionalization of Au-NPs. The unconjugated TEX 613 labeled barcode DNA was left in the supernatant solution after centrifugation at 13,000 rpm for 20 min. The control was the TEX 613 labeled barcode DNA in the same buffer solution without Au-NPs.

FIG. 3 shows that almost half of the thiolated barcode DNA has been immobilized on the surface of Au-NPs. The bio-barcoded Au-NPs were found to be stable in solution in terms of non-aggregation. This phenomenon is probably due to the negatively charged bar-code DNA repelling to each other, hence no aggregation occurs even at long storage time. The solution of bio-barcoded Au-NPs retained its deep red color under room temperature for over a month, demonstrating stability of conjugation. On the contrary, the Au-NPs in the solution without DNA probe aggregated to larger particles and precipitated finally under the same buffer condition.

FIG. 3 shows a schematic showing the bio-barcode assay. After immobilization of oligonucleotides on the surface of nanoparticles, both nanoparticles can bind with the target DNA to form a sandwich structure, due to the specificity of DNA probes. FIG. 3 shows the target DNA sample hybridizing with the DNA probe on the MNPs, forming a MNP-2nd DNA probe/target DNA complex. Then bio-barcoded Au-NPs are added to form a sandwich structure consisting of MNP-2nd DNA probe/target DNA/1st DNA probe-Au-NPs-barcode DNA (picture on the right). After hybridization is complete, the barcode DNA is released from the surface of Au-NPs and the fluorescence signal is measured by a fluorescence counter (FIG. 1).

FIG. 1 shows that the fluorescence signal of released barcode DNA has an exponential growth relationship with different concentrations of target DNA. At a concentration of 10 μg/mL target DNA, the fluorescence signal has an average count of 5711. The signal exponentially increases with increasing concentration of target DNA.

The fluorescence signal of released barcode DNA has an exponential growth relationship with different concentrations of target DNA. At a concentration of 10 μg/mL target DNA, the fluorescence signal has an average count of 5711. The signal exponentially increases with increasing concentration of target DNA. Table 4 shows the mean and variance of the signal. The results show that the assay can detect as low as 1 ng/mL target DNA (or 2.15×10-16 mol).

TABLE 3
Fluorescence signal of supernatant solution for evaluation of
conjugation efficiency between MNPs and 2^{nd} DNA probe.

| | MNPs + | | |
|---|---|---|---|
| | Sulfo- | | |
| | SMCC + | MNPs + 6- | 6-FAM |
| | 6-FAM | FAM DNA | DNA |
| | DNA | (control 2) | (control 1) |
| Fluorescence (0.1 s) (counts) | 72,735 | 293,003 | 368,146 |
| Standard deviation | 248.7 | 821.9 | 1942.1 |

TABLE 4
The relationship between target DNA and the fluorescence
signal of released barcode DNA.

| | Sample concentration (ug/mL) | | | | | |
|---|---|---|---|---|---|---|
| | 10 | 1 | 0.1 | 0.01 | 0.001 | 0 |
| Fluorescence | 5711 | 1440 | 777 | 426 | 270 | 117 |
| reading | | | | | | |
| (counts) | | | | | | |
| 3 × Standard | 380.88 | 209.16 | 18.52 | 67.26 | 28.84 | 12.01 |
| deviation (±) | | | | | | |

### Example III

This experimental study and Example III involves the development of a handheld multiplex DNA sensor for rapid differential diagnosis of pathogens using electrochemical detection. The DNA assay utilizes magnetic nanoparticles (MNPs) for target DNA (tDNA) separation from the sample and nanoparticle tracers (NPTs) conjugated to silent oligonucleotides attached to gold nanoparticles (AuNPs) for signal amplification. A disposable screen-printed carbon electrode (SPCE) and a handheld potentiostat powered by a pocket PC are contemplated as the signal generator and detection system.

DNA hybridization: A solution containing tDNA in PCR tube was put in the thermocycler at 95° C. for 10 min to separate the double-stranded DNA (dsDNA) into ssDNA. The serially diluted tDNA (40 μL) were mixed with AuNPs complex (40 μL) and 0.8 mg MNPs in 200 μL assay buffer (10 mM PBS buffer, 0.15M NaCl, 0.1% SDS, pH=7.4). The hybridization reaction was maintained at a temperature of 45° C. for 45 min in an incubator. After the sandwich complex (MNPs-2nd pDNA/tDNA/1st pDNA-AuNPs) was formed, the assay was put on the magnetic separator for 3 min and then the supernatant was removed. Unreacted solution components (DNA and AuNPs) were washed away 5 times with 500 μL assay buffer in order to effectively remove AuNPs that were not specifically bound to the MNPs through hybridization. Finally, the sandwich complex was resuspended in 100 μL of 0.1 M HCl solution. FIG. 7 shows a schematic of the DNA hybridization and a sandwich complex formation.

DPV detection of DNA sandwich complex: FIG. 7A shows the DPV response of the sandwich complex (MNPs-2nd pDNA/tDNA/1st pDNA-AuNPs) after hybridization for various tDNA concentrations (0.7-700 ng/mL). Following oxidative gold metal dissolution in the acidic solution, the Au3+ was reduced at the potential around 0.4V (vs. Ag/AgCl) on the SPCE. FIG. 7B shows that peak current has a log-linear relationship with increasing concentrations of tDNA. The detection limit is 0.7 ng/mL of target DNA. 2.6. Electrochemical detection on SPCE The SPCE biosensor is composed of two electrodes: carbon electrode (working electrode), and silver/silver chloride electrode (counter & reference electrode). The working area is limited by a meshed well. One hundred microliter of AuNPs solution was allowed to accumulate on the SPCE for 10 min. Cyclic voltammetry (CV) was used to characterize the AuNPs on SPCE. A constant 1.25V was applied to the electrode for 2 min to facilitate the oxidation of the AuNPs [Pumera, et al., Electrochimica Acta, 2005. 50(18):3702-3707]. Cyclic voltammetry (CV) was performed from 1.4V to 0.0V at a scan rate of 100 mV/s. The same procedure was also performed on the control group, a solution containing 0.1 M HCl. The pDNA-modified AuNPs in 100 μL of 0.1 M HCl was deposited onto the SPCE. The accumulation time of the AuNPs was varied for 0 min, 10 min and 20 min. The same CV procedure was performed to optimize the accumulation time of the electrochemical detection. Sandwich complexes from different tDNA were measured by differential pulse voltammetry (DPV) on SPCE under the optimum accumulation time. The potential was scanned from 1.25V to 0.0V with a step potential of 10 mV, modulation amplitude of 50 mV, and scan rate of 33.5 mV/s [Pumera, et al., Electrochimica Acta, 2005. 50(18):3702-3707].

FIGS. 7 and 8 show a schematic of the electrochemical detection of AuNPs. Following oxidative gold metal dissolution in an acidic solution, the released Au3+ ions were reduced on SPCE and indirectly quantified by differential pulse voltammetry (DPV). A potentiostat/galvanostat with PowerSuite software (Princeton Applied Research, TN) was used for electrochemical measurement and data analysis. when the accumulation time is longer. The electrooxidation process would generate more Au3+ ions in the solution, which could be translated to a higher DPV signal and thus improve the biosensor sensitivity. The sample solution was dried after 20 min at room temperature. The drying allowed most of the AuNPs to adsorb physically on the SPCE surface. Then 100 μL of 0.1M HCl was added again for DPV measurement. FIG. 8 shows the DPV current for 0, 10, 20 min of accumulation times. FIG. 8 has 20 min of accumulation time which allows for more AuNPs to be reduced or oxidized, leading to a comparatively high signal. With 10 min and 0 min accumulation times, peak current are at 0.86 mA and 0.36 mA, respectively. They are only 72% and 30% of the reduction peak current at 1.21 mA for the 20 min accumulation time. Drying led to more AuNPs available on the surface of SPCEs, resulting in the higher sensitivity of SPCE biosensors. Accumulation time of 20 min was used for the succeeding DPV detection of the DNA sandwich complex.

Universal (silent) Bio-Barcode DNA assay development—AUNT: The tDNA sample was allowed to hybridize with the 2pDNA on the MNPs forming a MNP-2pDNA-tDNA complex. Then bio-barcoded AuNPs were added to form a sandwich structure consisting of MNP-2pDNA/tDNA/1pDNA-AuNP-bDNA/NPT. After hybridization is complete, the sandwich structure was pulled to the bottom of the micro-tube by a magnet. A washing procedure separated the unreacted from the hybridized AuNPs. The whole complex was ready for electrochemical detection (FIG. 7).

Bio-barcode DNA assay confirmation: fluorescent detection of released bDNA that confirmed the formation of the sandwich complex, the bDNA was labeled with 6-carboxyfluorescein (6-FAM) and released using 0.5 M dithiothreitol (OTT) buffer solution after hybridization (FIG. 3).

The fluorescence signal of released bONA was measured by a fluorescent multi-label counter (Victor 3, PerkinElmer, MA). Electrochemical detection of Pb2+ and ccf+ on SPCE—The SPCEs were purchased from Gwent Inc. England.

FIG. 3C shows that the disposable 2 cm 2 electrochemical cell composed of two electrodes: carbon/graphite electrode (working electrode), and silver/silver chloride electrode (counter & reference electrode). The working area is limited by a meshed well. Serially diluted solution of Cd2+ and Pb2+ was prepared in acetate buffer (0.1 M, pH=4.5). The solution was used to evaluate the detection limit of metal ions on SPCE. Deposition potential (−1.2 V) was applied to the carbonworking electrode for 10 min. The metal irons in the solution were reduced on the SPCE. The voltammogram was recorded by applying a positive-going square-wave voltammetric potential scan from −1.4 V to 0 V (with a frequency of 20 Hz, amplitude of 25 mY, and potential step of 5 mY). This scan allowed the metal on SPCE to be oxidized and generated a characteristic peak current. A one minute conditioning step at +0.3 V was used to remove the target metals and bismuth, prior to the next cycle (Wang, Lu et al. 2000).

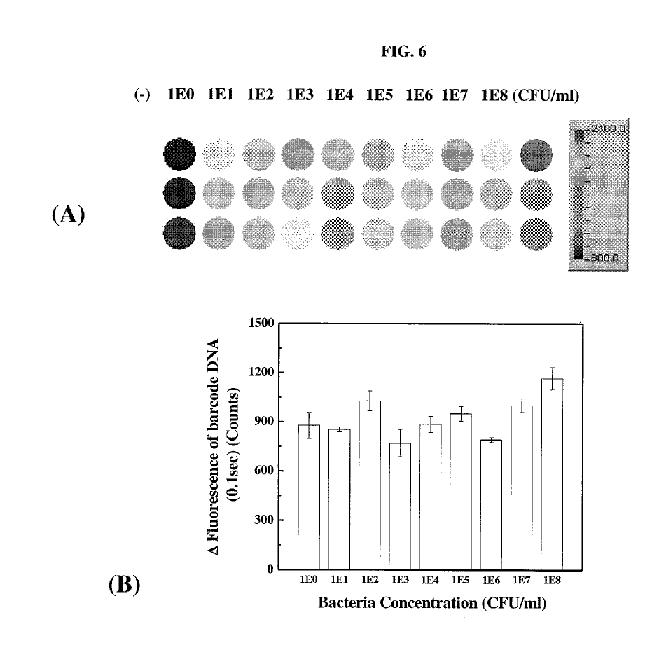

Herein is described a AuNP-based electrochemical biosensor for the detection of the insertion element gene of Salmonella Enteritidis. The biosensor takes advantage of the favorable electrochemical properties of AuNPs for a rapid and reliable detection on a disposable SPCE, and MNPs for easy and clean separation. The synthesized AuNPs were homogenous and stable, making them excellent materials for biosensing. The synthesized MNPs showed a good magnetic profile. The conjugation reaction between the two nanoparticles and thiolated oligonucleotides was efficient. After the pDNAs on nanoparticles hybridized with the nanoparticles, the sandwich complex formed, consisting of MNP-2nd pDNA-tDNA-1st pDNA-AuNPs. This complex was applied directly onto the SPCE after removing the unreacted AuNPs. The SPCE did not need surface modification. During the oxidation process, the existence of AuNPs suppressed carbon oxidation. Deposition of AuNPs on the SPCE for 20 min allowed more AuNPs to be oxidized and increased the sensitivity of the biosensor. The current peak of DPV for Au3+ reduction had a log-linear relationship with increasing tDNA concentration. The total detection time was about 1 hour. The results showed that the detection limit of the insertion element (Iel) gene of Salmonella Enteritidis was 0.7 ng/mL (or 1.5×10-16 mol). This disposable biosensor has great potential for quickly detecting bio-threat agents. Electrochemical detection of NPTs from bio-barcode assay—FIG. 6 shows the schematic of the SWASV measurement of multiplex DNA target. After a washing procedure to separate the unreacted from the hybridized AuNPs, the sandwich-conjugated complex was suspended in 20 J.1L 1 M HN03 solution for 3 min. After subsequent magnetic separation, the HN03 solution (containing the dissolved Cd and Pb) is transferred into 80 ilL acetate buffer (pH 4.5) containing 400 J.1g1L of bismuth nitrate. The solution was applied on the SPCE. After 10 min electrochemical deposition at −1.2 V, the voltammogram was recorded by applying a positive-going square-wave voltammetric potential scan from −1.4 V to 0 V (with a frequency of 20 Hz, amplitude of 25 mV, and potential step of 5 mV).

### Example IV

This example demonstrates the development and use of a miniaturized electrochemical sensor for use in embodiments of the present inventions.

Reagents and materials for an exemplary integrated miniaturized electrochemical sensor: Chemicals used in this study were of analytical reagent grade and used without further purification. Potassium ferricyanide, potassium chloride, lead chloride and cadmium chloride were purchased from Sigma (St. Louis, Mo., USA). Bismuth standard stock solution (1000 mg/L, atomic absorption standard solution) was obtained from Fisher Scientific (Hanover Park, Ill., USA). An acetate buffer solution (0.1 M, pH 4.5) served as the supporting electrolyte. Solutions were prepared with deionized water (approximately 18 MΩcm).

Apparatus for use in the methods of this example. Electrochemical measurement was performed with a potentiostat/galvanostat (263A, Princeton Applied Research, MA) that is connected to a personal computer. The electrochemical software operating system (PowerSuite, Princeton Applied Research, MA) was used for electrochemical measurement and data analysis. The integrated miniaturized electrochemical sensors were purchased from Gwent Inc. England. FIG. 12C shows a schematic of the integrated miniaturized electrochemical sensor, which was composed of two electrodes: carbon electrode (working electrode), and silver/silver chloride electrode (counter & reference electrode). The working area is limited by a meshed well. The volume capacity of the electrochemical cell was 100-200 μL.

Cyclic voltammetry was performed using a potentiostat/galvanostat (263A, Princeton Applied Research, MA) in the presence of dissolved oxygen. The screen-printed electrode was connected to the potentiostat/galvanostat with a specially adapted electrical edge connector. The scanning potential was between −0.4 V and 0.6 V at a scan rate of 50 mV/s.

Stripping voltammetric measurements were performed with an in situ deposition of the bismuth film and target metals in the presence of dissolved oxygen. Studies were carried out by dropping a 100 μL of sample solution in the well. Each electrochemical measurement was carried out in triplicate. A deposition potential of −1.2 V vs. Ag/AgCl was applied to the carbon working electrode without solution stirring. After deposition, the voltammogram was recorded by applying a positive-going square-wave voltammetric potential scan (with a frequency of 20 Hz, amplitude of 25 mV, and potential step of 5 mV). The scan was from −1.2 V to 0.0 V. To evaluate the reusability of electrochemical sensors, a conditioning step at +0.3 V without solution stirring was used to remove the target metals and bismuth prior to the next cycle. Bare electrochemical sensors without in situ bismuth film coating were employed for comparison, with measurement procedures similar to those employed with the bismuth film-coated electrodes (with the exception of using bismuth (III)). Experiments were carried out at room temperature.

Cyclic voltammetry (CV) of ferricyanide was performed as the initial electroanalytical technique for the electrochemical characterization of the sensor surface due to its versatility and relative ease in measurement. A well characterized redox couple (ferrocyanide/ferricyanide) was used to explore the electrochemical nature of the electrode surface.

FIG. 12D shows a cyclic voltammogram obtained on the electrochemical sensor covered by 100 μL unstirred solution of 5 mM ferricyanide. The ratio of the peak anodic current to the peak cathodic current was 0.95. The peak separation was much larger than the predicted value of the reversible voltammetric response and increased with scan rate (data not shown). The results suggest that voltammetric response on the electrochemical sensor can be classed as irreversible.

A reusability test was done to check for interference from the residue of target metals and/or bismuth during the test of reusability, various cleaning time (10 s, 30 s, 1 min) was applied after each SWASV measurement. After 30 s of cleaning, there was no oxidative signal of lead(II) and bismuth on the stripping voltammogram. The result showed that 30 sec was enough to oxidize and remove bismuth and metal ion on the surface of electrochemical sensor. Five milligram per liter of lead(II) solution was then used to evaluate the reusability of bismuth film-coated electrochemical sensor and bare electrochemical sensor.

FIG. 12F shows that the bismuth film coating improves peak current height of SWASV of lead(II), compared to bare electrochemical sensor. When both electrodes were reused, the peak current of lead(II) decreased gradually. When they were used for 5 times, the signal from bismuth film-coated electrochemical sensor was 66.7% only compared to the first measurement; the signal from the bare electrochemical sensor was 45.7% of that at the first time. The results show that the electrochemical sensor, with or without bismuth film coating, is only good for disposable use in SWASV measurement of heavy metal.

The following parameters were optimized for deposition time in order to perform the simultaneous determination of lead(II) and cadmium(II) on electrochemical sensors: deposition potential, concentration of bismuth(III) co-deposited in situ with the target metals (lead and cadmium), and deposition time. Deposition potential was chosen as −1.2V because further reduction of the deposition potential (to −1.4 V) would result in diminished stripping peak heights, multiple stripping peaks, and increased background current due to the hydrogen evolution (Reeder and Heineman, Sensors and Actuators, B: Chemical B52(1-2) (1998) 58-64). Therefore, deposition potential of −1.2V was used in subsequent SWASV measurements.

For the co-deposition of bismuth (III) ions with the target metals, the electrochemical sensor worked as a substrate on which a metallic film of bismuth was formed. The deposition of the bismuth film depends on its ion concentration and deposition (preconcentration) time. Various concentrations of bismuth (III) were chosen for co-deposition onto the bare electrochemical sensor with 5 mg/L of lead(II) at −1.2 V for 2 min of deposition time.

FIG. 12F shows the lead(II) reoxidation peak on electrochemical sensors under various concentrations of bismuth ion. The peak currents increased when the concentrations of bismuth ion increased from 0.1 mg/L to 1 mg/L, because more bismuth was available for co-deposition with lead(II) and improved the stripping signal of lead(II). When the concentration of bismuth was increased to 10 mg/L, beyond the solubility of bismuth in 0.1 M acetate buffer (pH 4.5), precipitation interfered with the co-deposition and then lowered the stripping signal of lead(II). So the 1 mg/L of bismuth was used for subsequent SWASV measurements. The longer the deposition step, the larger is the amount of analyte available on the electrode during the stripping analysis. Increasing the deposition time resulted in increased signal (height of peak current) for both the lead(II) and bismuth stripping waves due to the increased amount of lead ion and bismuth ion reduced on the electrode surface. Kadara and Tothill reported that the diffusion of ions was accelerated under stirring condition and deposition times between 60 and 90 s were sufficient to obtain well defined stripping peaks for lead(II) at this solution concentration (Kadara, et al., Analytical and Bioanalytical Chemistry 378(3) (2004) 770-775). However, in order to detect 100 μL sample on an electrochemical sensor, stirring solution was not practical so that extra deposition time is needed.

FIG. 12G shows that the peak currents of lead(II) increased rapidly with the deposition time in the time interval from 1 to 10 min. The signal from 10 min deposition is roughly twice compared to the 1 min deposition. For longer deposition time above 10 min, the peak current response of electrodes decreased gradually and the wave of lead(II) reoxidation became wider. This result is potentially due to a change in reference electrode potential and the formation of intermetallic compounds after excessive deposition. Thus 10 min of deposition time was used in the subsequence experiments. SWASV measurement of lead and cadmium were done since the unimpeded diffusion to the bismuth-solution interface was available during the reoxidation step, theoretically, sharper and better defined stripping peaks are expected on bismuth film-coated electrochemical sensors compared to bare electrochemical sensor. However, both bismuth film-coated electrochemical sensors and bare electrochemical sensors generated sharp and well-defined stripping peaks in this study.

FIG. 12H shows well-defined sharp peaks over a flat baseline are observed on bismuth film-coated electrochemical sensors, following 10 min of deposition. Peak currents of various lead(II) concentrations (0.5 mg/L, 2 mg/L and 5 mg/L) are 36 μA, 51 μA, and 75 μA, respectively. Peak currents of various cadmium(II) concentrations (0.5 mg/L, 2 mg/L and 5 mg/L) are 37 μA, 48 μA, and 74 μA, respectively. Bismuth shows a peak current around 39 μA at −0.26 V vs. Ag/AgCl.

FIG. 12I shows the stripping voltammograms for lead(II) and cadmium(II) obtained at bare electrochemical sensors. Peak currents of various lead(II) concentrations (0.5 mg/L, 2 mg/L and 5 mg/L) were 20 μA, 40 μA, and 67 μA, respectively. Peak currents of various cadmium(II) concentrations (0.5 mg/L, 2 mg/L and 5 mg/L) were 15 μA, 30 μA, and 40 μA, respectively. When the concentration of lead(II) and cadmium(II) was 0.1 mg/L, there was no current peak in stripping voltammogram on both bare electrochemical sensors and bismuth film-coated electrochemical sensors. The background noise was around 7 μA for both metal ions on bismuth film-coated and bare electrochemical sensors.

FIG. 12J shows that the stripping performance for the same concentration of lead(II) on bare electrochemical sensors exhibited a roughly comparable peak to that obtained on bismuth film-coated electrochemical sensors. However, the stripping performance for the same concentration of cadmium(II) on bare electrochemical sensors exhibited a much lower peak to that of bismuth film-coated electrochemical sensors. When the signal/noise was greater than 3, the detection limits of cadmium(II) were 0.5 mg/L on bismuth film-coated electrochemical sensors and 2 mg/L on bare electrochemical sensors in 100 μL of sample solution. The detection limits of lead(II) were 0.5 mg/L on bismuth film-coated electrochemical sensors and 0.5 mg/L on bare electrochemical sensors in 100 μL of sample solution. Cadmium(II) detection benefited from the addition of bismuth.

In summary, detection limits of cadmium(II) were 0.5 mg/L on bismuth film-coated electrochemical sensors and 2 mg/L on bare electrochemical sensors in 100 μL of sample solution. The detection limits of lead(II) were 0.5 mg/L on bismuth film-coated electrochemical sensors and 0.5 mg/L on bare electrochemical sensors in 100 μL of sample solution. Cadmium(II) detection benefited from the addition of bismuth.

### Example V

This example describes exemplary A) chemicals and reagents and B) bacterial isolates and culturing methods used herein for providing embodiments of the present inventions. Chemical reagents used herein were at least analytical grade.

A) Exemplary chemicals and reagents used herein were aniline, ammonium persulfate, iron (III) oxide, glutaraldehyde, tris buffer, peptone water, sodium phosphate (dibasic and monobasic), 1,4-DL-dithiothreitol (DTT) were purchased from Sigma-Aldrich (St. Louis, Mo.). AuNP (30 nm-diameter) was purchased from BB International (Cardiff, U.K.). Nap-5 column was purchased from GE HEALTHCARE (Piscataway, N.J.). Tryptic Soy Broth (Dickinson and company, MD) was used as the bacterial cultural media.

Probe nanoparticle antibody: Polyclonal goat antibodies highly specific for E. coli O157:H7, showing minimal cross-reactivity to non-pathogenic and non-O157 strains of E. coli (product description) were purchased from Kirkegaard & Perry Laboratories Inc. (Gaithersburg, Md.).

Magnetic nanoparticle antibody: Affinity purified goat monoclonal antibodies specific for E. coli O157:H7 (cross-reacts weakly with Salmonella sp. O antigens, Meridian Life Science, Inc. Specification Sheet) were purchased from Biodesign (Saco, Me.). Universal DNA (such as 5′-TTA TTC GTA GCT AAA AA AAA-3′ A, SEQ ID NO:01) labeled with 6-FAM™ (MW=537.5, Absorbance Max: 495 nm, Emission Max: 520 nm, Extinction Coefficient: 75,000; 5′-/56-carboxyfluorescein (FAM)/TTA TTC GTA GCT AAA AA AAA A/3ThioMC3-D/-3′) (SEQ ID NO:02) was purchased from Integrated DNA Technologies (Coralville, Iowa).

B) Exemplary bacterial isolates and culturing methods used herein during the development of the present inventions. Characterized strains of pathogenic E. coli O157:H7 (American Type Culture Collection (ATCC) #43895) were obtained from the Biosystems Engineering collection, Michigan State University. A 10 μl loop of the isolated was cultured in 10 ml of Tryptic Soy Broth and incubated for 24 h at 37° C. to make a stock culture. A series of 10-fold serial dilutions was prepared from the stock culture using 0.1% peptone water. Experiments were conducted in a certified Biosafety Level II laboratory. The number of target organisms present in the sample was determined by plating 100 μl of E. coli O157:H7 on MacConkey agar from Becton, Dickinson and Company (MD, USA). After 24 h of incubation at 37° C., the presence of the organisms was confirmed by the colors of the colonies on the media: E. coli O157:H7 was dark purple on MacConkey agar.

### Example VI

These Examples describe exemplary production and characterization of probe nanoparticles, in particular probe AuNP nanoparticles coated with probe antibody used in the development of the present inventions.

Determination of antibody binding and amounts to AuNPs: An optimal amount of polyclonal probe antibody (pAb) for binding to the probe AuNP was determined. Conjugation of antibodies to gold particles depends on at least three types of interactions: (a) the electronic attraction between the negatively charged gold particles and the abundant positively charged sites on the protein molecule, (b) an adsorption phenomena involving hydrophobic pockets on the protein binding to the metal surface, and (c) the potential for covalent binding of gold to free sulfhydrl groups [Hermanson, 1996, Bioconjugate technique (San Diego: Academic Press); herein incorporated by reference]. Typically for high-isoelectric point immunoglobulins, coupling at basic pH values increased the coupling yield. Therefore, at a standard basic pH, the inventors made the following concentrations of antibody for determining an optimal amount of pAb per amount of AuNPs. Specifically, 0, 1, 2, 3, 4, and 5 μg pAb was added into each of microcentrifuge tube containing 1 ml of 30 nm AuNP. Each tube was adjusted to a pH of 9.2. After incubation at room temperature for 30 min, 100 μl 2 M NaCl was added to each tube to monitor a red-shift, such that a red/pink (dark grey/light grey) color indicated aggregation of the particles. As shown in FIG. 1A, AuNPs were aggregated at concentrations of 3-5 ug/ml pAB/AuNP solutions (reddish-pink solution) however at a lower concentration of 2 μg/ml AuNP and less, the solutions were not red-shifted (grey).

Optical characteristics of compositions of the present inventions using UV-vis absorption spectrum analysis; Localized surface plasmon resonance (LSPR) wavelength of UV-vis absorption spectrum (a wavelength at the maximal absorption peak) was used for determining whether the elements were attaching (coating) a nanoparticle.

Localized surface plasmons occurs when the incident light frequencies match the collective oscillations of the conduction electrons of metal nanoparticles or nanoislands. Localized surface plasmons are sensitive to changes in the local dielectric environment caused by analyte adsorption. This leads to the following relationship [Willets, et al., 2007, Annu. Rev. Phys. Chem. 58 267-297]:

Δλₘₐₓ=mΔn[1−exp(−2d/l_{d})]. (1)

Here m is the bulk refractive-index response of the nanoparticle(s); Δn is the change in refractive index induced by the adsorbate; d is the effective adsorbate layer thickness; and l_{d} is the characteristic EM-field-decay length.

An analysis of the antibody coating indicated in FIG. 1A was determined by a comparison to pure 30 nm AuNP, showing a peak of approximately 524 nm, FIG. 1(B). After incubation of 0.1 mg/ml pAb (20 μl pAb), in 1 ml of 30 nm AuNP, the spectrum peak shifted to 528 nm, see FIG. 1(B). Therefore, the spectrum in FIG. 1B confirmed that pAb was conjugated with the 30 nm AuNP.

By extrapolation, increasing the amount of antibody was contemplated to increase the wavelength of the absorption peak.

However, to optimally prepare probe AuNPs there would be some surface space available for attaching thiolated oligonucleotide silent DNA after conjugation of the antibody to the probe nanoparticle. Since the aggregation red-shift occurred between 2 and 3 mg of antibody, 2.5 μg/ml of pAb was used as an exemplary amount for coating AuNPs for providing probe nanoparticles.

Similarly, the optical property of labeled silent DNA and nanoparticles coated with pAb and silent DNA conjugated to a fluouresent molecule, 6-FAM, were measured with UV-vis absorption spectrum. An absorption spectrum of fluorophore-labeled barcode DNA is shown in FIG. 1C (solid line) where two absorption peaks at 260 nm and 495 nm were observed. The absorption peak at 260 nm shows single-stranded DNA [Storhoff, et al., 1998, J. Am. Chem. Soc. 120 1959-1964; herein incorporated by reference]. The absorption peak at 495 nm is an optical characteristic of a fluorophore of 6-FAM, fluorescein, whose absorption maximal wavelength was 495 nm [Fischer, et al., 2003, Bioconjug Chem. 14(3) 653-660; herein incorporated by reference]. 6-FAM was chosen due to its relatively high absorptivity, excellent fluorescence quantum yield, and good water solubility. FIG. 1C also shows an absorption spectrum of the AuNP probe functionalized (coated) with the barcode DNA and pAb (dashed line). An absorption peak of 260 nm related to single-stranded DNA was still visible, however the absorption peak around 524 nm related to the fluorophore was no longer visible. The disappearance of this absorption peak using the AuNP complex (pAb and labeled silent DNA) was contemplated as the result of an offset between emission of fluorophore-labeled barcode DNA and absorption of AuNP around 520 nm ranges. Emission maximal wavelength of the barcode DNA was 520 nm and its wavelength range was overlapped with that of the absorption peak of 30 nm AuNP conjugated with pAb. Therefore, the probe AuNP comprised pAb and fluorophore-labeled barcode DNA.

Preparation of probe nanoparticles for use in an AUNT biosensor of the present inventions: The polyclonal antibody (pAb; 0.1 mg/ml, 25 μl) solution was mixed with the 30 nm AuNPs solution (1 ml) then adjusted pH to 9.2 with 2 mM borax and incubated for 30 minutes. Subsequently thiolated silent DNA (4 nmol) were attached to the pAb coated particles by slow salt aging (3 h) to a final concentration of phosphate-buffered saline (PBS; 0.15 M NaCl in 0.01 M of phosphate buffer) and incubated for 1 hour. The AuNPs were centrifuged for 30 min at 15,800 g at 4° C., the supernatant containing excess DNA was removed while the pellet of nanoparticles was resuspended with 1 ml of 0.15 M PBS (pH 7.4). This washing procedure was repeated two times. After washing, the coated probe AuNPs were resuspended in 200 μl of 0.15 M PBS (pH=7.4), and stored at 4° Celcius.

Testing for release of the thiolated barcode (silent) DNA: Due to the process of antibody coating followed by DNA attachment to the nanoparticle, the inventors next determined whether DTT would release the thiolated barcode (silent) DNA from the AuNP probe for accurate fluorescence measurements. DTT is a common disulfide reducing agent used to release covalently attached barcode DNA from AuNP probes and was used to efficiently remove thiolated oligonucleotides from gold surfaces Liu, et al., 2005, Analytica Chimica Acta 533 (1) 3-9; Ninfa, et al., 1998, Fundamental Laboratory Approaches for Biochemistry and Biotechnology (Bethesda: Fitzgerald Science Press)].

In order to measure fluorescence intensity of released barcode DNA from AuNP probe, 60 μl probe AuNP was mixed with 100 μl of 0.5 M DTT and incubated for 15 min at 50° C. and 1 h 30 min at room temperature. To separate released thiolated barcode DNA from the surface of the 30 nm AuNP, the AuNP probe mixture was centrifuged at 15,800 g for 30 min. The supernatant of the mixture was used for fluorescence measurements. FIG. 1D shows a fluorescence reading from released the barcode DNA is much higher than that from unreleased DNA still attached to the probe AuNP, a 30 nm AuNP solution was used as a reference (control, con). These results showed that a fluorophore was released from the AuNP probe by using DTT.

### Example VII

These Examples describe exemplary synthesis and characterization of compositions comprising polyaniline-coated magnetic nanoparticles and compositions comprising antibody coated magnetic polyaniline probes.

Polyaniline-coated magnetic nanoparticles were synthesized according to Pal, et al., 2007, Biosens. Bioelectron. 22 2329-2336; and Sergeyeva, et al., 1996. Polyaniline label-based conductimetric sensor for IgG detection Sens. Actuators B Chem. 34 283-288; each of which are herein incorporated by reference.

In brief, Gamma iron (III) oxide (γ-Fe₂O₃) nanoparticles were obtained from a commercial source (such as Sigma-Aldrich, MO). These nanoparticles were dispersed in a mixture of 50 ml HCl, 10 ml deionized water and 0.4 ml aniline by sonication for 1 h at 0° Celcius. This was followed by a slow addition of 20 ml of the oxidant, ammonium persulfate, at a rate of 0.1 ml/min with continuous stirring of the above solution mixture which resulted in the coating of polyaniline on the smaller γ-Fe₂O₃ nanoparticles. The above reaction was continued for 4 h in an ice water bath and was filtered followed by subsequent washings with 1 M HCl, methanol and diethylether. The powder obtained was dried for 48 h at room temperature. The concentration of γ-Fe₂O₃: aniline monomer (1:0.6) was used herein with increasing concentrations of the aniline monomer. The morphology of magnetic polyaniline was investigated by a transmission electron microscope (TEM) (JEOL JEM-100CX II, Japan).

Polyaniline-coated magnetic nanoparticles fractions (20 mg) obtained as a result of filtration were incubated overnight with 1 ml of 0.8% glutaraldehyde. The polyaniline-coated magnetic nanoparticles were then separated by magnetic field from the liquid supernantent which was discarded.

Functionalization of polyaniline-coated magnetic nanoparticles with mAb.: Antibody coating (conjugated) to polyaniline-coated magnetic nanoparticles (PANI) was evaluated by using UV-vis spectrum. Polyaniline has very high electrical conductivity in the protonated state and is bound easily to most proteins [Braakman, et al., 1992, Nature 356:260; herein incorporated by reference]. However since in the compositions of the present inventions, the polyaniline was coating nanoparticles, it was not known whether monoclonal antibodies were capable of binding, especially due to the potential altering in binding capabilities for the alinine in the presence of iron and due to the small size of the nanoparticles. Therefore the inventors first determined whether these mAb would efficiently bind to the polyaniline-coated magnetic nanoparticles.

A transmission electron microscopy (TEM) image in FIG. 2(A) shows that polyaniline-coated magnetic nanoparticles are formed. The appearance of these nanoparticles appeared to be similar to those in Sergeyeva, et al., 1996, Sens. Actuators B Chem. 34:283-288; herein incorporated by reference. FIG. 2(B) shows absorption spectra before and after conjugation of the monoclonal antibody with polyaniline-coated magnetic nanoparticles. There was no absorption peak observed on the graph at 280 nm for the polyaniline-coated magnetic nanoparticles which did not contain monoclonal antibody (dotted line). However, an absorption peak was present after conjugation of the monoclonal antibody with polyaniline-coated magnetic nanoparticles that were modified with glutaraldehyde as a crosslinker (solid line). A general reference of optical properties of polyaniline is found in Granholm, et al., 1997, Synthetic Met. 84 783-784; herein incorporated by reference. It is well known that amino acids with aromatic rings exhibit a diagnostic absorbance at 280 nm, which is used as a confirmation method for the conjugation of proteins [Hsing, et al., 2007, Electroanalysis 19:755-768; herein incorporated by reference].

Gluteraldehyde apparently produces ring-like cross-linking of the monoclonal antibodies with the polyaniline on the magnetic nanoparticle, therefore the inventors determined the polyaniline-coated magnetic nanoparticles were conjugated with mAb by using glutaraldehyde as a crosslinker through the appearance of the peak near 280 nm.

Preparation of polyaniline-coated magnetic nanoparticle compositions: Five hundred μl aliquots of the monoclonal antibody (mAb; 400 μg/ml) were added to the polyaniline-coated magnetic nanoparticles modificated with glutaraldehyde. The incubation procedure was performed with shaking for 3 h at room temperature and 500 μl of 100 mM Tris buffer containing 0.05% of Tween 20 (pH 7.4) was added to the reaction mixture in order to inactivate non-reacted aldehyde groups for 30 min. Finally, polyaniline-coated magnetic nanoparticles conjugated with mAb (hereafter referred to as MPANI probe) was separated by the magnetic field and were mixed with 2 ml of 150 mM PBS (pH 7.4).

### Example VIII

The following examples describe an exemplary biosensor composition and method of use as a diagnostic system (assay). Specifically, in one embodiment, a biosensor is a combination of a probe AUNT nanoparticle (a silent DNA conjugated to a label attached to a nanoparticle) comprising a probe antibody, wherein said probe antibody is a polyclonal anti-E. coli O157:H7, and a polyaniline-coated magnetic nanoparticle, wherein said magnetic nanoparticle further comprises a monoclonal antibody for binding to an E. coli O157:H7 bacterium. This biosensor was tested for pathogen detection by evaluating the detection of an exemplary pathogen, E. coli O157:H7 bacterium in a sample. See, schematic provided in FIG. 3.

Further, this example demonstrated that the reproducibility and reliability of polymeric wire biosensor was enhanced by using screen-printed silver electrodes and pulse mode measurement. The screen-printed electrodes had a uniform distance of 550 μm between two electrodes and an average resistance of 1.4Ω. Formation of polyaniline-coated magnetic nanoparticles was confirmed by a TEM image. The polyaniline was used as an electric signal label of the biotin for the biospecific binding counterpart of streptavidin. The pulse mode polymeric wire biosensor showed more reliable electrical response induced by streptavidin-biotin interactions by reducing the resistance related to the interfacial capacitance. A dose-dependent response was also successfully measured by the pulse mode polymeric wire biosensor. Thus, it is suggested that the performance enhanced disposable polyaniline-based biosensor has great potential for field test with real-time measurements.

Specific concentrations of E. coli O157:H7 bacteria were prepared by a 10-fold serial dilution process. Each concentration of E. coli O157:H7 in 100 μl was mixed with 200 μl of the MPANI probe and 700 μl of 0.1% peptone water, then incubated for 20 minutes, See, FIG. 3.

The target complex (the MPANI probe bound with E. coli O157:H7) was magnetically separated, then resuspended with 160 μl of 150 mM PBS (pH 7.4). The target complex was incubated with 600 μl of 150 mM PBS (pH 7.4) containing 0.025% Tween 20 and 0.1% BSA, and 40 μl of AuNP probe for 1 h 30 minutes. The sandwich complexes were magnetically separated and washed with 500 μl of 150 mM PBS (pH 7.4) containing 0.025 Tween 20 and 0.1% BSA. In the final step, 200 μl of 0.5 M DTT was added and the solutions were incubated at 50° C. for 15 min to allow for complete dehybrization of the barcode DNA with fluorophore. The sandwich complexes were again separated magnetically and the supernatant was used for detection of E. coli O157:H7 with fluorescence. A VICTOR³ multilabel plate reader was used for measuring 6-FAM™-functionalized barcode DNA which was excited at 488 nm with the emission measured at 535 nm.

Next the inventors investigated whether the biosensor assay based on a polyaniline-coated magnetic nanoparticles was actually capable of detecting E. coli O157:H7 in a specific range of concentrations of the bacterium. MPANI probes without E. coli O157:H7 bacterium were incubated with probe AuNPs, which were used as a reference to monitor fluorescence signal changes. A sandwich complex was prepared with various concentrations of E. coli O157:H7 which were serially diluted as 10-fold from the stock culture.

FIG. 4(A) shows that fluorescence intensity from the various concentrations of E. coli O157:H7 is much higher than that from the reference mixture without bacterium. For quantitative analysis, a pure increment of fluorescence intensity from the references was calculated from the FIG. 4(A). Δ fluorescence was not proportional to the concentration of E. coli O157:H7 (FIG. 4(B)). Thus an unexpected parabolic signal response was observed correlated with the concentrations of E. coli O157:H7 if signals were neglected at a concentration of 10² and 10⁶ CFU/ml. Conversely, an inverted parabolic signal response was observed in a capillary action based disposable immunosensor [Muhammad-Tahir, et al., 2003, Biosens. Bioelectron. 18, 813-819]. Therefore, even though this experiment failed to show a direct dose-dependent signal response for detecting E. coli O157:H7 with an assay based on this combination biosensor, E. coli O157:H7 bacterium was detected in order of single bacterium per CFU/ml concentration establishing the capability of this type of biosensor for detecting a highly pathogenic bacterium.

In summary, the experiments described herein demonstrated that a version of an AUNT assay, comprising a probe nanoparticle and a polyaniline-coated magnetic nanoparticle, wherein the probe molecules attached to the nanoparticles were antibodies that detected E. coli O157:H7 in order of ones CFU/ml concentration. AuNP probes were made by 2.5 μg/ml pAb and 4 nmol barcode (silent) DNA. Conjugation of pAb and fluorophore-labeled barcode DNA with the AuNP. Polyaniline-coated magnetic nanoparticles modified with glutaraldehyde were conjugated with mAb. The fluorophore-labeled barcode DNA was released from the AuNP probes by 0.5 M DTT. E. coli O157:H7 bacterium was detected in order of ones CFU/ml. Thus, the bio-barcode assay based on polyaniline-coated magnetic nanoparticles has good potentials for detection of foodborne and waterborne pathogens such as E. coli O157:H7.

### Example IX

The following examples describe an exemplary electrically active polyaniline coated magnetic (EAPM) nanoparticle-based biosensor developed for the detection of Bacillus anthracis endospores in contaminated food samples

The following examples describe an exemplary electrically active polyaniline coated magnetic (EAPM) nanoparticle-based biosensor developed for the detection of Bacillus anthracis endospores in contaminated food samples (Pal, et al., Electrically active polyaniline coated magnetic (EAPM) nanoparticle as novel transducer in biosensor for detection of Bacillus anthracis spores in food samples. Biosens Bioelectron. 2009 Jan. 1; 24(5):1437-44. Epub 2008 Aug. 22); herein incorporated by reference in its entirety.

This Example describes a novel integrated design of using electrically active polyaniline coated magnetic nanoparticles as a magnetic concentrator and biosensor transducer in one system. The biosensor detection system is fast and includes a magnetic concentration time of 10 min followed by a signal detection time of 6 minutes. The sensitivity of the biosensor in the detection of B. anthracis spores from complex food matrices was found to be 4.2×10² spores/ml in lettuce and ground beef, and 4.2×10³ spores/ml in whole milk. The EAPM nanoparticles also showed excellent capture efficiency from the food samples with a highest capture ratio of 0.97. Results indicate that the EAPM nanoparticles have great potential in biosensing applications. Future research work will involve in advancing the electrical and magnetic properties of the nanoparticles for biosensor detection of diverse targets. Furthermore, the easy-to-use EAPM biosensor described herein has the potential to be applied as a rapid detection device in biodefense and diagnostics.

One hundred nm-diameter EAPM nanoparticles were synthesized from aniline monomer (made electrically active by acid doping) coating the surface of gamma iron oxide cores. The magnetic, electrical, and structural characteristics of the synthesized EAPM nanoparticles were studied using superconducting quantum interference device (SQUID), four-point probe, and transmission electron microscopy (TEM). Room temperature hysteresis of the synthesized nanoparticles shows a saturation magnetization value of 44.1 emu/g. The EAPM nanoparticles were biologically modified to act as an immunomagnetic concentrator of B. anthracis spores from lettuce, ground beef and whole milk samples and are directly applied to a direct-charge transfer biosensor. The detection mechanism of the biosensor depends on the capillary flow of the captured spores on the biosensor surface along with direct-charge transfer across the EAPM nanoparticles. Experimental results indicate that the biosensor is able to detect B. anthracis spores at concentrations as low as 4.2×10² spores/ml from the samples. The EAPM-based biosensor detection system is fast and reliable with a total detection time of 16 min.

Exemplary A) chemicals and reagents and B) bacterial isolates and culturing methods used herein for providing embodiments of the present inventions are provided below. Chemical reagents used herein were at least analytical grade.

A) Exemplary chemicals and reagents used herein were aniline, iron(III) oxide nanopowder, glutaraldehyde, polysorbate 20 (Tween 20), hydrochloric acid (1N), ammonium peroxy disulfate, tris buffer, peptone water, and sodium phosphate (dibasic and monobasic) were purchased from Sigma-Aldrich (St. Louis, Mo.). Mouse monoclonal anti-anthrax IgG (clone 2C3) and poly-clonal goat anti-anthrax combo IgG molecules were obtained from Chemicon International (Millipore, CA) whereas anti-goat IgG-FITC (whole molecule) was obtained from Sigma-Aldrich (St. Louis, Mo.). Nitrocellulose and cellulose acetate membrane pads for the biosensor were purchased from Millipore (Bedford, Mass.). Conductive micro-tip pen used for dispensing silver electrodes were procured from Chemtronics (Kennesaw, Ga.). De-ionized water from Millipore Direct-Q system was used for preparing reagents.

B) Exemplary bacterial isolates and spores used herein were characterized strains of B. anthracis (Sterne) obtained from the Michigan Depth linent of Community Health (Lansing, Mich.). Generic Escherichia coli and Salmonella enterica serovar Enteritidis (S-64) strains were procured from the collection of the Biosensors Laboratory at Michigan State University. The B. anthracis and E. coli strains were grown in trypticase soy broth (BD Biosciences, MD), while lactose broth (BD Biosciences, MD) was used for growing S. Enteritidis cultures. The cultures were grown at 37° C. for 24 hour. Sporulation of B. anthracis Sterne strain was promoted following a previously published protocol (Pezard et al., 1991). The spore count was estimated using a cell counting chamber (Hemocytometer) and viable spores were enumerated by microbial plating in trypticase soy agar(II) with 5% sheep blood plates (BD Bio-sciences, MD). The plating and colony counting experiments were performed using automated spiral plater and colony counter equipments (Microbiology International, MD).

EAPM nanoparticle synthesis and characterization studies: The EAPM nanoparticles were synthesized from aniline monomer (made electrically active by acid doping) and gamma-iron(III) oxide (γ-Fe2O3) nanoparticles using a chemical synthesis procedure (Sharma et al., 2005). The γ-Fe2O3 to monomer weight ratio was maintained at 1:0.6 in the synthesis procedure. A superconducting quantum interference device (Quantum Design MPMS SQUID) magnetometer was used for magnetic characterization studies of the synthesized EAPM nanoparticles. For hysteresis loop measurement, the nanoparticles were subjected to a magnetic field cycling between +15 and −15 kOe; while the temperature was maintained constant at 300 K. Electrical conductivity of the synthesized EAPM nanoparticles was evaluated in solid form. The nanoparticles were compressed into pellets and electrical conductivity was measured at room temperature using a Four Point Probe (Lucas/Signaton Corporation, Pro4; CA). The structural morphology and size distribution of the nanoparticles were studied using a 200 kV field emission transmission electron microscope (JEOL 2200 FS).

FIG. 2 shows the magnetization versus magnetic field, i.e. the M-H loop measurements of both unmodified Fe₂O₃ and synthesized EAPM nanoparticles, using a DC SQUID magnetometer at 300 K. Both systems showed tendency to saturate at 15 kOe. The saturation magnetization (MS) for the Fe₂O₃ nanoparticles was found to be 64.4 emu/g, whereas for the EAPM nanoparticles, the saturation magnetization reached 44.1 emu/g. The decrease in MS value for the EAPM nanoparticles is expected due to surface interactions between the polymer (polyaniline), which is diamagnetic in nature, and iron oxide nanoparticles (Alam et al., 2007, Journal of Magnetism and Magnetic Materials 314(2):93-99). The remanent magnetization Mr for the Fe₂O₃ and the EAPM nanoparticles were found to be 14.2 and 10.4 emu/g, respectively, and the coercive force Hc for both nanoparticles were 200 Oe.

The low values of Mr and Hc suggest that the nanoparticles are still in the ferromagnetic phase but approached superparamagnetic behaviour (Kryszewski and Jeszka, 1998). FIG. 2 shows electrical conductivity measurements of both synthesized EAPM and unmodified Fe₂O₃ nanoparticles compressed into pellets of 2000-μm thickness at room temperature. The Fe₂O₃ nanoparticles have an electrical conductivity as low as 3.4×10-5 S/cm whereas, conductivity of the EAPM nanoparticles increases by five orders of magnitude to 3.3 S/cm. This increase in electrical conductivity is expected and confirms the presence of electrically active polyaniline in the nanoparticles. FIG. 2 and d reveal transmission electron microscope (TEM) images of the unmodified Fe₂O₃ nanoparticles and the polyaniline coated EAPM nanoparticles. The iron oxide nanoparticles have an average diameter of 20 nm according to manufacturer's specifications which is found to be consistent with the TEM image in FIG. 2, whereas the polyaniline coated EAPM nanoparticles shows diameter ranging from 50 to 100 nm (FIG. 2).

Bio-modification of EAPM nanoparticles: The synthesized EAPM nanoparticles were conjugated with mouse monoclonal anti-B. anthracis IgG molecules by direct physical adsorption of the antibodies on the nanoparticles. Anti-B. anthracis IgG (150 μg/ml) was mixed with the EAPM nanoparticles (100 mg/ml) in 100 mM phosphate buffer (pH 7.4). The reaction mixture was incubated for 1 h at 25° C. in a rotational hybridization oven (Amerex Instruments, Inc. CA). After incubation, the IgG modified nanoparticles (immuno-EAPMs) were magnetically separated to remove the supernatant using a magnetic separator (Spherotech, IL). The immuno-EAPMs were washed three times with a blocking buffer consisting of 100 mM Tris-HCl buffer (pH 7.6) and 0.1% (w/v) casein. Finally, the immuno-EAPMs were suspended in 100 mM phosphate buffer (pH 7.4) and stored at 4° C. The bio-modification of the EAPM nanoparticles was investigated by measuring the absorption of IgG molecules before and after reaction with the nanoparticles using a UV-vis-NIR scanning spectrophotometer (UV-3101PC, Shimadzu, Kyoto, Japan).

Physical adsorption has been used for bio-modification of the EAPM nanoparticles with IgG molecules because the conjugation procedure is simple and allows IgG molecules to retain their activity and conformation (Zhou et al., 2004,). The UV spectrum of pure anti-B. anthracis IgG (150 μg/ml) molecules and the unreacted IgG molecules in the supernatant after magnetic separation of immuno-EAPMs was measured by a UV-vis-NIR scanning spectrophotometer. Literature suggests that IgG adsorption occurs preferentially through Fc fragment of the molecule (Buijs et al., 1996,).

Fabrication of the biosensor: The nitrocellulose biosensor capture membrane pads were first cleaned with de-ionized water followed by 10% methanol (v/v) for 45 mM and dried at room temperature. The cleaned membranes were treated with 0.5% glutaraldehyde solution (v/v) for 1 h. The glutaraldehyde activated capture membranes were coated with polyclonal goat anti-B. anthracis IgG (500 mg/ml) using a reagent dispensing module (Matrix 1600, Kinematic Automation Inc., CA) and the antibody treated membranes were then incubated for 1 h at 37° Celcius. Finally, the membrane surface was washed with blocking buffer consisting of 100 mM Tris-HCl buffer (pH 7.6) and 0.1% (v/v) Tween 20 and incubated for 45 mM at 37° Celcius. The antibody modified membranes were dried at room temperature and stored at 4° C. The anti-B. anthracis IgG attachment on the capture pad was confirmed by secondary FITC anti-goat IgG molecules as label using a laser scanning confocal microscope (Olympus Fluoview 1000). The application and the absorption cellulose membrane pads were washed with de-ionized water to remove dirt and surface residues and dried at room temperature.

Biosensor design and detection: The biosensor design shown herein was modified from a direct-charge transfer biosensor previously developed by the authors (Pal et al., 2007, Biosensors & Bioelectronics 22(9/10):2329-2336). In the modified design (FIG. 13), the biosensor consists of three disposable membrane pads: sample application pad, capture pad, and absorption pad. The overall biosensor dimension along with the dimensions of each membrane pad has also been tabulated in FIG. 13. Silver electrodes were fabricated along both sides on the capture pad leaving an electrode gap of 0.5 mm. For data acquisition, the biosensor units were connected to a handheld multimeter linked with a computer. FIG. 13 is a schematic representation of the immunomagnetic separation and biosensor detection procedure. The biosensor detection involves a sandwich immunoassay, with a capture anti-body (IgG) immobilized on the biosensor capture pad and a detector antibody (IgG) conjugated with synthesized EAPM nanoparticles. The detector antibody conjugated EAPM nanoparticles are added to the B. anthracis spore contaminated food samples (FIG. 13, step 1) and used to immunomagnetically concentrate the spores from the complex food matrices (step 2). The concentrated targets are then washed to remove unbound materials (step 3) and applied to the sample application pad of the biosensor (step 4). The antigen-antibody-EAPM complex flows to the capture pad, where the antigen is anchored by the capture antibodies present and a sandwich complex is formed (step 5). The conductive EAPM nanoparticles bound to the antigens in the sandwich act as a voltage controlled “ON” switch resulting in decreased resistance across the silver electrodes which is recorded electrically (step 6) (see, Pal, et al., 2007. Biosens. Bioelectron. 22, 2329-2336, Pal, et al., 2008, IEEE Sensors Journal 8(6): 647-654).

Target capture and biosensor detection: The immuno-EAPMs were added to different concentrations of the B. anthracis spore inoculated food samples (4.2×10¹ to 4.2×10⁷ spores/ml) in order to have a final immuno-EAPM concentration of 20 mg/ml. This was followed by 10-min incubation with gentle shaking at room temperature. The immuno-EAPM-spore complexes formed were magnetically separated from the unbound spores and food matrices, to remove the supernatant, and washed twice with sterile de-ionized water before re-suspending in 10 ml 0.1% (w/v) peptone water. A control or blank solution containing the same concentration of the immuno-EAPMs but with no B. anthracis spores in 0.1% (w/v) peptone water was simultaneously prepared for each food sample. For the detection process, the biosensor units were connected to a hand-held multimeter (BK Precision, AK-2880A, MA) with RS-232 interface and linked to a computer. One hundred microliters of the captured immuno-EAPM-spore complexes was applied to the application pad on the biosensor and the resistance signal across the silver electrodes was recorded. Resistance measurements were made both before and after sample application. The flow time of the sample from application membrane to capture membrane was approximately 1 min and data recording for each sample was carried through 6 min. For each experiment, a minimum of three replications were performed. The biosensors were calibrated against corresponding control solutions. Biosensor experiments were performed in a Biosafety Level II Laboratory.

Biosensor specificity study: Pure spore suspensions of B. anthracis, and pure cultures of generic E. coli and S. Enteritidis were used for evaluating specificity of the EAPM biosensor. The immunoEAPMs were added to 10 ml solutions of the appropriate concentration of the desired bacteria to a final immuno-EAPM concentration of 20 mg/ml. The immunomagnetic concentration and biosensor detection procedure was similar to that as described in Section 2.8.

Generic E. coli cell concentrations ranging from 1.7×10¹ to 1.7×10⁵ CFU/ml in sterile 0.1% (w/v) peptone water, S. Enteritidis cell concentrations ranging from 1.6×101 to 1.6×10⁵ CFU/ml in sterile 0.1% (w/v) peptone water, and B. anthracis spore concentrations ranging from 4.2×10¹ to 4.2×10⁵ in sterile water were used for specificity studies. The control solutions consisted of EAPM nanoparticles suspended in 0.1% (w/v) peptone water or sterile water to a final concentration of 20 mg/ml.

Food sample preparation: Romaine lettuce, lean ground beef, and ultra-pasteurized whole milk, was purchased from a local grocery store. For the lettuce and beef samples, 25-g samples were weighed, mixed with 225 ml of 0.1% (w/v) peptone water in a Whirl-Pak plastic bag, and stomached in a stomacher (Microbiology International, MD) for 1 min. The whole milk samples were used as purchased. Nine milliliters of the three kinds of liquid samples were thoroughly mixed with 1 ml of appropriate concentrations of B. anthracis spore stock solution in a Vortex mixer (Fisher Scientific, IA). Finally, a series of 10 ml samples inoculated with B. anthracis spores at concentrations ranging from 10¹ to 10⁷ spores/ml were obtained.

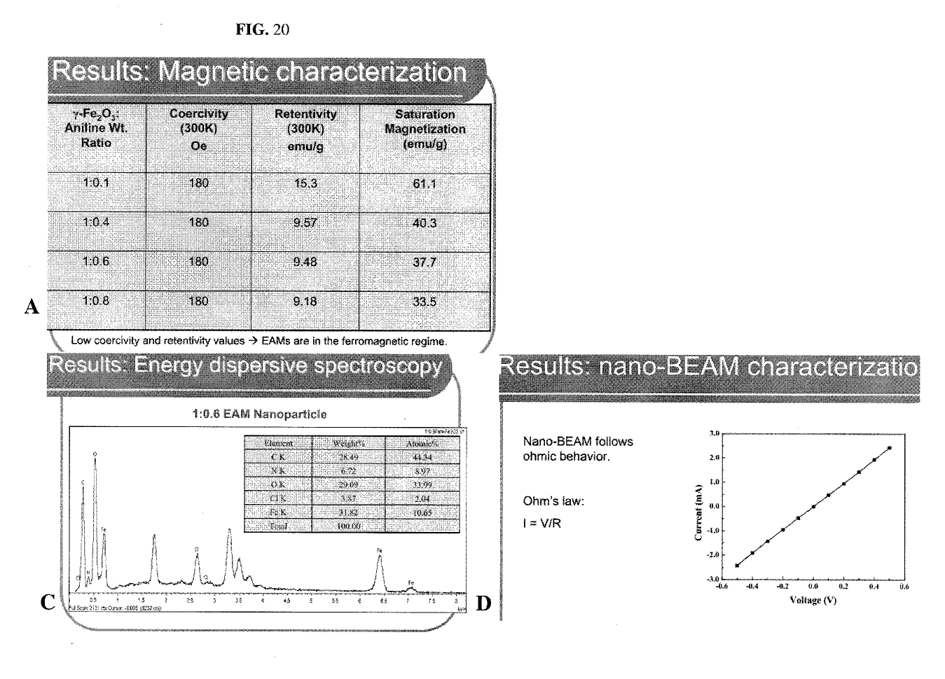

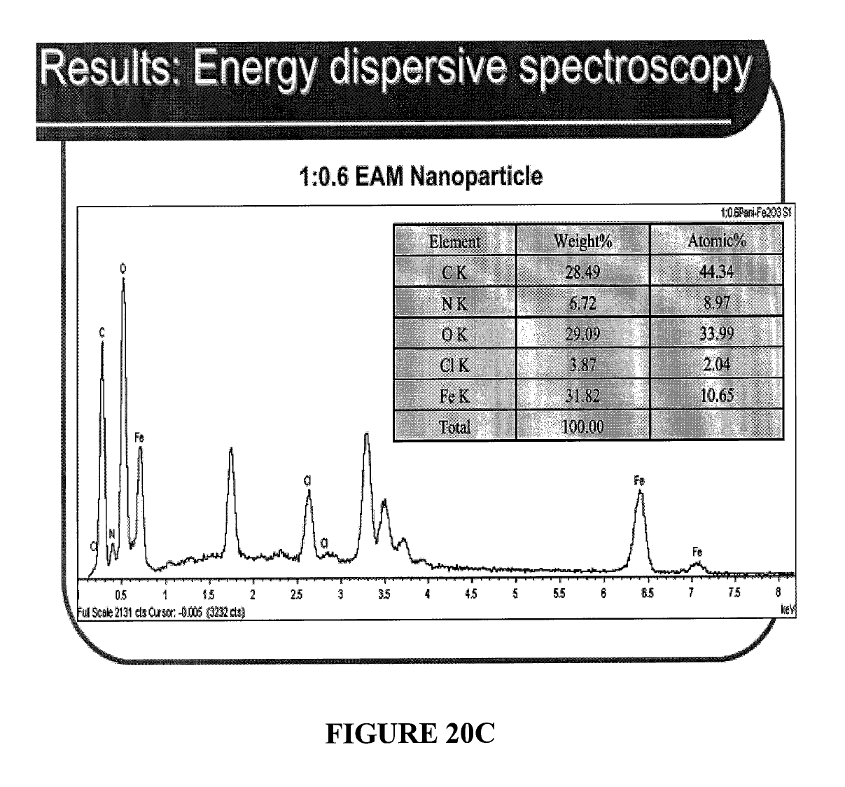

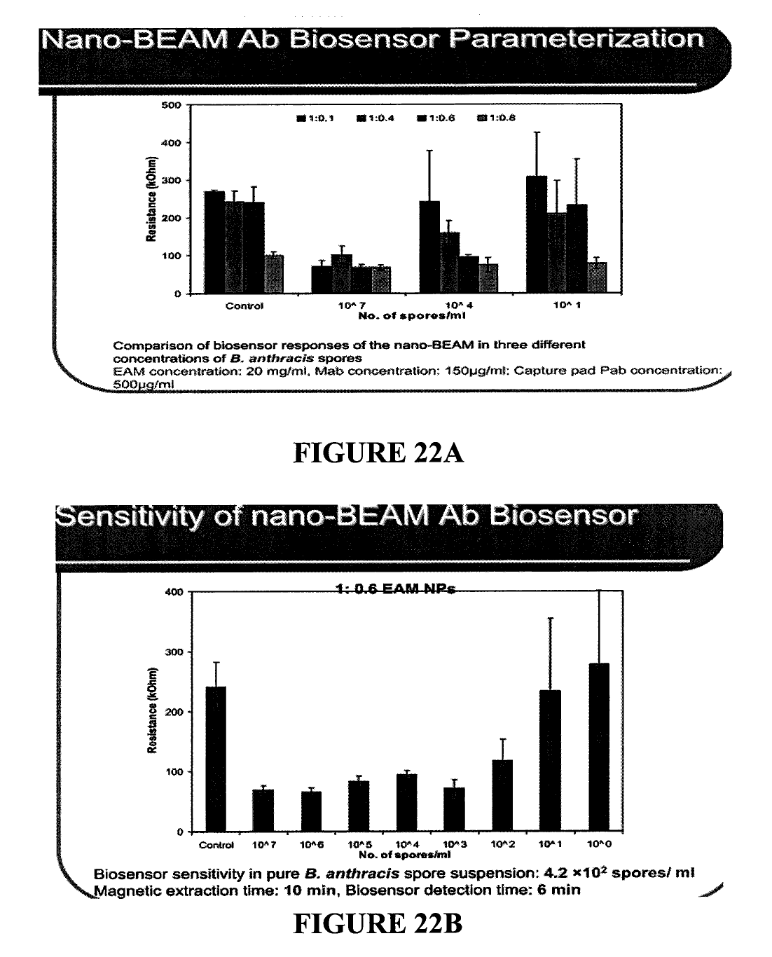

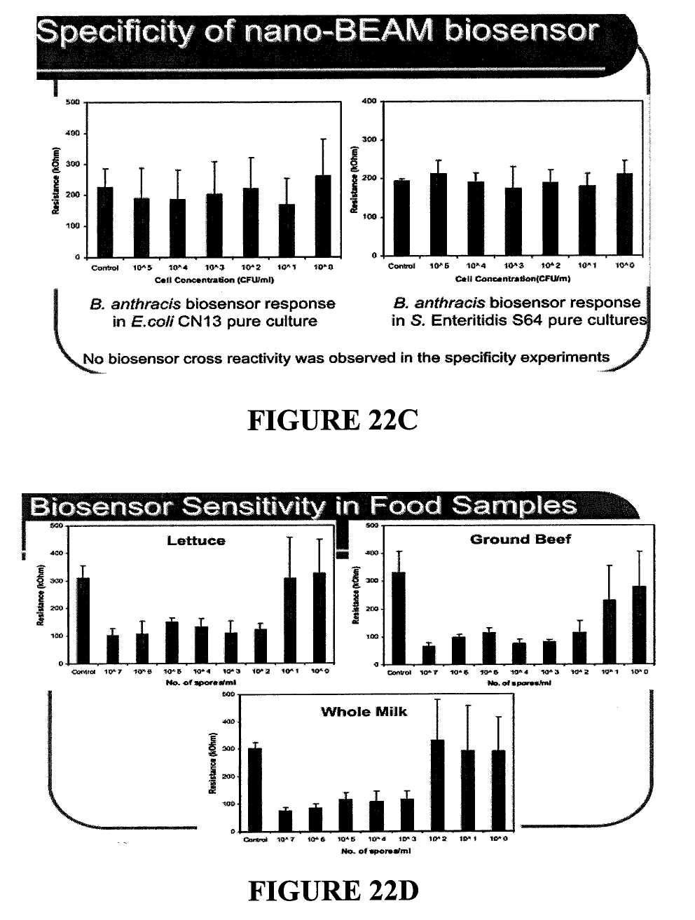

Hence, electrostatic interactions between negatively charged Fc fragment of IgG molecules and positively charged polymer surfaces in the EAPM nanoparticles might play a significant role in the adsorption process. The immobilization of polyclonal goat anti-B. anthracis IgG on the biosensor capture pad was detected by secondary FITC anti-goat IgG and confirmed by fluorescent images obtained from a laser scanning confocal microscope (FIG. 20). FIG. 22 show the average resistance readings measured with the EAPM nanoparticle-based direct-charge transfer biosensor in lettuce, whole milk, and ground beef samples inoculated with B. anthracis spores. The average resistance signals obtained from three replicates were plotted for the control and the food samples, contaminated with spore concentrations ranging from 4.2×10¹ to 4.2×10⁷ spores/ml.

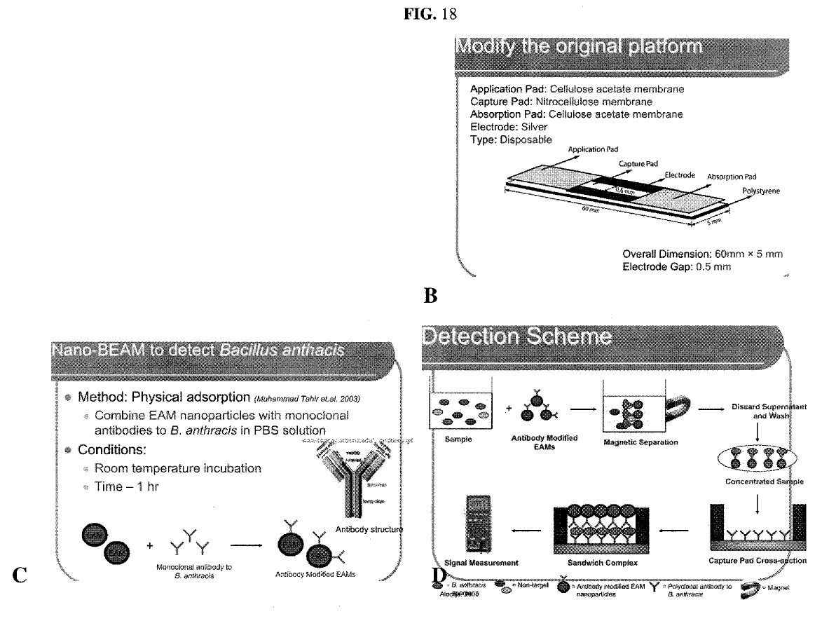

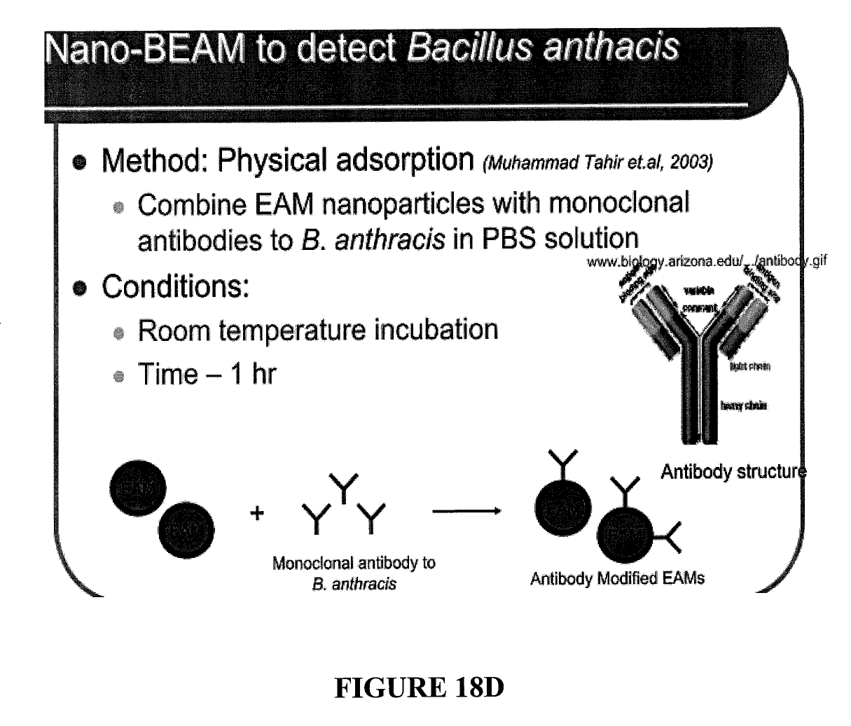

As observed, the resistance values recorded for the different spore concentrations are much lower than the values for the control solution that has no spores in it. The average resistances for the control solutions for at least three food samples are in the range of 288±50 and 353±51 kΩ, whereas the average resistances for the different spore concentrations in the three food samples vary from 75.1±14 to 132.6±19 kΩ. The reduced resistance values support the formation of a sandwich complex on the capture pad (FIG. 18), where the conductive EAPM nanoparticles act as a charge transfer agent causing a drop in the resistance signal across the silver electrodes (Kim et al., 2000; Pal et al., 2007). Single factor analysis of variance (ANOVA) to a significance of 95% (P<0.05) was used to compare the differences in the resistance values between the control and the different spore concentrations. The lowest spore concentration that produced a resistance signal significantly different (P<0.05) from the control was considered to be the sensitivity or detection limit of the biosensor. For the lettuce and ground beef samples, the biosensor sensitivity was determined to be 4.2×10² spores/ml with statistically significant differences from the control (P-value for lettuce at 102 spores/ml was 1.79E-05; P-value for ground beef at 10² spores/ml was 2.63E-06). For whole milk samples, the biosensor could reach a sensitivity of 4.2×10³ spores/ml where statistically significant differences could be observed from the control (P-value at 10³ spores/ml was 8.47E-08). The reduced biosensor sensitivity in the whole milk samples could be attributed to the high fat content in these samples.

TABLE 5
Capture efficiency of the immuno-EAPM conjugates (Electrically active
polyaniline coated magnetic (EAPM) nanoparticle) in romaine lettuce, whole milk, and
ground beef contaminated with Bacillus anthracis spores.

| | | Romaine | | | | Ground | |
|---|---|---|---|---|---|---|---|
| | | Lettuce | | | | beef | |
| | | No. of | | | | No. of | |
| | | captured | | | | captured | |
| | Actual | viable | | Whole milk | | viable | |
| | viable spore | spores ± | Capture | No. of captured | Capture | spores ± | Capture |
| Dilution | count | S.D. (n = | ratio | viable spores ± | ratio | S.D. (n = | ratio |
| factor | (CFU/ml) | 3), | (CR) | S.D. (n = 3), | (CR) | 3), | (CR) |
| 101 | 1.52 × 107 | 6.07 × 106 ± | 0.40 | 1.41 × 106 ± | 0.09 | 3.02 × 106 ± | 0.20 |
| | | 55.3 | | 10.5 | | 32.5 | |
| 102 | 1.52 × 106a | 2.30 × 105 ± | 0.15 | 1.63 × 105 ± | 0.11 | 3.17 × 105 ± | 0.21 |
| | | 15.8 | | 10.3 | | 34.4 | |
| 103 | 1.52 × 105a | 3.40 × 104 ± | 0.22 | 1.22 × 104 ± | 0.08 | 2.85 × 104 ± | 0.19 |
| | | 17.6 | | 8.2 | | 6.5 | |
| 104 | 1.52 × 104a | 2.23 × 103 ± | 0.15 | 9.10 × 102 ± | 0.06 | 1.38 × 103 ± | 0.09 |
| | | 11.5 | | 19.5 | | 14.8 | |
| 105 | 1.52 × 103a | 5.53 × 102 ± | 0.36 | 1.40 × 102 ± | 0.09 | 5.10 × | 0.34 |
| | | 7.1 | | 2.6 | | 102 ± 9.5 | |
| 106 | 1.52 × 102a | 1.47 × 102 ± | 0.97 | 1.0 | 0.07 | 1.10 × 102 ± | 0.72 |
| | | 1.1 | | | | 0.6 | |
| 107 | 1.52 × 101a | 1.00 × 101 ± | 0.66 | ×101 ± 0.5 × | 0.00 | 1.00 × | 0.66 |
| | | 1.1 | | 101 ± 0.5 | | 101 ± 1.2 | |
| 108 | 1.52 × 100a | 0 | 0 | 0 | 0 | 0 | 0 |
| Control | 0 | 0 | 0 | 0 | 0 | 0 | 0 |

S.D. = standard deviation and n = no. of replicates.
aEstimated spore concentrations.

As observed in FIG. 22, although the biosensor resistance readings recorded for the different spore concentrations were different from the control, statistical analysis did not reveal any significant differences between the concentrations. Artifacts in biosensor fabrication, probabilistic antigen-antibody interactions, antibody orientations, and stability of the sandwich complex on the capture pad might be some of the factors behind such biosensor performance. At this stage the biosensor is only considered to be as a qualitative device for a yes/no diagnosis of B. anthracis spores. However, the biosensor shows excellent sensitivity and fast detection time in comparison to the very few rapid detection systems for B. anthracis in food matrices that have been reported in the literature (Tims and Lim, 2004, Journal of Microbiological Methods 59(1):127-130; Cheun et al., 2001, Journal of Applied Microbiology 91(3):421-426).

FIG. 22 shows the specificity analysis of the EAPM nanoparticle-based direct-charge transfer biosensor. A comparison of the biosensor resistance responses in pure cultures of E. coli with cell concentrations ranging from 1.7×10¹ to 1.7×10⁵ CFU/ml, in pure cultures of S. Enteritidis with cell concentrations ranging from 1.6×10¹ to 1.6×10⁵ CFU/ml, and pure spore suspensions of B. anthracis with spore concentrations ranging from 4.2×10¹ to 4.2×10⁵ spores/ml, is presented in this figure.

As evident in FIG. 22, the biosensor average resistance values for different concentrations of the non-target bacteria (i.e. E. coli and S. Enteritidis) are similar to the values observed for the control. Single factor analysis of variance tests to a significance of 95% (P<0.05) showed no statistically significant differences between the control and different cell concentrations of E. coli and S. Enteritidis with P-values ranging from 0.278 to 0.887 for E. coli, and from 0.348 to 0.981 for S. Enteritidis. The results indicate that the effects of non-specific interactions are not significant for the range of cell concentrations tested on the biosensor. In comparison, for pure B. anthracis spore suspensions (FIG. 22), the biosensor average resistance responses show significant differences between the control and spore concentrations ranging from 10² to 10⁵ spore/ml (P-value range: 0.009-0.0009) which is expected since the antibodies used in the biosensor are specific for B. anthracis.

However, at a low B. anthracis spore concentration of 10¹ spores/ml, the biosensor resistance response is similar to that of the control which implies no detection at this concentration (P-value: 0.7). The immunomagnetic capture of B. anthracis spores using EAPM nanoparticles from the three different food matrices was confirmed by microbial plating in TSA II blood agar plates and the capture efficacy of the nanoparticles was evaluated. Results of the microbial plating data have been elaborated in Table 1. One hundred microliters of the captured immuno-EAPM-spore complexes were used for microbial plating and the viable spore count was enumerated after 16-36 hour. The capture ratio (CR) of the EAPM nanoparticles was evaluated using the formula CR=Ccaptured/Cactual, where, Cactual is the actual concentration of viable spores in the sample, and Ccaptured is the concentration of viable spores extracted from the food samples.

As seen in Table 1, the capture effects of the EAPM nanoparticles on B. anthracis spores from the lettuce and ground beef samples are quite similar. In both samples, the capture effect is highest at a viable spore concentration of 1.52×10² CFU/ml with a CR value of 0.97 for lettuce and that of 0.72 for ground beef. Also, the EAPM nanoparticles could achieve an immunomagnetic capture at viable spore concentration as low as 1.52×10¹ CFU/ml from both lettuce and ground beef samples. However, for whole milk samples, the immunomagnetic capture of the viable B. anthracis spores could only go as low as 1.52×10² CFU/ml and the capture effects are observed to be similar for viable spore concentrations with CR values in the range of 0.06 and 0.11. This could be explained by the high fat content of the whole milk samples which might have interfered in the immunomagnetic capture process. The capture efficiency of the EAPM nanoparticles as observed from Table 1 is found to be better than that of commercially available immunomagnetic beads which can capture only as low as 10⁴ CFU/ml (Madonna et al., 2003, Rapid Communications in Mass Spectrometry 17 (3):257-263).

In summary, this example describes a novel integrated design of using electrically active polyaniline coated magnetic nanoparticles as a magnetic concentrator and biosensor transducer in one system. The biosensor detection system was fast and includes a magnetic concentration time of 10 min followed by a signal detection time of 6 minutes. The sensitivity of the biosensor in the detection of B. anthracis spores from complex food matrices was found to be 4.2×10² spores/ml in lettuce and ground beef, and 4.2×10³ spores/ml in whole milk. The EAPM nanoparticles also showed excellent capture efficiency from the food samples with a highest capture ratio of 0.97. Results indicate that the EAPM nanoparticles have great potential in biosensing applications. Future research work will involve in advancing the electrical and magnetic properties of the nanoparticles for biosensor detection of diverse targets. Furthermore, the easy-to-use EAPM biosensor described herein is contemplated to be applied as a rapid detection device in biodefense and diagnostics.

### Example X

The following examples describe an exemplary performance enhancement of a disposable polyaniline-based polymeric wire biosensor by using screen-printing technology for the fabrication of electrodes and pulse mode measurement techniques (Yuk et al., Performance enhancement of polyaniline-based polymeric wire biosensor, Biosensors and Bioelectronics, Volume 24, Issue 5, 1 Jan. 2009, Pages 1348-1352); herein incorporated by reference in its entirety.

Exemplary chemicals and reagents used were; aniline, ammonium persulfate, iron(III) oxide, glutaraldehyde, streptavidin and biotin conjugate goat anti-mouse IgG (whole molecule) were purchased from Sigma-Aldrich (St. Louis, Mo.). NC membranes (8-10 μm pore size and flow rate of 135 s/4 cm, with polyester backing), cellulose fiber sample pads, and absorbent pads were purchased from Millipore (Bedford, Mass.).

Preparation of polyaniline-coated magnetic nanoparticles and their conjugation with biotinylated anti-mouse IgG: A water-soluble polyaniline was synthesized based on a standard procedure of oxidative polymerization of aniline monomer in the presence of ammonium persulfate as an oxidizing agent (for example, Sergeyeva et al., 1996, Sens. Actuators B Chem. 34:283-288, herein incorporated by reference). Hydrochloric acid (HCl) was used as the protonating acid, and iron(III) oxide nanopowder to be coated with polyaniline was added into the polymerization vessel in a weight ratio of 1:0.6 (γ-Fe₂O₃:aniline monomer) as optimized in previous studies (Pal et al., 2007, Biosens. Bioelectron. 22:2329-2336, herein incorporated by reference). The morphology of polyaniline-coated magnetic nanoparticles (hereafter referred to as magnetic composite Fe₂O₃/polyaniline particles) was investigated by a transmission electron microscope (TEM) (JEOL JEM-100CX II, Japan). FIG. 18.

The magnetic composite Fe₂O₃/polyaniline particles fractions (0.2 g) obtained as a result of filtration were incubated with 1 ml of 0.1% glutaraldehyde for 30 min. The magnetic composite Fe₂O₃/polyaniline particles were separated using a magnetic separator and the liquid was discarded. Four hundred microliteraliquots of the biotinylated IgG (200 μg/ml) were added to the magnetic composite Fe₂O₃/polyaniline particles-glutaraldehyde complex. The incubation procedure was performed with shaking for 30 mM at room temperature and 500 μl of 100 mM Tris buffer containing 0.1% casein (pH 7.4) was added to the reaction mixture in order to inactivate non-reacted aldehyde groups. Finally, conjugated biotinylated IgG with magnetic composite Fe₂O₃/polyaniline particles was separated by the magnetic separator and was mixed with 1 ml of phosphate buffer (8.1 mM Na₂HPO₄, 1.2 mM KH₂PO₄, pH 7.4). The magnetic composite Fe₂O₃/polyaniline particles-biotinylated IgG was serially diluted with phosphate buffer (pH 7.4) in 3 dilutions and then stored at 4° C. until use.

Fabrication and characterization of electrodes: A nitrocellulose membrane (NC) (60 mm×301 mm) with a backing support was washed with 10% (v/v) methanol and then dried in the air. A silver polymer ink (Gwent Electronic Materials Ltd., UK) was printed on the NC membrane through a patterned screen of two electrode system with a 500 μm space (Ryonet Corporation, WA, USA) and baked at 60° C. for 30 min. The screen-printed NC membrane was cut into a 25 mm×5 mm² size using Matrix 2360 (Kinematic Automation Inc., CA, USA). Resistance of the silver screen-printed electrodes was measured by a 4192A LF Impedance Analyzer (Agilent Technologies Inc., CA, USA) with two-point probe measurement technique for a distance of 20 mm. The distance between the two screen-printed silver electrodes was the current path whose measurement was confirmed by a standard laboratory microscope BX41 (OLYMPUS, Germany). See, FIG. 18.

Preparation of polyaniline-based biosensor: The surface of NC membranes (20 mm×0.5 mm) was modified with 1% (v/v) glutaraldehyde solution for 30 min and then rinsed with deionized water for 15 min and flushed with N₂ gas to dry. After drying, 500 μg/ml of streptavidin was prepared in phosphate buffer (8.1 mM Na₂HPO₄, 1.2 mM KH₂PO₄, pH 7.4) and 12 μl of the protein solution was applied by using a micro-pipette to the surface of glutaraldehyde modified NC membrane. The NC membrane was washed with 0.05% Tween 20 in phosphate-buffered saline (8.1 mM Na₂HPO₄, 1.2 mM KH₂PO₄, 138 mM NaCl, 2.7 mM KCl, pH 7.4) (PBS) for 15 min, rinsed with deionized water and flushed with N₂ gas to dry. The NC membrane was then incubated with 10 mg/ml of bovine serum albumin in PBS for 30 min to reduce nonspecific interactions. After incubation, the NC membrane was washed with 0.05% Tween 20 in PBS for 15 min, rinsed with deionized water, dried under N₂ gas, and immediately assembled with sample and absorbent pads on the NC membrane as shown in FIG. 20 and installed to an etched copper printed circuit board (PCB).

Electrical measurement system: The electrical two-point probe measurement consisted of a measurement cell, a multimeter model 2000 (Keithley Instruments, Inc., OH, USA) connected with a general purpose interface bus (GPIB) cable, and a personal computer. To monitor the electrical response of the polyaniline-based biosensor, voltage generated between the two electrodes was measured and displayed as resistance using Ohm's law. When the DC resistance was measured with a pulse mode voltammetry, DC current of 10 μA was applied every 1 s interval. Data acquisition and display were performed by a customized program based on LabVIEW.

Testing of the biosensor: Interactions of streptavidin with biotinylated IgG conjugated with magnetic composite Fe₂O₃/polyaniline particles were analyzed after applying 80 μl of the analyte onto the sample pad. See, FIG. 20. After biotinylated IgG conjugated with the magnetic composite Fe₂O₃/polyaniline particles solution was applied onto the sample pad, a resistance change was measured by pulse mode voltammetry.

Resistance decreased with time and became stable after about 2 min with a reading of approximate 4.3 kΩ (solid line in FIG. 20).

The distance between screen-printed electrodes was approximately 550 μm and more uniform compared to that of the manually fabricated electrodes as shown in FIG. 20). Electrical characteristics of the electrodes fabricated with a silver polymer ink on NC membranes were studied by I-V measurements. Silver has a pure crystal structure that provides low residual currents and high signal-to-noise ratio (Ashcroft and Mermin, 1976, Solid State Physics. Holt, Rinehart and Winston, New York; Ivnitski et al., 1999, Biosens. Bioelectron. 14:599-624, all of which are herein incorporated by reference) so that silver was chosen as the electrode of the electrical biosensor. The I-V characteristics of the silver electrode showed an ohmic behavior as shown in FIG. 20, which means that the electrode would work well as a good conductor with a constant resistance (Halliday et al., 2001, Fundamentals of Physics. John Wiley & Sons, New York). Resistance of the silver electrode was calculated as 1.6Ω from the I-V graph. To investigate the resistance fluctuation of screen-printed electrodes, the resistance of 30 strips were randomly selected and measured. FIG. 20 shows the histogram of measured electrodes and the resistance spread out from 0.5 to 2.5Ω with an average of 1.4Ω. From the above results, it is found that the screen-printed electrodes have a uniform distance.

Next, it was investigated whether magnetic composite Fe₂O₃/polyaniline particles was formed by a TEM image. Magnetic nanoparticles (<50 nm) were mixed with aniline monomer in a weight ratio of 1:0.6 to synthesize the magnetic composite Fe₂O₃/polyaniline particles. The polyaniline was polymerized based on a standard procedure of oxidative polymerization of aniline monomer in the presence of ammonium persulfate as oxidizing agent. FIG. 2 shows that polyaniline-coated magnetic nanoparticles have an average size of less than 100 nm. A polymeric wire was formed by a string of polyaniline-coated magnetic nanoparticles.

The signal generation was explained by current flow induced by the streptavidin-biotin interaction. The analyte carrying the biotin molecules flowed into the NC membrane with immobilized streptavidin where the streptavidin-biotin complex was formed. The polyaniline in the complex bridged the two electrodes and provided a current path. The unbound analyte at the liquid-solid interface was subsequently washed away to the absorbing pad by capillary action so that an electrical response due to the pure streptavidin-biotin interaction was measured by the DMM. To compare the resistance change measured by pulse mode with that by non-pulse mode, the same experiment was repeated. Except for the initial ramping, the signal response of the non-pulse mode (dashed line) was similar to the pulse mode (solid line) as shown in FIG. 2.

However, the resistance obtained by the non-pulse mode was higher than the resistance obtained by the pulse mode which was attributed to the resistance related to the interfacial capacitance (t/Cd). The interfacial capacitance (Eq. (2)) was contemplated to be less affected by the pulse mode measurement compared to the non-pulse mode measurement. The resistance of Rs expressed in Eq. (2) was generated by the streptavidin-biotin interaction on the biosensor. From these results, it was found that the pulse mode measurement was a more reliable measure of the electrical response induced by the biospecific interaction by reducing the resistance related to the interfacial capacitance.

Further investigated was whether resistance changes by interaction of various concentrations of magnetic composite Fe₂O₃/polyaniline particles—conjugated biotinylated IgG could be analyzed by the pulse mode measurement. Resistance of phosphate buffer (pH 7.4) was used as a reference signal for monitoring resistance changes induced by the streptavidin-biotin interaction in the polyaniline-based biosensor. After preparing the various concentration of analyte with 3 serial term fold dilutions, each analyte was applied onto the sample pad and the resistance change was monitored in real-time. Resistance measured at 3 min was used to investigate a dose-dependent response. FIG. 3 shows an exemplary resistance that increased and then saturated according to the concentrations of the analyte. Increment of resistance at the diluted concentrations means that the biotin complex with polyaniline was less bound with streptavidin so that the voltage across the electrodes increased. The dose-dependent response obtained by pulse mode could be explained by the relative amount of magnetic composite Fe₂O₃/polyaniline particles—biotinylated IgG complex bound on the streptavidin surface of the biosensor.

Thus, this study demonstrated that the reproducibility and reliability performance of polyaniline-based polymeric wire biosensor was enhanced by using screen-printed silver electrodes and pulse mode measurement.

### Example XI

The following examples describe an exemplary embodiment for use of a polyaniline-based polymeric wire biosensor using hand-printing for the fabrication of electrodes (i.e. a 1 mm wide capture channel) for use in veterinary medicine, such as Johne's disease (JD), a chronic gastrointestinal disease of ruminants caused by Mycobacterium avium subsp. paratuberculosis (MAP), (Okafor, Fabrication of a Novel Conductometric Biosensor for Detecting Mycobacterium avium subsp. paratuberculosis Antibodies Sensors 2008, 8, 6015-6025; Published: 26 Sep. 2008; herein incorporated by reference in its entirety).

A fabricated conductometric biosensor developed herein differentiated between the relative MAP antibodies concentration of JD positive and negative samples in about 2 minutes. The assay was portable for adaptation in on-site diagnosis and supported animal point-of-sale testing, routine herd testing, and clinical animal testing, especially in places with limited access to the currently available JD diagnostic tests. Such test results are contemplated to provide a basis for timely management decisions that would control the spread of JD among animals. Hence, a conductometric biosensor is contemplated as a diagnostic assay for improving JD control.

The biosensor used herein comprises an immunosensing component and a signal detector system. The immunosensing component of the biosensor consisted of four individual membranes: sample application, conjugate, capture, and absorption membranes (Hi-Flow Plus Assembly Kit, Millipore, Bedford, Mass., U.S.A.). These membranes were prepared, fabricated, and assembled to form a functional biosensor. The choice of these membranes was based on previous studies [Sergeyeva, Sensor Actuat. B-Chem. 1996, 34:283-288, Muhammad-Tahir, et al., IEEE Sens. J. 2003, 3:345-351, Kim, et al., Biosens. Bioelectron. 2000, 14:907-915]. The sample application membrane, made of cellulose, provides a quick flow of the sample with no or minimal interference; the conjugate membrane, made of fiberglass, adsorbs the polyaniline-conjugated antibody and allows easy flow of fluid; the pore size of the capture membrane, made of nitrocellulose, allows the flow of non-target molecules while providing good adsorption properties for the immobilized molecule; and the absorption membrane, a cellulose membrane, absorbs and retains the fluid from the capture membrane. For a functional conductometric biosensor, the capture membrane is printed with silver electrodes, yielding a 1 mm wide capture channel. The electrodes are connected to an etched copper wafer, and finally the wafer is connected to the signal detector system, an ohmmeter. Silver electrodes and the etched copper wafer was demonstrated to possess good electrical and easy fabrication properties [Sergeyeva, Sensor Actuat. B-Chem. 1996, 34:283-288, Muhammad-Tahir, et al., IEEE Sens. J. 2003, 3:345-351, Kim, et al., Biosens.Bioelectron. 2000, 14:907-915].

Capture membrane preparation: The capture membrane was prepared at room temperature under a clean biosafety cabinet unless otherwise stated. The capture membrane was first flushed with distilled water, to remove any debris, and air-dried for 0.5 h. To activate the membrane surface, it was flushed with 10% methanol and air-dried for 0.5 h. To provide a crosslink between the nitrocellulose membrane and the biological receptor molecule, the membrane was washed with 0.5% glutaraldehyde solution (1.2 mL) and air-dried for 1 h. A total volume of 1.2 mL of 1 mg/mL Mycobacterium avium purified protein derivative (MAPPD) (AntelBio, East Lansing, Mich.) was pipetted on the membrane. The membrane was placed in a closed plastic container and incubated (Isotemp incubator, Fisher Scientific) at 35° C. for 1 h. MAPPD was used as the antigen because Mycobacterium avium is antigenically similar to MAP. Afterwards, the membrane was washed with 1.2 mL of 0.1 M Tris buffer containing 0.1% (v/v) Tween-20, to remove non-specifically absorbed MAPPD. Finally, the membrane, placed in a closed plastic container, was incubated at 35° C. for 0.75 h, air-dried for 0.5 h, and was set to be fabricated.

Polyaniline—Anti bovine IgG conjugation: AquaPass polyaniline (Mitsubishi Rayon Co, Japan) was diluted to 0.001% with 0.1 M phosphate buffer solution. Purified mouse clone BG-18 monoclonal anti-bovine IgG (Sigma-Aldrich, St Louis, Mo.) was added to the 0.001% AquaPass polyaniline (Pani) solution to produce a final monoclonal anti-bovine antibody (AB/IgG*) concentration of 0.0115 mg/ml. To form Pani-AB/IgG* conjugate, 4 ml of the AB/IgG* solution was left to conjugate with the Pani in a hybridization oven at 27° C. for 1.0 h. Afterwards, 0.5 ml of 0.1M Tris buffer containing 0.1% casein (pH 9.0), a blocking agent, was added to the Pani-AB/IgG* conjugate solution and left to react in a hybridization oven at 27° C. for 0.5 h.

Conjugate membrane immobilization: To immobilize Pani-AB/IgG* conjugate on the conjugate membrane, a conjugate membrane was immersed in the Pani-AB/IgG* conjugate solution until saturated and then air-dried at room temperature under a clean biosafety cabinet for 0.75 h.

Immunosensor fabrication: The capture membrane, besides the prepared portion at the center, has waterproof adhesives at both ends, and provides the backing for attachment of the other immunosensing membranes. These waterproof adhesives were peeled and the other membranes were attached to the waterproof ends during fabrication.

First, the conjugate membrane was attached to one end of the prepared portion of the capture membrane, and then the application membrane was attached overlaying a portion of the conjugate membrane. The absorption membrane was attached on the opposite end of the capture membrane, to complete the immunosensor fabrication. The fabricated immunosensor was cut into 5 mm wide immunosensor strips. With a silver-microtip conductive pen (MG Chemicals, Surrey, B.C., Canada), silver electrodes were hand-printed on both sides of the capture membrane, such that an approximate 1 mm wide capture channel was produced (FIG. 13).

Conductometric biosensor assembly: Each silver electrode, flanking the capture membrane, was connected to a copper wafer (FIG. 13); connection was hand-printed with a silver-microtip conductive pen. The two ends of the copper wafer were connected to an ohmmeter (Model: 2880A BK Precision multimeter, Worchester, Mass., U.S.A.).

One hundred microliters of sample is applied to the application membrane and is drawn into the entire channel of the immunosensor strip by capillary action. The sample passes the conjugate membrane, where serum IgG, both MAP and non-MAP, are bound to the Pani-AB/IgG* conjugate, forming Pani-AB/IgG*-IgG complex (FIG. 13). The complex is drawn into the capture membrane, where immobilized MAPPD captures the MAP specific IgG (JD positive serum) and allow the non-MAP IgG to flow to the absorption membrane. As more and more MAP IgG are captured, the Pani in the Pani-AB/IgG*-IgG complex forms a bridge between the silver electrodes, flanking the capture membrane. Pani causes an electrical conductance through the electrodes; a higher electrical conductance is recorded as a reduced resistance.

Samples: The developed biosensor was tested with both JD positive and JD negative bovine serum samples. The positive samples were collected from clinical JD cows housed at the Michigan State University Veterinary Research Farm, while the negative samples were collected from cows at the Michigan State University Dairy Teaching and Research Center, who had been tested negative for JD a minimum of three times. JD status of the samples was determined by a commercially available MAP ELISA (PARACHEK, Prionics, Schlieren-Zurich, Switzerland), performed at the Diagnostic Center for Population and Animal Health, Michigan State University. The ELISA interpretation was based on the optical density (OD) values, a reflection of the MAP antibody concentration in each sample. ELISA OD values<1.0 are considered JD negative and >1.0 are considered JD positive.

Sample testing and signal measurement: Testing was conducted using purified serum of animals tested to be JD positive and JD negative. The serum was purified with Melon™ gel IgG purification kit (Pierce Biotechnology, Rockford, Ill., U.S.A.), according to the manufacturer's specification. The purification step was aimed at removing the non-relevant serum proteins that could compete with IgG during Pani conjugation. Subsequent testing was aimed at verifying the response of the biosensor with unpurified field samples, which meets the objectives of developing a non-laboratory based assay. After each sample application, the resistance value (Kiloohms) was recorded at 2, 4, and 6 minutes. Eight replications were performed for each sample in the preliminary testing while three replications were performed on each sample of the subsequent testing. The mean values of each sample and their standard deviations were calculated.

Statistical analyses: A 2-way ANOVA was used to analyze if the mean resistance values were significantly different among the sample groups, adjusting for the effects of different ELISA OD values and different reading times. Holm-Sidak test was used to isolate which group(s) differed from the others. The statistical analyses were performed with SigmaStat 3.1 software. Intra-assay coefficient of variation of the biosensor was calculated to evaluate the precision of the biosensor assay.

A testing result of a JD-infected animal against a JD-free animal, with ELISA OD values of 3.270 and −0.048 respectively, is shown in FIG. 13. For each time interval, significant lower resistant values were recorded for the JD-infected animal as compared to the JD-free animal.

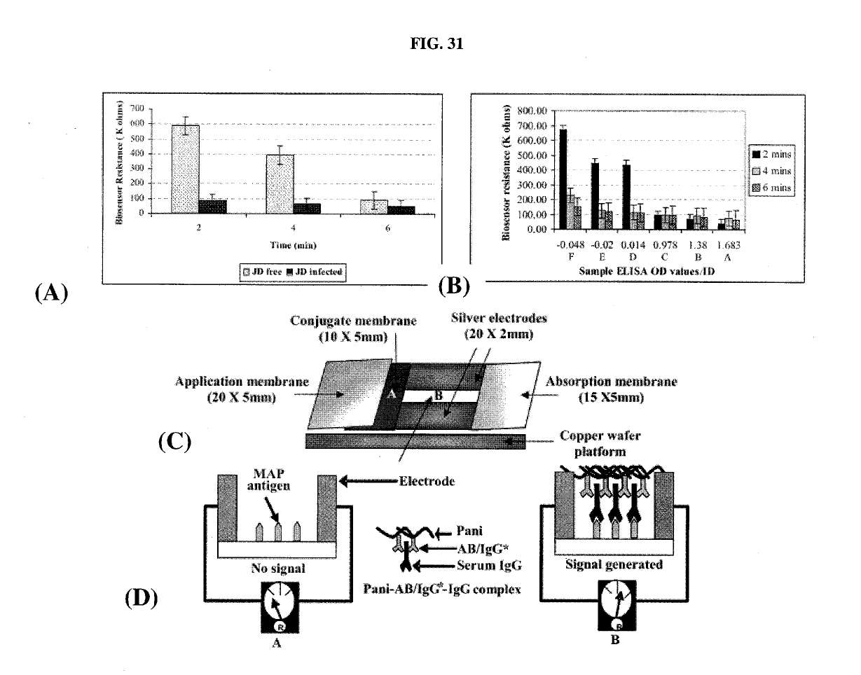

Testing of biosensor with unpurified serum. Results for each sample tested in the biosensor at different time points are shown in Table 7. The conductometric biosensor evaluation of the samples showed that at each 2-minute interval, the JD positive samples had numerically lower resistance values than the JD negative samples. A 2-way ANOVA showed that the difference in mean resistance values of the samples was significantly affected by the ELISA OD values, the reading times, as well as their interactions (P<0.05) (FIG. 31). Hence, the effect of ELISA OD values on the biosensor resistance depended on what time the reading was taken.

TABLE 7
Conductometric biosensor analysis of bovine
serum samples at 3 time intervals.

| | | Conductometric biosensor mean (n = 3) resistance | | |
|---|---|---|---|---|
| | Sample | (Kilo ohms) at 3 time intervals | | |
| Sample | ELISA | 2 min | 4 min | 6 min |
| ID | OD values | Mean ± SD | Mean ± SD | Mean ± SD |
| A | 1.683** | 43.47 ± 4.76a | 75.63 ± 32.20a | 66.63 ± 24.66a |
| B | 1.380** | 70.33 ± 3.95a | 93.43 ± 33.50ab | 81.20 ± 33.98a |
| C | 0.978** | 95.43 ± 12.58a | 97.60 ± 30.19ab | 97.13 ± 24.94a |
| D | 0.014* | 437.00 ± 33.29b | 114.73 ± 23.97ab | 112.83 ± 20.87a |
| E | −0.020* | 448.37 ± 99.41c | 125.83 ± 19.69ab | 117.73 ± 20.85a |
| F | −0.048* | 672.33 ± 101.93c | 228.53 ± 162.9b | 152.13 ± 20.33a |

**Johne's disease (JD) positive,
*JD negative, the negative OD values are from samples that have less background optical density than the negative serum control,
SD = standard deviation.
Different superscripts a b c within columns indicate significant differences between the mean resistance of the samples (p < 0.05).

The Holm-Sidak test showed that at 2 minutes, the mean resistance value of each of the JD positive samples (A, B, C) was significantly different (P<0.05) from the mean resistance of each of the JD negative samples (D, E, F). At 4 minutes, the difference in mean resistance was statistically significant only between sample A and sample F. And at 6 minutes, the difference in mean resistance between the JD positive samples and the JD negative samples was not statistically significant. The intra-assay coefficient of variation of the biosensor at 2 minutes was 14.48%.

### Example XII

The following example describes an exemplary multiplexed biosensor based on Biomolecular Nanowires for detecting detecting multiple pathogens in a biological sample (Liu, et al., A Multiplexed Biosensor Based on Biomolecular Nanowires IEEE International Symposium on Circuits and Systems, pp. 2006-2009, Seattle, USA, 2008); herein incorporated by reference in its entirety).

The fabrication and characterization of a multiplexed biosensor based on molecular bio-wires that were used for detecting multiple pathogens in a biological sample. At the core of the proposed device is a biosensor that operates by converting binding events between antigen and antibody into a measurable electrical signal using polyaniline nanowires as transducers. By mixing and patterning antibodies at different spatial locations of the biosensor, the response of the biosensor is contemplated for configuration to detect the presence of either one of several pathogens present in the analyte, thus making it ideal for rapid environment screening applications. Experiments using the biosensor array specific to B. cereus and E. coli bacteria validate the functionality of the proposed multiplexed architecture.

A prototype of a multiplexed biosensor contemplated for use for rapid screening of multiple pathogens in an analyte is described below. The core principle behind the proposed architecture is a transduction mechanism that relies on interaction between antigens and a mixture of antibodies. Presence of any of the target pathogens leads to a change in conductance across the biosensor that can be measured using a data acquisition system. One of the limitations in using mixture of antibodies for screening of multiple pathogens is the reduced sensitivity due to limited availability of binding sites for target antigens. However, this artifact can be compensated for by enhancing the sensitivity of the measurement instruments. Also, analyte reaching the antibody strips located at spatially disjoint locations contain variable concentration of pathogens which produce a weaker measurable signal. This observation was reported in [Malamud, et al., “Point Detection of Pathogens in Oral Samples,” Proceeding of a Symposium on Saliva/Oral-fluid-based Diagnostic Markers of Disease, Mar. 12, 2004], and a solution proposed to remedy this problem was a symmetric architecture that ensures a uniform flow of pathogens towards the antibody sites. Streamline fabrication process is also expected to improve the response of the “OR” logic operation.

Materials and Fabrication Procedures: Purified rabbit polyclonal antibodies against B. cereus and E. coli were obtained from Meridian Life Science (Saco, Me., USA). The antibodies were suspended in phosphate buffer solution (pH 7.6) and stored at 4° C. B. cereus and E. coli strains were obtained from the National Food Safety and Toxicology Center (Michigan State University) and the Michigan Department of Community Health (East Lansing, Mich., USA). A 10 μL loop of each isolate was cultured in 10 ml of nutrient broth and incubated for 24 h at 37° C. to prepare stock cultures. Polyaniline was purchased from Sigma-Aldrich (St. Louis, Mo., USA). The sample pads (size: 20 mm×5 mm) capture pads (size: 20 mm×5 mm) and absorption pads (size: 15 mm×5 mm) were made of nitrocellulose membrane and the conjugate pads (size: 10 mm×5 mm) were made of fiberglass membrane (grade G6). The porous nitrocellulose substrate ensures good adsorption properties for immobilized antibodies and allows non-target antigens to flow through. The conjugate pad was designed to allow maximal adsorption and flow of polyaniline-conjugated antibodies. Antibody concentration used for conjugate pad was 150 μg/ml and for the capture pad was 500 μg/ml. The polyaniline concentration in the conjugate pad was 1 mg/ml. These values were found to be optimal, resulting in the highest ratio between the number of captured cells and the actual cell concentration tested.

The polyaniline antibody conjugates were prepared by suspending 800 μl of polyclonal antibodies against B. cereus and E. coli (concentration 150 μg/ml) in a 8 ml of polyaniline solution in phosphate buffer (pH 7.6) containing 10% dimethylformamide (DMF) (v/v) and 0.1% LiCl (w/v). The solution was incubated at room temperature for 1 h to allow the binding of the antibodies with polyanilines and then treated with a blocking reagent (Tris buffer containing 0.1% casein). The polyaniline antibody conjugates were then precipitated by centrifugation at 12,000 rpm for 5 min. The supernatant fluid was discarded and the pellets were mixed with the blocking reagent and centrifuged again. The centrifugation step was repeated three times. The conjugates were finally suspended in phosphate buffer solution containing 0.1% LiCl (w/v) and 10% DMF (v/v) and stored at 40 C until used. The conjugate pads were prepared by soaking the fiber-glass strip into the conjugation solution till homogenous dispersion is achieved. Extensive characterization of a single strip, single pathogen biosensor was performed elsewhere [Muhammad-Tahir, et al., IEEE Sensors Journal, vol. 5, pp. 757-762, 2005], [Liu, et al., Nanotechnology, vol. 18, No. 42, 2007]. The focus of this paper is to extend this architecture to a multiplexed configuration. The sample pads and the absorption pads were washed using distilled water for removing surface residues and then left to dry. Before dispensing antibodies, the capture pads were pre-treated with distilled water and 10% (v/v) methanol sequentially and left to dry for 1 h. Then, the pad was treated with 0.5% (v/v) glutaraldehyde and left to dry again. Subsequently, polyclonal E. coli and B. cereus antibodies were immobilized on the capture pads using the dispensing machine (Kinematics Automation Inc.) and incubated at 37° C. for 1 h. 0.1 mM tris buffer (pH 7.6, containing 0.1% (v/v) Tween-20) were dispensed onto the capture pads to remove and block residual functional groups. The pads were then incubated at 37° C. for 45 minute.

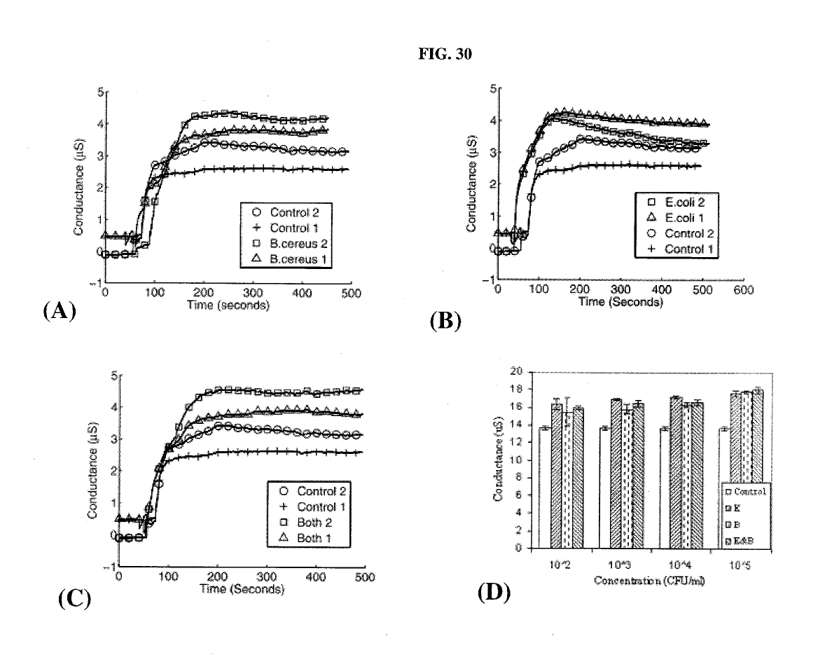

Characterization of multiplexed biosensor: For preparing the multiplexed biosensor, a mixture of poly-clonal antibodies against B. cereus and E. coli (concentration 500 μg/ml) was dispensed onto the surface of the nitrocellulose substrate. A copper mask as shown in FIG. 31 was used to fabricate electrodes onto the capture pad using aluminum evaporation technique. Even though the experiments in this paper used only two pathogens (B. cereus and E. coli), the proposed principle can be extended to include antibodies specific to other pathogens as well. The inventors have patterned the mixtures at two spatial locations on the capture pad which were designated by 1 and 2 as indicated in the measurement results. Strip 1 is antibody capture zone, which is close to the conjugation pad while strip 2 is a little bit far from the conjugation pad. FIG. 30 show typical responses where the conductance across the two antibody capture zones are simultaneously measured (marked by 1 and 2 in FIG. 9A) as a function of time. For comparison, conductance corresponding to a control solution (analyte without pathogens) is also plotted in the FIGS. In FIG. 30, Control 1, E. coli 1, B. cereus 1, and Both 1 represent the conductance measured across antibody capture zone 1 (marked in FIG. 30) when blank solution, E. coli, B. cereus, and the mixture of E. coli and B. cereus solution are applied. Similar notations (Control 2, E. coli 2, B. cereus 2, and Both 2) also hold for strip 2 in the FIG. 30. For these experiments, the concentrations of B. cereus and E. coli were calibrated to be 6×10⁷ CF U/ml and 5×10⁷ CF U/ml respectively. Confirmation of the pathogens followed standard microbiology protocols [FDA-Food and Drug Administration, Bacteriological Analytical Manual. AOAC International, Gaithersburg, Md. 2000]. For this purpose, colonies were counted using an automated platter-counter in a biosafety level II environment.

As shown in FIG. 30 the conductance measured across both of the antibody capture zones are higher when either of the pathogen is present compared to the control. This verifies the functionality of the multiplexed biosensor. Because the antibody zones are located at different spatial locations, the conductance measured across zone 1 and zone 2 changes sequentially between 40 to 90 seconds. FIG. 30 also show the interference of measured conductance due to electrical interaction between the two electrodes. However, this interference does not significantly impact the ability of the biosensor to discriminate between pathogenic and non-pathogenic samples. FIG. 30 shows the conductance measurement across a single antibody capture zone when samples containing different concentrations of the pathogens are applied. Each test is repeated three times and the error bars show the standard deviation. The results indicate that logic “OR” operation is consistent across different concentration levels of the pathogens, thus verifying the functionality of the mixed antibody strip to detect either one of the two pathogens.

### Example XIII

The following example describes an exemplary nanoporous Silicon-Based DNA Biosensor for detecting Salmonella Enteritidis.

Reagents and Materials Boron-doped, one-side polished p-type silicon wafers (100 orientation, 0.01-0.02 Ωcm) were purchased from Wafernet, Inc. (San Jose, USA). The anodizing solution consisted of hydrofluoric acid (48%, Columbus Chemical Industries, Inc.) and ethanol (99.5%, Sigma). The electrolyte for the cyclic voltammetry was composed of potassium ferrocyanide (99%, Sigma) and potassium nitrate (99.7%, J. T. Baker). Sulfochromic acid was used to provide a homogenous hydroxide layer on the silicon surface so that the silanization process using 3-glyci-doxypropyltrimethoxysilane (GOPS) (98%, Sigma-Aldrich) could proceed. Glycine (98% Sigma-Aldrich) was used to block the unspecific sites of the aldehyde groups. Acetone (99.4%, J. T. Baker), methanol (99.9%, Fisher Scientific) and toluene (99.5% Sigma-Aldrich) were used as rinsing solutions. Sodium hydroxide (97%, Spectrum) was used to provide the DNA probe with a basic environment. The functionalized biosensor was rinsed using sodium chloride-sodium citrate buffer (SSC, Sigma-Aldrich) and sodium dodecyl sulfate (SDS, Pierce, Inc.). Horseradish peroxidase (HRP) conjugated streptavidin and ABTS Kits (Invitrogen Corporation) were used to verify successful hybridization. Biotinylated dNTP mixture (New England Biolabs) was used to amplify the target DNA.

Apparatus: The electrochemical analysis and porous silicon formation were performed at an electrochemical workstation. The station included a Potentiostat/Galvanostat (Princeton Applied Research, Versastat II), a N₂ bubbling/purging system and a Teflon electrochemical cell. A source meter (Keithley, Model 2400) and a resistivity system (Lucas Lab, Pro4) were used to determine the resistivity of silicon wafers. The anaerobic environment was provided by the polymer anaerobic chamber (Coy Lab Product, Inc.). The target DNA was amplified by a PCR machine (Mastercycler Personal, Eppendorf). Gel electrophoresis (Runone System) and a spectrophotometer (SmartSpec 3000, Bio-Rad Laboratories, Inc.) were used to quantify DNA samples as well as the PCR product. The porous layer of the silicon chip was characterized by scanning electron microscopy [JSM-6300F]. An UV-Vis-NIR Scanning Spectrophotometer (UV-3101PC, Shimadzu, Inc.) was used to determine the absorbance of the HRP catalyzed reaction in the characterization experiments.

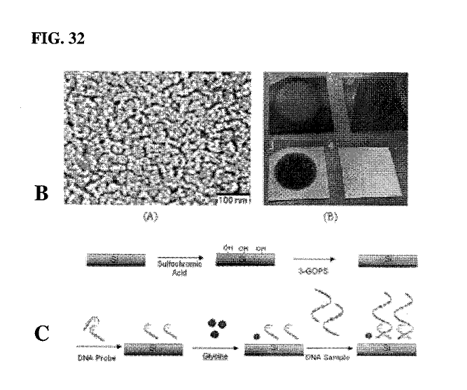

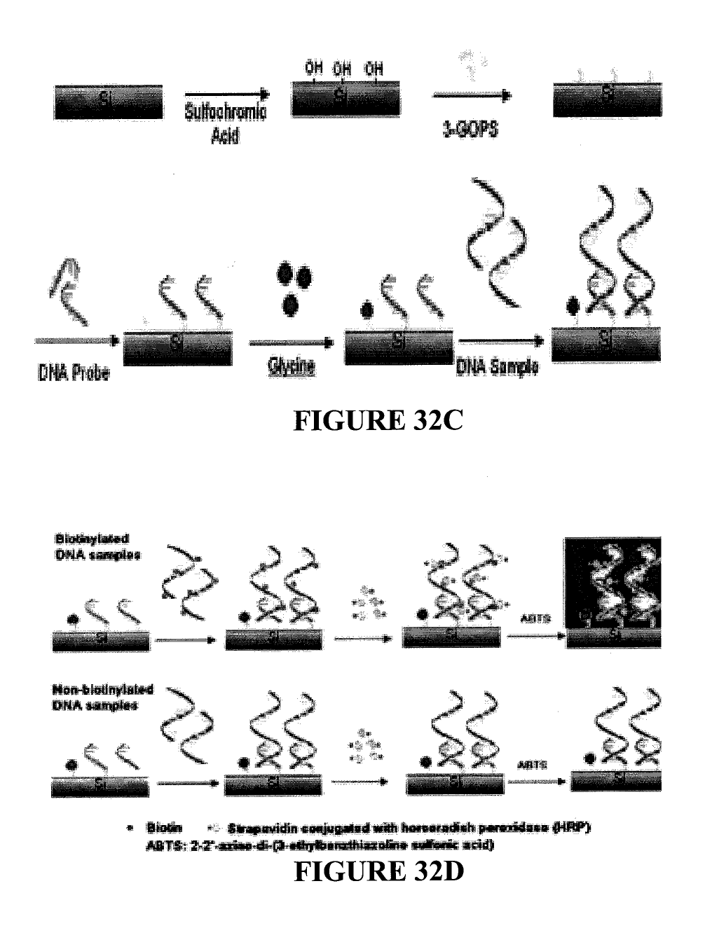

Porous silicon was fabricated using an anodization process in an electrochemical Teflon cell. The silicon wafer (p-type, 100 orientation, 0.01 Ωcm was cut into 1.8 cm×1.8 cm square chips with a diamond knife. The oxidized layer of the chip was removed by dipping it into 48% hydrofluoric acid. After rinsing with deionized water, the chip was dried with nitrogen, placed between two O-rings on the bottom hole of the Teflon cell, and sealed with a copper rod (FIG. 32) Hydrofluoric acid in ethanol (14.2%) was poured into the cell. A platinum wire served as the cathode with the silicon chip as the anode. A Versastat II Potentiostat/Galvanostat (Princeton Applied Research) was used to provide a constant current supply for anodization. Based on a previous study [Mathew and Alocilja, Biosens. Bioelectr., 20:1656-1661:2005], the anodizing current density of 5 mA/cm² was selected and the formation elapsed time was set for 1 h. After PS formation, luminescence was observed under UV light (incident wavelength nm) and characteristic images were taken using a scanning electron microscope (SEM).

Bacteria Culture, DNA Isolation, and PCR: A clinical strain of Salmonella Enteritidis (strain S-64) was used in this study. The pathogen was grown in trypticase soy broth (TSB) at 37° C. for 16 h. After enrichment, the cells were enumerated by spiral plating appropriately diluted cultures on bismuth sulfite agar and brilliant green agar. DNA isolation was performed from 1 mL S. Enteritidis culture using the QiaAmp DNA Mini Kit (Qiagen, Inc., Valencia). Primers used for PCR were designed for the detection of S. Enteritidis using the insertion element (Iel) gene [9]. The single stranded forward and reverse primers were IelL-5′-CTAACAGGCGCATACGATCTGACA-3′ (positions 542 to 565, 24 bases) and IelR-5′-TACGCATAGCGATCTCCTTC GTTG-3′ (positions 1047 to 1024, 24 bases). The capture probe used was 5′-[Amino link] AATATGCTGCCTACT GC-CCTACGCTT-3′ (positions 919 to 944 of the target, 26 bases). Using this set of primers and the extracted DNA template, the biotinylated and nonbiotinylated DNA samples were amplified with biotinylated dNTP mixture and common dNTP mixture, respectively. To verify the availability of the nanopore surfaces, three DNA samples were used, including biotinylated and nonbiotinylated DNA samples from PCR amplification, and biotinylated noncomplementary DNA sample. Biotinylated noncomplementary DNA (5′-[biotin]-CCT GTA CTT TGC TAC TTG CTG CAC AA-3′) was used for evaluating the specificity of the DNA probe. To evaluate the hybridization improvement of porous silicon compared to planar silicon, biotinylated PCR product on planar silicon was utilized as the control. The oligonucleotides were synthesized by the Research Technology Support Facility at Michigan State University. The PCR product was identified by gel electrophoresis and a UV transilluminator. Its concentration and quality were measured with a Bio-Rad SmartSpec 3000 spectrophotometer. After determining the concentration, the DNA solution was diluted serially with hybridization buffer for detection by the PS biosensor.

Functionalization of Porous Silicon: Two main steps were involved in the functionalization of the porous silicon [Mathew and Alocilja, Biosens. Bioelectr., 20:1656-1661:2005]: silanization of the chip surface followed by DNA probe immobilization. For silanization, the porous silicon chips were first cleaned in boiling acetone for 5 min, then boiled in methanol for 5 min, and finally dried with nitrogen. The chips were immersed in sulfochromic acid (a strong oxidizing agent) for 15 min, washed twice in deionized water and dried under nitrogen flow. The chips were oven dried for 1 h at 140° C., and immediately transferred to the anaerobic chamber. A 10% 3-gly-cidoxypropyltrimethoxysilane (GOPS) solution (in dry toluene) was placed on the chips. After reacting under nitrogen for 4 h, the chips were rinsed with toluene and dried. Immobilization of the probe layer was performed by coating each chip with 50 L of 1 M aqueous DNA probe solution, and leaving them to react overnight at room temperature in the anaerobic chamber. Finally, the chips were placed in boiling water for 2 min to remove excess unreacted DNA probe and dried with a nitrogen flow. The unreacted aldehyde groups of GOPS were then saturated by dipping the chips in a 0.1 M glycine solution for 20 min. The chips were washed using a washing solution (1×SSC) and dried with a nitrogen flow. The biosensors were stored under nitrogen for maximum stability. FIG. 32 shows a schematic diagram of the preparation and detection schemes of the biosensor.

DNA Hybridization and Verification of Nanopore Surface: The target DNA from S. Enteritidis in the hybridization buffer solution was heated to 95° C. to separate the double stranded DNA (dsDNA), and then cooled to 59° C. (the melting temperature of the capture probe). Hybridization of complementary DNA strands with the immobilized oligonucleotide probe layer was performed by coating the 50 uL DNA sample (or single DNA strands for the control) onto the DNA probe modified chip at different DNA concentrations in hybridization buffer for 60 min at 59° C. The nonspecifically adsorbed strands were removed with washing solution A (0.1×SSC, 0.1% SDS) followed by washing solution B (0.1×SSC). The chips were then dried under nitrogen.

Electrochemical Analysis: Before electrochemical analysis, 7 mL of the electrolyte (5 mM K Fe(CN) and 1 M KNO) was poured into a Teflon cell. The PS acted as the working electrode and a platinum wire as the counter electrode. An Ag/AgCl electrode acted as the reference electrode. Cyclic voltammetty was performed using a Versastat II Potentiostat/Galvanostat (Princeton Applied Research). The scanning potential was between −1.2 and 1.5 V at a scan rate of 50 mV/s. Before electrochemical analysis, the Teflon cell was purged with nitrogen for 10 min.

Porous Silicon Characterization: The formation process of porous silicon is complex, depending on many factors—HF concentration, type of silicon, current density, and illumination. The properties of PS, such as porosity, thickness, pore diameter, and microstructure, depend on the anodization conditions. These conditions include HF concentration, current density, wafer type and resistivity, anodization duration, illumination (n-type mainly), temperature, ambient humidity, and drying conditions. Some basic requirements must be fulfilled for electrochemical pore formation to occur: 1) holes must be supplied by bulk Si, and be available on the surface; 2) in the dissolution reaction, the pore tips must be active while the wall of the pore is passivated; and 3) the current density should be lower than the electropolishing critical value [Bisi, et al., Surf. Sci. Rep., 38:1-126, April 2000]. If the applied current density is higher than this value, the reaction is under ionic mass transfer control, which leads to surface-charged holes and to a smoothing of the silicon surface. Based on previous work on p-type silicon with a resistivity of 0.01 Ωcm [Mathew and Alocilja, Biosens. Bioelectr., 20:1656-1661:2005], the experimental conditions were established as follows: HF concentration in ethanol, anodization duration=1 h, and current density 5 mA/cm² without additional illumination. The anodization conditions provided the desired porous silicon chip characteristics with a uniform, repeatable nanoscale pore structure.

SEM was utilized to observe pore dimensions. FIG. 32 (C) shows a SEM top view of a p-type silicon chip (0.01 Ωcm). The anodization condition of 5 mA/cm² for 1 h provided the desired porous silicon chip characteristic of relatively uniform pore structure, with pore sizes ranging from 10-30 nm. Some pores were connected and interpore space was 10-30 nm. FIG. 32(D) shows the photoluminescence of porous silicon. The luminescence from porous silicon is red under UV light (wavelength=254 nm).

The porosity is an important parameter of porous material, and can be determined by using weight measurements. The wafer was weighed before anodization (m₁), just after anodization (m₂), and after a rapid dissolution of the whole porous layer in a 3% KOH (m₃). Percent porosity is given by an equation (Zhang and Alocilja, 2008).

P(%)=(m₁−m₂)/(m₁−m₃) (1)

The porosity of the PS is influenced by the drying conditions of the chips. When the electrolyte evaporates from the pores, the capillary stress can crack the layer. The chips' porosity is 63.1% after fabrication. Nitrogen gas was used to dry PS chips and no cracking was observed under SEM.

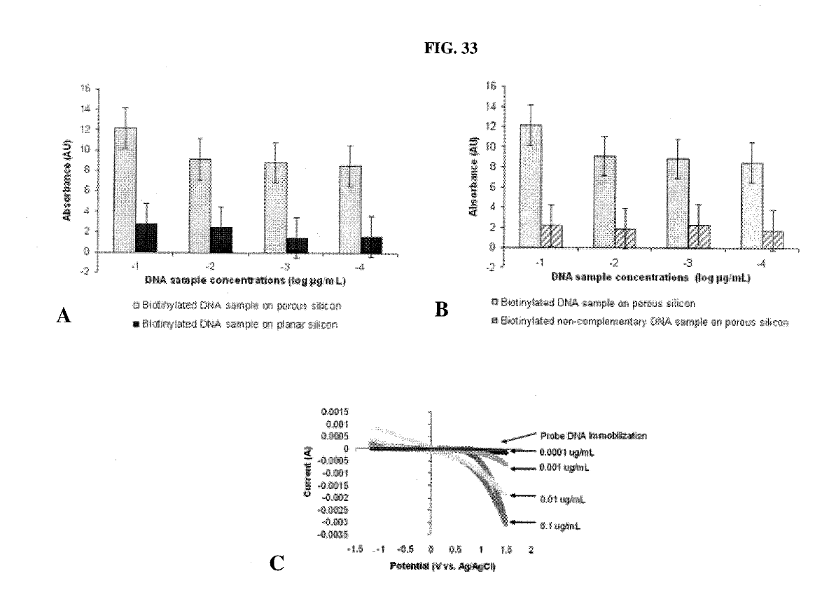

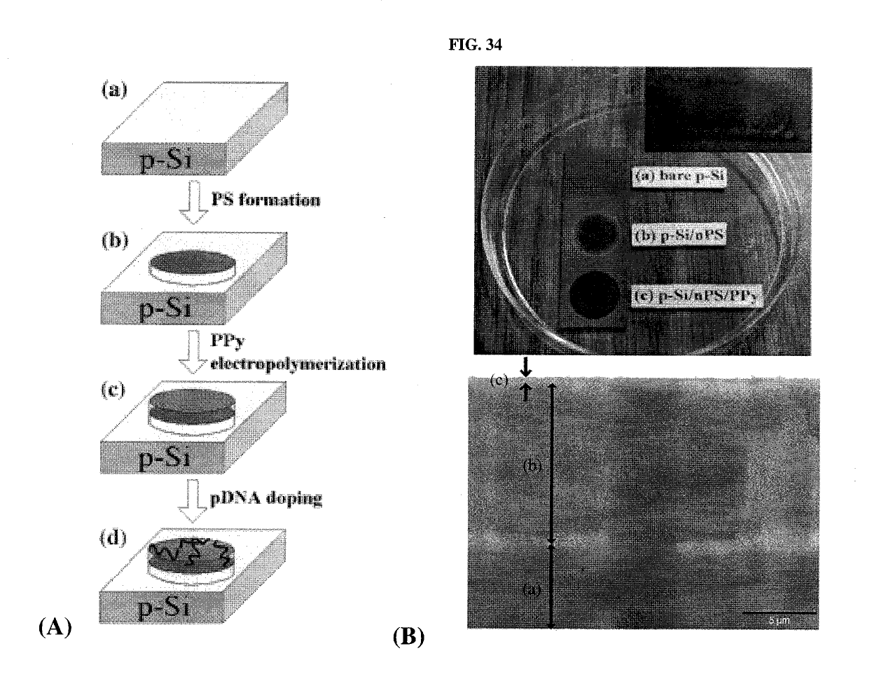

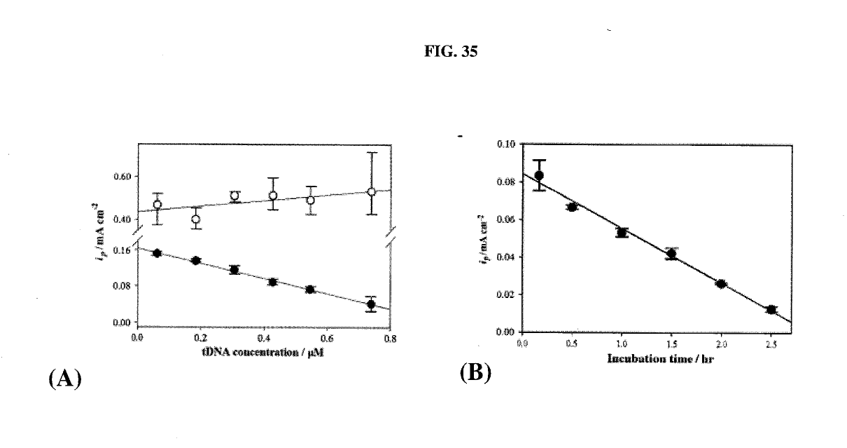

Verification of Nanopore Availability and DNA Probe Specificity: To verify the availability of nanopores on the biosensor surface, biotin-dATP was added to the target DNA from S. Enteritidis during PCR amplification in a thermal cycler. The amplified DNA sequence was conjugated with biotin, which can bind to streptavidin-horseradish peroxidase (HRP). The HRP could then catalyze the 2′-2′-azino-di-(3-ethylbenzthiazoline sulfonic acid) (ABTS) to a soluble blue product. Nonbi-otinylated DNA samples were synthesized from the PCR amplification with dATP and used as control. FIG. 32-33 shows the schematic of a biotin-streptavidin system used for the evaluation of DNA hybridization. UV-Vis-NIR Scanning Spectrophotometer (UV-3101PC, Shimadzu, Inc.) was used to determine the target DNA concentration. FIG. 32-33 shows the absorbance at 410 nm of biotinylated target DNA on porous silicon chips. The result shows that the biotinylated DNA samples were able to hybridize with the DNA probe immobilized on the porous silicon surface. This result further shows that the nanopore surface was available and successfully functionalized. The absorbance signals were directly proportional to the target DNA concentration. The nonbiotinylated DNA samples can hybridize with the immobilized DNA probe, but they have no biotin to bind the streptavidin-HRP and catalyze the ABTS. The absorbance signal from the nonbiotinylated DNA sample is about 0.5 AU and stable, which shows that it is from the physical absorption of streptavidin-HRP on the porous silicon surface. FIG. 32-33 shows that between the biotinylated target DNA on porous silicon and that of planar silicon, the absorbance signal from porous silicon is much higher than that of planar silicon when applied with the same concentration of DNA sample. The increased surface area in porous silicon is about four times that of planar silicon. However, although the absorbance signals of the biotinylated DNA samples on porous silicon for 10⁻², 10⁻³, and 10⁻⁴ microgram per ml of DNA are slightly decreasing in value, they are not statistically different. There could be stoichiometric reactions of the reactant sites in this range of DNA concentration.

FIG. 32-33 shows that the absorbance signals from biotinylated target DNA samples are higher than that from biotinylated noncomplementary DNA samples on porous silicon chips. This result shows that the immobilized DNA probe is specific to the DNA samples. Although noncomplementary DNA samples were conjugated with biotin, the DNA probe on the porous silicon chips could not bind with the target DNA because they are not complementary to the immobilized DNA probe. The DNA targets are washed away and there is no biotin available for the binding of streptavidin-HRP. Because of physical adsorption, the absorbance reading of biotinylated noncomplementary DNA sample is around 2 AU, but it does not change with sample concentration. Therefore, this signal is considered as part of the background noise.

Cyclic Voltammetry: FIG. 32-33 shows a cyclic voltammogram of various nonbiotinylated target DNA concentrations on porous silicon chips. When hybridization occurs, the Fe(CN) has a greater affinity for dsDNA and, therefore, a greater electrochemical response is observed compared with when only the single strand of probe DNA is bound to the electrode. When the voltage scans to the positive region, the Fe(CN) begins to be oxidized. The current peak at 1.5 V decreases as the target DNA concentration decreases. This demonstrates that a higher concentration allows redox reagents a greater charge transfer from the electrolyte to the modified electrode (PS chip). As a result, a higher current is observed.

Compared with biotinylated DNA samples on porous silicon to that on planar silicon, the current of the former is larger than the latter and shows that porous silicon has much more electrochemically active surface area for charge transfer than planar silicon. Noncomplementary DNA samples can result in some background but the peak current is still much lower than the complementary hybridization. Table I shows that the peak current of the CV at 1.5 V is mA and the charge density is 0.001378 mC/cm after immobilization of the DNA probe (DNA sample concentration). When the DNA sample concentration is 1 ng/mL, the peak current of the CV at 1.5 V is −0.37 mA and the charge density is 0.01461 mC/cm.

### Example IVX

The following example describes an exemplary multiplexed biosensor based on Label-Free DNA Sensor on Nanoporous Silicon-Polypyrrole Chip for Monitoring Salmonella Species in a biological sample (Jin, et al., IEEE SENSORS JOURNAL, 8(6):2008; herein incorporated by reference in its entirety). Salmonella species are classified as bioterrorism threat agents by the Centers for Disease Control and Prevention (CDC).

Specifically, Label-free DNA sensors based on nanoporous silicon (nPS) substrate were fabricated and electrochemically characterized. In brief, a low resistivity (0.01-0.02 Ω×cm) p-type silicon wafer (100 orientation) was electrochemically anodized in an ethanoic hydrofluoric acid (HF) solution containing ethanol to construct the nPS layer with pore diameter of about 10 nm. This nano structure was compatible with organic polymeric conductors.

Polypyrrole (PPy) film was directly electropolymerized on the nPS substrate without pre-deposition of any metallic thin-film underlayer. The rough surface of the nPS layer was favorable for strong adsorption of the PPy film. The intrinsic negative charge of the DNA backbone was exploited to adsorb 26 base pairs of probe DNA (pDNA) into the PPy film by applying positive bias forming the nPS/PPy+pDNA layer. DNA from Salmonella enterica serovar Enteritidis (tDNA) was extracted using standard protocol and, subsequently, amplified by polymerase chain reaction (PCR).

Results from the scanning electron microscopy (SEM) image of the cross section of the nPS/PPy multilayered film shows successful direct electropolymerization of PPy on the nPS substrate. Results also show that the tDNA concentration is inversely related to the peak current (iₚ) at 0.2 V versus Ag/AgCl. The plot of iₚ versus incubation time showed that current density (J) decreases by 29 μA×cm² per hour. The sensitivity obtained from the plot of iₚ versus tDNA concentration is −166.6 μA×cm⁻²×μM⁻¹. Current density decreases with increasing incubation time and tDNA concentration. These results demonstrate that the nPS substrate with PPy+pDNA+tDNA film has been successfully developed for a label-free DNA sensor in rapidly and specifically detecting select and threat agents.

A. Materials and Reagents.

A clinical strain of Salmonella Enteritidis (strain S-64) was grown on trypticase soy agar containing 0.6% yeast extract (TSAYE) and/or broth (TSBYE) at 37° C., as appropriate. In broth culture, cells were grown to exponential phase, and enumerated by spiral plating appropriately diluted cultures on Bismuth Sulfite Agar and Brilliant Green Agar. The cultures were serially diluted for DNA extraction so that the number of bacterial cells ranged from 10⁰ to 10⁸ colony forming units per milliliter (CFU/mL). Primers used for the polymerase chain reaction (PCR) were designed for the detection of Salmonella Enteritidis from the insertion element (Iel) gene. The single strand forward and reverse primers were Iel L-5′CTAACAGGCGCATACGATCTGACA 3′ (positions 542-565, 24 bases) and Iel R 5′TACGCATAGCGATCTCCTTCGTTG 3′ (positions 1047-1024, 24 bases). Probe DNA (pDNA) used was 5′ Amino link AATATGCTGCCTACTGCCCTACGCTT 3′ (positions 690-716 of target, 26 bases). PCR was performed with Taq DNA polymerase in a DNA thermal cycler, Promega Master Mix (Promega Corporation, Madison, Wis.), containing the proprietary reaction buffer (pH 8.5), 200 μM deoxynucleotide triphosphates (dNTP), 0.5 μM of the above primers set, 3 mM MgCl2, and 2.5 U of Taq DNA polymerase. A colony of Salmonella grown overnight in TSAYE was suspended in 0.5 mL of DNA grade water (Fisher Scientific, Pittsburg, Pa.) containing 5 mM of NaOH and boiled for 10 min to rupture the cells. One L was added to the PCR reaction mixture. After the final cycle, the target DNA (tDNA) sample was maintained at 72° C. for 10 min to complete the DNA strands synthesis and cooled to 4° C. A boron-doped, one-side-polished p-Si wafer (0.01-0.02 Ω×cm, 100 orientation, Montco Silicon Technologies, Inc., PA) was employed as a starting material. Pyrrole monomer and 70% solution of HclO4 (Aldrich Chemical Company, WI) were used for electropolymerization without further purification. Pt wire (99.99%) and a Ag/AgCl double junction probe with porous ceramic wick (Aldrich Chemical Company, WI) were used for counter and reference electrodes, respectively. Milli-Q pure water system was used for the preparation of deionized water (D.I. water). Acetonitrile (HPLC grade, EMD Science) was used as a nonaqueous media for the PPy electropolymerization. Highly purified KClO (99.99%) (Aldrich Chemical Company, WI) was used as a supporting electrolyte of aqueous media. Ethanol (>99.5%, Aldrich Chemical Company, WI) was used for etchant preparation and provisional storage of PS substrate before annealing. 10 M NaOH (Spectrum Quality Products, Inc., CA) was used for stabilizing pDNA. Glasswares was washed in either acidic or basic cleaning solution for at least 24 h before use. Reagents were used without further purification.

B. Chip Fabrication.

Silicon wafer was cut into 19×19 mm2 squares [FIG. 32A(a)] and the surface was cleaned with hydrofluoric acid (48% HF solution) and ethanol (100% purity). Since the working size in the Teflon electrochemical cell has ca. 1.2 cm diameter, the actual area of the silicon chip exposed to the etching solution is about 1.5 cm. Ethanolic HF solution, a 14.4% HF solution (30 mL of HF (48% purity) +70 mL of CH3CH2OH (100% purity), was used as an etchant using −5 mA×cm-2 of constant current for 30 min (2400 SourceMeter, Keithley). Once the PS layer was formed, as shown in FIG. 32A(b), the chip was stored in pure ethanol before annealing to prevent the surface from being oxidized in atmospheric condition. After annealing the PS chip at 110° C. for 1 h (multiblock heater; Lab-Line Instruments, Inc., IL, USA), the PPy film was anodically electropolymerized on the PS substrate, as shown in FIG. 32A(c), by scanning a linear potential with a scan rate of 25 mV s-1 (PrincetonApplied Research, Model 263A). The electrolyte used for the PPy electropolymerization was composed of an acetonitrile solution containing 0.02 M pyrrole and 0.1 M HclO4. Pt tip and wire were used for quasi-reference electrode (QRE) and counter electrode, respectively. Nitrogen gas was bubbled into the 5 mL electrolyte solution for 10 min to remove dissolved oxygen and purged during the potential scanning. Ten nanomolar pDNA was immobilized on the PPy matrix by applying +0.6 V versus Pt (QRE) to the PPy-coated PS substrate (p-Si/PS/PPy) for 20 min [FIG. 32A(d)]. Even though 10 nM pDNA was used as a solution for doping pDNA into the polypyrrole matrix, the actual concentration of the pDNA in the polypyrrole film as not determined.

However, it appeared that the amount of pDNA from sensor to sensor was identical because the same constant potential was applied over the same time. Double-stranded tDNA from the target organism prepared earlier was mixed with the hybridization buffer (QuikHyb, Stratagene) and preheated to 95° C. for 20 min in a water bath, and again cooled to 59° C. (melting temperature of pDNA). Hybridization of complementary DNA strands extracted from Salmonella Enteritidis with the pDNA layer was performed in a water bath (Isotemp 288, Fisher Scientific) by dropping a mixture of tDNA and hybridization buffer on the pDNA-PS substrate. After hybridization, the surface was thoroughly rinsed with a washing solution composed of 0.1× saline-sodium citrate (SSC) buffer and 0.1% (w/v) of sodium dodecyl sulfate (SDS) to remove nonspecifically adsorbed DNA strands. After rinsing again with a 0.1×SSC buffer, the tDNA-hybridized PS chip was dried under nitrogen and prepared for measurement. Peak current outputs obtained from cyclic voltammograms of each PS chip in a solution containing 0.01 M potassium perchlorate were recorded for calibration of the chip.

Results: Surface Morphology of Label-Free DNA Sensor on nPS-PPy Chip FIG. 32B(a) showed a bare p-Si chip. A low-resistivity silicon wafer (0.01-0.02 Ω×cm) was selected as a starting material to achieve better electrical current flow. FIG. 32B(b) is the nPS formed by electrochemical anodization on the low-resistivity p-Si chip (p-Si/nPS) in a 14.4% ethanolic HF solution. Current density (J) applied was −5 mA×cm-2 for 32 min. For comparison, two other types of PS layers were also formed under the same experimental conditions with different resistivities such as 14-17 Ω×cm and 0.1-0.9 Ω×cm. While the former showed a macro PS layer, the latter was overpolished. The major role of the PS layer herein is to increase the surface roughness for direct PPy electropolymerization. The nPS formed by using the p-Si chip with 0.01-0.02 Ω×cm gave better results for electropolymerized PPy film on the PS substrate than the other two types. As can be seen from the inset of FIG. 32, formation of the nPS layer was confirmed by observing visible light emission from the nPS surface when the PS surface was excited with a UV light (254 nm). Porous silicon (PS) is very sensitive to atmospheric conditions and is readily oxidized to silicon dioxide. This oxide layer thickness would grow over time, and hence would affect the emission property of the PS surface with time and, consequently, the electrochemical property of the PPy film on the PS surface would be altered. To minimize uncontrolled oxide formation, the nPS substrate was annealed. The annealed nPS substrates were stored in a vacuum desiccator before use. FIG. 32B(c) is the PPy-coated p-Si PS chip (p-Si/nPS/PPy). The positively charged black PPy film is an excellent doping matrix for the negatively charged pDNA. Hybridization reaction with the tDNA also occurs on the pDNA-doped p-Si/nPS/PPy substrate (p-Si/nPS/PPy/pDNA). FIG. 32B shows a cross-sectional SEM image (JSM-6300F Scanning Microscope, JEOL) of the p-Si/nPS/PPy substrate shown in FIG. 32B(c), where 10 nm gold layer was predeposited (SC500, Emscope sputter) for imaging. Layer (a) is low-resistivity p-Si substrate. Layer (b) is the PS layer in which columnar micro pores are distributed homogeneously. Pore depth and diameter are 12 m and ca. 10 nm, respectively. The high roughness of PS substrate is compatible for direct electropolymerization of PPy on the PS substrate. Layer (c) is the PPy film directly electropolymerized on the rough PS substrate with thickness of ca. 200 nm. High surface energy of Si substrate enhanced by forming PS structure makes quality control or sensitivity measurement of PS-based DNA sensors to be possible in an aqueous media.

2. Electrochemical Calibration of Label-Free DNA Sensor on nPS-PPy Chip

There are various reports about DNA conductivity, from a superconductor to an insulator, and is still being discussed [Endres, et al., Rev. Mod. Phys., vol. 76, pp. 195-214, 2004].

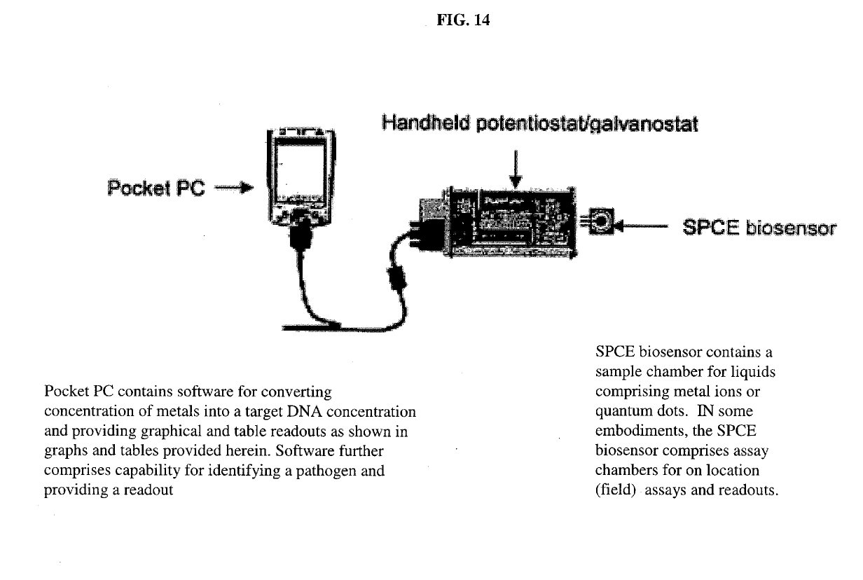

Some studies indicated that DNA is electrically insulator when the length of DNA strand is longer than a few nanometers [Ye, et al., Anal. Chim. Acta, vol. 568, pp. 138-145, 2006]. Considering that the length of a 10-base-pair DNA is about 3.4 nm, a 26-base-pair DNA is ca. 8.84 nm long. PPy film is a semiconductor and the level of a dopant will strongly affect the resistivity of PPy film, i.e., insulating DNA strands doped to PPy can potentially increase the depleted region of PPy and, thus, conductivity would decrease. FIG. 33 shows the iₚ change with various tDNA concentrations after 1 h of incubation time for tDNA hybridization. The observed at ca. 0.2 V versus Ag/AgCl in a CV diagram was selected for the calibration. The iₚ decreased with increasing tDNA concentration due to the decreasing conductivity of the PPy matrix. Sensitivity obtained from the plot of iₚ versus tDNA concentration was—166.6 μA×cm⁻²×μM⁻¹ within the experimental range of tDNA concentration. Bare PPy film without pDNA dopant was also used for recording background signals. Since the conductive PPy film is a semiconductor, its conductivity depends on the doping level. Single and double stranded DNA can act as a dopant. However, they are electrical insulators compared with normal anions or cations composing an electrolyte. When a tDNA hybridizes to its complementary pDNA, some anions that have been doped into the PPy matrix will be repelled out of the PPy film for neutralization. If there is no hybridization process at all, conductivity of the PPy film will not change. However, some ions dissolved in a hybridization buffer solution can still move in the PPy film at 59° C. electrolyte phase. The lowest detection limit was 0.167 μM for this experimental tDNA range. FIG. 14B shows the dependence of on the incubation time for tDNA hybridization. The iₚ at 0.2 V versus Ag/AgCl was plotted as a function of incubation time and the slope was −29 μA×cm⁻²×hour⁻¹.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A composition, comprising a silent DNA sequence attached to a nanoparticle tracer.

2. The composition of claim 1, wherein said silent DNA sequence is selected from the group consisting of SEQ ID NOs:1-2, 6, 8, and 9.

3. The composition of claim 1, further comprising a detection nanoparticle, wherein said detection nanoparticle is selected from the group consisting of gold, polystyrene, and silicon.

4. The composition of claim 3, wherein said detection nanoparticle further comprises a first probe DNA molecule capable of hybridizing to a 5′ area of a single strand of a target DNA.

5. The composition of claim 4, wherein said first probe DNA molecule is selected from the groups consisting of SEQ ID NO:14 and SEQ ID NO:17.

6. The composition of claim 4, wherein said first probe DNA molecule is attached to said detection nanoparticle.

7. The composition of claim 3, wherein said detection nanoparticle ranges from 5 nm to 25 nm in diameter.

8. The composition of claim 1, wherein said composition is soluble.

9. The composition of claim 1, wherein said composition is in solution.

10. The composition of claim 1, wherein said nanoparticle tracer is selected from the group consisting of a fluorescence molecule and a metal particle.

11. The composition of claim 10, wherein said metal particle is capable of conducting electricity.

12. The composition of claim 10, wherein said metal particle is capable of releasing metal cations.

13. The composition of claim 10, wherein said metal is selected from the group consisting of a base metal, a precious metal, a metal composite, a metal ion, a metal salt, and an alloy.

14. The composition of claim 13, wherein said base metal is selected from the group consisting of Lead, Cadmium, Zinc, Copper, sulfates therof, chlorides therof, salts therof, ions thereof, and iostopes therof.

15. The composition of claim 13, wherein said precious metal is selected from the group consisting of gold, silver, sulfates therof, chlorides therof, salts therof, ions thereof, and iostopes therof.

16. The composition of claim 10, wherein said fluorescence molecule is selected from the group consisting of carboxyfluoresceins.

17. The composition of claim 1, wherein said nanoparticle tracer is a quantum dot.

18. A composition, comprising a magnetic nanoparticle attached to a probe DNA, wherein said probe DNA is named a second probe DNA.

19. The composition of claim 18, wherein said second probe DNA is selected from the group consisting of SEQ ID NO:15 and SEQ ID NO:18.

20. The composition of claim 18, wherein said magnetic nanoparticle comprises iron.

21. The composition of claim 18, wherein said magnetic nanoparticle ranges from 75 nm to 125 nm in diameter.

22. The composition of claim 18, wherein said composition is soluble.

23. The composition of claim 18, wherein said composition is in solution.

24. A silent DNA sequence selected from the group consisting of SEQ ID NO:1-11 and 21.

25. A probe DNA sequence selected from the group consisting of SEQ ID NOs:14-16.

26. A complex comprising a magnetic nanoparticle, a target DNA molecule, and a detection nanoparticle, wherein said detection nanoparticle further comprises a silent DNA sequence attached to nanoparticle tracer, wherein said complex is soluble.

27. The complex of claim 26, wherein said complex is in solution.

28. The complex of claim 26, wherein said nanoparticle tracer is selected from the group consisting of a tracer fluorescence molecule and a tracer metal particle.

29. The complex of claim 28, wherein said tracer metal particle is selected from the group consisting of Lead, Cadmium, Zinc, Copper, isotopes, salts, and derivatives thereof.

30. The complex of claim 28, wherein said fluorescence molecule is selected from the group consisting of carboxyfluoresceins.

31. The complex of claim 26, wherein said nanoparticle tracer is a quantum dot.

32. The complex of claim 26, wherein said silent DNA sequence is selected from the group consisting of SEQ ID NOs:1-2, 6, 8, and 9.

33. The complex of claim 26, wherein said detection nanoparticle is selected from the group consisting of gold, silicon and polystyrene.

34. The complex of claim 26, wherein said detection nanoparticle comprises a first probe DNA sequence, wherein said first sequence is capable of hybridizing to a target DNA sequence.

35. The complex of claim 34, wherein said first probe DNA sequence is selected from the group consisting of SEQ ID NO:14 and SEQ ID NO:17.

36. The complex of claim 34, wherein said magnetic nanoparticle comprises a second probe DNA sequence, wherein said second probe DNA sequence is capable of hybridizing to a target DNA sequence simultaneously with said first probe DNA sequence.

37. The complex of claim 36, wherein said second probe DNA sequence is selected from the group consisting of SEQ ID NO:15 and SEQ ID NO:18.

38. The complex of claim 36, wherein said magnetic nanoparticle is attached to the detection nanoparticle by the hybridization of the target DNA to the first DNA sequence of the detection nanoparticle and the hybridization of the target DNA to the second DNA sequence of the magnetic nanoparticle.

39. The complex of claim 26, wherein said target DNA molecule is derived from a pathogen.

40. The complex of claim 39, wherein said derived from is selected from the group consisting of DNA isolated from a pathogen, DNA synthetically duplicated from pathogen DNA, and DNA representative of DNA from a pathogen.

41. The complex of claim 39, wherein said pathogen is selected from the group consisting of a bacterium, a virus, and a fungi.

42. A method for detecting a target DNA, comprising,
a) providing:
i) a sample comprising a target DNA, wherein said sample is treated under conditions for providing single stranded target DNA,
ii) a detection nanoparticle, wherein said nanoparticle comprises a silent DNA sequence attached to a nanoparticle tracer and a first probe DNA sequence complimentary to a portion of the target DNA, and
iii) a magnetic nanoparticle, wherein said magnetic nanoparticle comprises a second probe DNA sequence which is different from the first probe DNA sequence, complimentary to a portion of the target DNA,
iv) a solution,
b) mixing at least a portion of the treated sample, the detection nanoparticle, and the magnetic nanoparticle under DNA-DNA hybridization conditions such that said single stranded molecule of said target DNA hybridizes to both the first probe DNA sequence of said detection nanoparticle and the second probe DNA sequence of said magnetic nanoparticle forming a complex in said solution.

43. The method of claim 42, further comprising step c) isolating said complex from said solution using a magnetic field.

44. The method of claim 42, further providing a wash solution, and step d) releasing the nanoparticle tracer into solution from isolated complexes.

45. The method of claim 42, further providing a potentiostat and step e) measuring the concentration of nanoparticle tracer in said nanoparticle solution.

46. The method of claim 42, wherein said silent DNA sequence is selected from the group consisting of SEQ ID NOs:1-2, 6, 8, and 9.

47. The method of claim 42, wherein said first probe DNA sequence is selected from the group consisting of SEQ ID NO:14 and SEQ ID NO:17.

48. The method of claim 42, wherein said second probe DNA is selected from the group consisting of SEQ ID NO:15 and SEQ ID NO:18.

49. The method of claim 42, wherein said nanoparticle tracer is selected from the group consisting of a tracer fluorescence molecule and a tracer metal particle.

50. The method of claim 45, wherein said tracer fluorescence molecule is selected from the group consisting of carboxyfluoresceins.

51. The method of claim 45, wherein said tracer metal particle is selected from the group consisting of Lead, Cadmium, Zinc, Copper, ions therof, salts thereof, and iostopes thereof.

52. The method of claim 42, wherein said nanoparticle tracer is a quantum dot.

53. The method of claim 47, further comprising, providing, a handheld potentiostat comprising a disposable screen-printed carbon electrode (SPCE) for electrochemically measuring the concentration of tracer metal particles in said wash solution.

54. The method of claim 49, wherein handheld potentiostat comprises software capable of converting the measured concentration of metal particles into a target DNA concentration.

55. The method of claim 49, wherein handheld potentiostat comprises software capable of identifying a pathogen.

56. The method of claim 49, wherein said handheld potentiostat is a device attached to computer selected from the group consisting of a pocket, laptop, netbook, and desktop computer.

57. The method of claim 42, wherein the sample is turbid.


SEQUENCE LISTING (30 sequences)

SEQ ID NO: 1 - DNA, 20 bp, Artificial Sequence
  Synthetic
  misc_feature (1)..(1): The residue in this position is sometimes a variation of fluorescent molecule (examples, carboxyfluorescein, 6-FAM).
  misc_feature (20)..(20): The residue in this position is sometimes a thiol/sulfhydryl (SH) group.
ttattcgtagctaaaaaaaa

SEQ ID NO: 2 - DNA, 22 bp, Artificial Sequence
  Synthetic
  misc_feature (1)..(1): The residue in this position is is sometimes a biotin molecule or thiol/sulfhydryl (SH) group.
  misc_feature (22)..(22): The residue in this position is sometimes a thiol/sulfhydryl (SH) group.
ttattcgtagctaaaaaaaaaa

SEQ ID NO: 3 - DNA, 21 bp, Artificial Sequence
  Synthetic
  misc_feature (1)..(1): The residue in this position has an attached biotin.
  misc_feature (21)..(21): The residue in this position has an attached thiol/sulfhydryl (SH) group.
ttattcgtagctaaaaaaaaa

SEQ ID NO: 4 - DNA, 22 bp, Artificial Sequence
  Synthetic
  misc_feature (1)..(1): The residue in this position sometimes has an attached biotin and/or a variation of fluorescent molecule (examples, carboxyfluorescein, 6-FAM).
  misc_feature (22)..(22): The residue in this position sometimes has an attached thiol/sulfhydryl (SH) group group.
ttattcgtagctaaaaaaaaaa

SEQ ID NO: 5 - DNA, 22 bp, Artificial Sequence
  Synthetic
  misc_feature (1)..(1): The residue in this position is sometimes a variation biotin.
  misc_feature (22)..(2): The residue in this position is sometimes a variation of fluorescent molecule (examples, carboxyfluorescein, 6-FAM).
ttattcgtagctaaaaaaaaaa

SEQ ID NO: 6 - DNA, 12 bp, Artificial Sequence
  Synthetic
  misc_feature (1)..(1): The residue in this position is sometimes a fluorescent molecule (examples, carboxyfluorescein, 6-FAM) or a biotin molecule.
  misc_feature (12)..(12): The residue in this position sometimes has an attached thiol/sulfhydryl (SH) group.
agctacgaataa

SEQ ID NO: 7 - DNA, 22 bp, Artificial Sequence
  Synthetic
  misc_feature (1)..(1): The residue in this position is thiol/sulfhydryl (SH) group.
aaaaaaaaaattattcgtagct

SEQ ID NO: 8 - DNA, 39 bp, Artificial Sequence
  Synthetic
  misc_feature (1)..(1): The residue in this position is sometimes a fluorescent molecule (examples, carboxyfluorescein, 6-FAM) or a biotin molecule.
  misc_feature (39)..(39): The residue in this position sometimes has an attached thiol/sulfhydryl (SH) group.
cgtcgcattcaggattctcaactcgtagctaaaaaaaaa

SEQ ID NO: 9 - DNA, 25 bp, Artificial Sequence
  Synthetic
  misc_feature (1)..(1): The residue in this position is sometimes a fluorescent molecule (examples, carboxyfluorescein, 6-FAM) or a biotin molecule.
  misc_feature (25)..(25): The residue in this position sometimes has an attached thiol/sulfhydryl (SH) group.
tctcaactcgtagctaaaaaaaaaa

SEQ ID NO: 10 - DNA, 22 bp, Artificial Sequence
  Synthetic
  misc_feature (1)..(1): The residue in this position is sometimes a fluorescent molecule (examples, carboxyfluorescein, 6-FAM) or a biotin molecule.
  misc_feature (22)..(22): The residue at this position sometimes has an attached thiol/sulfhydryl (SH) group.
ttattcgtagctaaaaaaaaaa

SEQ ID NO: 11 - DNA, 22 bp, Artificial Sequence
  Synthetic
  misc_feature (1)..(1): The residue at this position is sometimes a thiol/sulfhydryl (SH) group.
  misc_feature (22)..(22): The residue at this position is sometimes a fluorescent molecule (examples, carboxyfluorescein, 6-FAM) or a biotin molecule.
ttattcgtagctaaaaaaaaaa

SEQ ID NO: 12 - DNA, 40 bp, Artificial Sequence
  Synthetic Bacillus anthracis
agaggaatgtataaggatgttccgggcgtgtgggtaagtc

SEQ ID NO: 13 - DNA, 50 bp, Artificial Sequence
  Synthetic Bacillus anthracis
  misc_feature (1)..(1): The residue at this position is sometimes PO(4)
ggaagagtgagggtggatacaggctcgaactggagtgaagtgttaccgca

SEQ ID NO: 14 - DNA, 26 bp, Artificial Sequence
  Synthetic Salmonella enteritidis
  misc_feature (26)..(26): The residue at this position sometimes has an attached thiol/sulfhydryl (SH) group.
aatatgctgcctactgccctacgctt

SEQ ID NO: 15 - DNA, 21 bp, Artificial Sequence
  Synthetic Salmonella enteritidis
  misc_feature (1)..(1): The residue at this position sometimes has an attached thiol/sulfhydryl (SH) group
ttattcgtagctaaaaaaaaa

SEQ ID NO: 16 - DNA, 26 bp, Artificial Sequence
  Synthetic Salmonella enteritidis
  misc_feature (1)..(1): The residue at this position sometimes has an attached thiol/sulfhydryl (SH) group.
tttatgtagtcctgtatcttcgccgt

SEQ ID NO: 17 - DNA, 25 bp, Artificial Sequence
  Synthetic Salmonella enteritidis
  misc_feature (1)..(1): The residue at this position sometimes has an attached thiol/sulfhydryl (SH) group.
ttatgtagtcctgtatcttcgccgt

SEQ ID NO: 18 - DNA, 25 bp, Artificial Sequence
  Synthetic Bacillus anthracis
  misc_feature (1)..(1): The residue at this position sometimes has an attached thiol/sulfhydryl (SH) group.
ggaagagtgagggtggatacaggct

SEQ ID NO: 19 - DNA, 25 bp, Artificial Sequence
  Synthetic Bacillus anthracis
  misc_feature (1)..(1): The residue at this position sometimes has an attached thiol/sulfhydryl (SH)group
agatttaaatctggtagaaaggcgg

SEQ ID NO: 20 - DNA, 24 bp, Artificial Sequence
  Synthetic Salmonella enteritidis
ctaacaggcgcatacgatctgaca

SEQ ID NO: 21 - DNA, 24 bp, Artificial Sequence
  Synthetic Salmonella enteritidis
tacgcatagcgatctccttcgttg

SEQ ID NO: 22 - DNA, 21 bp, Artificial Sequence
  Synthetic
  misc_feature (1)..(1): The residue at this position is sometimes a fluorescent molecule (examples, carboxyfluorescein, 6-FAM).
  misc_feature (21)..(21): The residue at this position is sometimes a ThioMC3-D modifier.
ttattcgtagctaaaaaaaaa

SEQ ID NO: 23 - DNA, 26 bp, Artificial Sequence
  Synthetic Bacillus anthracis
cctgtactttgctacttgctgcacaa

SEQ ID NO: 24 - DNA, 23 bp, Artificial Sequence
  Synthetic Bacillus anthracis
ttgaaaatggaagagtgagggtg

SEQ ID NO: 25 - DNA, 30 bp, Artificial Sequence
  Synthetic Bacillus anthracis
taaatctggtagaaaggcggatagcggcgg

SEQ ID NO: 26 - DNA, 50 bp, Artificial Sequence; identical to SEQ ID NO: 13
  Synthetic Bacillus anthracis

SEQ ID NO: 27 - DNA, 120 bp, Artificial Sequence
  Synthetic Bacillus anthracis
tgaaaatggaagagtgagggtggatacaggctcgaactggagtgaagtgttaccgcaaattcaagaaacaactgcacgtatcatttttaatggaaaagatttaaatctggtagaaaggcg

SEQ ID NO: 28 - DNA, 1115 bp, Artificial Sequence
  Synthetic Bacillus anthracis
tatccttctaaaaacttggcgccaatcgcattaaatgcacaagacgatttcagttctactccaattacaatgaattacaatcaatttcttgagttagaaaaaacgaaacaattaagattagatacggatcaagtatatgggaatatagcaacatacaattttgaaaatggaagagtgagggtggatacaggctcgaactggagtgaagtgttaccgcaaattcaagaaacaactgcacgtatcatttttaatggaaaagatttaaatctggtagaaaggcggatagcggcggttaatcctagtgatccattagaaacgactaaaccggatatgacattaaaagaagcccttaaaatagcatttggatttaacgaaccgaatggaaacttacaatatcaagggaaagacataaccgaatttgattttaatttcgatcaacaaacatctcaaaatatcaagaatcagttagcggaattaaacgcaactaacatatatactgtattagataaaatcaaattaaatgcaaaaatgaatattttaataagagataaacgttttcattatgatagaaataacatagcagttggggcggatgagtcagtagttaaggaggctcatagagaagtaattaattcgtcaacagagggattattgttaaatattgataaggatataagaaaaatattatcaggttatattgtagaaattgaagatactgaagggcttaaagaagttataaatgacagatatgatatgttgaatatttctagtttacggcaagatggaaaaacatttatagattttaaaaaatataatgataaattaccgttatatataagtaatcccaattataaggtaaatgtatatgctgttactaaagaaaacactattattaatcctagtgagaatggggatactagtaccaacgggatcaagaaaattttaatcttttctaaaaaaggctatgagataggataaggtaattctaggtgatttttaaattatctaaaaaacagtaaaattaaaacatactctttttgtaagaaatacaaggagagtatgttttaaacagtaatctaaatcatcataatcctttgagattgtttgtaggatcc

SEQ ID NO: 29 - DNA, 20 bp, Artificial Sequence
  Synthetic Bacillus anthracis
aaaatggaagagtgagggtg

SEQ ID NO: 30 - DNA, 23 bp, Artificial Sequence
  Synthetic Bacillus anthracis
ccgcctttcttctaccagattta